(19) United States
(12) Patent Application Publication
(10) Pub. No.: US 2021/0285050 A1
(43) Pub. Date: Sep. 16, 2021

(54) PDE3B VARIANTS

(71) Applicant: UNITED STATES GOVERNMENT AS REPRESENTED BY THE DEPARTMENT OF VETERANS AFFAIRS, Washington, DC (US)

(72) Inventors: Derek Klarin, Boston, MA (US); Scott M. Damrauer, Philadelphia, PA (US); Christopher J. O'Donnell, Boston, MA (US); Philip S. Tsao, Palo Alto, CA (US); Sekar Kathiresan, Boston, MA (US); Daniel J. Rader, Philadelphia, PA (US); Peter W.F. Wilson, Decatur, GA (US); Themistocles L. Assimes, Palo Alto, CA (US)

(21) Appl. No.: 17/258,084

(22) PCT Filed: Jul. 3, 2019
(86) PCT No.: PCT/US19/40472

§ 371 Date: Jan. 5, 2021

Related U.S. Application Data
(60) Provisional application No. 62/694,949, filed on Jul. 6, 2018.

(51) Int. Cl. C12Q 1/6883; A61K 31/4709; C12N 9/22; C12N 15/11; A61K 31/7088; A61K 38/46; C12N 15/113

(52) U.S. Cl. CPC C12Q 1/6883; A61K 31/4709; C12N 9/22; C12N 15/11; A61K 31/7088; C12N 2310/14; C12N 15/1137; C12Q 2600/156; C12N 2800/80; C12N 2310/20; A61K 38/465

(57) ABSTRACT

Disclosed are chimeric antigen receptor (CAR) polypeptides comprising a T cell receptor (TCR) antigen binding domain, a transmembrane domain, and an intracellular signaling domain. Disclosed are methods of making a CART cell comprising obtaining a cell from a subject diagnosed with T cell lymphoma; determining the sequence of the TCR on the cell; and transducing a T cell with a vector comprising a nucleic acid sequence that encodes a CAR polypeptide, wherein the CAR polypeptide comprises a TCR antigen binding domain, a transmembrane domain, and an intracellular signaling domain, wherein the TCR antigen binding domain is specific to a subsequence of the sequence of the TCR on the cell identified in the step of determining the sequence of the TCR

### STATEMENT REGARDING FEDERALLY SPONSORED RESEARCH

This invention was made with government support under #G002, I01-01BX03340, I01-BX002641, and I01-CX001025 awarded by Veterans Administration (VA) Cooperative Studies Program (CSP), T32 HL007734 and R01HL127564 awarded by the National Institute of Health. The government has certain rights in the invention.

### BACKGROUND

Large-scale biobanks offer the potential to link genes to health traits documented in electronic health records (EHR) with unprecedented power. In turn, these discoveries are expected to improve our understanding of the etiology of common and complex diseases as well as our ability to treat and prevent these conditions. To this end, the Million Veteran Program (MVP) was established by the U.S. Veterans Health Administration in 2011 as a nationwide research program within the Veteran Administration (VA) healthcare system. The overarching goal of MVP is to reveal new biologic insights and clinical associations broadly relevant to human health and enhance the care of veterans through precision medicine.

Blood concentrations of total cholesterol (TC), low-density lipoprotein (LDL) cholesterol, high-density lipoprotein (HDL) cholesterol, and triglycerides (TG) are heritable risk factors for cardiovascular disease, a highly prevalent condition among U.S. veterans. Genome-wide association studies (GWAS) to date have identified at least 268 loci that influence these levels, many of which are under investigation as potential therapeutic targets. However, off-target effects have dampened enthusiasm for some of these molecules, and understanding the full spectrum of clinical consequences of a given DNA sequence variant through phenome-wide association scanning (“PheWAS”) may shed light on potential unintended effects as well as novel therapeutic indications.

### BRIEF SUMMARY

A GWAS was perfromed, including a discovery phase in MVP and a replication phase in the Global Lipids Genetics Consortium (GLGC) (FIG. 1). In the discovery phase, an association testing was performed among 297,626 white, black, and Hispanic MVP participants with lipids stratified by ethnicity followed by a meta-analysis of results across all three groups. Replication of MVP findings was conducted in one of two independent studies from the GLGC. Genome-wide lipid-associated, low-frequency missense variants were examined unique to black and Hispanic individuals. Additionally, a PheWAS was performed for a set of DNA sequence variants within genes that have already emerged as therapeutic targets for lipid modulation, leveraging the full catalog of ICD-9 diagnosis codes in the VA EHR to better understand the potential consequences of pharmacologic modulation of these genes or their products.

Disclosed are methods for determining a subject's susceptibility to having or developing coronary artery disease comprising determining in the subject the presence of one or more PDE3B loss of function or damaging variants, and wherein the presence of the variant indicates the subject's decreased susceptibility for having or developing coronary artery disease.

In some aspects, the PDE3B loss of function or damaging variant is Arg783Ter or rs150090666. In some aspects, the PDE3B loss of function or damaging variant results in a truncated PDE3B protein. In some aspects, the truncated PDE3B protein has the mutation Arg783Ter.

In some aspects, the PDE3B loss of function or damaging variant is determined from a sample obtained from the subject.

In some aspects, the PDE3B loss of function or damaging variant is determined by amplifying or sequencing a nucleic acid sample obtained from the subject. In some aspects, the amplifying is performed using polymerase chain reaction (PCR).

The method of claims 6-7, wherein the amplifying or sequencing comprises using primers having sequences complementary to PDE3B DNA or RNA sequences. For example, disclosed are primers and probes having sequences complementary to a portion of the PDE3B nucleic acid sequence found in accession number NM_000922.3.

Disclosed are methods of detecting one or more PDE3B loss of function or damaging variants in a subject, said method comprising: obtaining a biological sample from a subject; detecting whether a PDE3B loss of function variant is present in the biological sample by performing whole genome or whole exome sequencing.

Disclosed are methods comprising: obtaining a biological sample from a subject; detecting whether one or more PDE3B loss of function variants are present in the sample; diagnosing the subject as having a greater likelihood of responding to PDE3B inhibitors when there is an absence of the one or more PDE3B loss of function variants; and administering an effective amount of a PDE3B inhibitor/antagonist to the subject.

Disclosed are methods of treating a patient with coronary artery disease comprising administering an effective amount of a PDE3B inhibitor/antagonist.

Disclosed are methods of treating/preventing coronary artery disease in a subject comprising administering a composition that antagonizes/inhibits PDE3B to the subject, wherein the subject has been determined to lack one or more loss of function mutations in PDE3B.

Disclosed are methods of screening for test compositions that cause a loss of function mutation in PDE3B comprising: contacting a PDE3B gene with a test composition; detecting the presence of one or more mutations in the PDE3B gene; and determining if the one or more mutations are loss of function mutations, wherein the presence of one or more loss of function mutations in PDE3B indicates a test composition that causes a loss of function in PDE3B.

Disclosed are methods of screening for therapeutic candidates for treating coronary artery disease compositions comprising: contacting a cell lacking one or more loss of function or damaging mutations in PDE3B with a test composition; and determining if the test composition inhibits PDE3B in the cell, wherein if the test composition inhibits PDE3B then it is a therapeutic candidate for treating coronary artery disease.

Disclosed are vectors comprising a loss of function or damaging PDE3B variant, wherein the PDE3B variant comprises a mutation that results in a truncated PDE3B protein.

Disclosed are cells comprising any of the disclosed vectors.

Disclosed are methods for identifying a subject in need of treatment for coronary artery disease comprising determining in the subject the presence of a PDE3B loss of function or damaging variant, wherein the presence of a PDE3B loss of function or damaging variant indicates that the subject is not in need of treatment for a coronary artery disease.

Disclosed are methods of identifying a subject in need of screening for the development of a coronary artery disease comprising determining in the subject the absence of a PDE3B loss of function or damaging variant, wherein the absence of a a PDE3B loss of function or damaging variant indicates a subject in need of screening for the development of coronary artery disease.

Disclosed are engineered, non-naturally occurring CRISPR-CAS systems comprising: a guide RNA that hybridizes with a target sequence, wherein the target sequence comprises a PDE3B loss of function variant, and a Cas protein or gene encoding a Cas protein.

Disclosed are methods of altering expression of at least one gene product, wherein the at least one gene product is a gene product from a PDE3B loss of function variant, wherein the method comprises administering a) a guide RNA that hybridizes with a target sequence, wherein the target sequence comprises the PDE3B loss of function variant, and b) a Cas protein or gene encoding a Cas protein, whereby the guide RNA targets the target sequence and the Cas9 protein cleaves the nucleic acid molecule which comprises the PDE3B loss of function variant, whereby expression of the at least one gene product is altered.

Disclosed are methods of altering expression of at least one gene product, wherein the at least one gene product is a gene product from a PDE3B loss of function variant, wherein the method comprises administering a vector that comprises a) a first regulatory element operable in a eukaryotic cell operably linked to at least one nucleotide sequence encoding a CRISPR-Cas system guide RNA that hybridizes with a target sequence, wherein the target sequence comprises the PDE3B loss of function variant, and b) a second regulatory element operable in a eukaryotic cell operably linked to a nucleotide sequence encoding a Cas9 protein, whereby the guide RNA targets the target sequence and the Cas9 protein cleaves the target sequence, whereby expression of the at least one gene product is altered.

Disclosed are methods of silencing or inhibiting expression of wild type PDE3B in a cell comprising providing at least one silencing agent to the cell, wherein said silencing agent silences or inhibits expression of the wild type PDE3B in the cell.

Disclosed are methods of silencing or inhibiting expression of wild type PDE3B in a cell comprising providing at least one RNA to the cell in an amount sufficient to inhibit the expression of PDE3B, wherein the RNA comprises or forms a double-stranded structure containing a first strand comprising a ribonucleotide sequence which corresponds to a nucleotide sequence of PDE3B and a second strand comprising a ribonucleotide sequence which is complementary to the nucleotide sequence of PDE3B, wherein the first and the second ribonucleotide sequences are separate complementary sequences that hybridize to each other to form said double-stranded structure, and the RNA comprising the double-stranded structure inhibits expression of PDE3B.

Disclosed are RNAs comprising a double-stranded structure containing a first strand comprising a ribonucleotide sequence which corresponds to a nucleotide sequence of PDE3B and a second strand comprising a ribonucleotide sequence which is complementary to the nucleotide sequence of PDE3B, wherein the first and the second ribonucleotide sequences are separate complementary sequences that hybridize to each other to form said double-stranded structure.

Disclosed are methods of inhibiting expression of PDE3B in a cell comprising: (a) isolating the cell; (b) contacting the cell with a RNA comprising a double-stranded structure comprising a first strand comprising a ribonucleotide sequence which corresponds to a nucleotide sequence of PDE3B and a second strand comprising a ribonucleotide sequence which is complementary to the nucleotide sequence of PDE3B, wherein the first and the second ribonucleotide sequences are separate sequences that hybridize to each other to form said double-stranded structure, and (c) subsequently introducing the cell into a host, wherein said RNA comprising the double-stranded structure inhibits expression of the target gene in the cell in the host.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B show a diagram of a GWAS Study Design. a) DNA sequence variants across 3 separate ancestry groups in the Million Veteran Program were meta-analyzed using an inverse-variance weighted fixed effects meta-analysis in the discovery phase. Variants with suggestive association were then brought forward for independent replication. b) DNA sequence variants with suggestive association (P<10⁻⁴) in discovery were brought forward for independent replication and tested using summary statistics from either 1) the 2017 exome-array focused GLGC meta-analysis (exome chip replication) or 2) the 2013 “joint meta-analysis” (joint meta-analysis replication) from the GLGC. Abbreviations: MVP, Million Veteran Program; GWAS, genome-wide association study; EHR, electronic health record; GLGC, Global Lipids Genetics Consortium

FIG. 2 shows Predicted Loss of Function (pLoF). Variation in Million Veteran Program Participants. The number of pLoF variants passing quality control for white, black, and Hispanic participants in MVP. Each pLoF annotation (frameshift, splice donor/acceptor, stop gain) is depicted by a separate color. Abbreviations: MVP, Million Veteran Program; pLoF, predicted Loss of Function

FIGS. 3A-3D show a comparison of 354 Independent Lipid Associated Variants Across Ethnicities. Allele frequencies observed in white individuals (x-axes) compared to black (a, R=0.72,) or Hispanic (b, R=0.96) individuals for lipid-associated variants. Effect estimates for LDL cholesterol association in white individuals (x-axes) compared to black (c, β=1.07) or Hispanic (d, β=1.06) individuals. Abbreviations: SD, Standard Deviations; LDL, Low-Density Lipoprotein; R=Pearson correlation coefficient

FIGS. 4A-4C show PDE3B Loss of Gene Function, Lipids, and Coronary Disease. Results for the association of the predicted loss of function mutation p.Arg783Ter in PDE3B with HDL cholesterol (a) and TG (b) for white veterans in MVP with independent replication in the DiscovEHR study. c) Meta-analysis of the association of damaging PDE3B mutations and coronary disease across five studies, including three (MIGen, PMBB, DiscovEHR) with exome sequencing. Results were pooled in an inverse-variance weighted fixed effects meta-analysis. Abbreviations: MVP, Million Veteran Program; HDL, High-Density Lipoprotein; TG, Triglycerides; UKBB, UK Biobank; MIGen, Myocardial Infarction Genetics Consortium; PMBB, Penn Medicine Biobank

FIG. 5 shows a supervised ADMIXTURE analysis was performed on all MVP samples using 1000 Genomes Project reference samples as the reference panel. Following training of the ADMIXTURE model on 5 populations representing East Asia (CHB), Europe (GBR), East Africa (LWK), South American (PEL), and West Africa (YRI), individuals with at least 50% African (LWK or YRI) ancestry and self-identifying as “non-Hispanic” and “black” were assigned to a separate MVP “black” population. The x-axis depicts each of the 57,332 samples assigned to this group, the Y-axis shows the percentage of each reference population per sample.

FIG. 6 shows a supervised ADMIXTURE analysis was performed on all MVP samples using 1000 Genomes Project reference samples as the reference panel. Following training of the ADMIXTURE model on 5 populations representing East Asia (CHB), Europe (GBR), East Africa (LWK), South American (PEL), and West Africa (YRI), individuals self-identifying as “Hispanic” were assigned to a separate MVP “Hispanic” population. The x-axis depicts each of the 24,743 samples assigned to this group, the Y-axis shows the percentage of each reference population per sample.

FIG. 7 shows a plot of the Z score of association (β/SE) for 444 independent lipid exome-wide associated (P<2.2×10-7) DNA sequence variants per trait as reported in the published GLGC 2017 exome chip analysis3 and in our MVP discovery GWAS analysis aligned to the lipid raising allele. A strong association (linear regression P<1.0×10-100) between published (GLGC) and MVP Z scores was observed for each trait. Abbreviations: SE, standard error; GLGC, Global Lipids Genetics Consortium; MVP, Million Veteran Program; HDL-C, High-Density Lipoprotein Cholesterol; LDL-C, Low-Density Lipoprotein Choleterol; TG, Triglycerides; TC, Total Cholesterol

FIG. 8 shows a plot of the effect estimates (β) for 444 independent lipid exome-wide associated (P<2.2×10-7) DNA sequence variants per trait as reported in the published GLGC 2017 exome chip analysis3 and in our MVP discovery GWAS analysis. The effect estimate between MVP discovery and published (GLGC) β values demonstrated evidence of the winner's curse (β=0.72, 0.90, 0.85, 0.96 for LDL-C, TG, TC, and HDL-C, respectively after exclusion of extreme outliers). Abbreviations: GLGC, Global Lipids Genetics Consortium; MVP, Million Veteran Program; HDL-C, High-Density Lipoprotein Cholesterol; TG, Triglycerides; LDL-C, Low-Density Lipoprotein Cholesterol; TC, Total Cholesterol

FIG. 9 shows the expected association P values versus the observed distribution of P values for LDL cholesterol, TG, TC, and HDL cholesterol association are displayed. Quantile-quantile plots were inspected for ancestry specific analyses, and genomic control values were <1.20 for each racial group (data not shown). The inflation observed (λGC=1.08-1.13) is comparable to that observed in other studies of polygenic traits with similar large sample sizes (n>300,000)4,5. Abbreviations: HDL, High-Density Lipoprotein Cholesterol; TG, Triglycerides; LDL, Low-Density Lipoprotein Cholesterol; TC, Total Cholesterol; MVP, Million Veteran Program

FIGS. 10A-10F show a,b) Effect estimates for TG association in white individuals (x-axes) compared to black (a, β=0.76) or Hispanic (b, β=0.91) individuals. c,d) Effect estimates for TC association in white individuals (x-axes) compared to black (c, β=0.95) or Hispanic (d, β=1.08) individuals. e,f) Effect estimates for HDL-C association in white individuals (x-axes) compared to black (e, β=0.88) or Hispanic (f, β=1.04) individuals. Abbreviations: SD, Standard Deviations; HDL-C, High-Density Lipoprotein Cholesterol; TG, Triglycerides; TC, Total Cholesterol.

FIG. 11 shows a flow chart for generation of summary statistics used in GCTA-COJO approximate stepwise conditional analysis. The GCTA-COJO software requires GWAS summary statistics and an LD-matrix of a representative group of samples with similar genetic ancestry to those used for the GWAS. As such, summary statistics were combined from MVP (European ancestry subgroup), the GLGC 2017 exome chip analysis (predominantly European ancestry) and the GLGC 2013 “joint meta-analysis” (predominantly European ancestry) via an inverse-variance weight fixed effects meta-analysis. These combined results were then used with an LD-matrix of 10,000 randomly selected European samples from the UK Biobank interim release6 for GCTA-COJO stepwise conditional analysis.

FIG. 12 shows a plot of −log 10(P) for lipid-gene associations by chromosomal position for all genes analyzed in the TWAS. The genes nearest to the top associated variants are displayed.

FIG. 13 shows a graph of the 655 genome-wide (P<5×10⁻⁸) gene-lipid associations for each of four tissues (adipose, liver, tibial artery, and whole blood) resulting from the lipids TWAS analysis.

FIG. 14 shows the association results of previously reported genome-wide significant loci for HDL cholesterol in the MVP lipids discovery (trans-ethnic) analysis. * Genes for variants that are outside the transcript boundary of a protein-coding gene are shown with nearest the gene in parentheses [eg, (CETP)] if applicable. ** Refers to the 1 million base-pair window around a previously described lipid variant. Abbreviations: EA, Effect Allele; NEA, Non-effect Allele; EAF, Effect Allele Frequency; SE, Standard Error; I, Insertion; D, Deletion.

FIG. 15 shows the association results of previously reported genome-wide significant loci for LDL cholesterol in the MVP lipids discovery (trans-ethnic) analysis. * Genes for variants that are outside the transcript boundary of a protein-coding gene are shown with nearest the gene in parentheses [eg, (CYP26A)] if applicable. ** Refers to the 1 million base-pair window around a previously described lipid variant. Abbreviations: EA, Effect Allele; NEA, Non-effect Allele; EAF, Effect Allele Frequency; SE, Standard Error; I, Insertion; D, Deletion.

FIG. 16 shows the association results of previously reported genome-wide significant loci for TG in the MVP lipids discovery (trans-ethnic) analysis. * Genes for variants that are outside the transcript boundary of a protein-coding gene are shown with nearest the gene in parentheses [eg, (CETP)] if applicable. ** Refers to the 1 million base-pair window around a previously described lipid variant. Abbreviations: EA, Effect Allele; NEA, Non-effect Allele; EAF, Effect Allele Frequency; SE, Standard Error; I, Insertion; D, Deletion.

FIG. 17 shows the association results of previously reported genome-wide significant loci for TC in the MVP lipids discovery (trans-ethnic) analysis. * Genes for variants that are outside the transcript boundary of a protein-coding gene are shown with nearest the gene in parentheses [eg, (CETP)] if applicable. ** Refers to the 1 million base-pair window around a previously described lipid variant. Abbreviations: EA, Effect Allele; NEA, Non-effect Allele; EAF, Effect Allele Frequency; SE, Standard Error; I, Insertion; D, Deletion.

FIG. 18 shows the novel genome-wide significant loci for HDL in the MVP lipids GWAS following independent replication. * Genes for variants that are outside the transcript boundary of a protein-coding gene are shown with nearest the gene in parentheses [eg, (BDNF)]. Abbreviations: EA, Effect Allele; NEA, Non-effect Allele; EAF, Effect Allele Frequency; EAF SE, Standard Error in Allele Frequency; Het 12, Heterogeneity I-Sqaured Statistic; SE, Standard Error.

FIG. 19 shows the novel genome-wide significant loci for LDL in the MVP lipids GWAS following independent replication. * Genes for variants that are outside the transcript boundary of a protein-coding gene are shown with nearest the gene in parentheses [eg, (THOP1)]. Abbreviations: EA, Effect Allele; NEA, Non-effect Allele; EAF, Effect Allele Frequency; EAF SE, Standard Error in Allele Frequency; Het 12, Heterogeneity I-Sqaured Statistic; SE, Standard Error.

FIG. 20 shows the novel genome-wide significant loci for TG in the MVP lipids GWAS following independent replication. * Genes for variants that are outside the transcript boundary of a protein-coding gene are shown with nearest the gene in parentheses [eg, (BDNF)]. Abbreviations: EA, Effect Allele; NEA, Non-effect Allele; EAF, Effect Allele Frequency; EAF SE, Standard Error in Allele Frequency; Het 12, Heterogeneity I-Squared Statistic; SE, Standard Error.

FIG. 21 shows the novel genome-wide significant loci for TC in the MVP lipids GWAS following independent replication. * Genes for variants that are outside the transcript boundary of a protein-coding gene are shown with nearest the gene in parentheses [eg, (ARL11)]. Abbreviations: EA, Effect Allele; NEA, Non-effect Allele; EAF, Effect Allele Frequency; EAF SE, Standard Error in Allele Frequency; Het 12, Heterogeneity I-Sqaured Statistic; SE, Standard Error.

FIG. 22 shows the 223 variants (across 223 distinct loci) used for a weighted genetic risk score. Effect estimates/P values are taken from 2017 GLGC exome array analysis. * Genes for variants that are outside the transcript boundary of a protein-coding gene are shown with nearest the gene in parentheses [eg, (ARL11)]. Abbreviations: EA, Effect Allele; NEA, Non-effect Allele; EAF, Effect Allele Frequency; SE, Standard Error.

FIG. 23 shows the increase in variance explained as a function of the number of repeated measures in MVP non-Hispanic whites (for a fixed sample size of 171,314 MVP participants; only individuals with five or more measures were included). Variance explained was calculated using a genetic risk score of 223 previously described lipid hits with previous effect sizes.

FIG. 24 shows examples of PDE3B variants.

FIG. 25 shows examples of PDE3B variants.

FIG. 26 shows a transcriptome-wide association study (TWAS) results for HDL Cholesterol in 4 tissues. Abbreviations: Chr, Chromosome; Pos, Position; Top GWAS rsid, rsID of the most significant GWAS variant in locus; Top GWAS Zscore, Z-score of the most significant GWAS variant in locus; Top eQTL rsid, rsID of the best eQTL in the locus; Top eQTL Zscore, Z-score of the best eQTL in the locus; GWAS Zscore for Top eQTL Variant, GWAS Z-score for this eQTL.

FIG. 27 shows a transcriptome-wide association study (TWAS) results for LDL Cholesterol in 4 tissues. Abbreviations: Chr, Chromosome; Pos, Position; Top GWAS rsid, rsID of the most significant GWAS variant in locus; Top GWAS Zscore, Z-score of the most significant GWAS variant in locus; Top eQTL rsid, rsID of the best eQTL in the locus; Top eQTL Zscore, Z-score of the best eQTL in the locus; GWAS Zscore for Top eQTL Variant, GWAS Z-score for this eQTL.

FIG. 28 shows a transcriptome-wide association study (TWAS) results for TG in 4 tissues. Abbreviations: Chr, Chromosome; Pos, Position; Top GWAS rsid, rsID of the most significant GWAS variant in locus; Top GWAS Zscore, Z-score of the most significant GWAS variant in locus; Top eQTL rsid, rsID of the best eQTL in the locus; Top eQTL Zscore, Z-score of the best eQTL in the locus; GWAS Zscore for Top eQTL Variant, GWAS Z-score for this eQTL.

FIG. 29 shows a transcriptome-wide association study (TWAS) results for TC in 4 tissues. Abbreviations: Chr, Chromosome; Pos, Position; Top GWAS rsid, rsID of the most significant GWAS variant in locus; Top GWAS Zscore, Z-score of the most significant GWAS variant in locus; Top eQTL rsid, rsID of the best eQTL in the locus; Top eQTL Zscore, Z-score of the best eQTL in the locus; GWAS Zscore for Top eQTL Variant, GWAS Z-score for this eQTL.

FIG. 30 shows a transcriptome-wide association study (TWAS) results in loci not identified in previous GLGC or current MVP Lipids GWAS.

FIG. 31 shows genome-wide significant pLoF variants for lipids in the MVP discovery analysis. * pLoF Confidence reflects the reported annotation by the VEP software (PMID: 20562413), LOFTEE Plugin in which a series of filters are applied to candidate pLoF variants. Confident means the variant does not fail any filters. Not Confident means the mutation fails at least one of these filters. A full list of filters is provided at https://github.com/konradjk/loftee. ** Sub-genome-wide in the MVP discovery analysis, brought over the genome-wide threshold with replication from DiscovEHR Study.

Abbreviations: EA, Effect Allele; NEA, Non-effect Allele; EAF, Effect Allele Frequency; SE, Standard Error.

FIG. 32 shows CAD association statistics for 118 novel genome-wide significant loci in the MVP lipids GWAS analysis. * Binomial Test Group Refers to the Lipid Group Each Variant Falls within based on P thresholds: LDL, LDL P<10-E4; TG, TG P<10-E4; HDL, HDL P<10-E4 & TG P>0.05 & LDL P>0.05. Abbreviations: EA, Effect Allele; NEA, Non-effect Allele; EAF, Effect Allele Frequency; CAD, Coronary Artery Disease; SE, Standard Error.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a variant” includes a plurality of such variants, reference to “the variant” is a reference to one or more variants and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range¬from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods for Determining Risk of Coronary Artery Disease

Disclosed are methods for determining a subject's risk for having or developing coronary artery disease comprising determining in the subject the presence of one or more PDE3B loss of function or damaging variants, and wherein the presence of the variant indicates the subject's reduced risk for having or developing coronary artery disease.

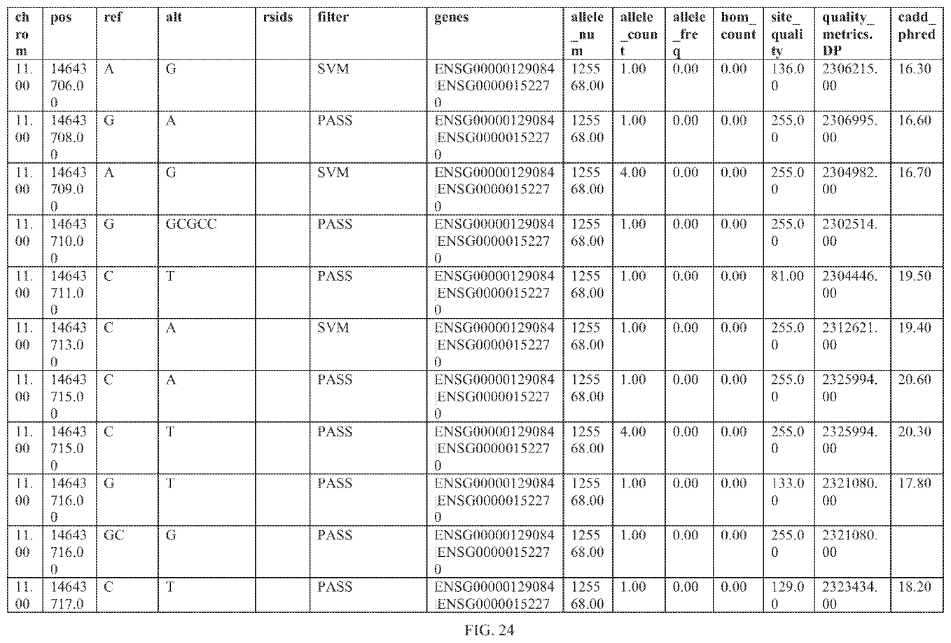

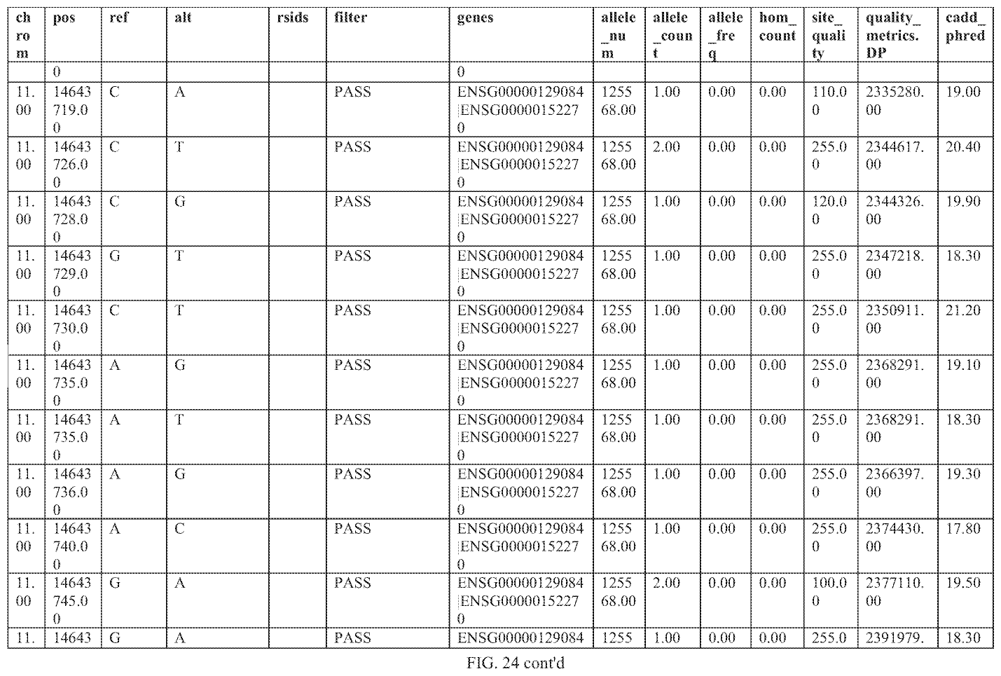

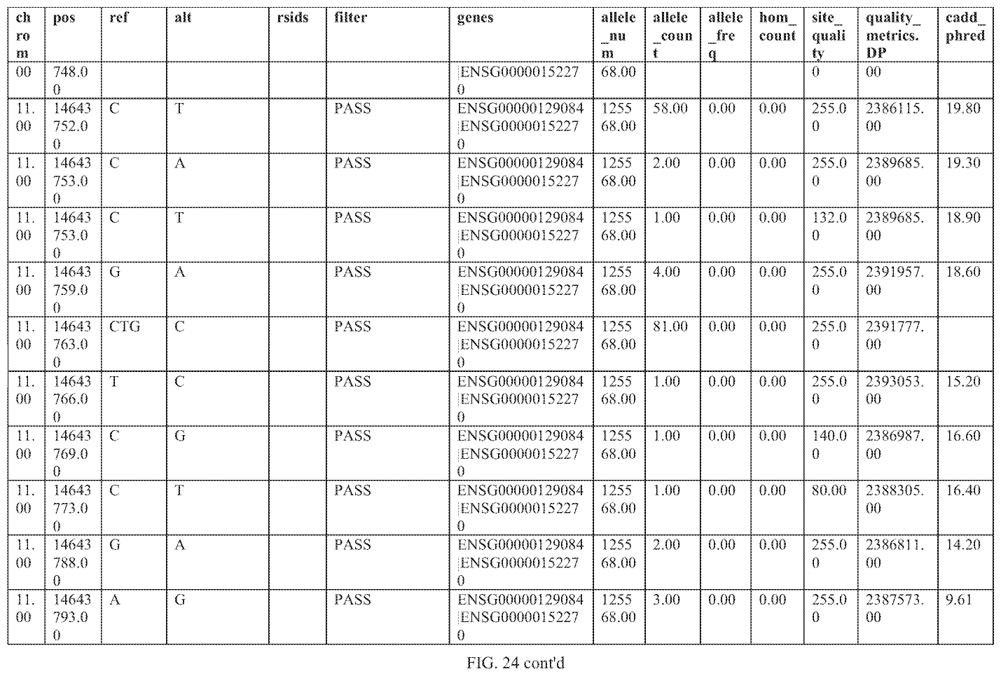

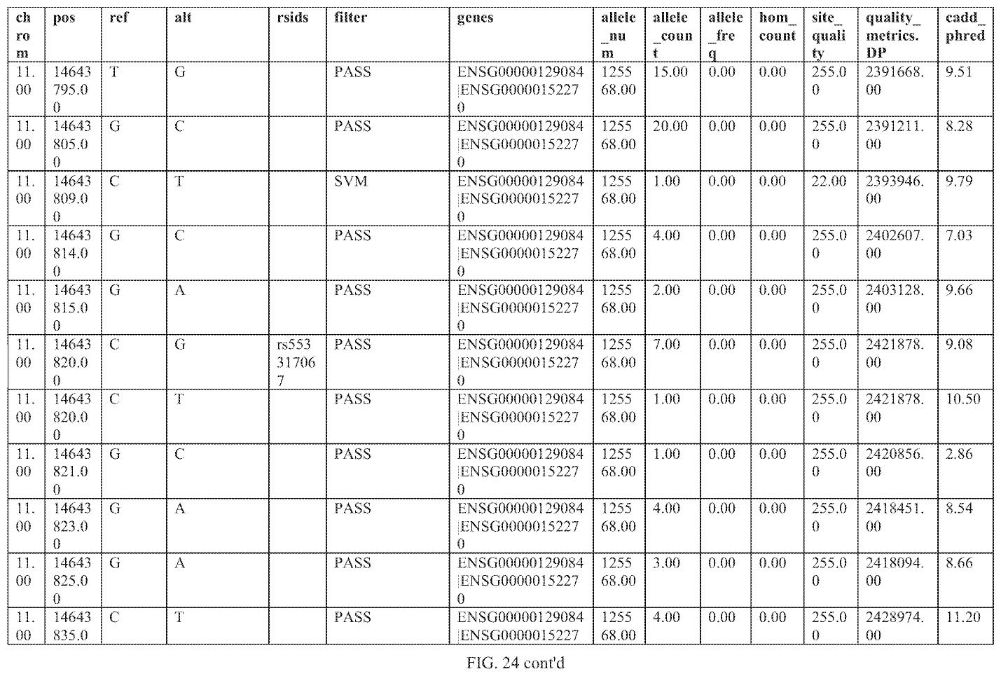

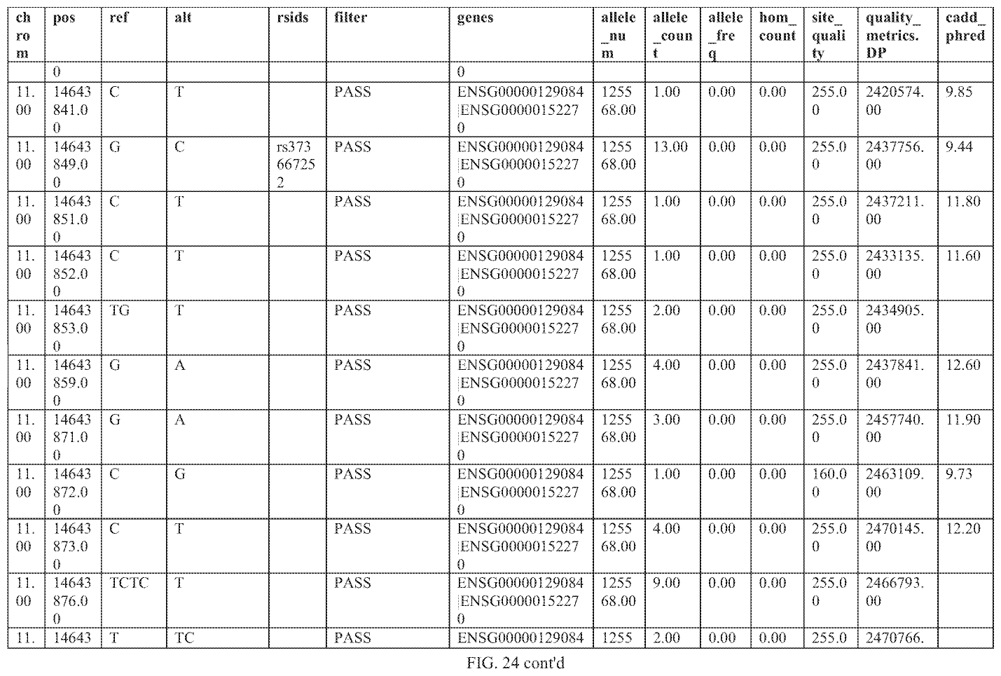

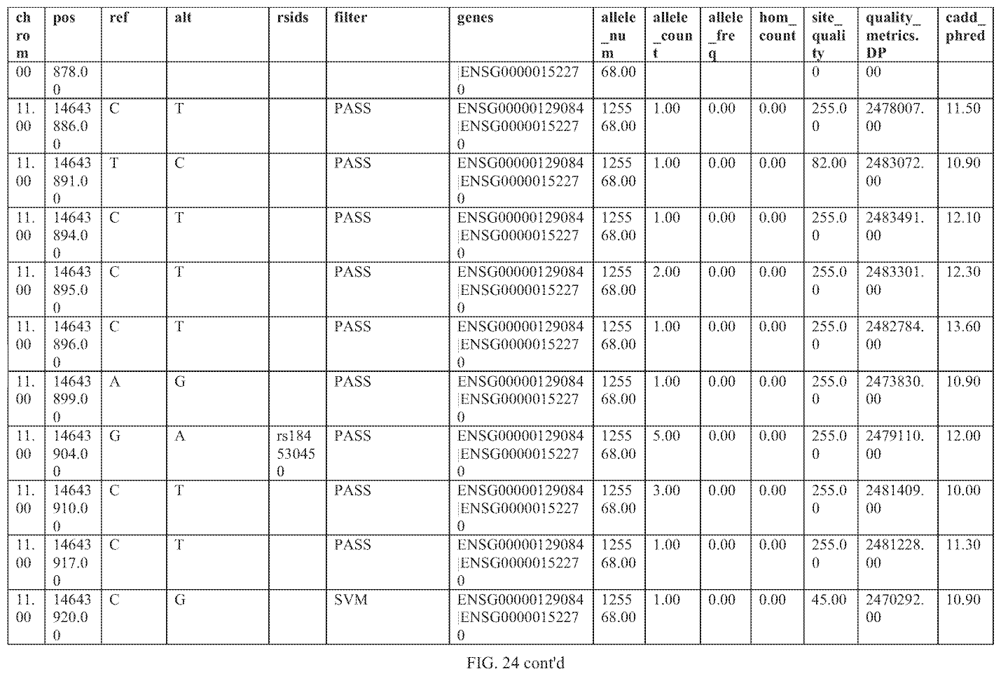

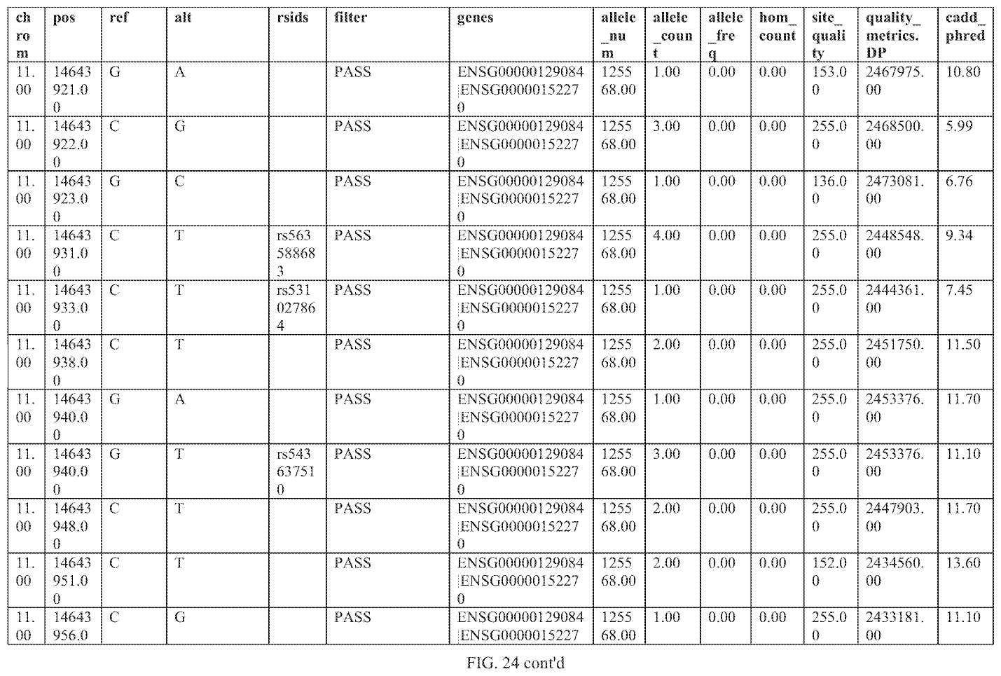

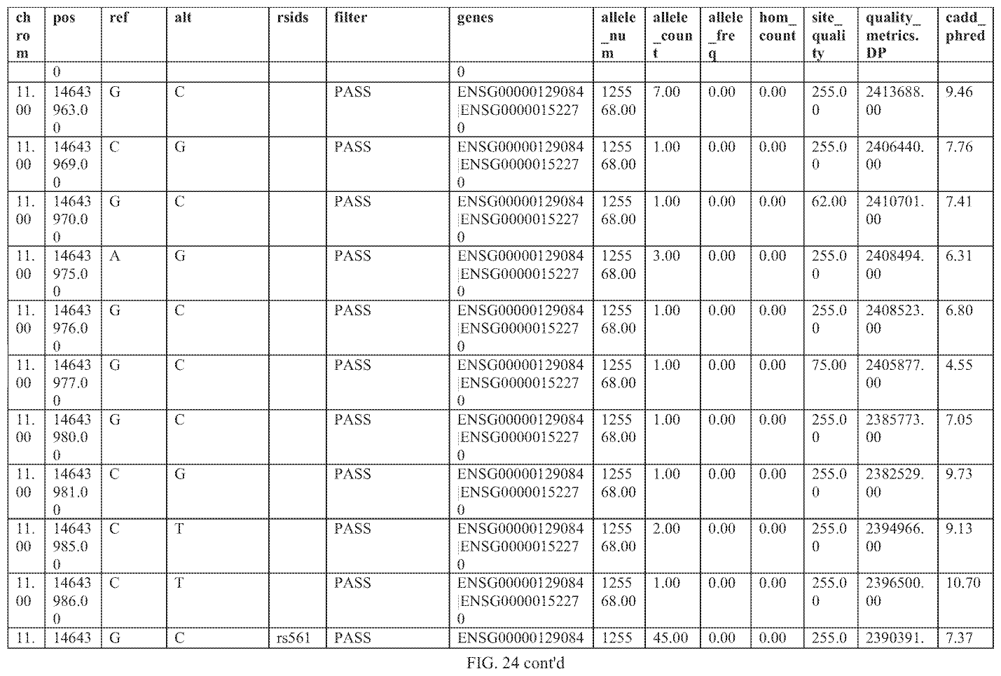

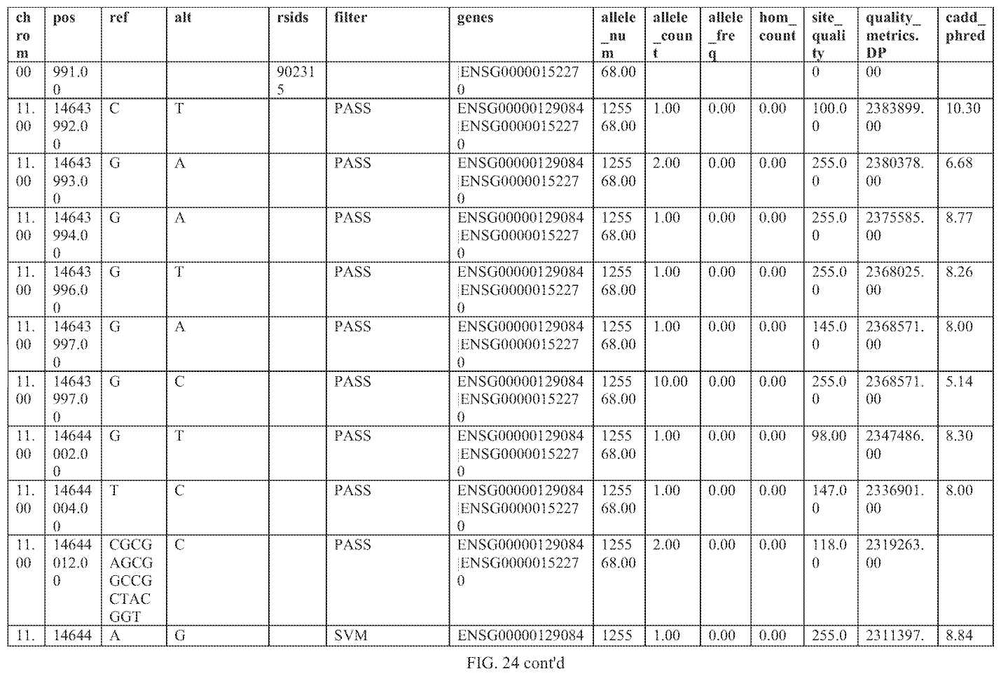

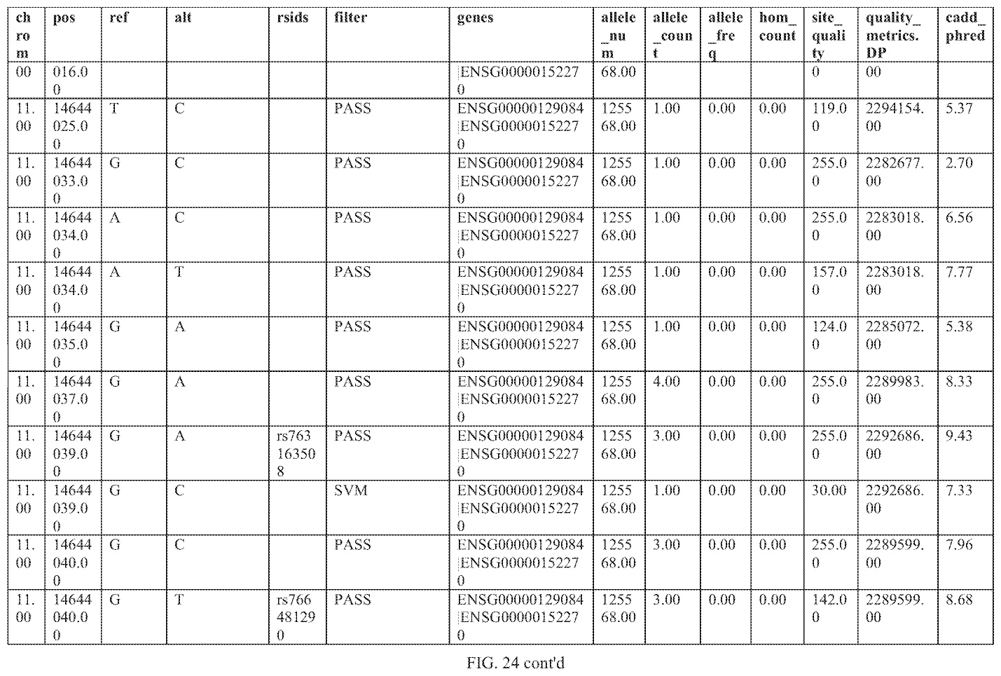

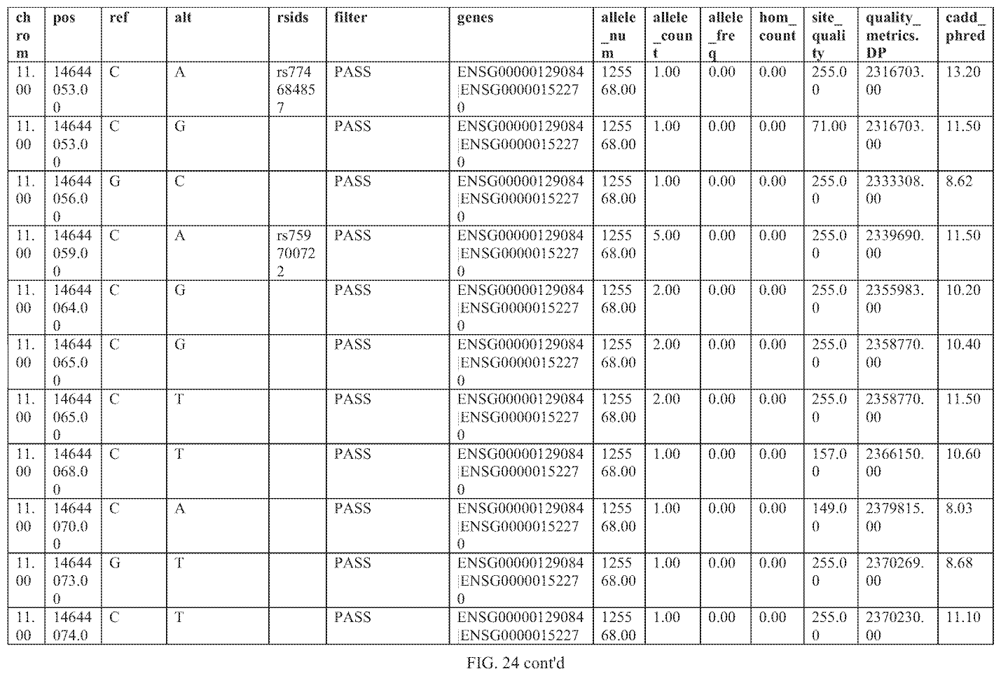

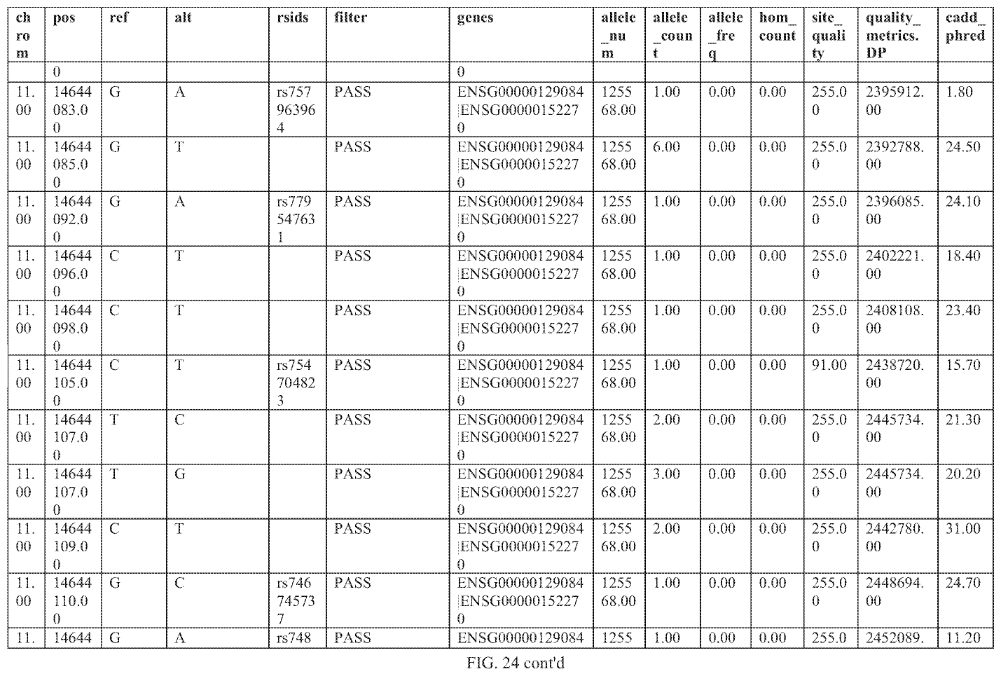

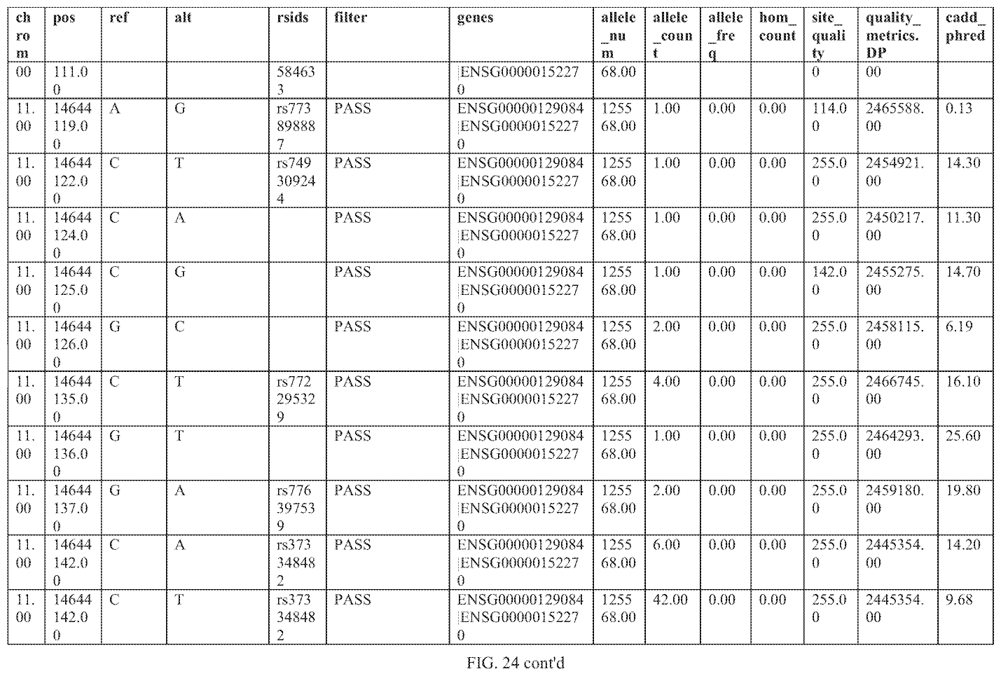

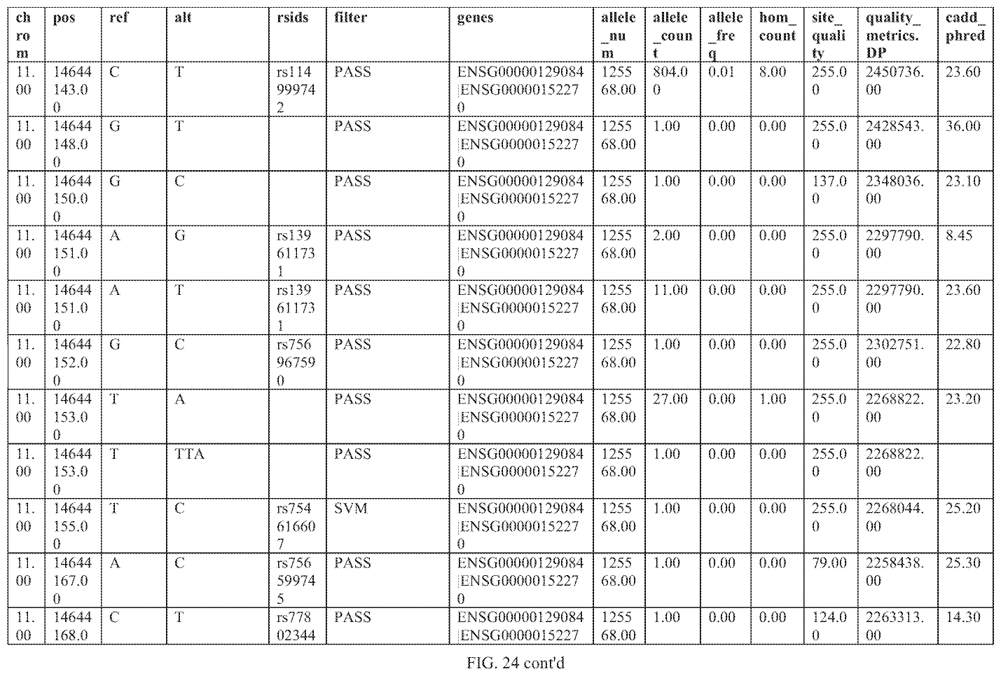

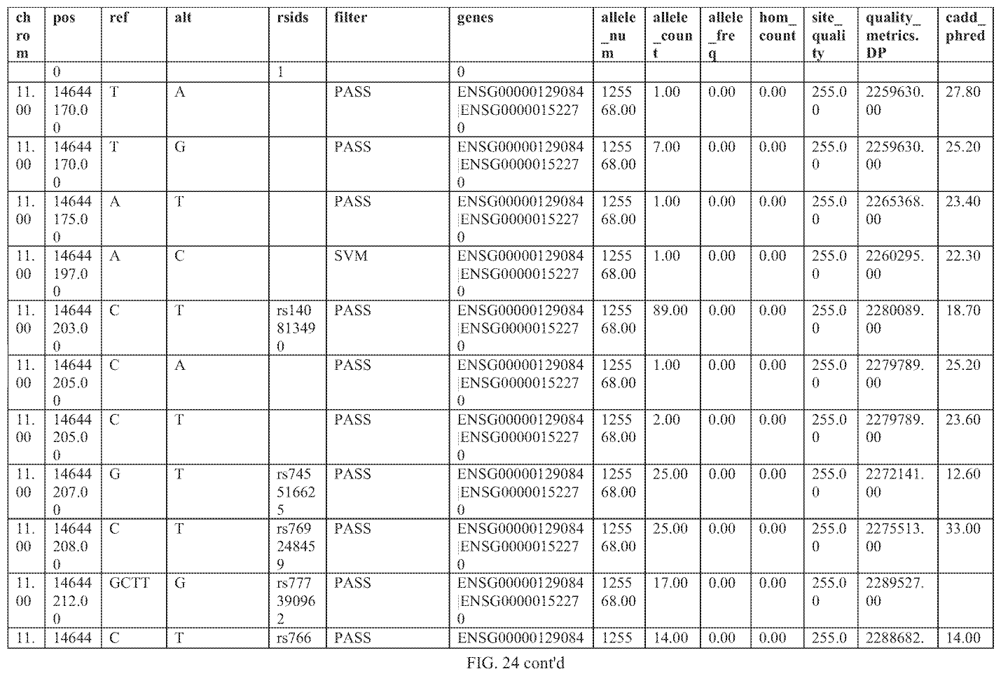

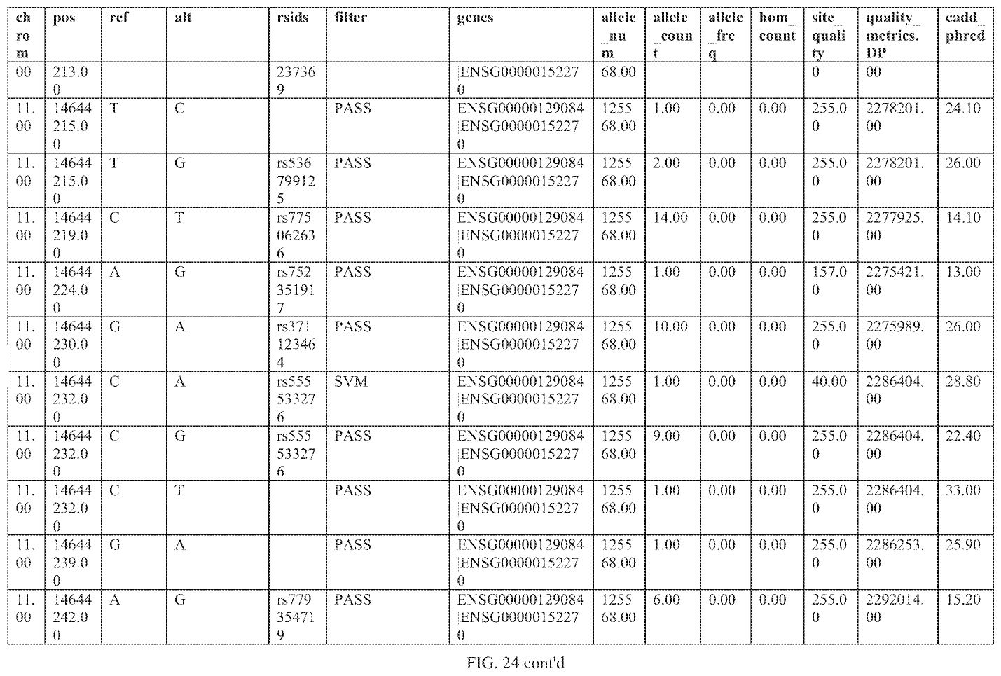

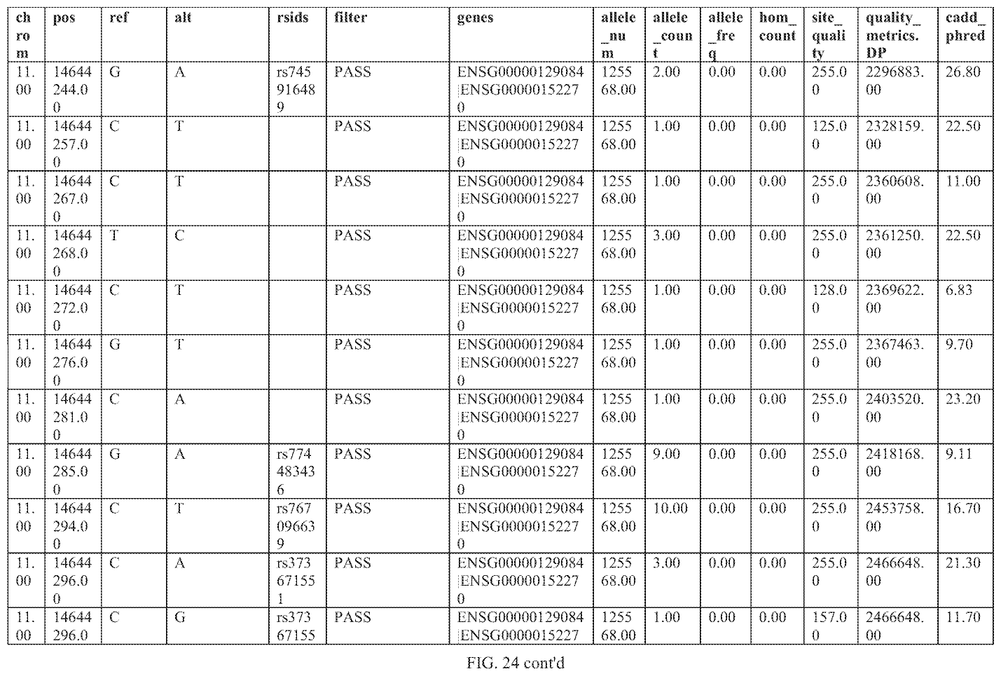

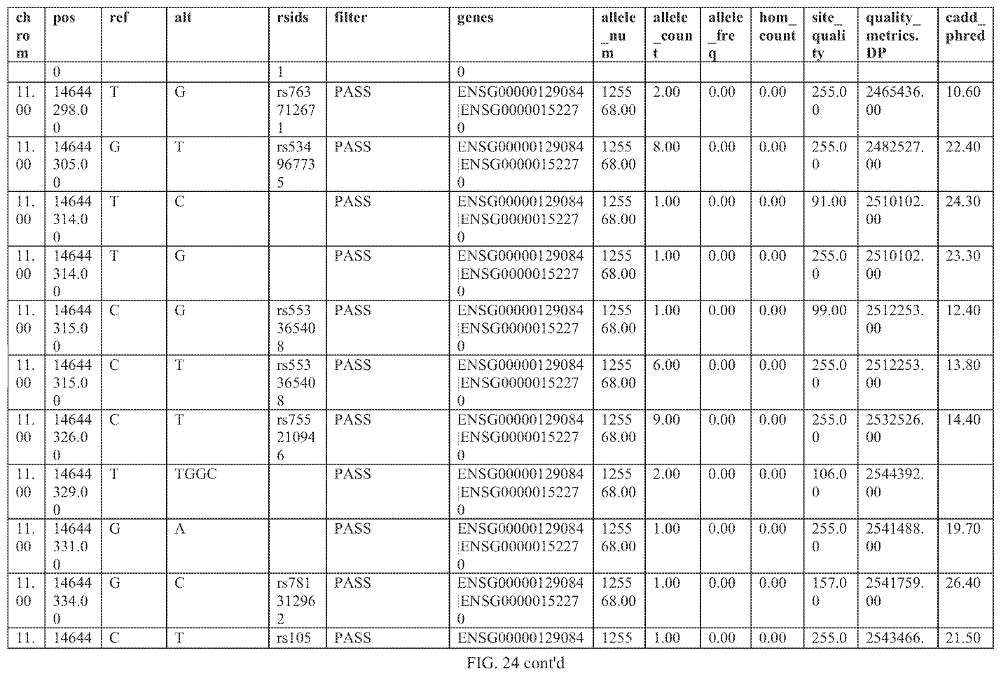

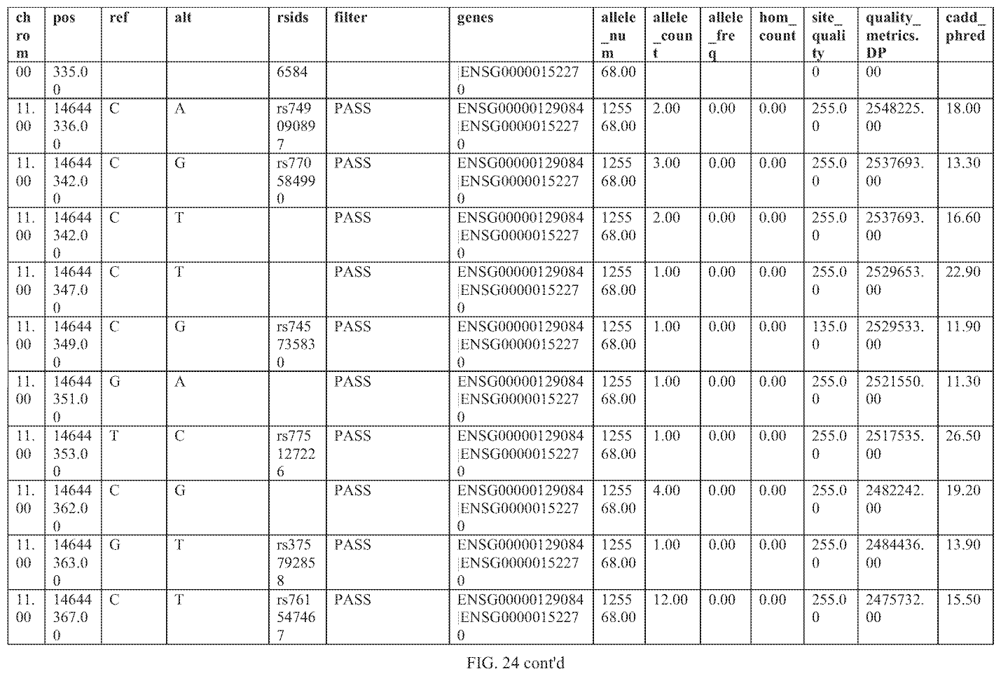

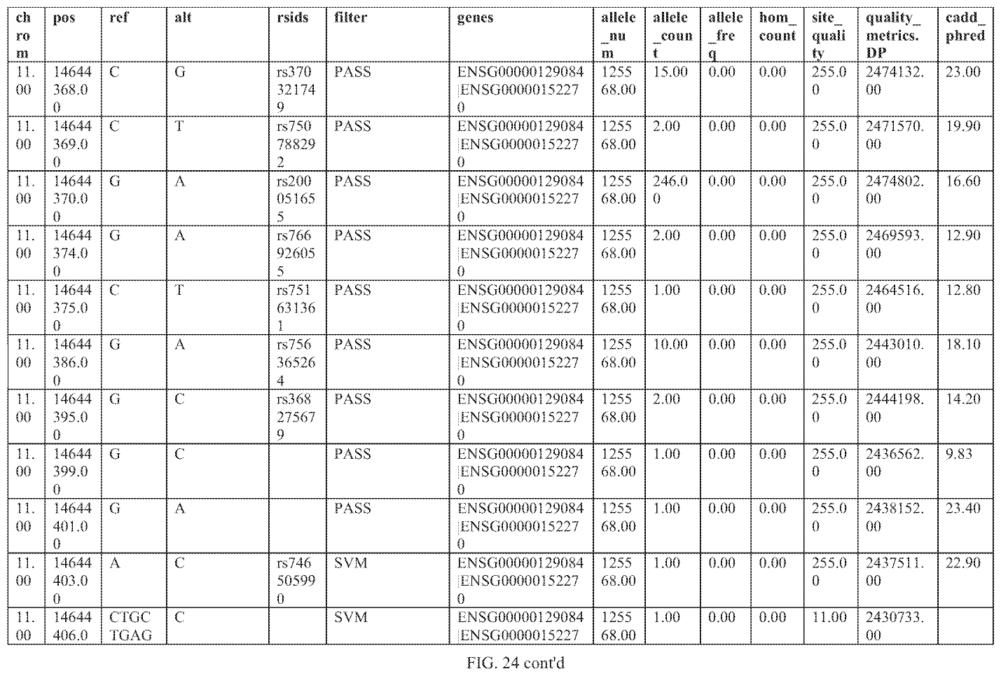

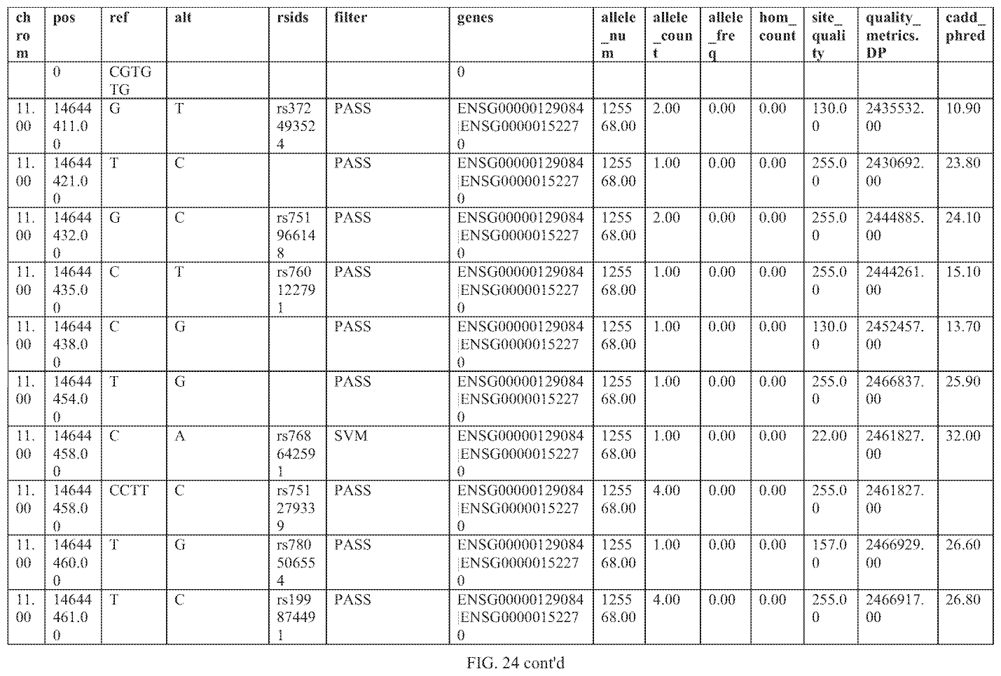

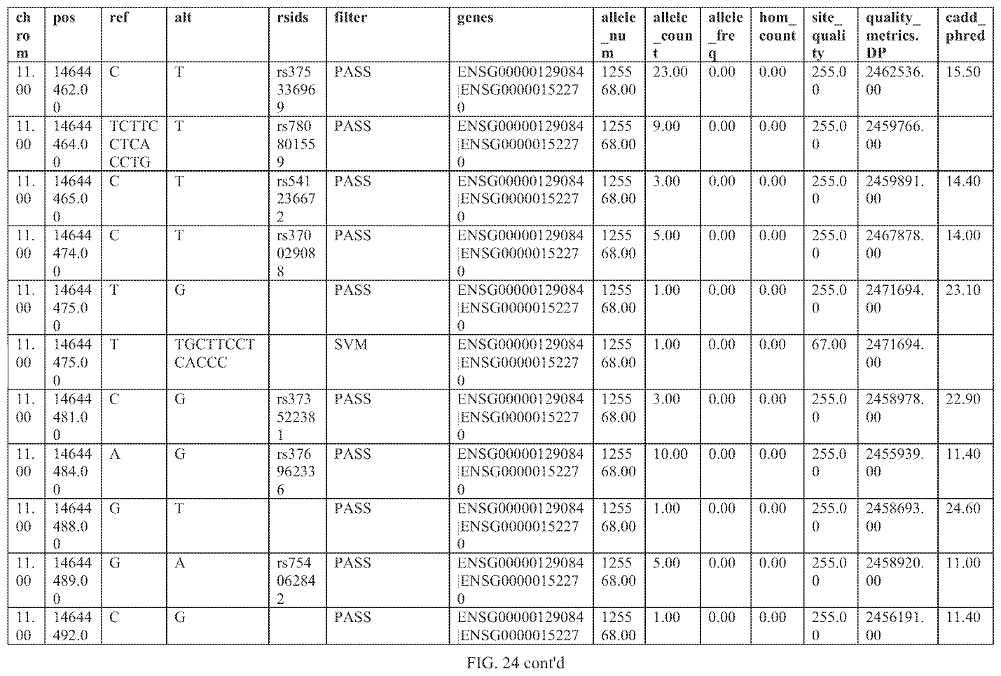

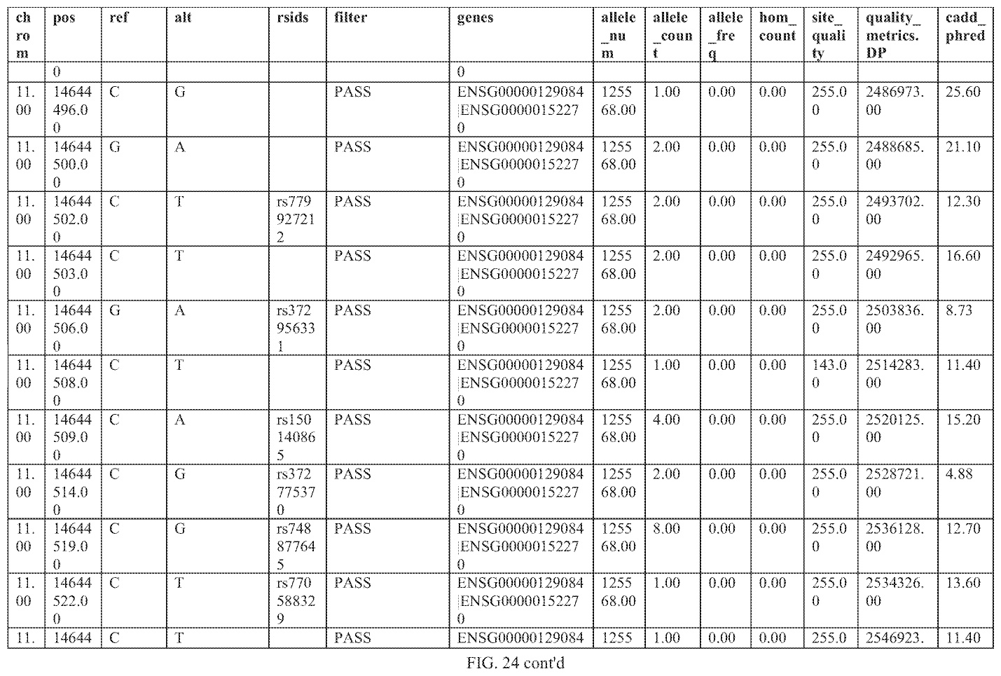

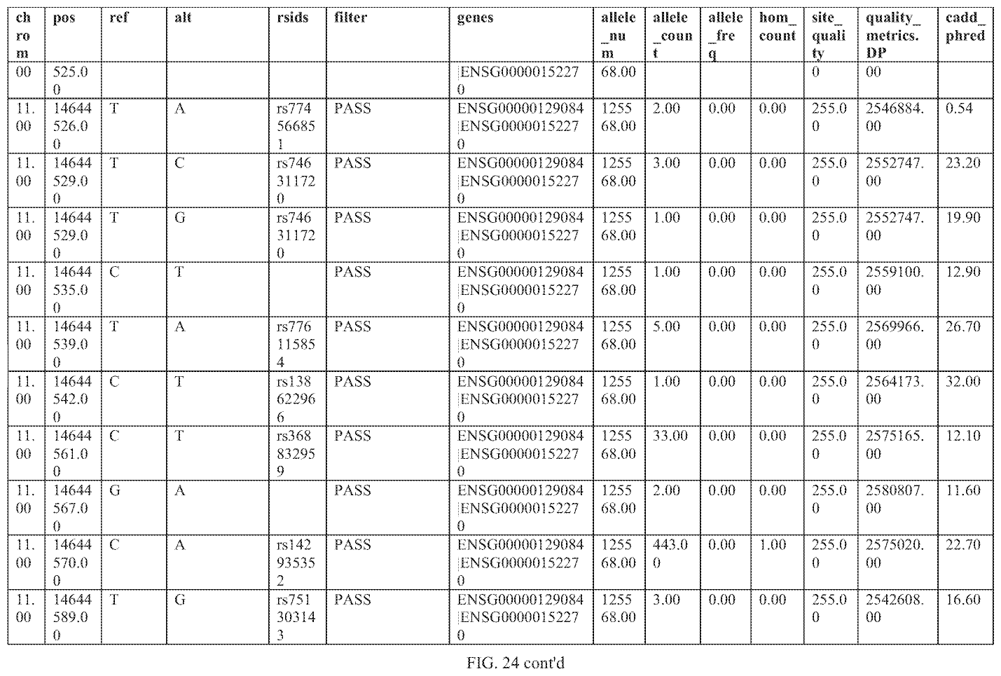

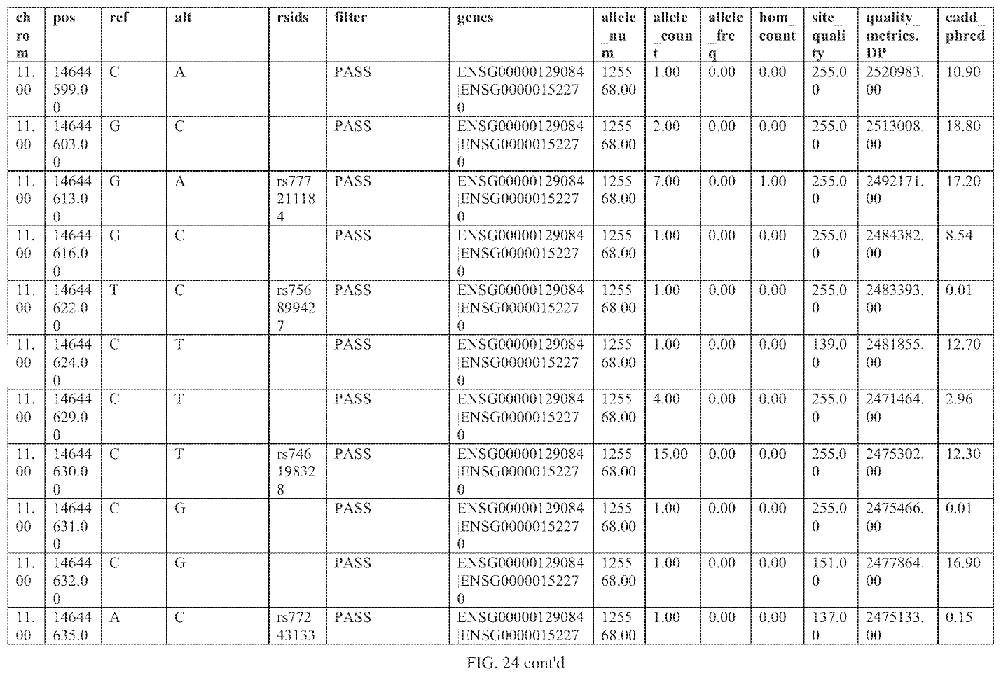

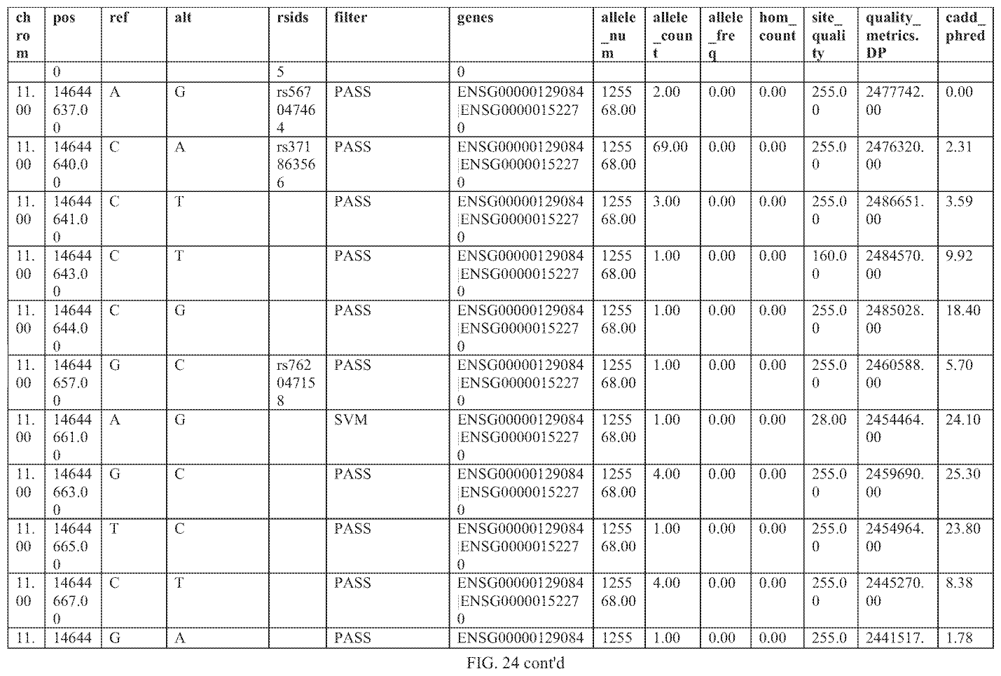

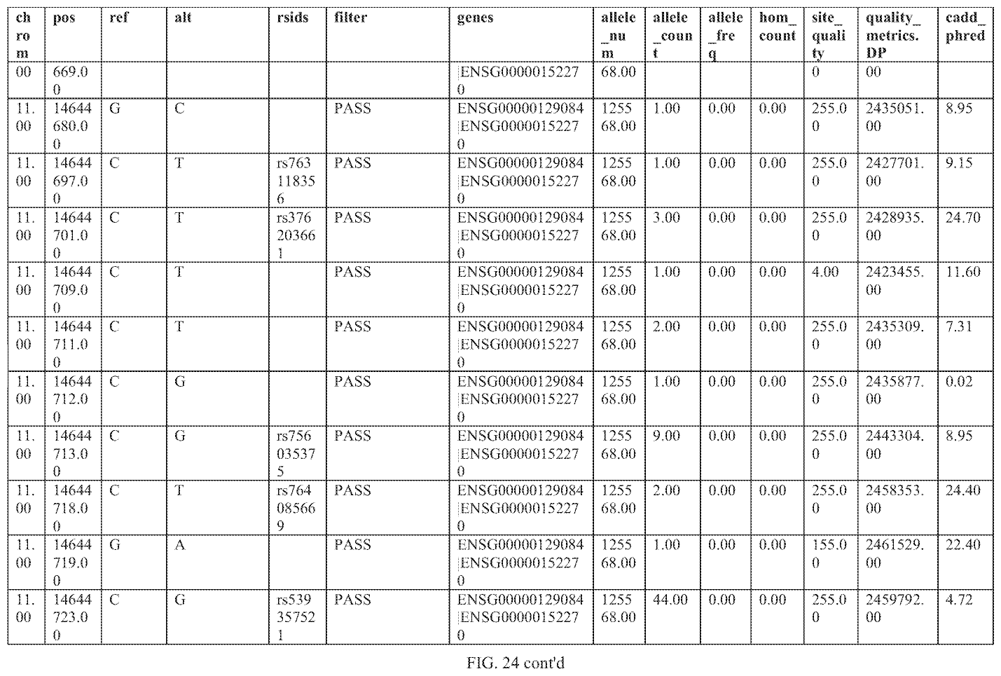

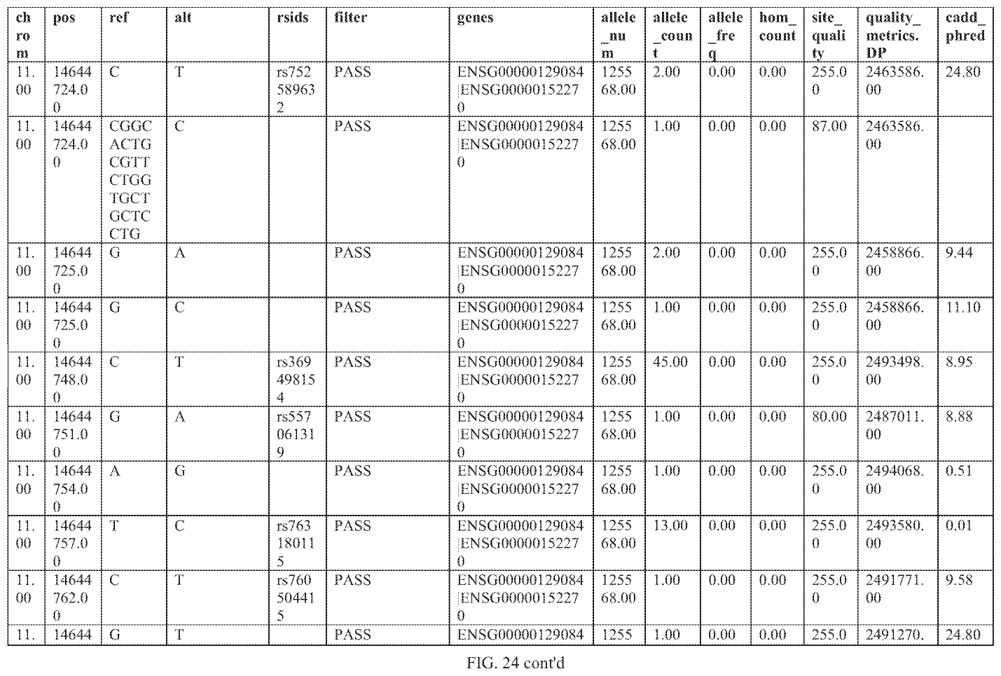

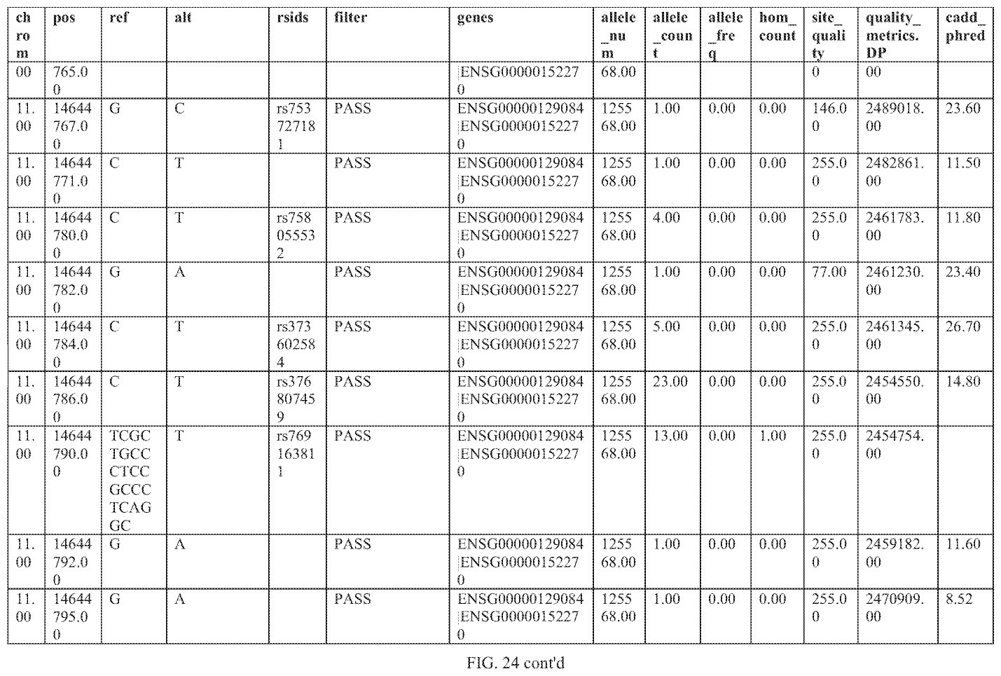

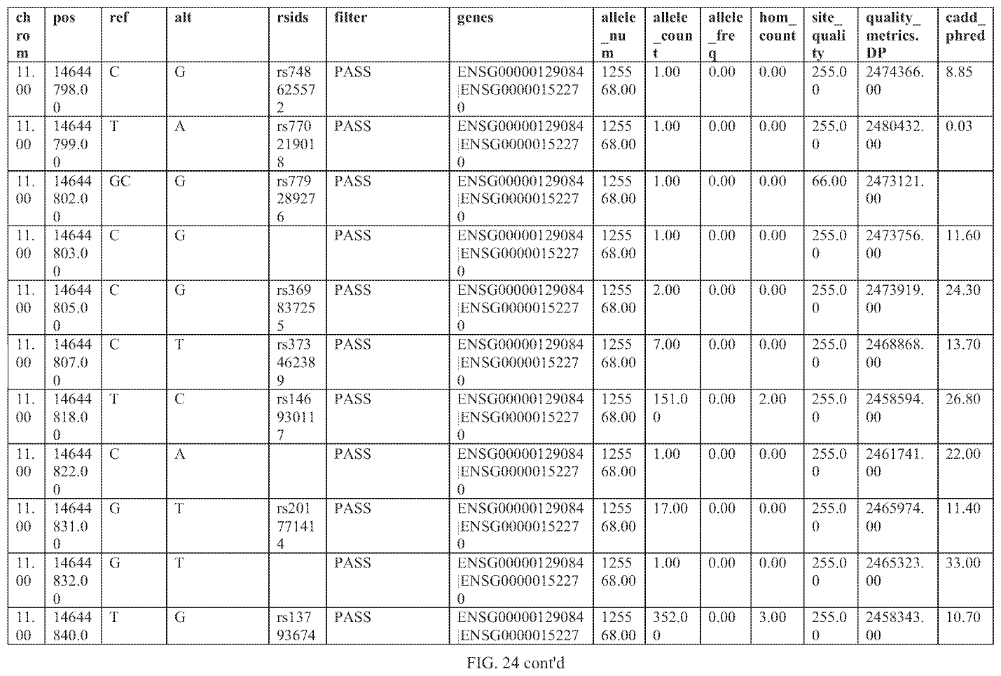

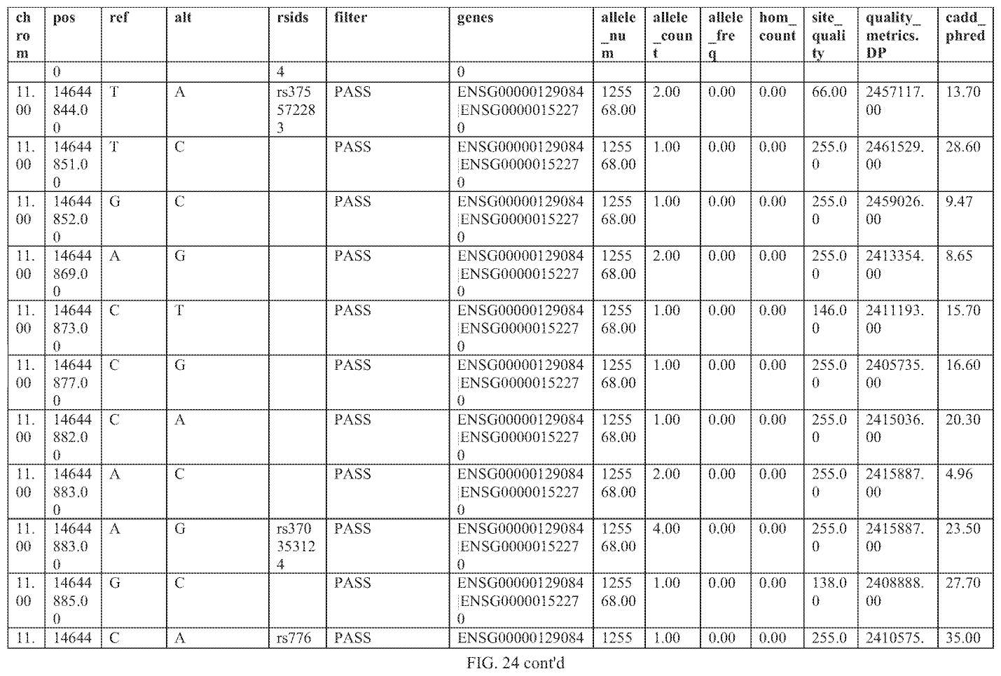

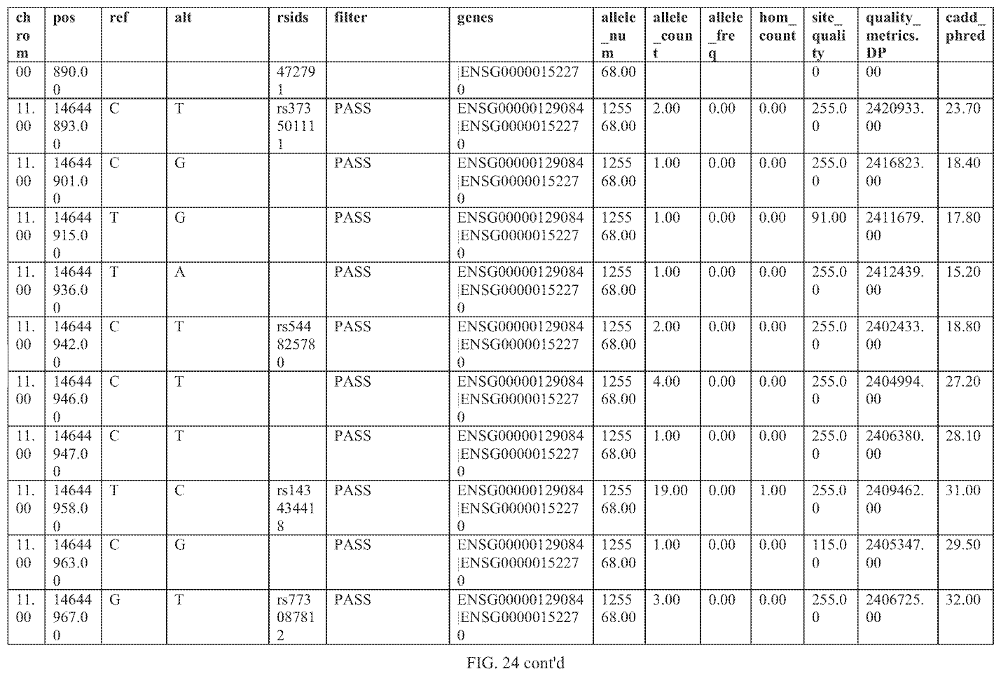

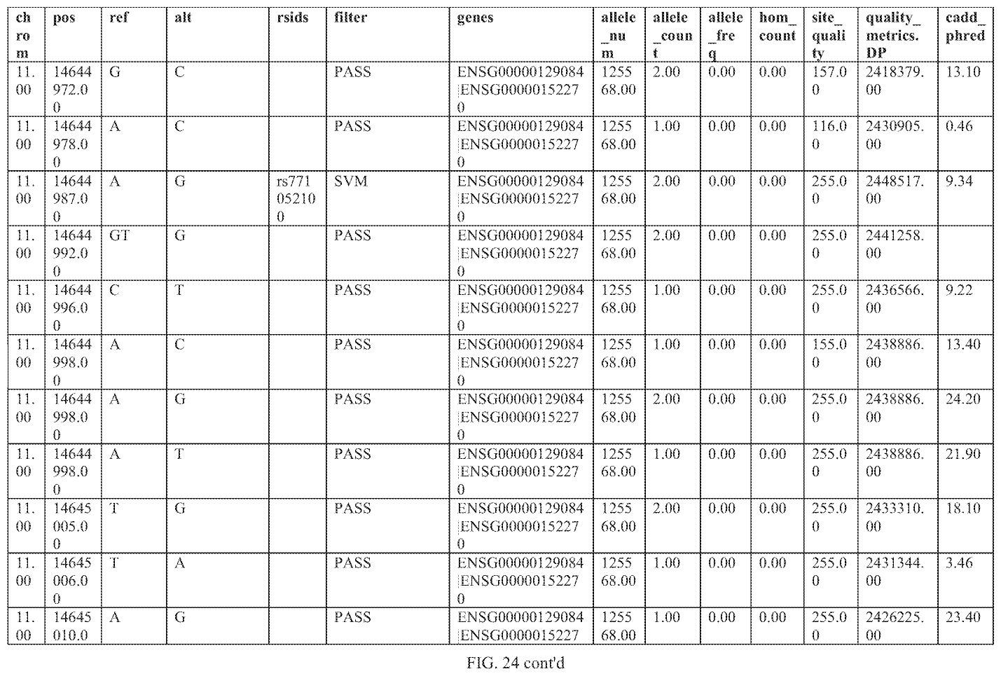

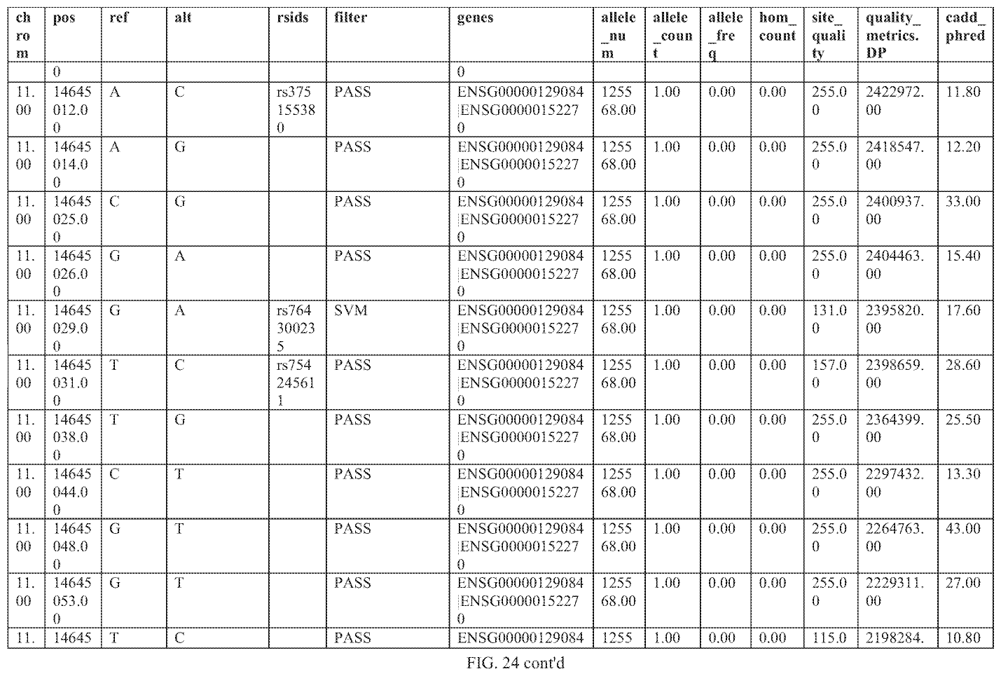

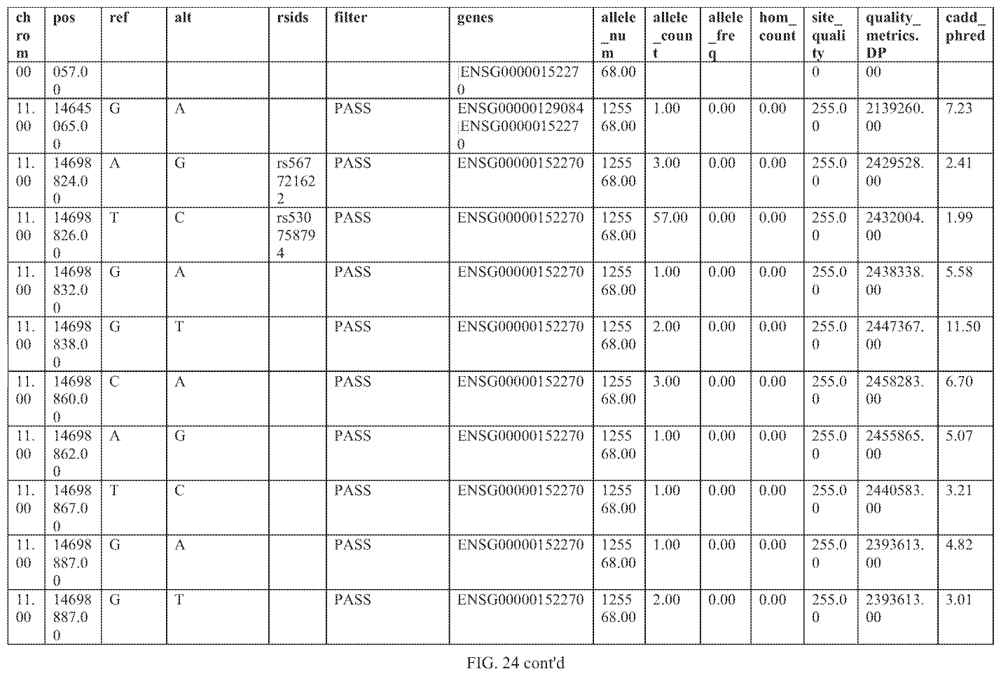

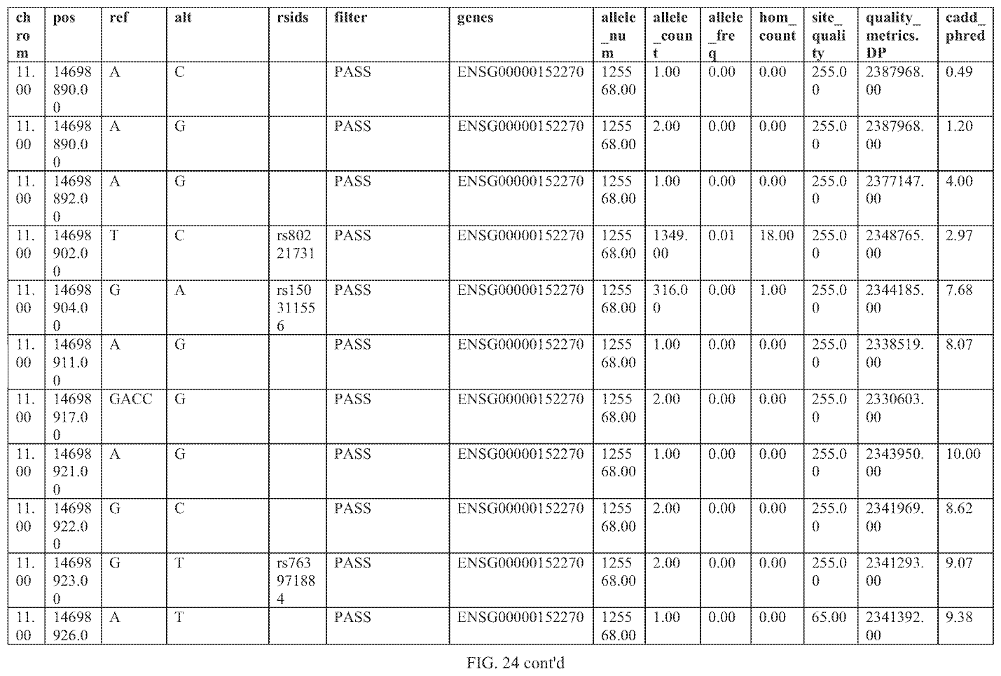

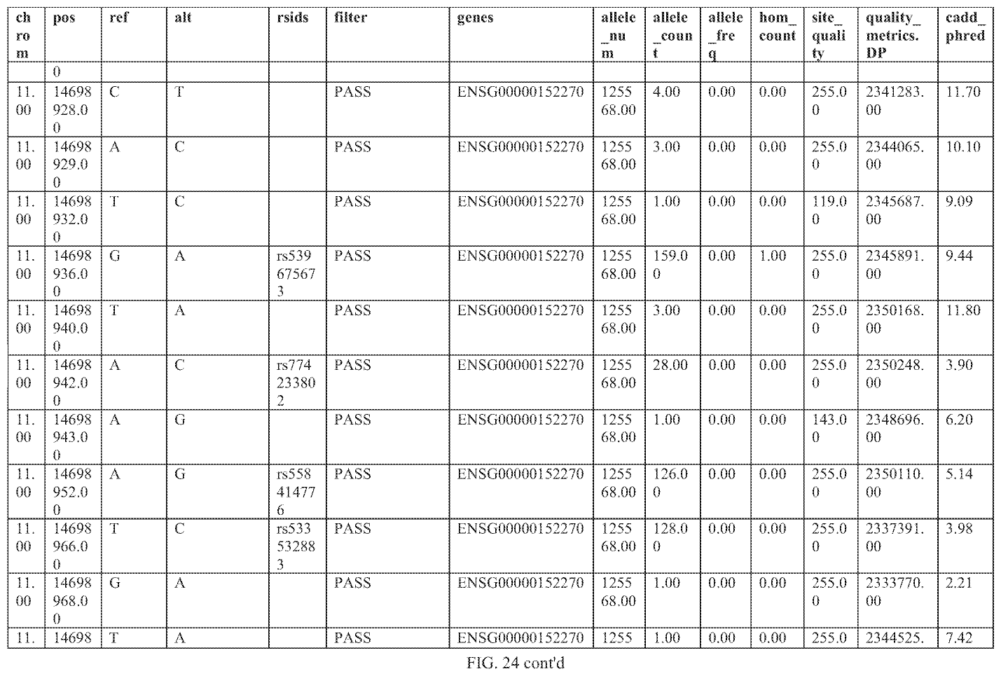

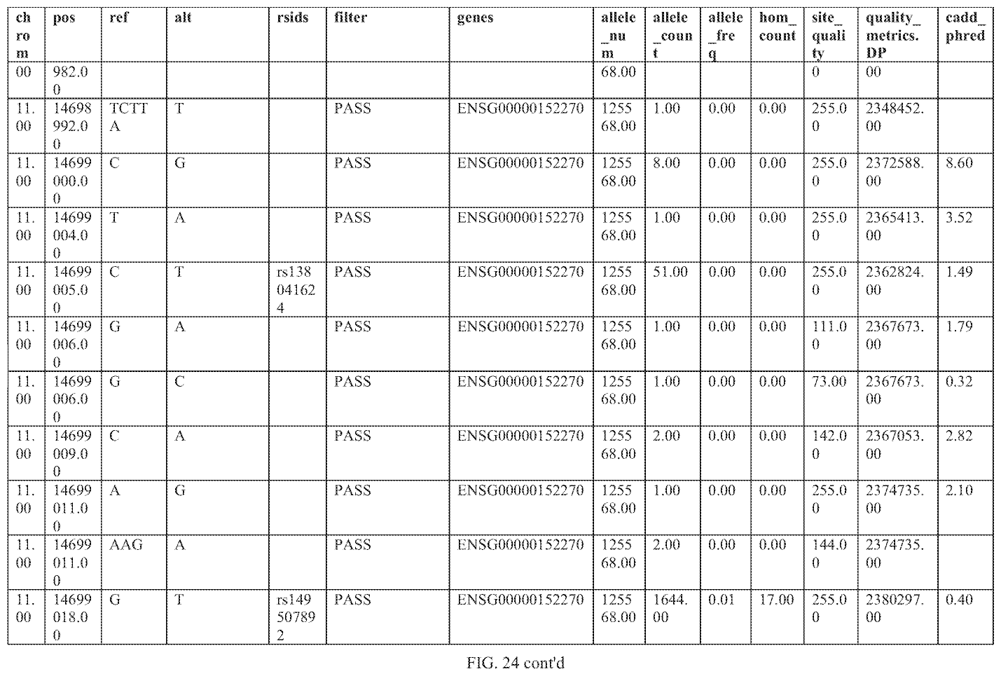

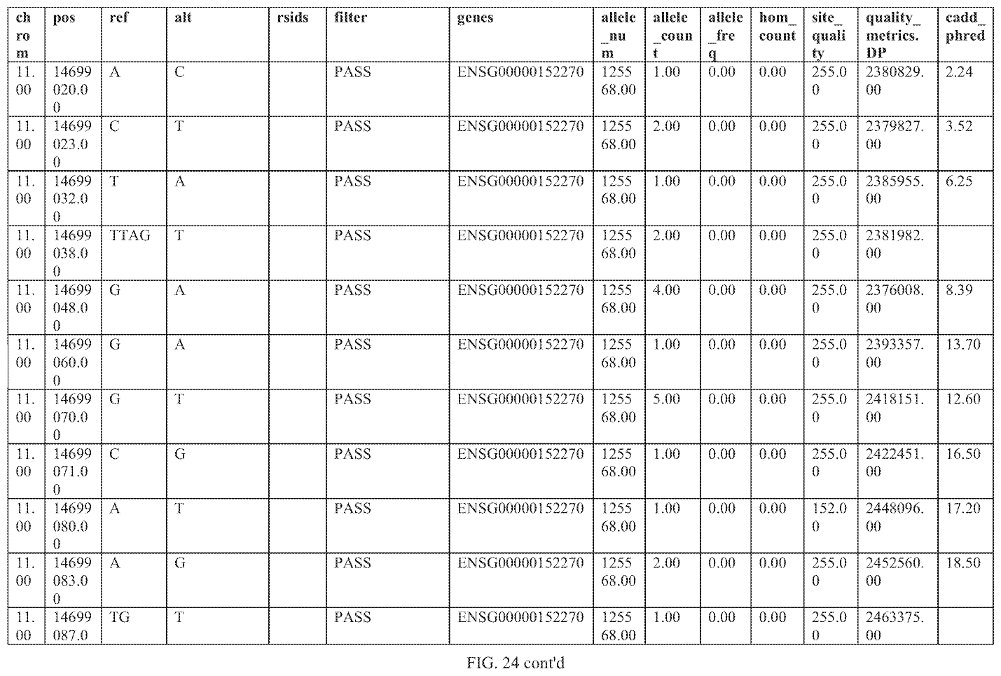

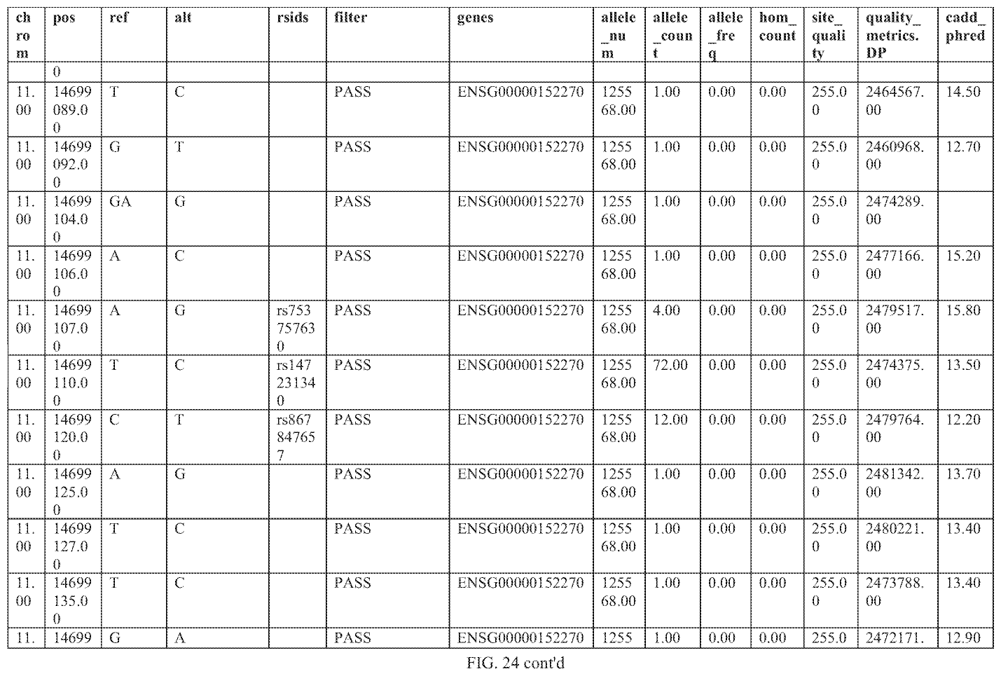

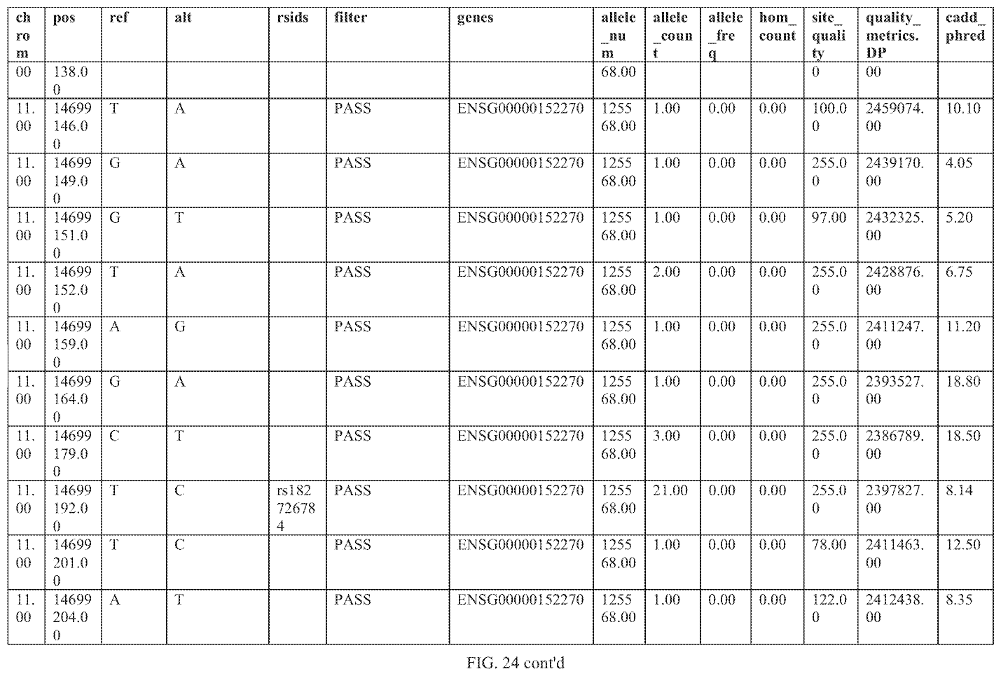

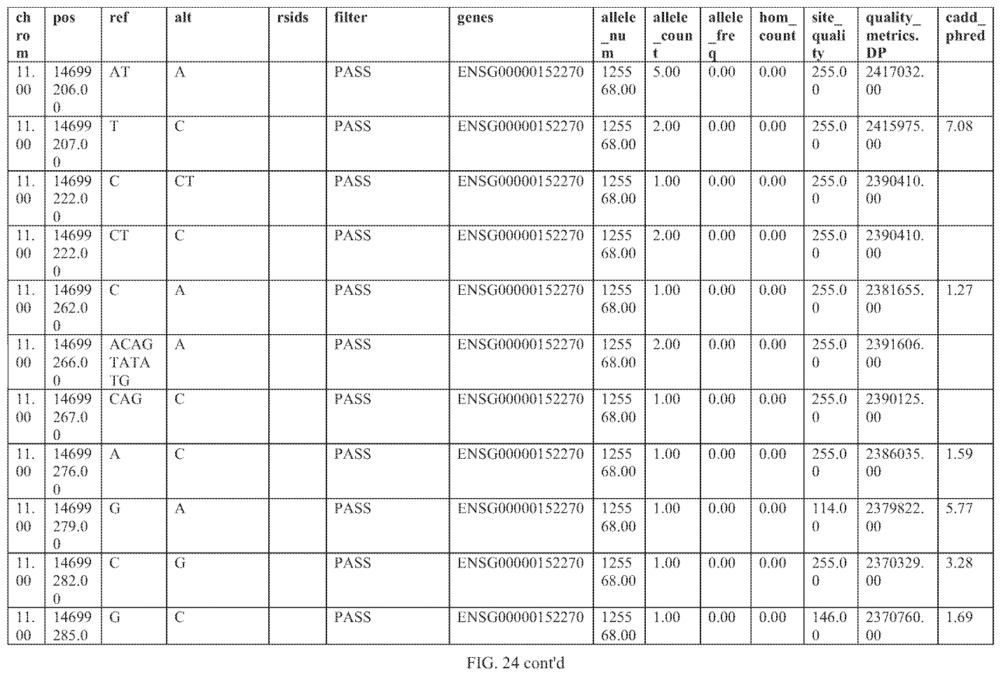

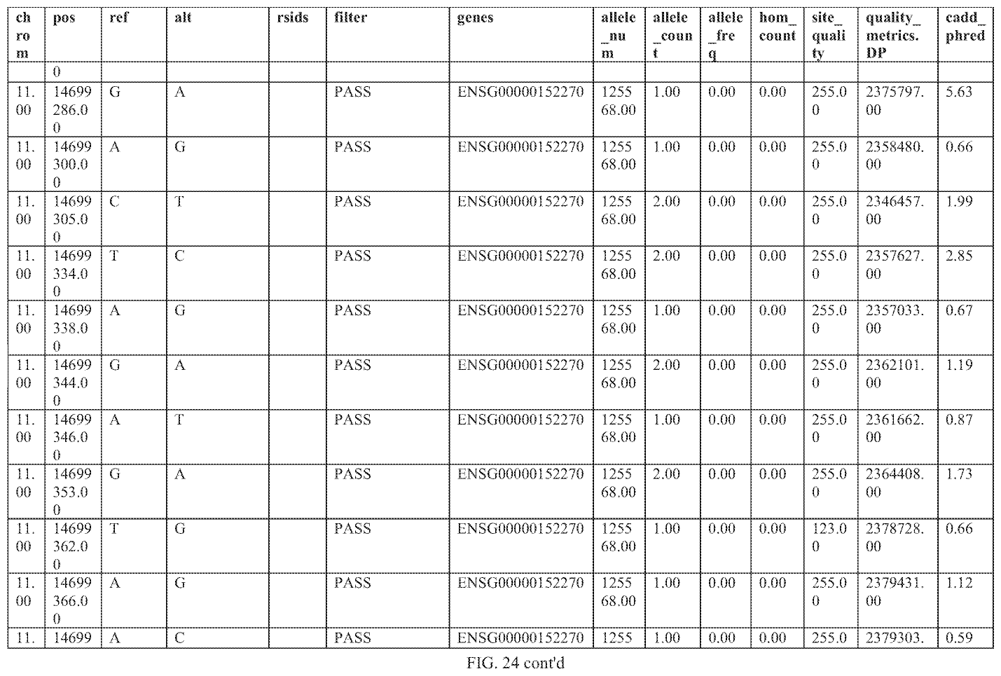

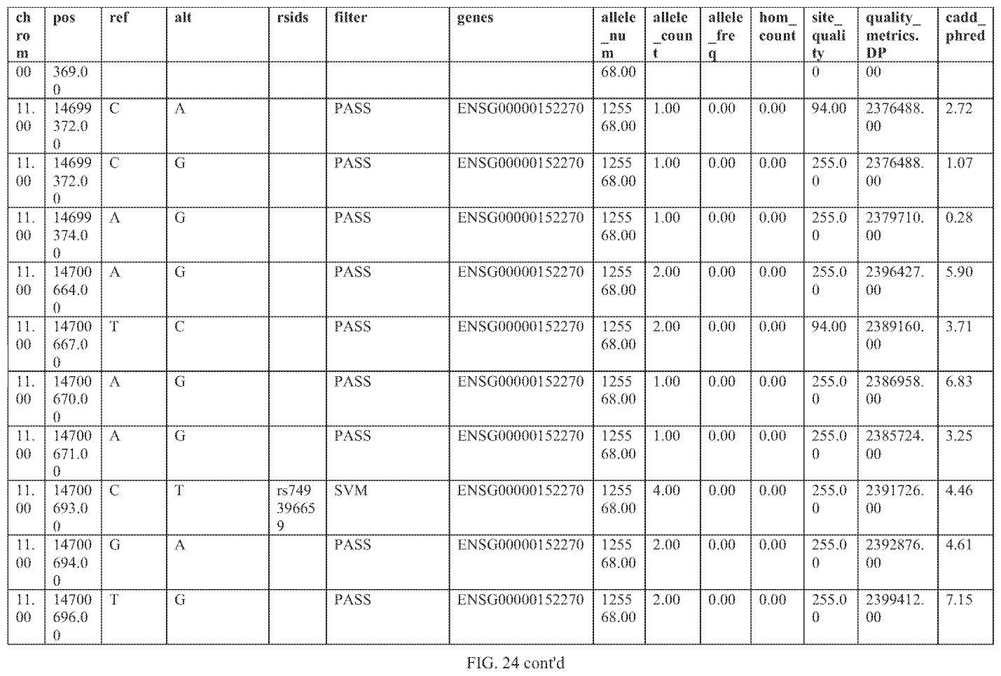

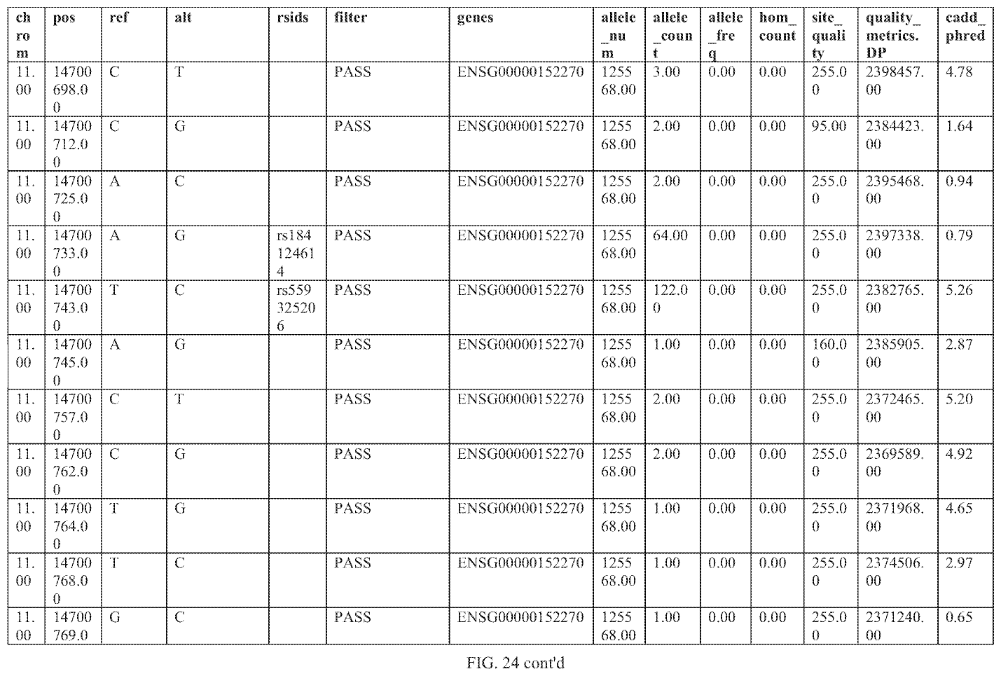

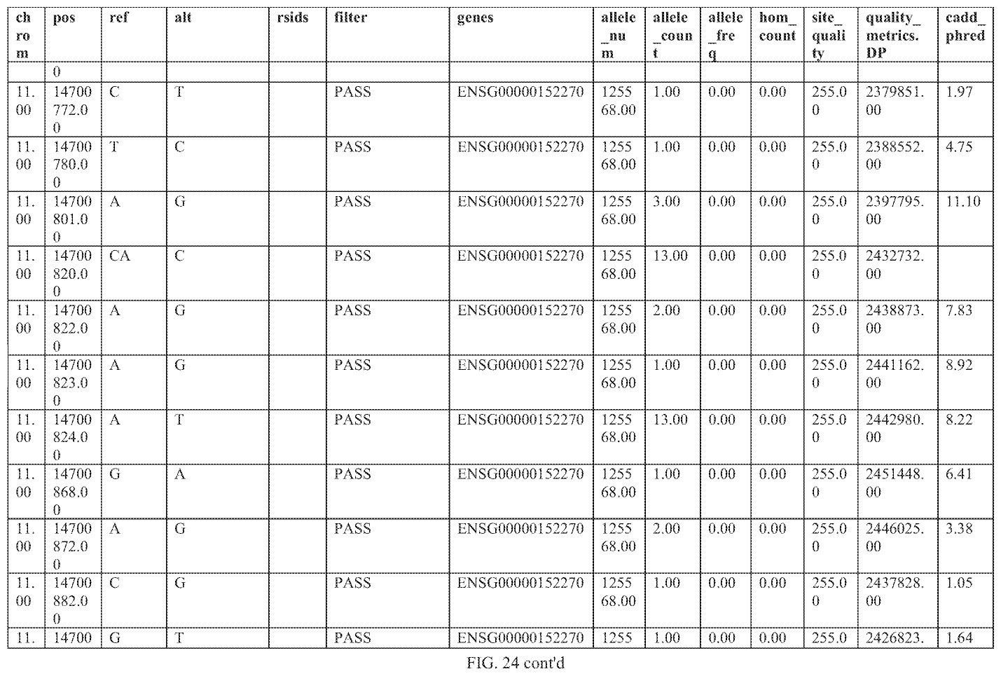

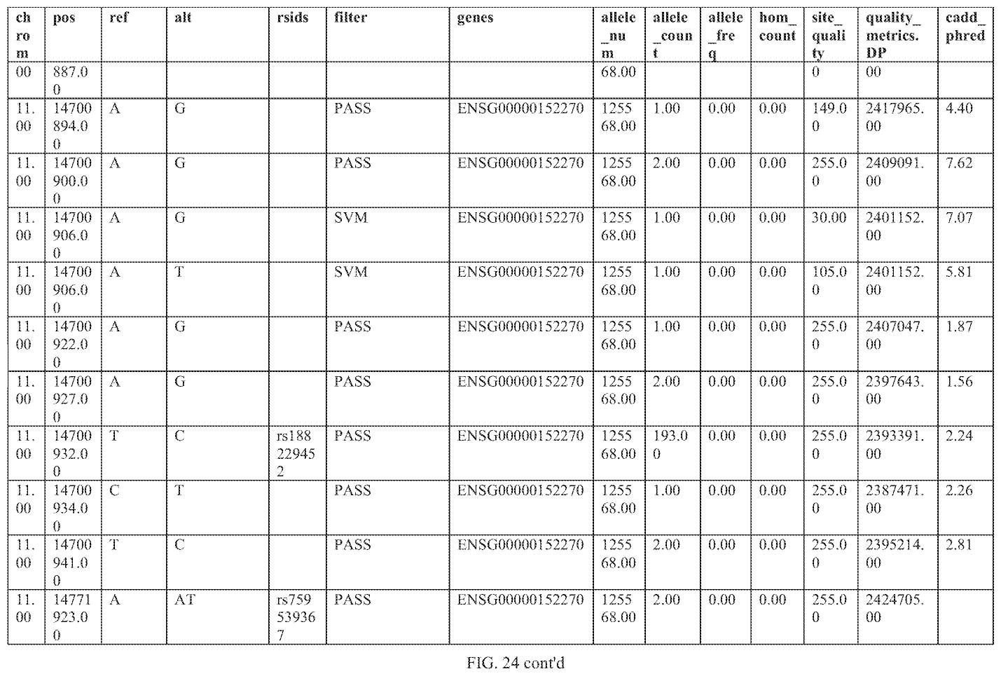

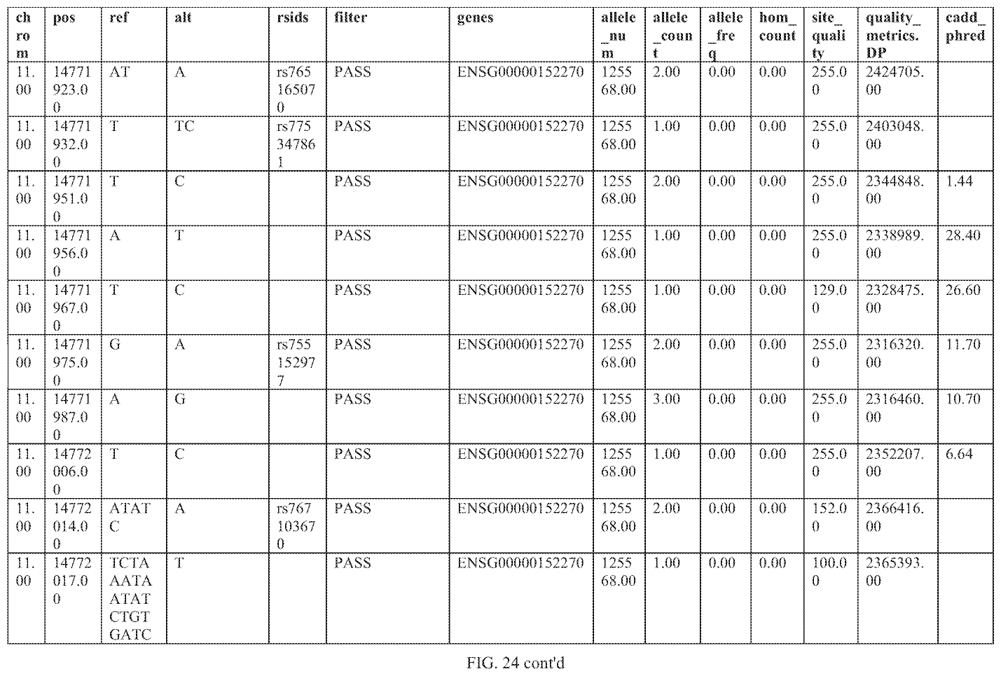

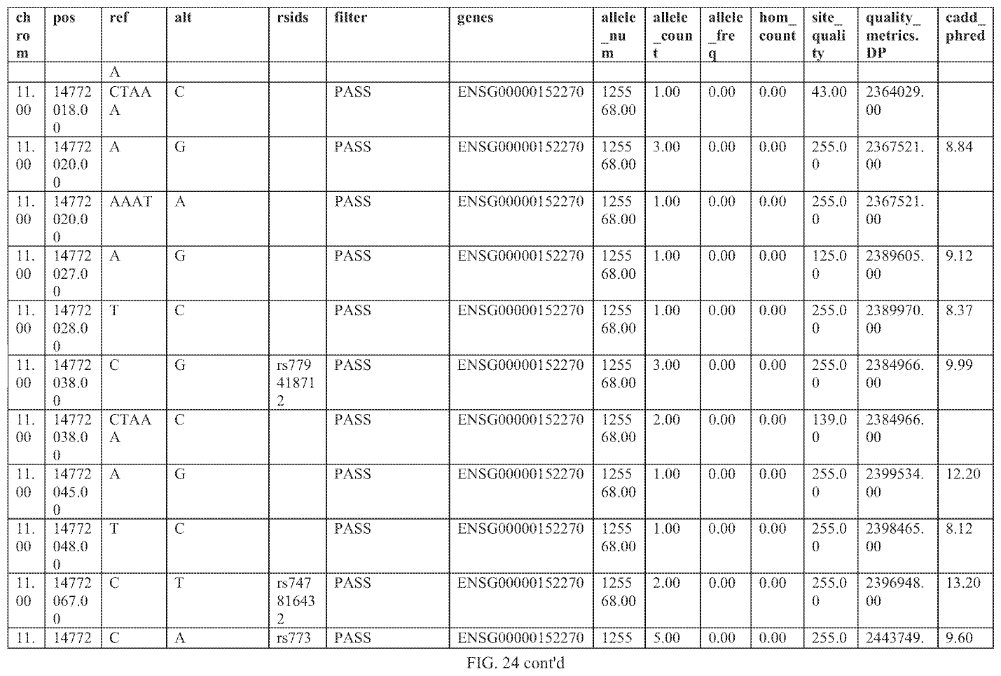

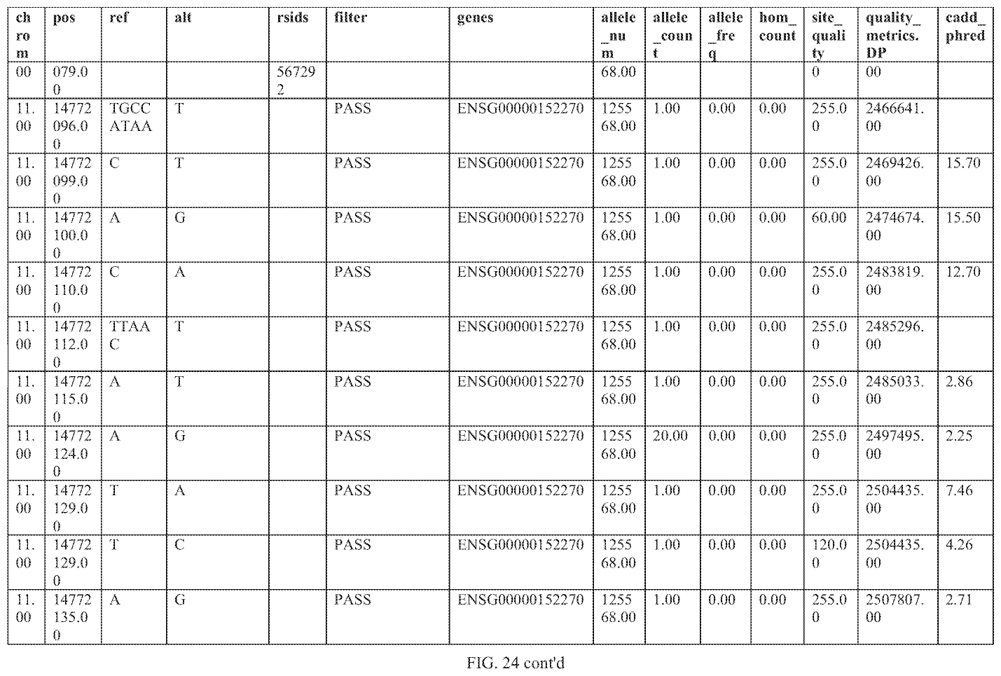

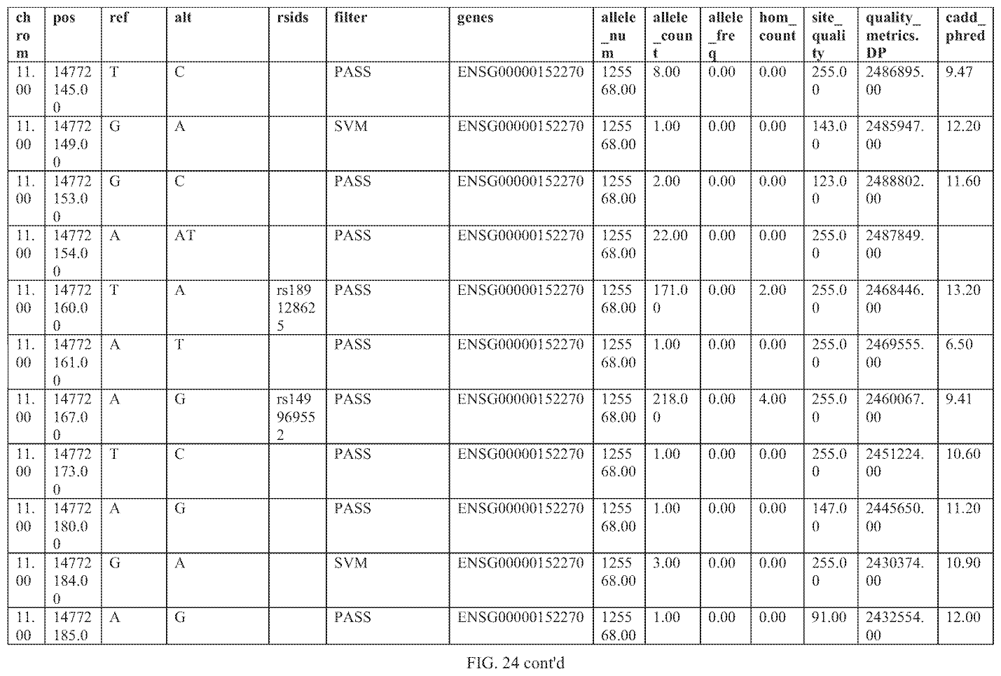

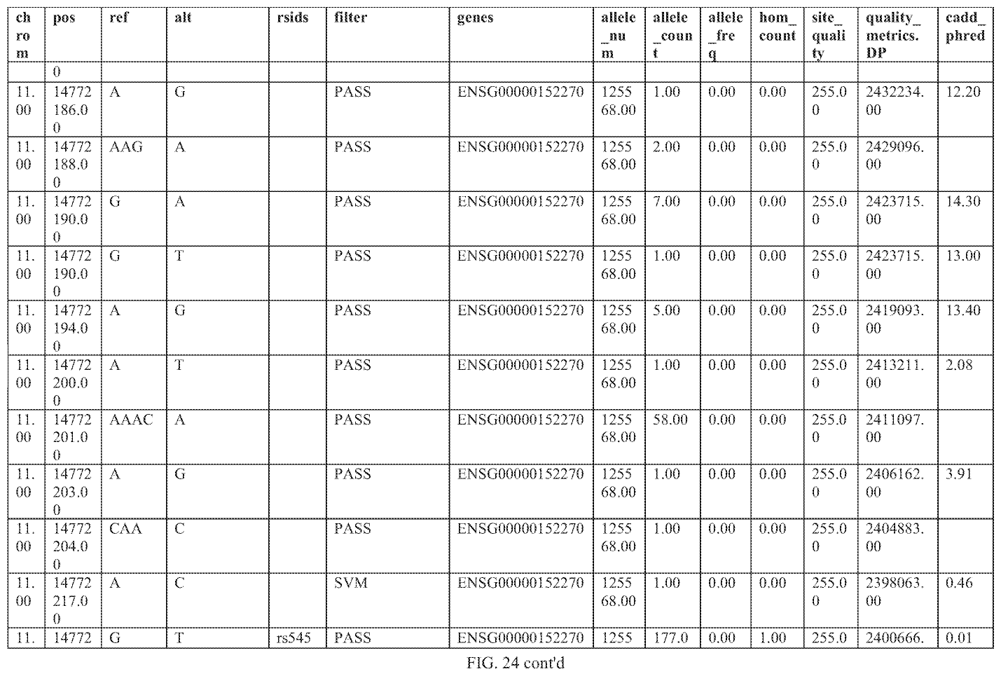

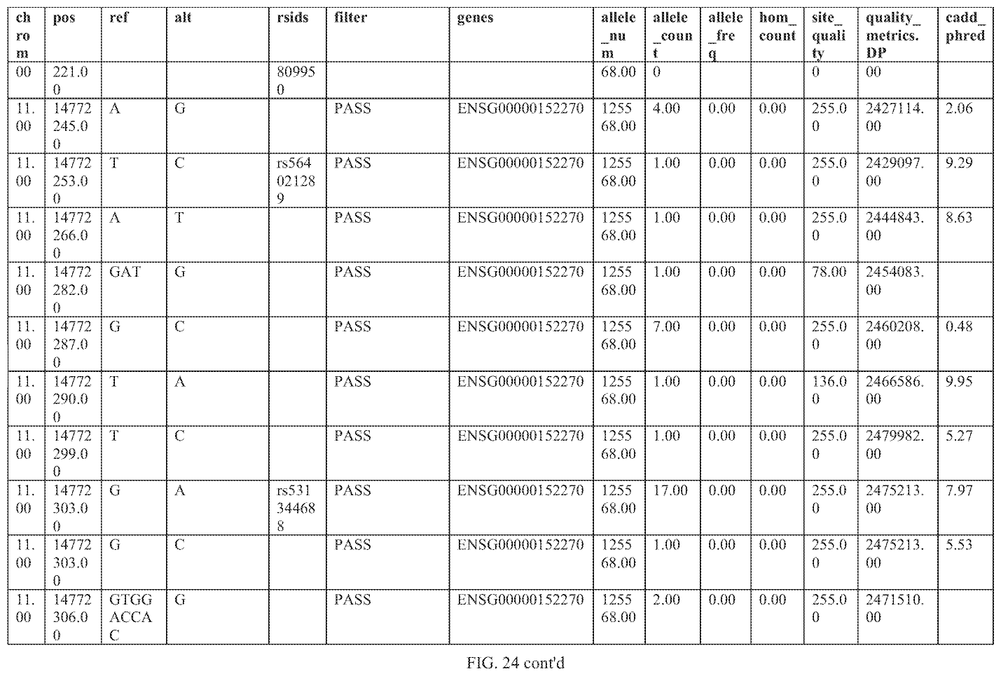

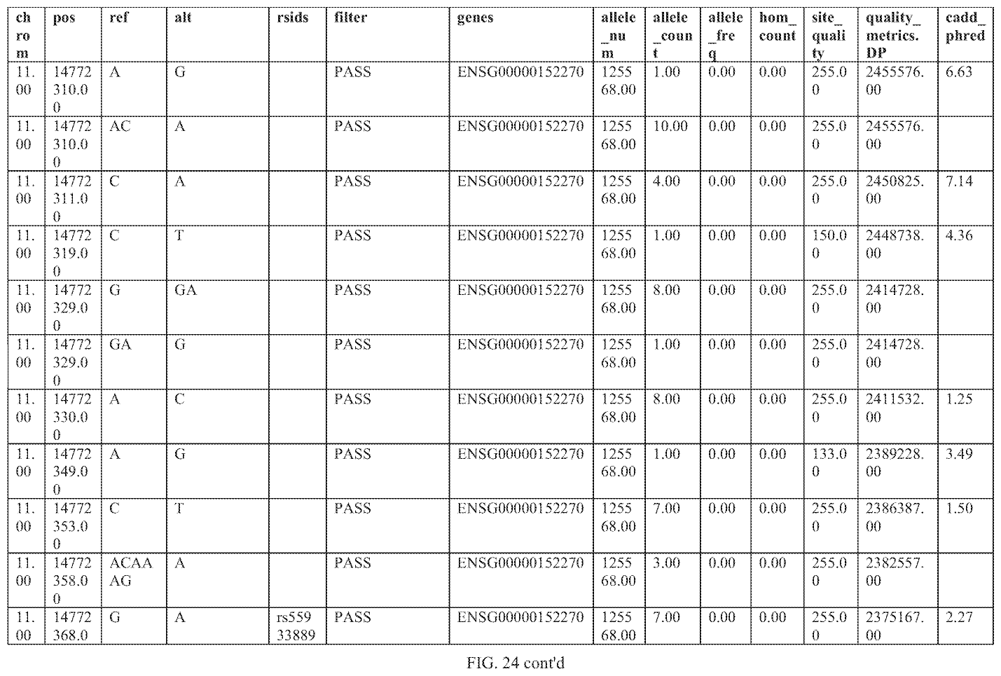

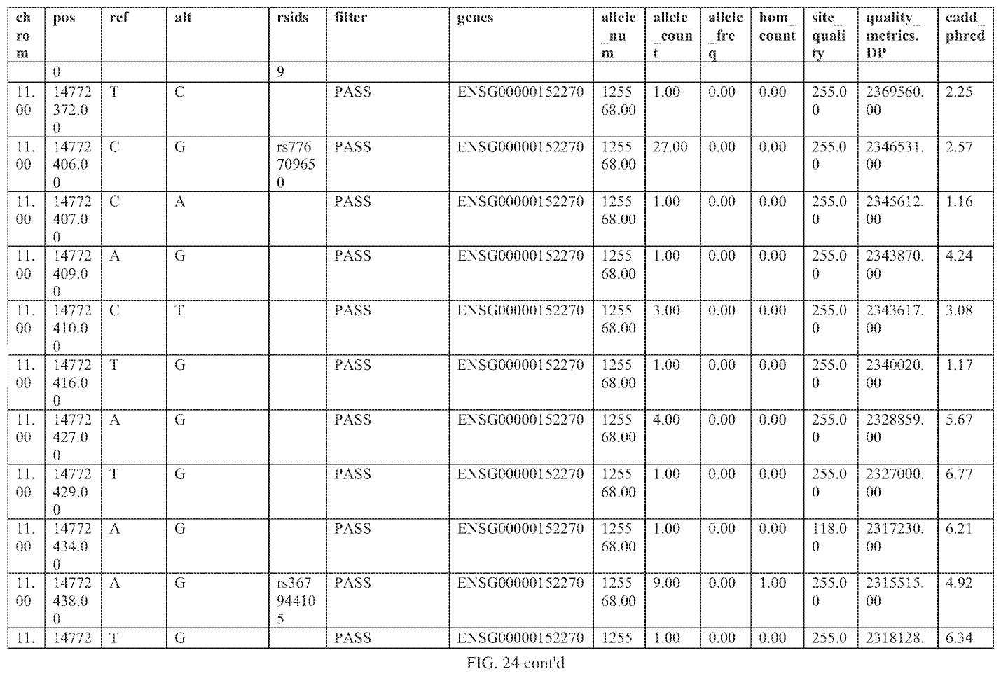

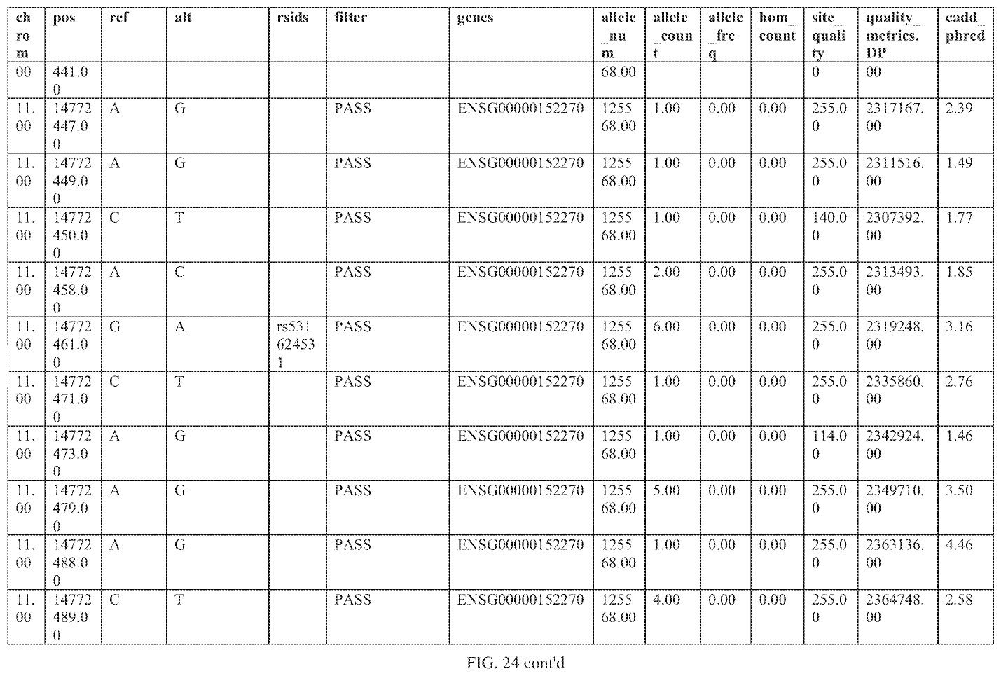

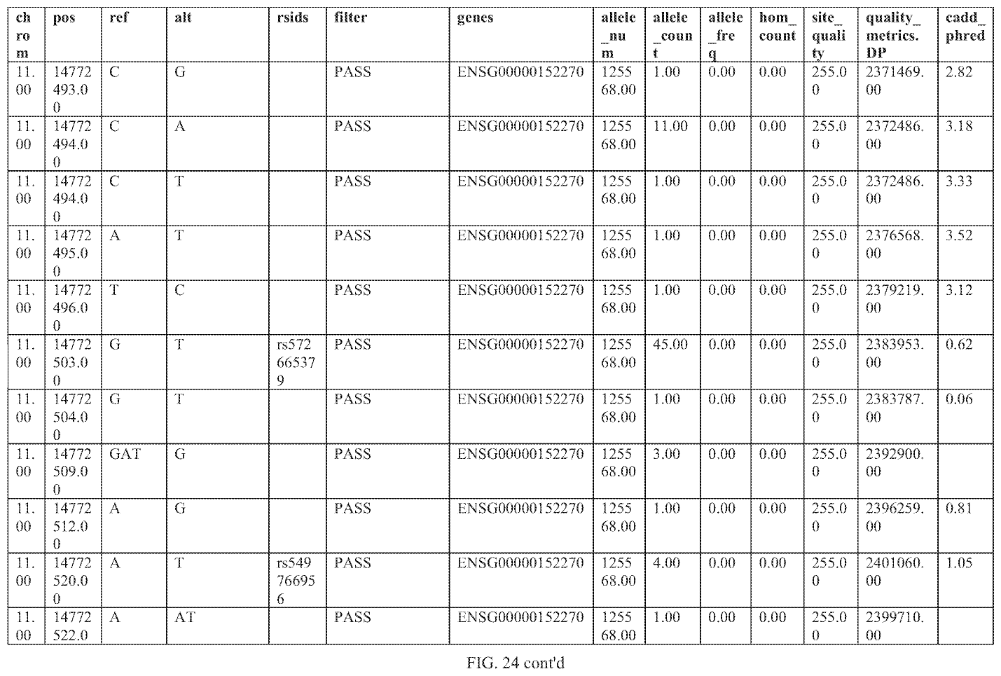

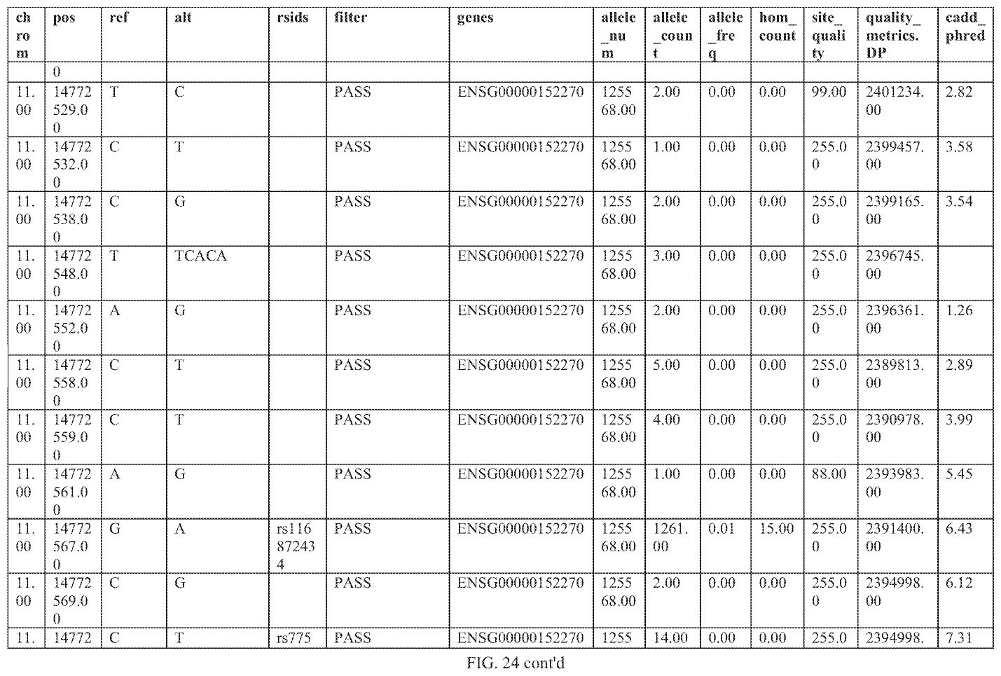

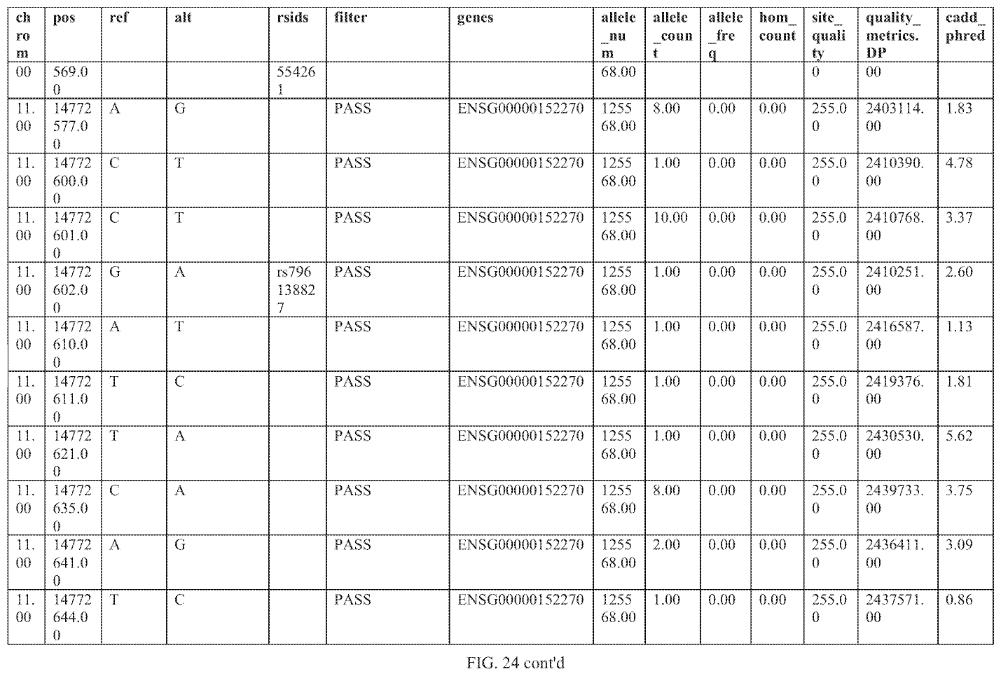

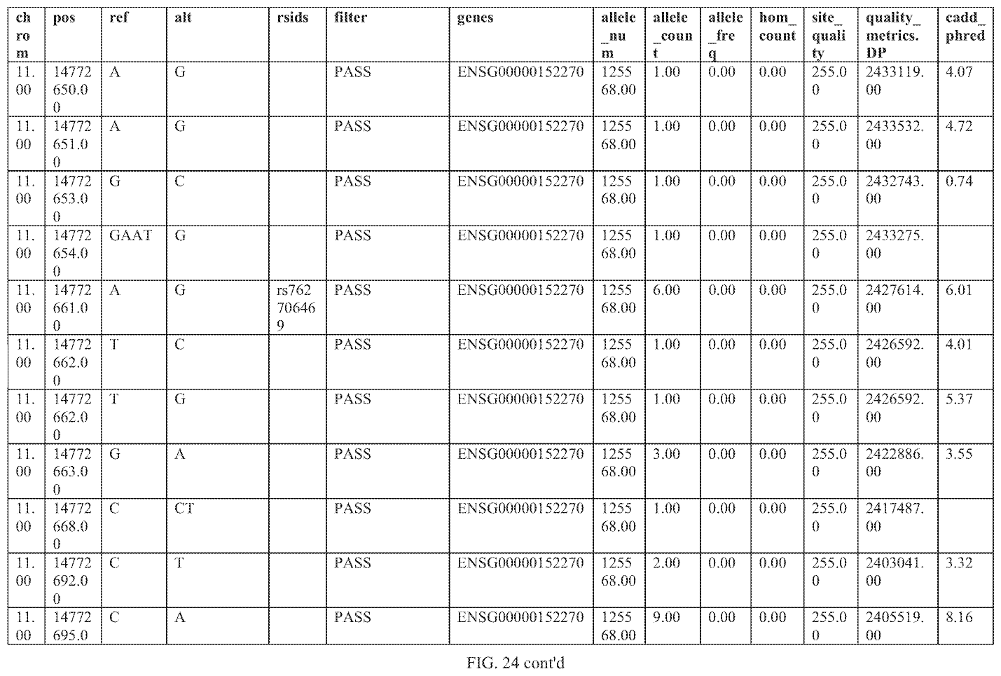

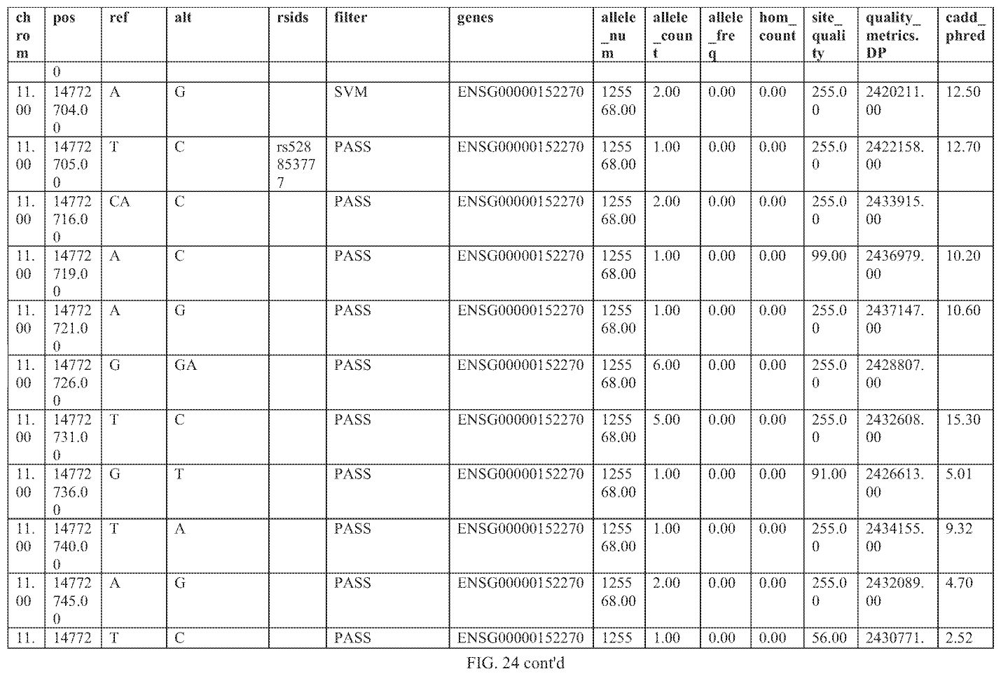

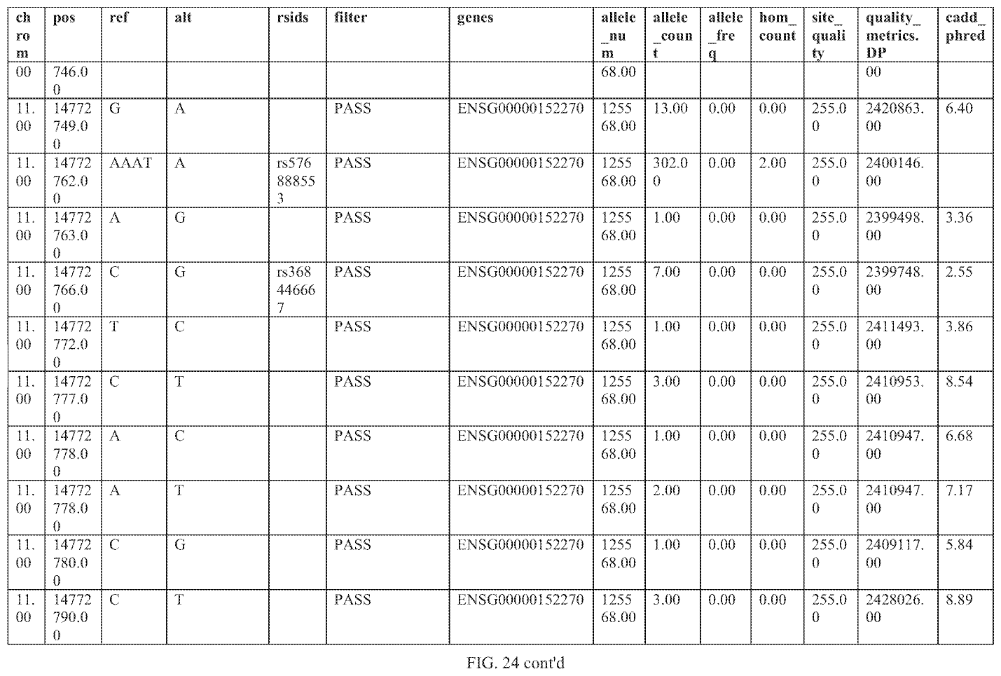

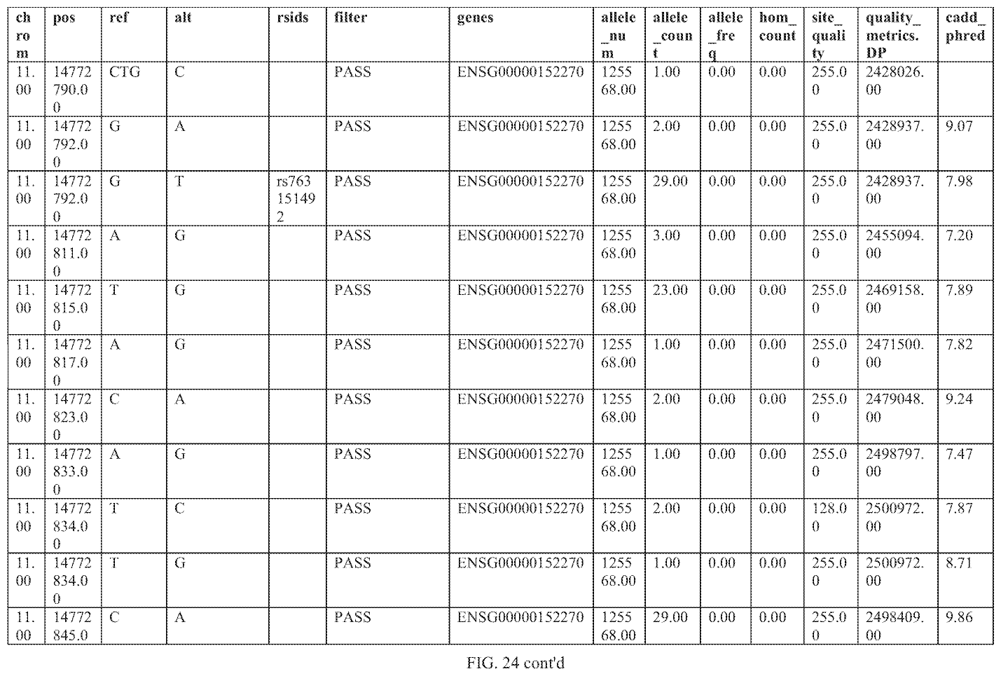

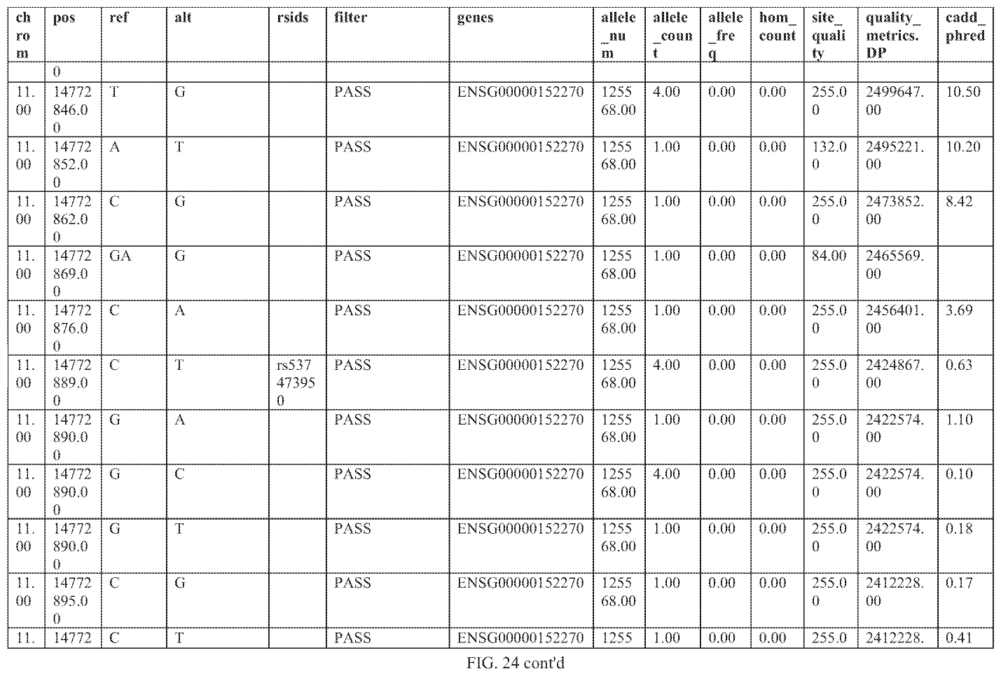

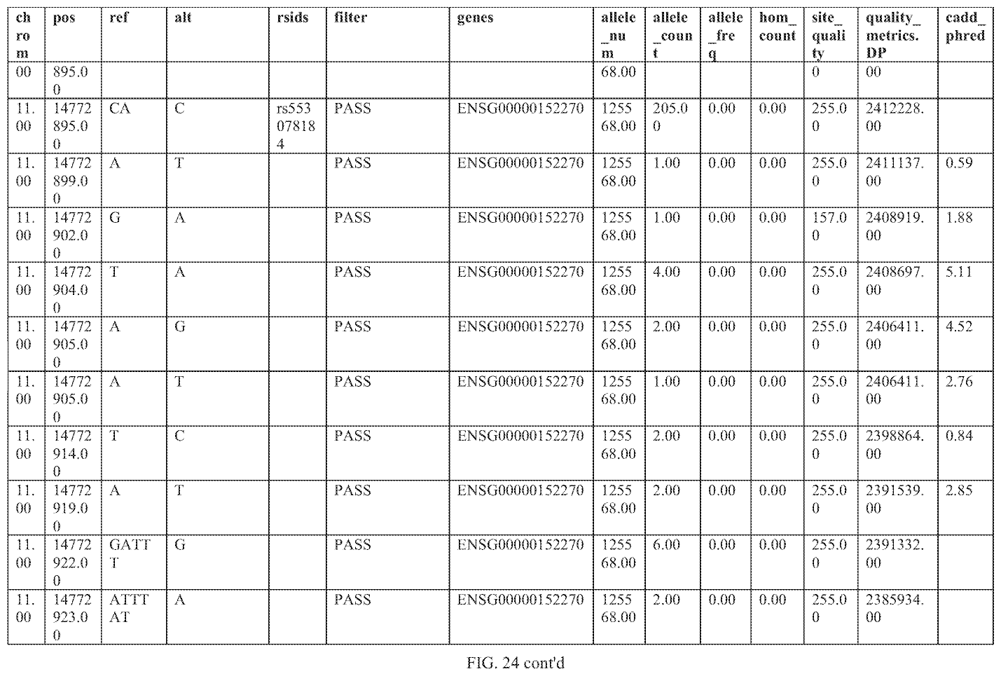

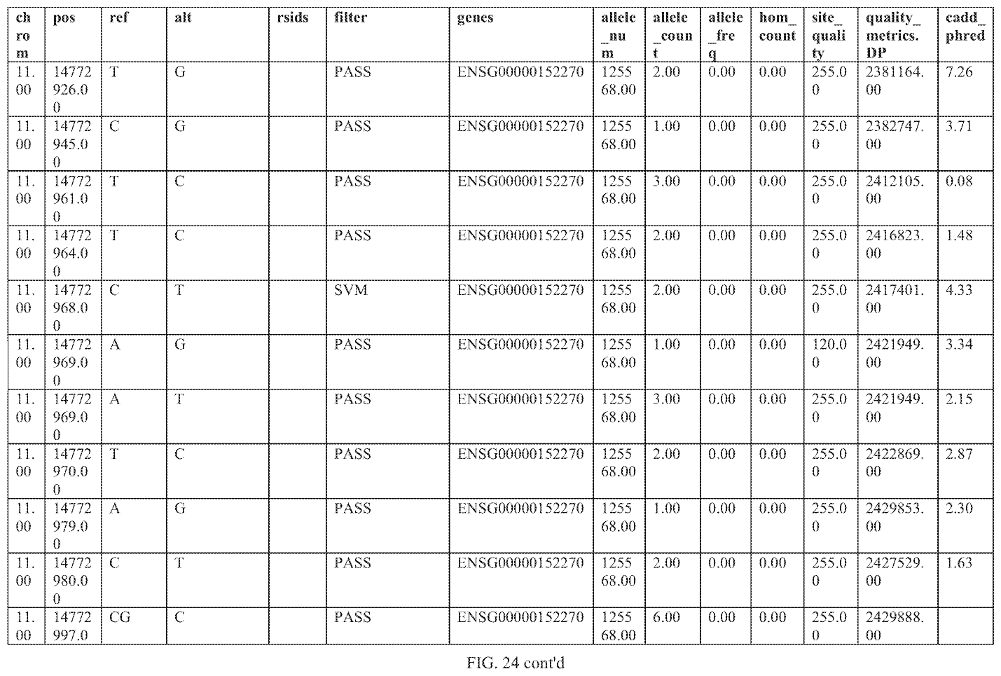

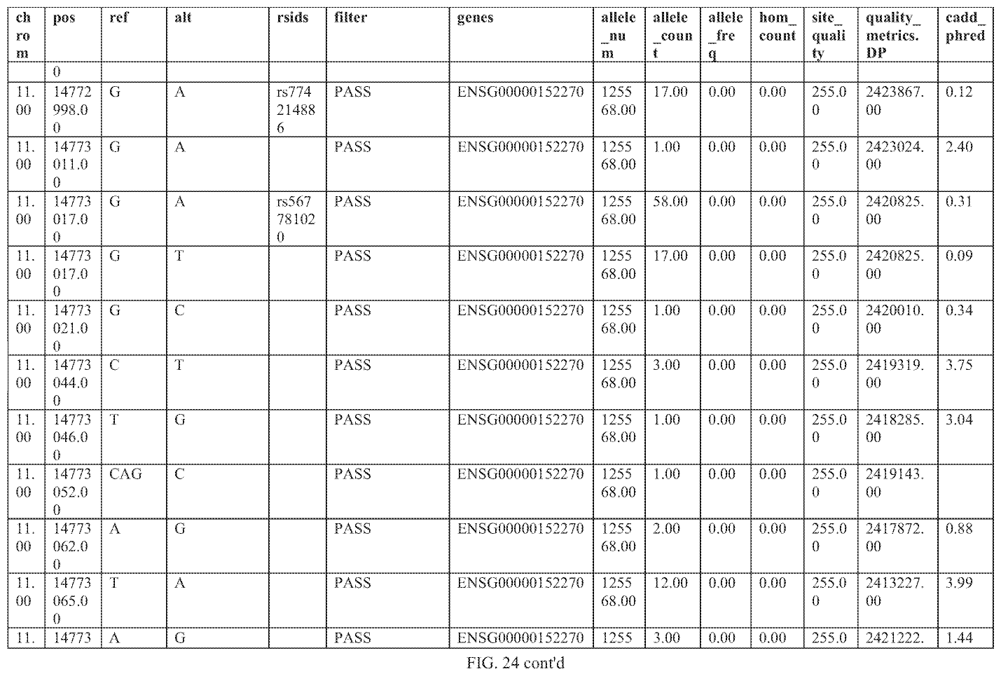

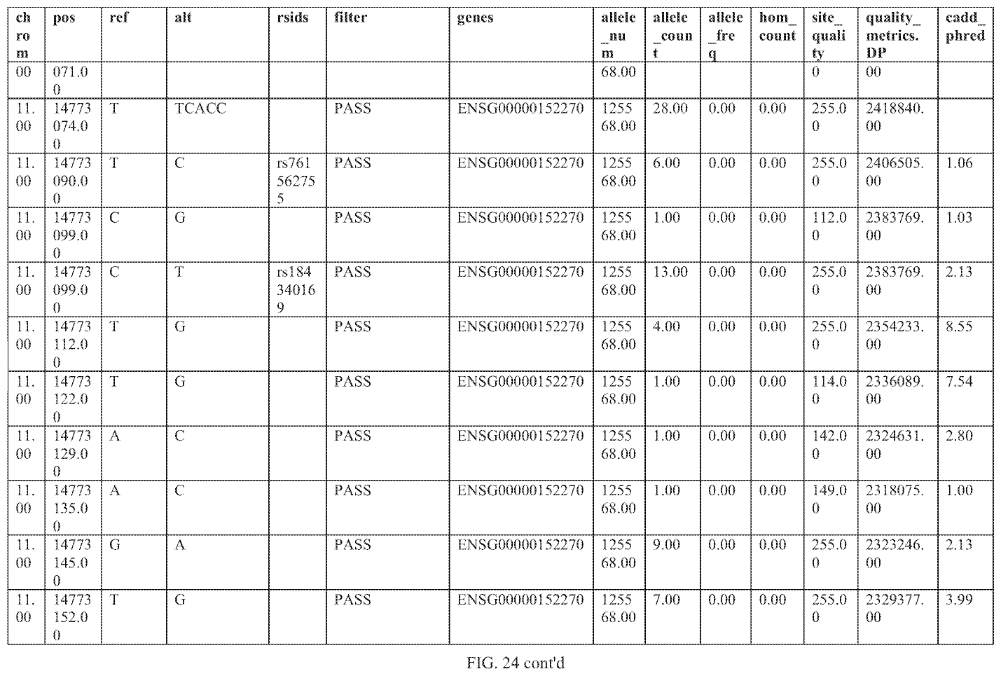

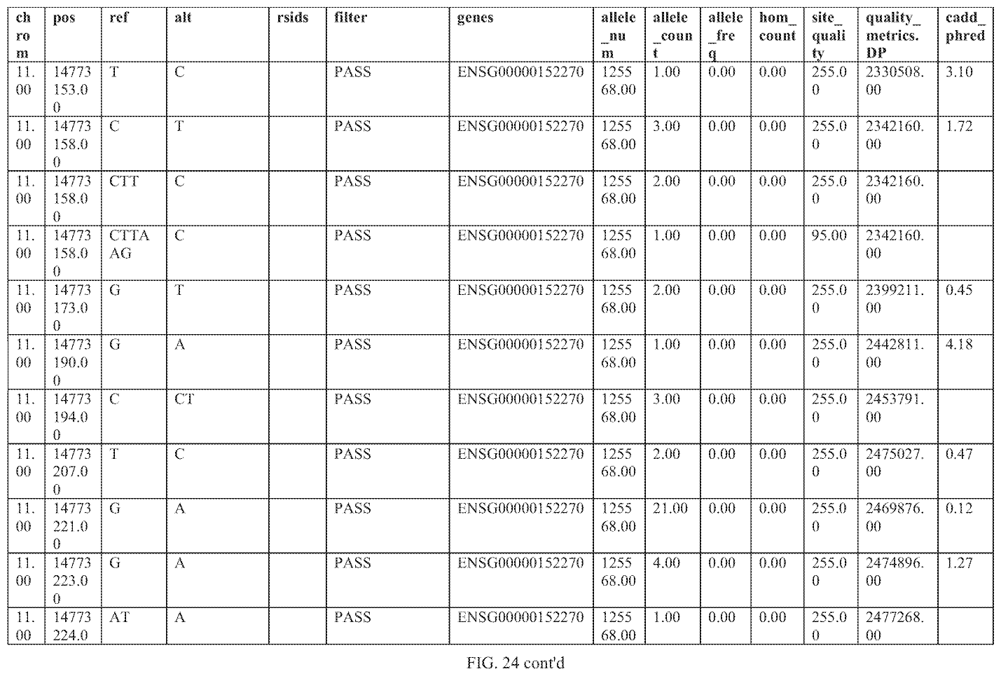

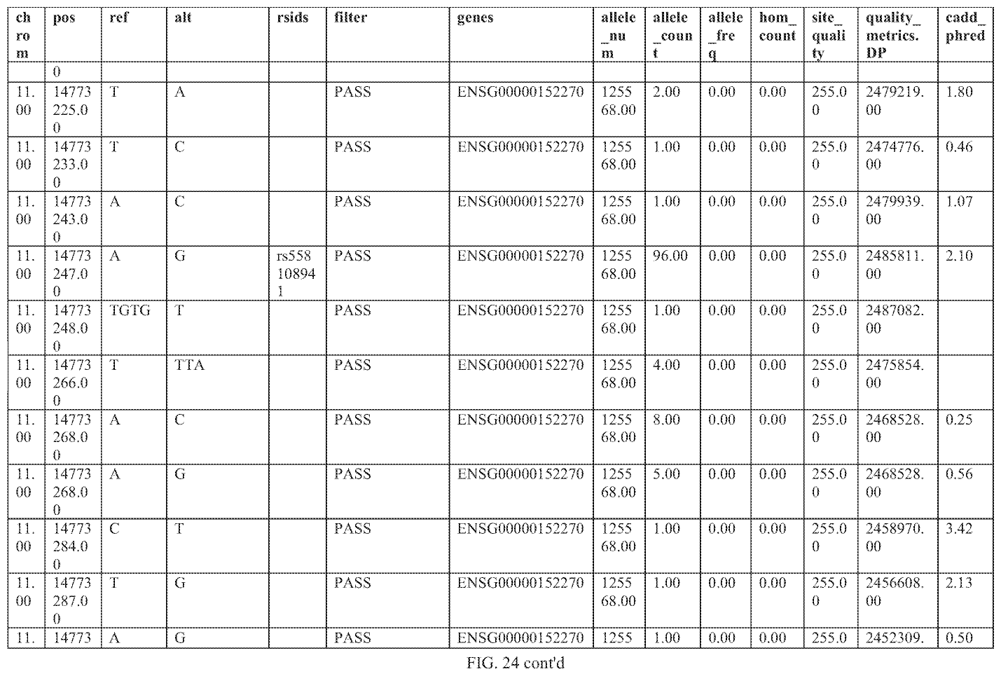

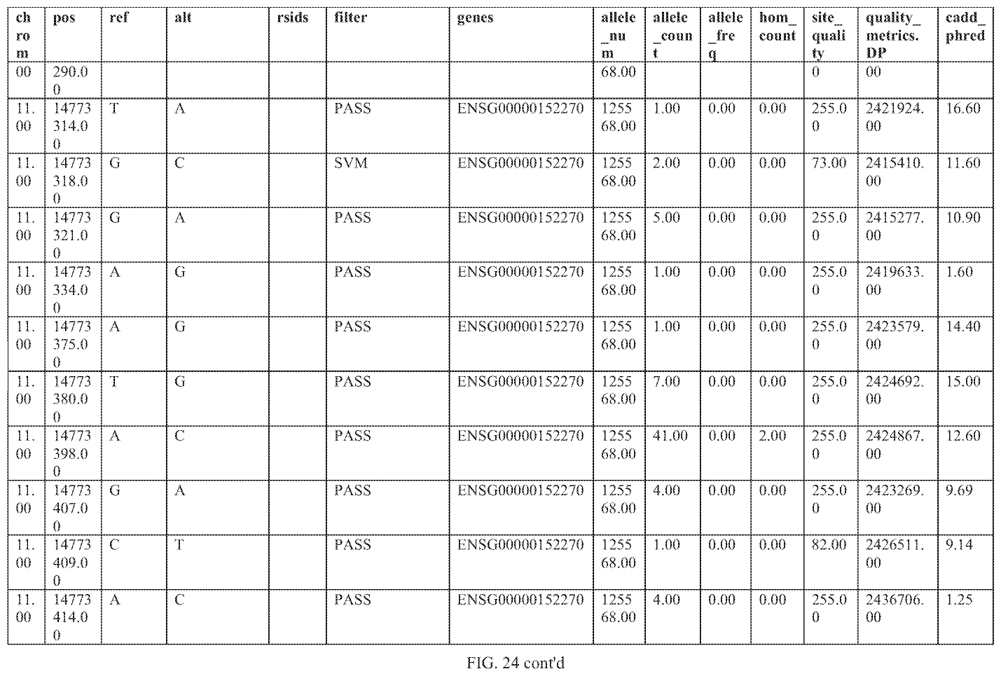

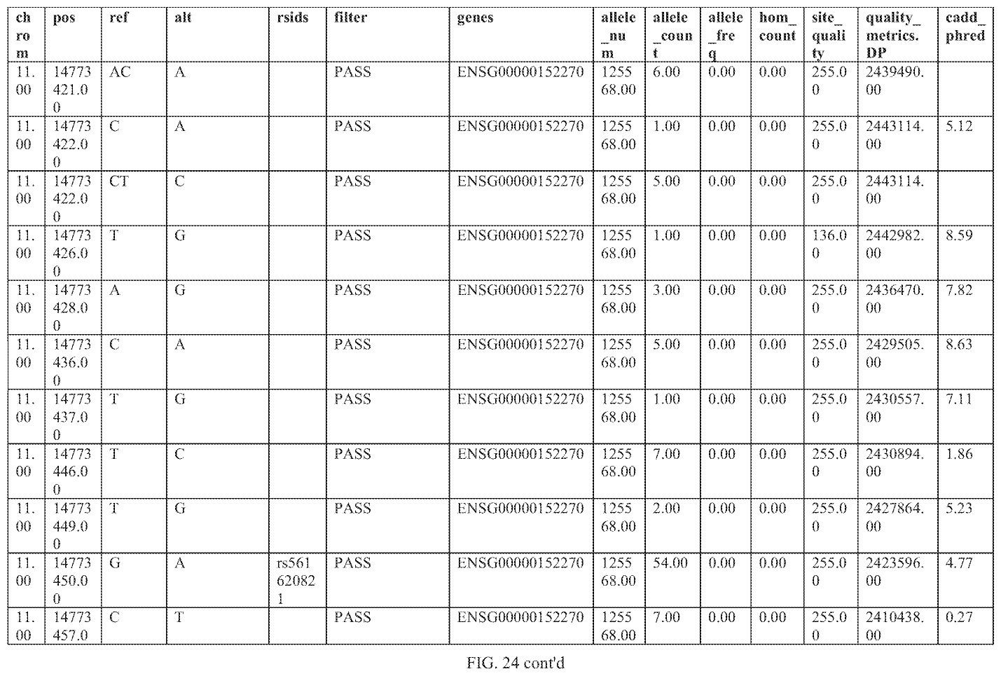

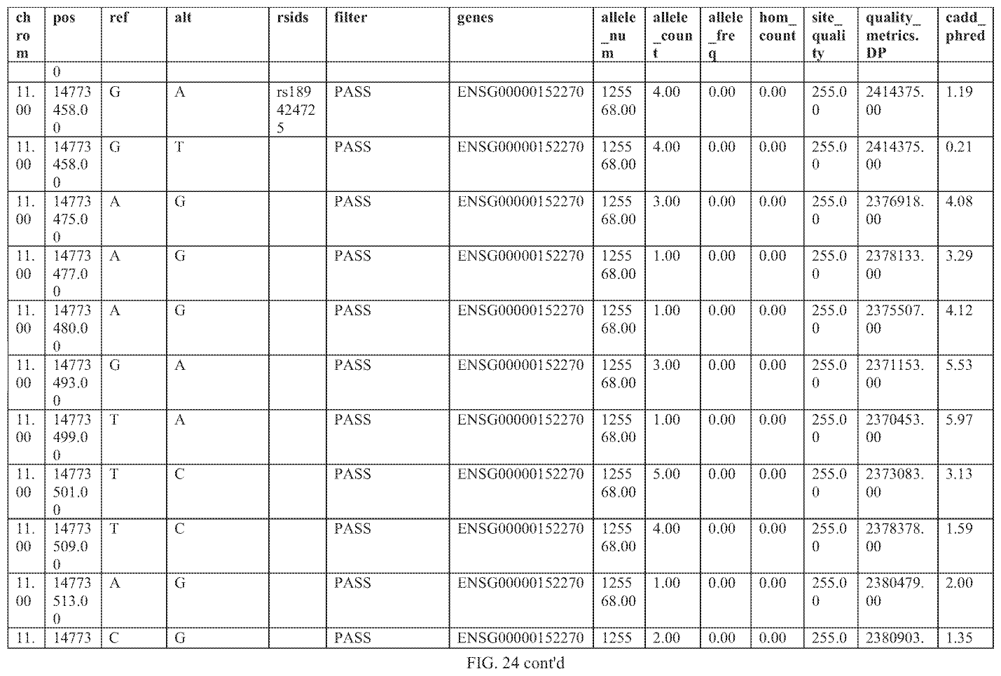

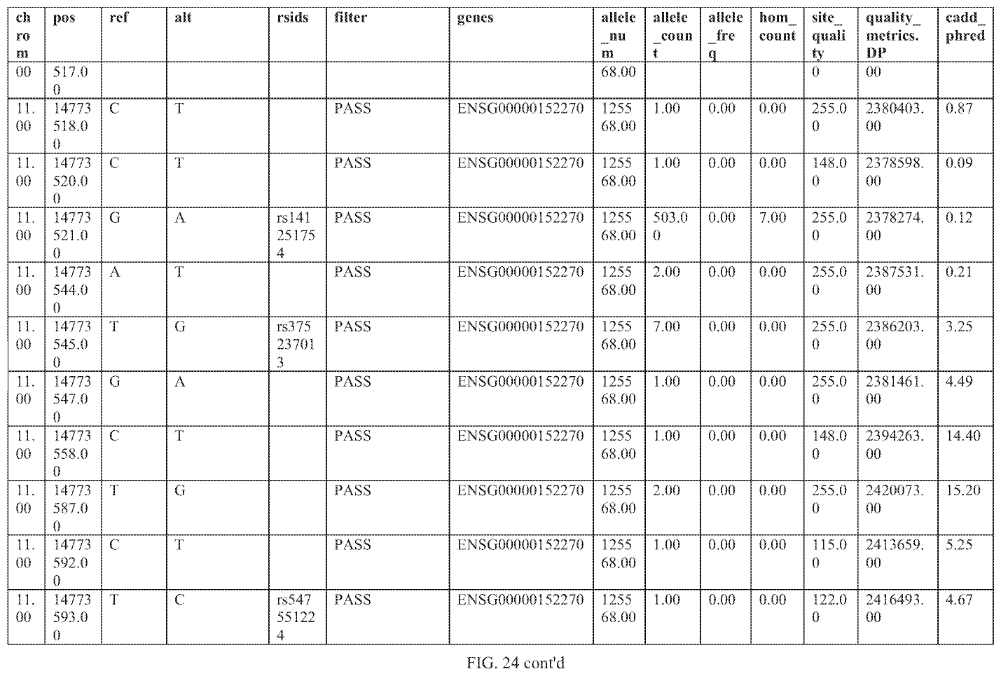

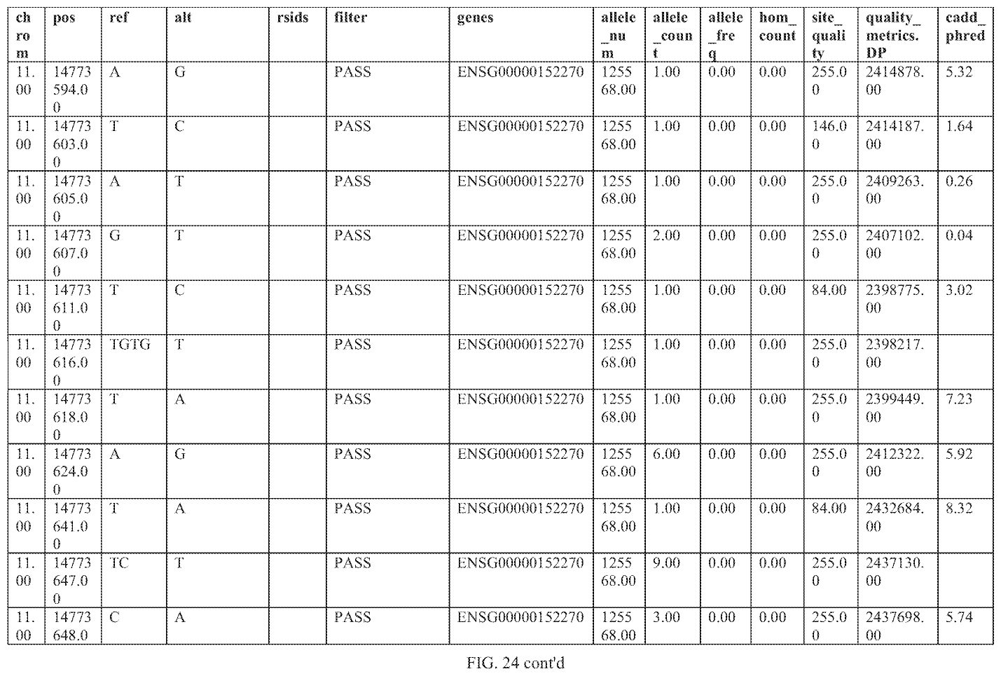

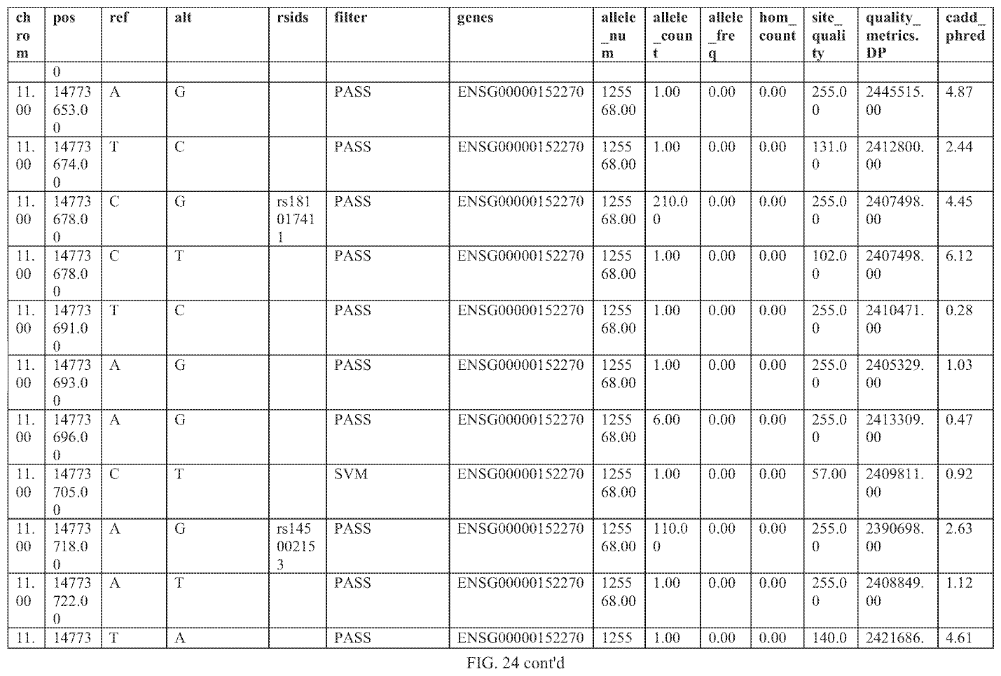

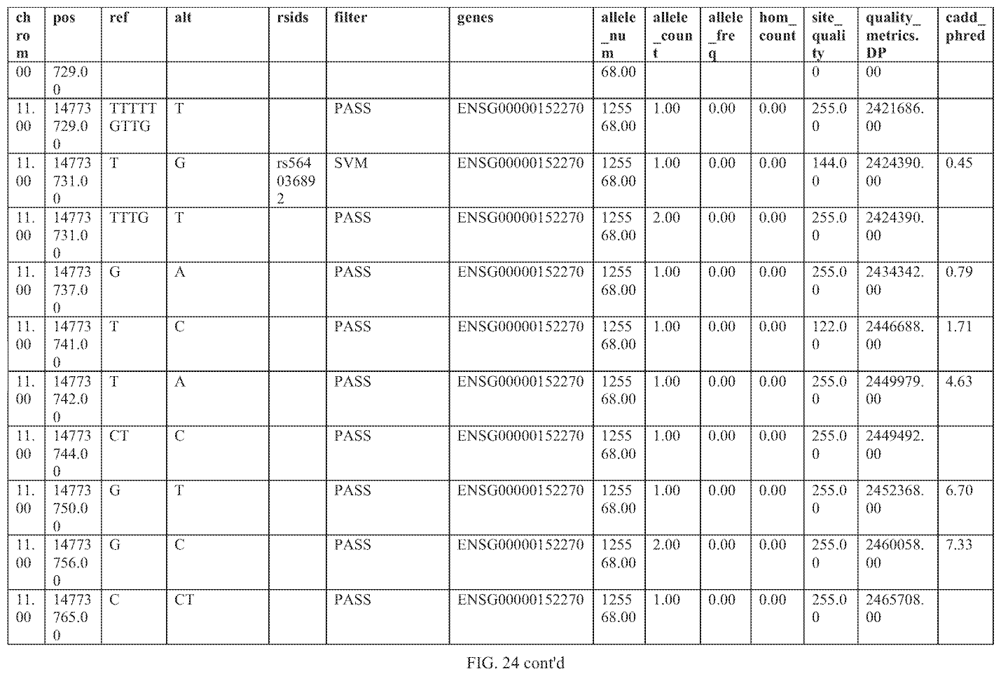

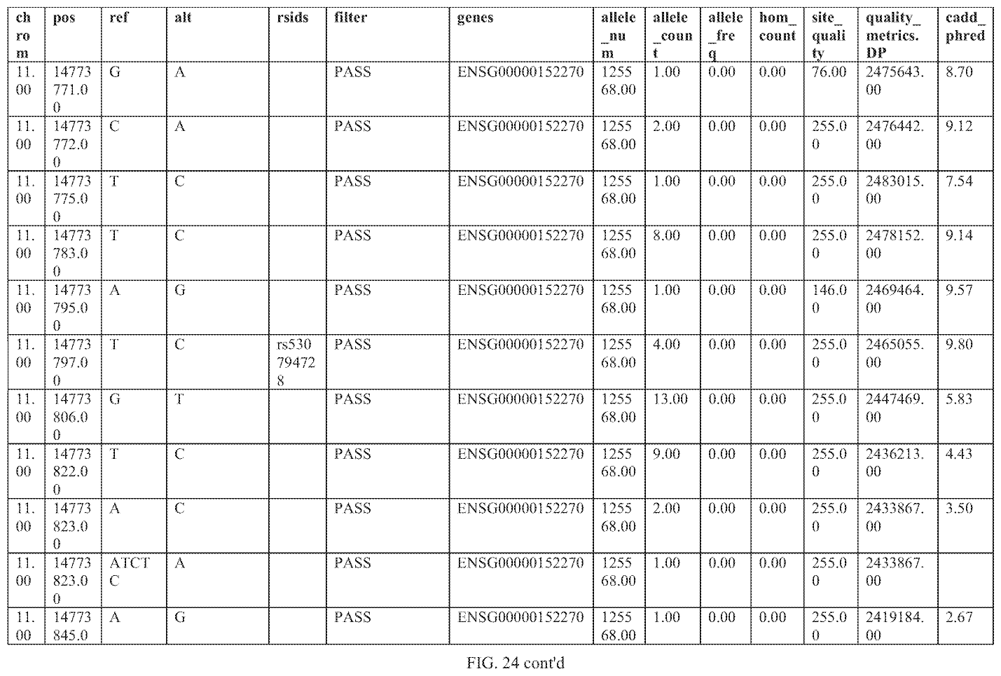

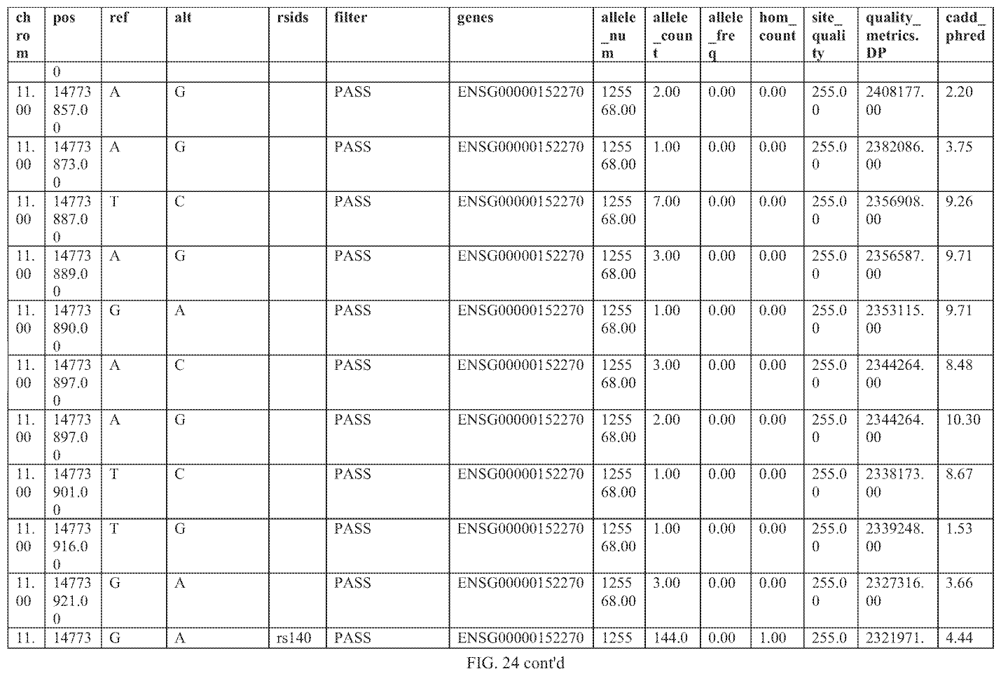

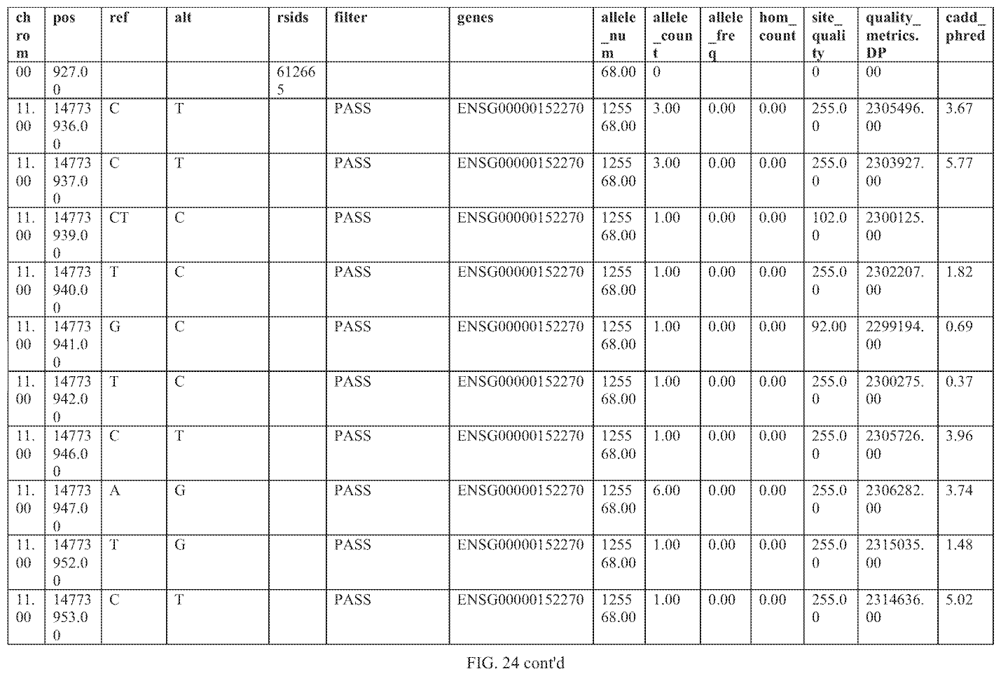

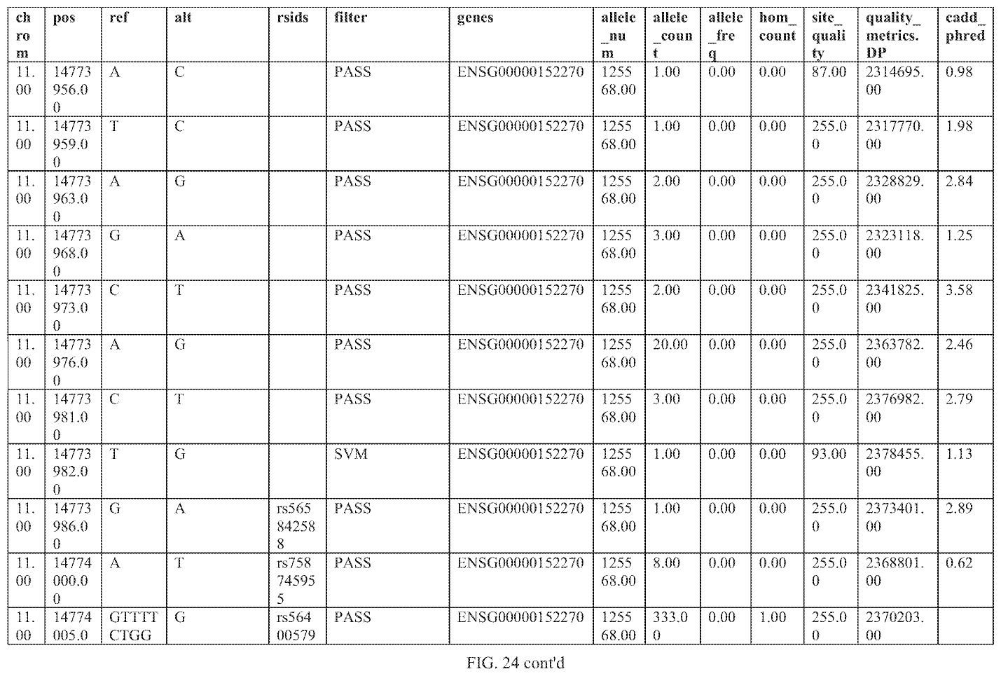

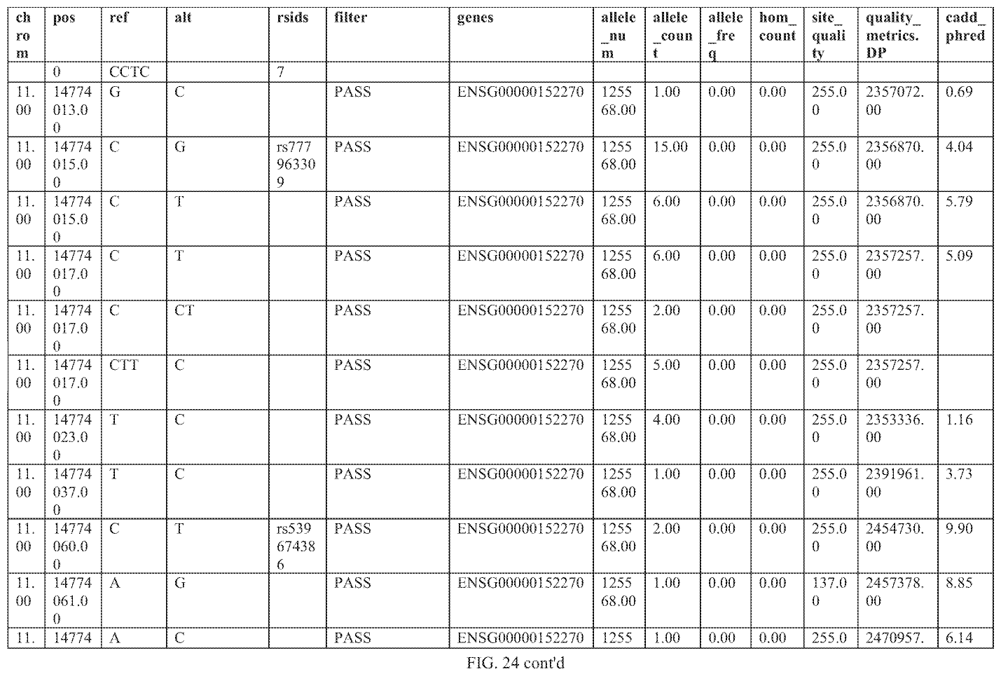

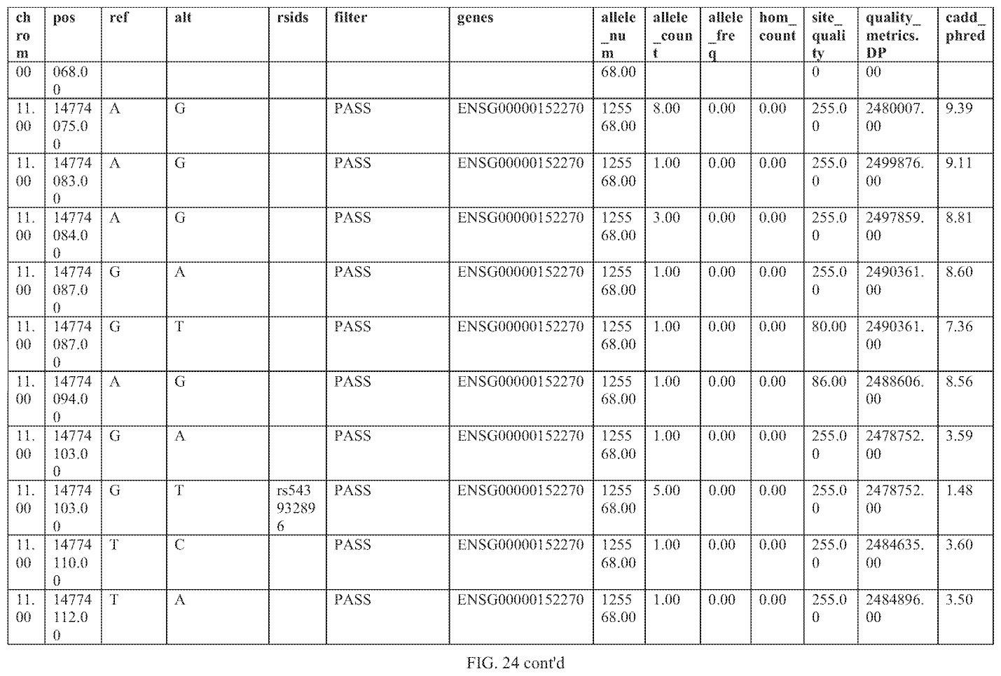

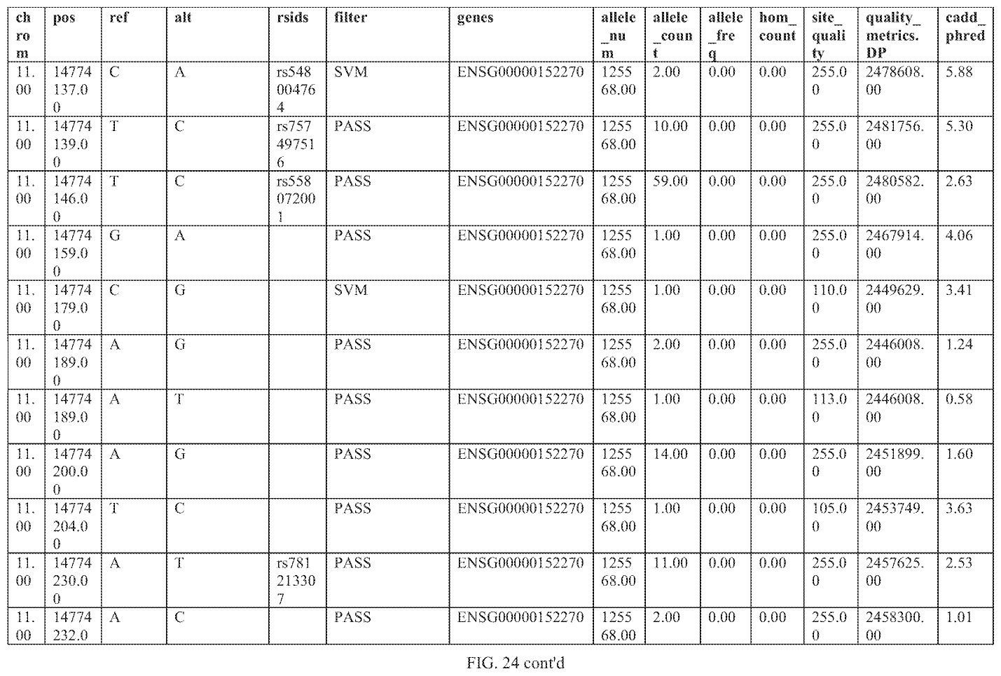

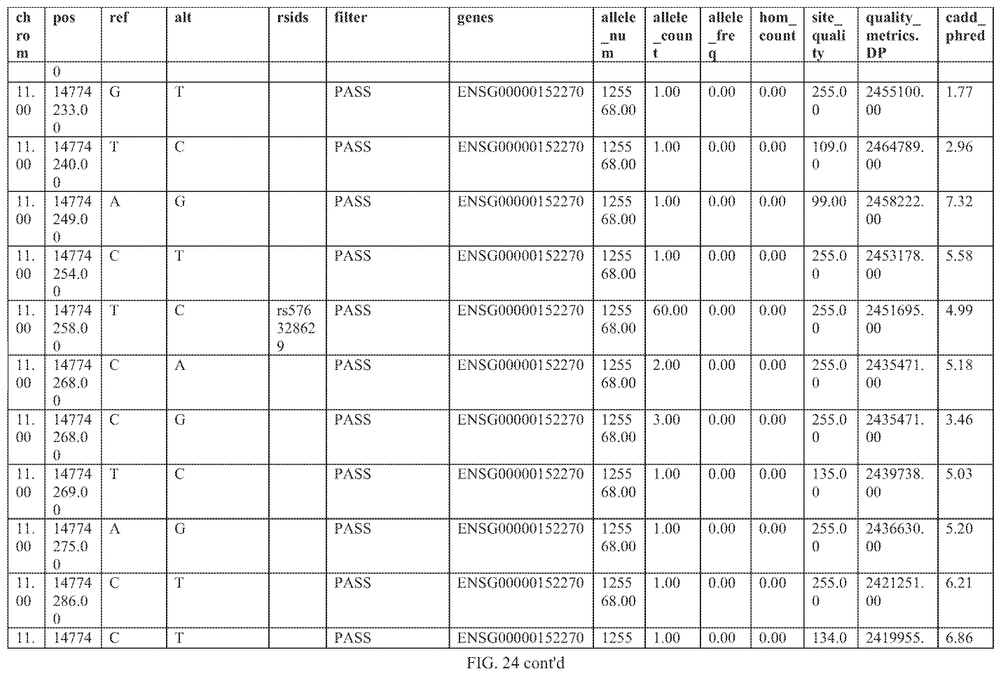

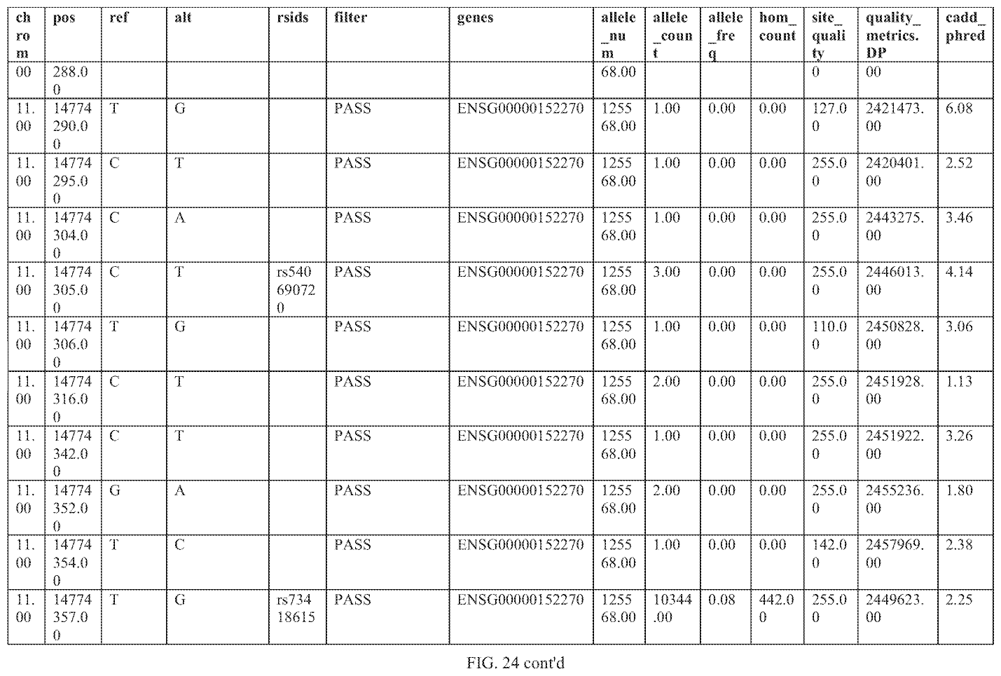

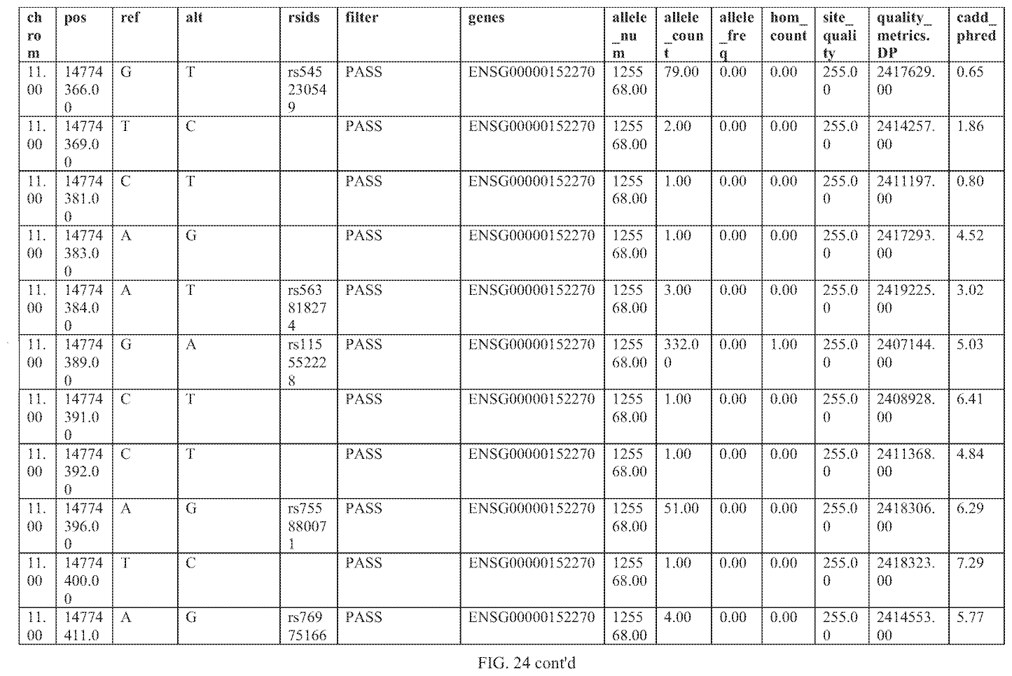

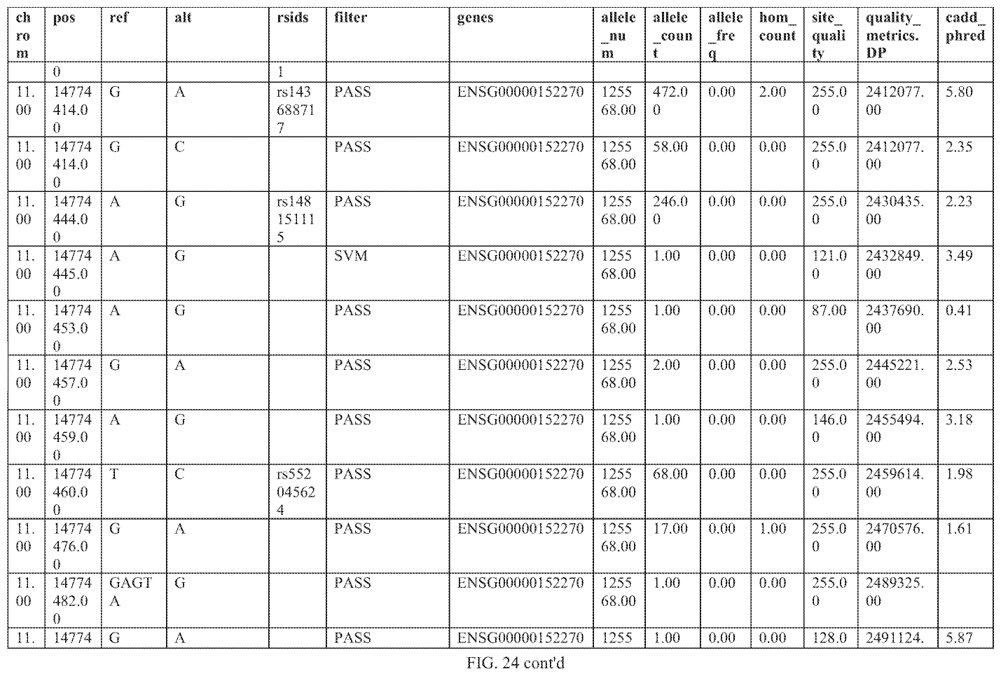

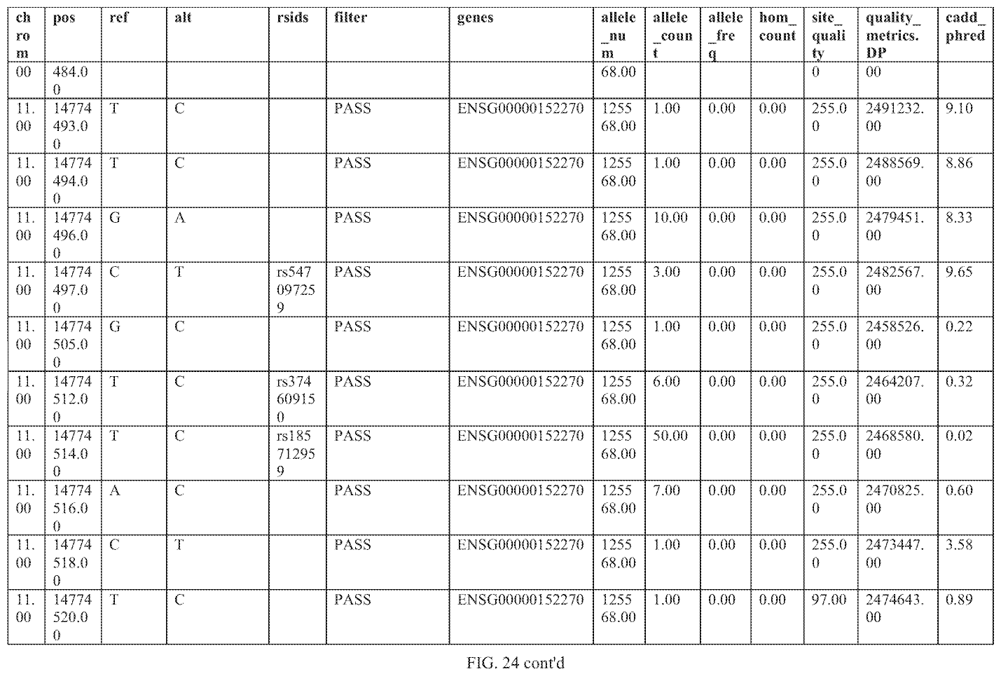

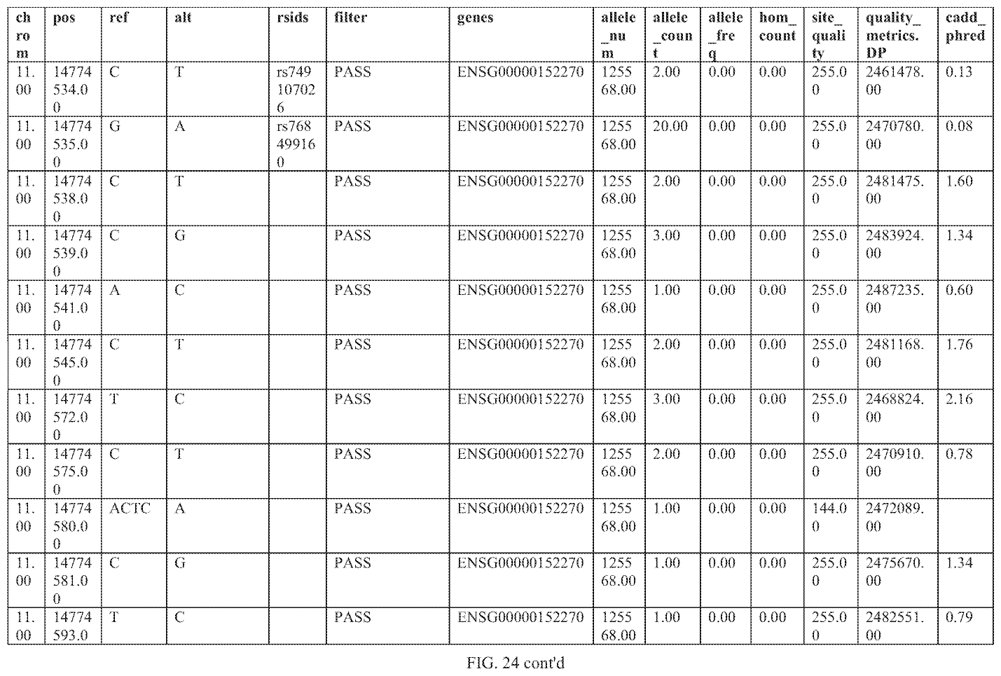

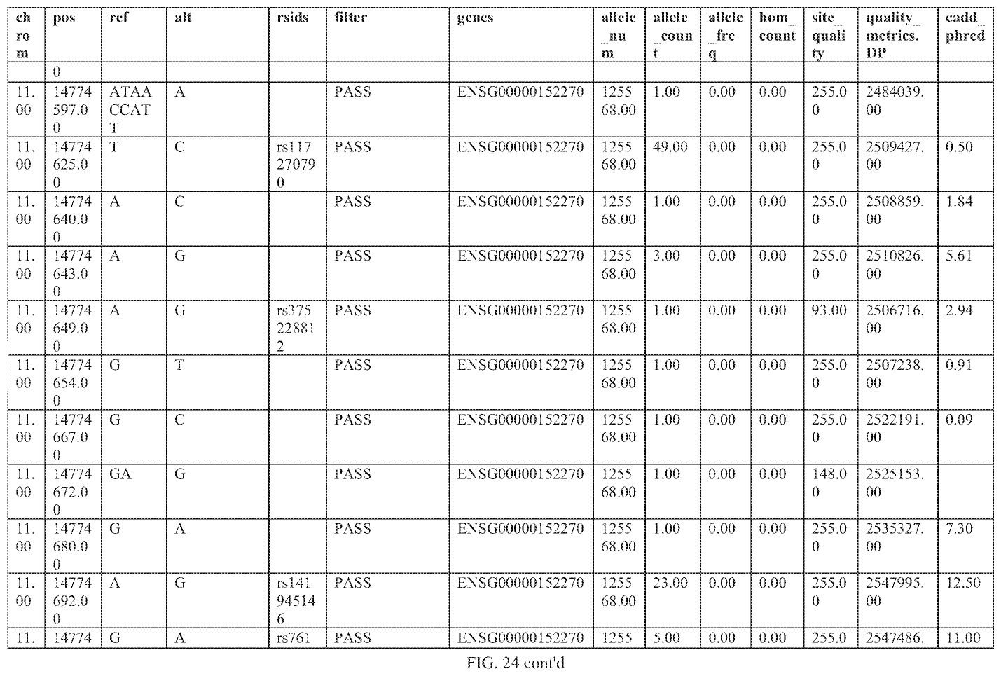

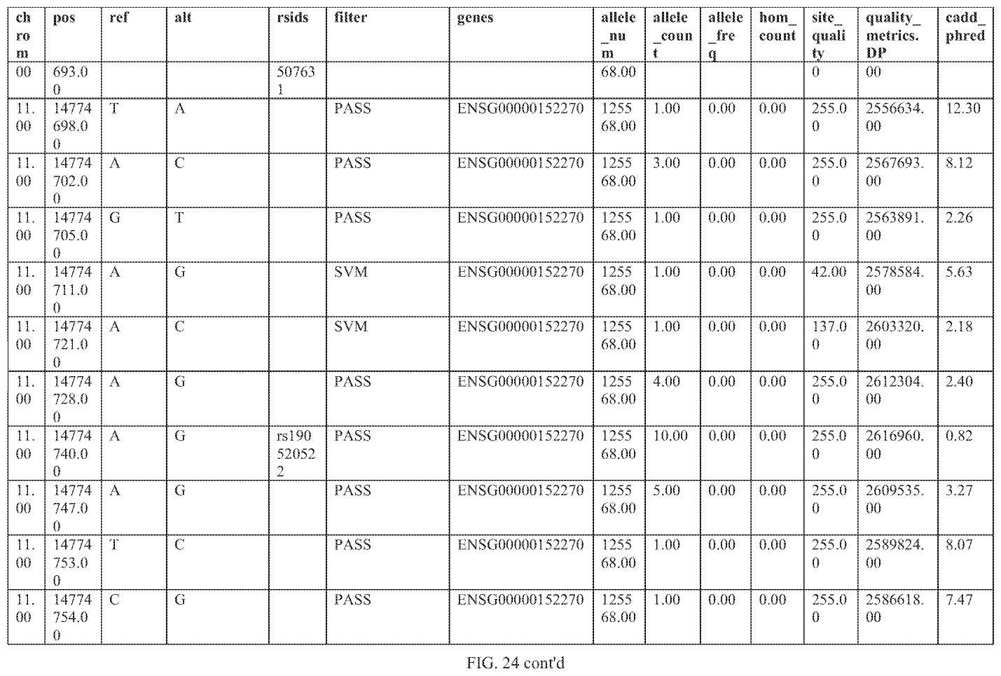

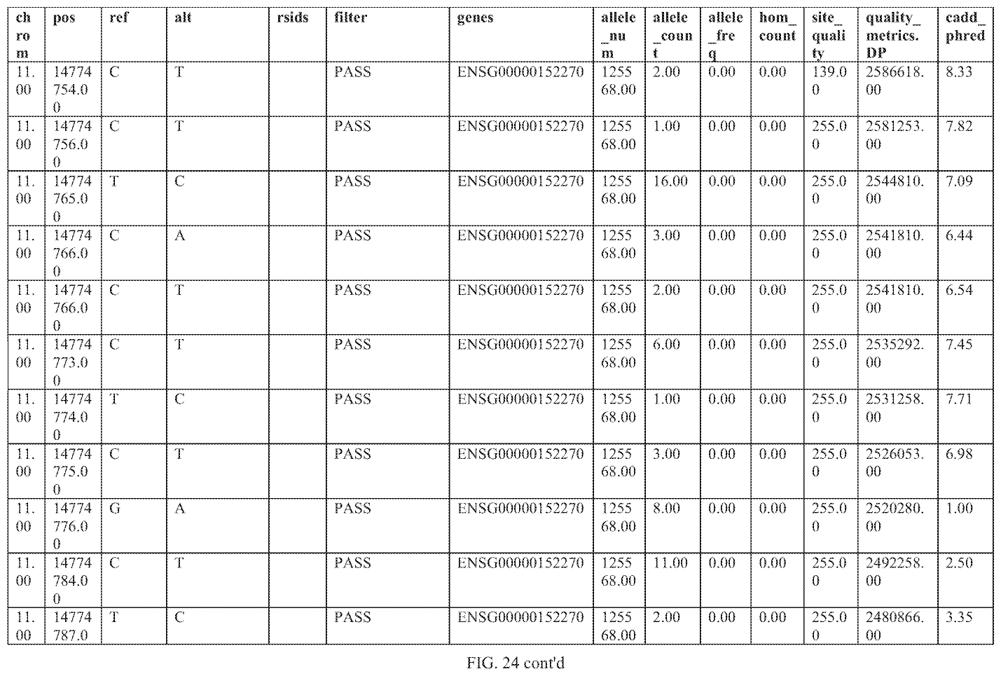

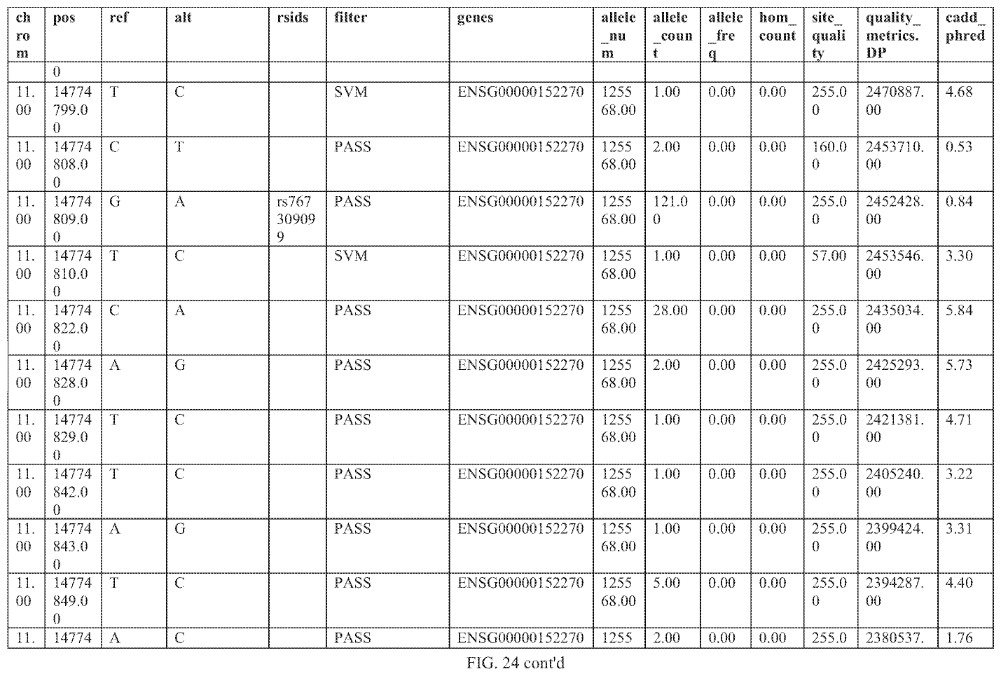

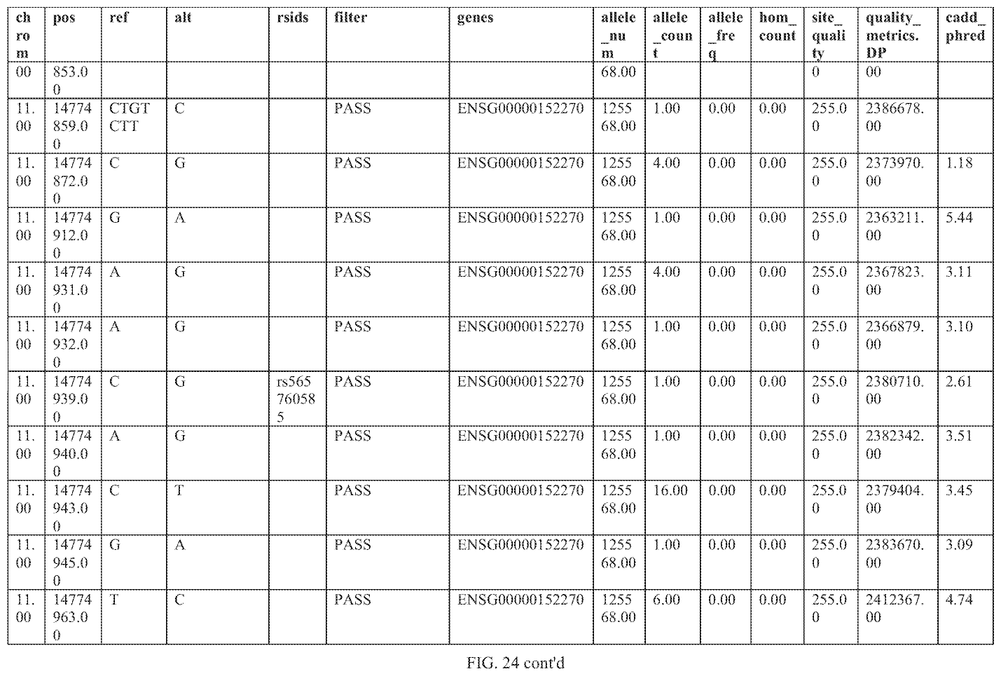

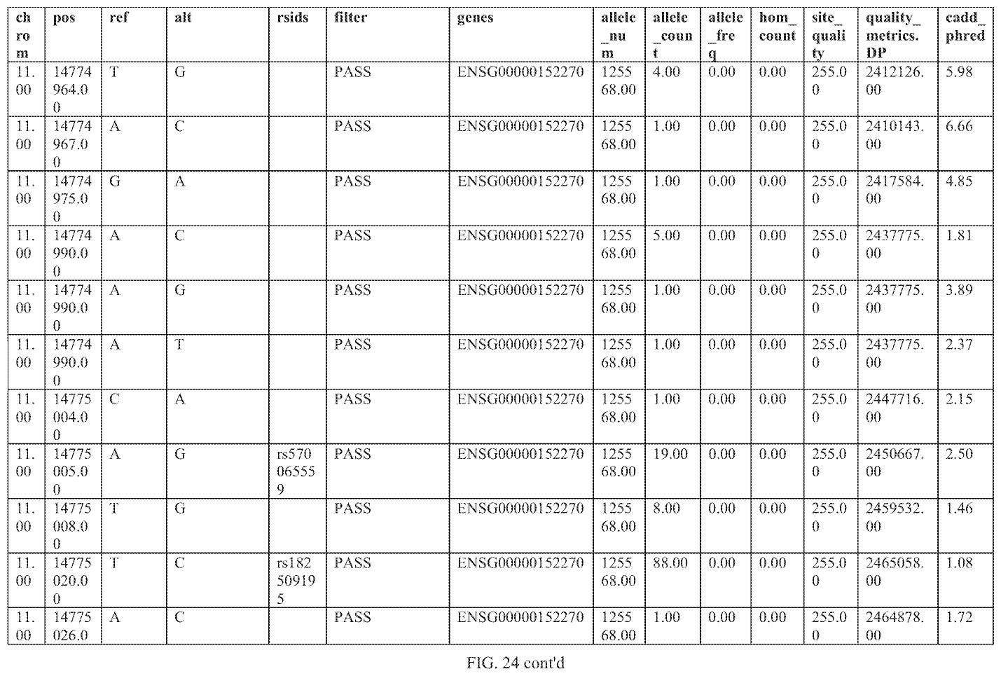

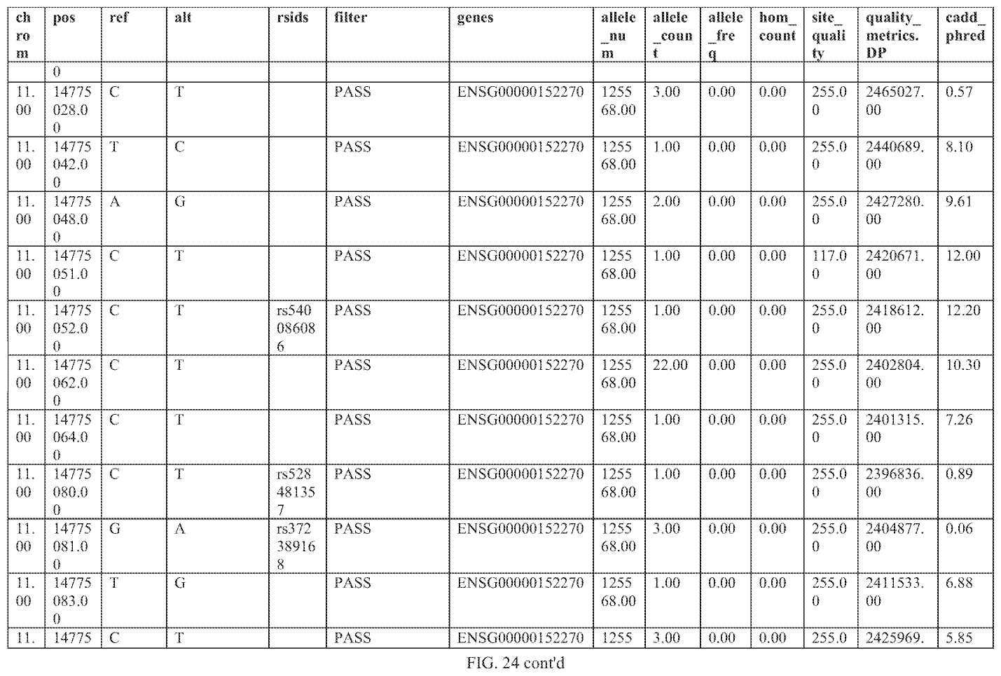

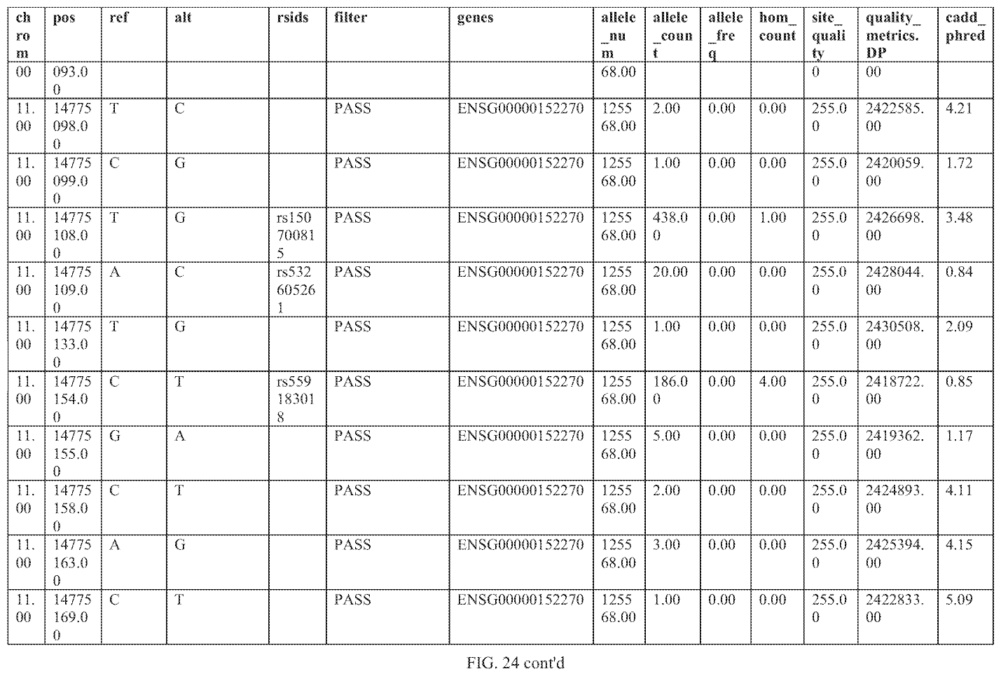

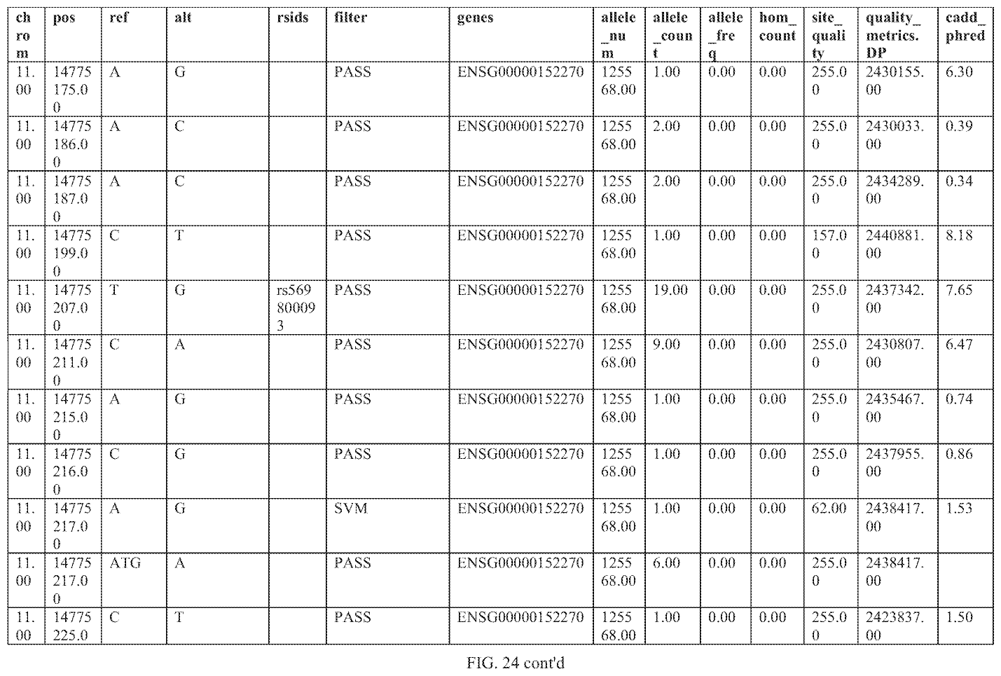

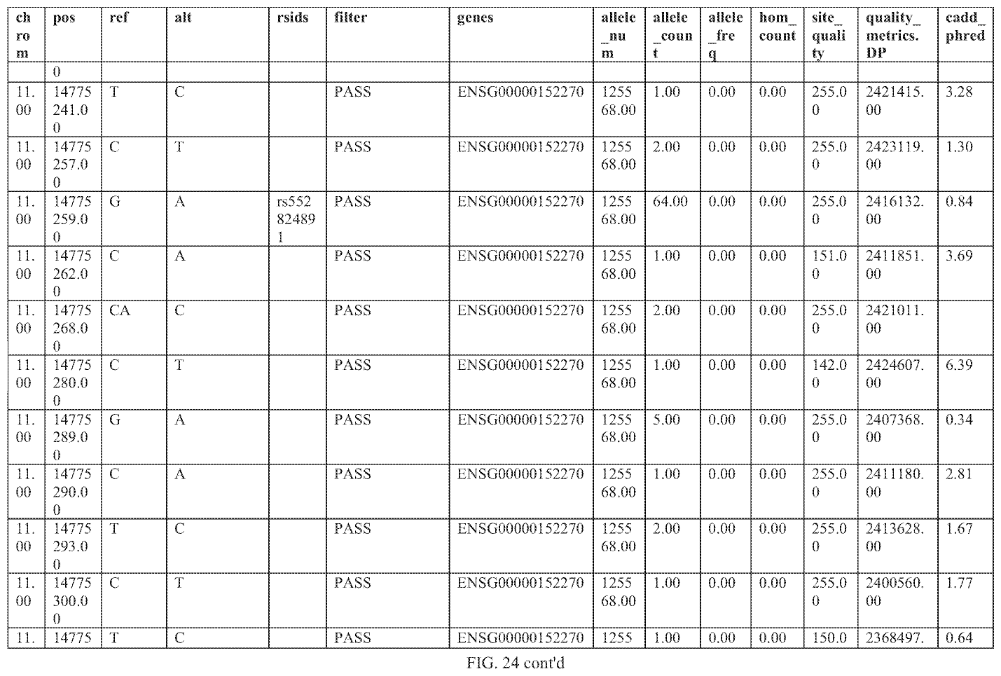

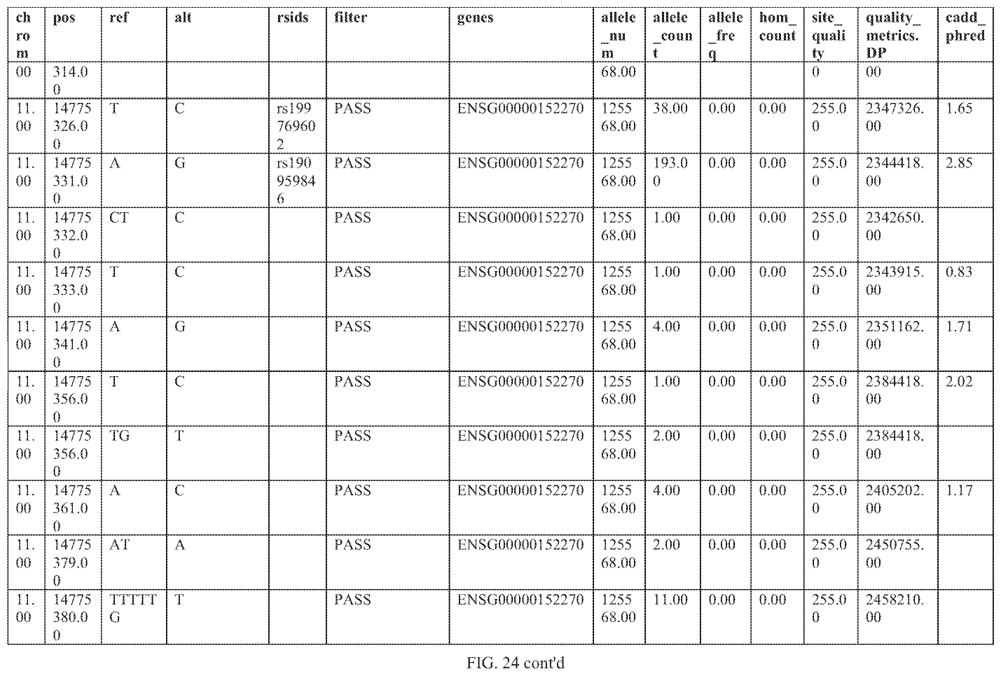

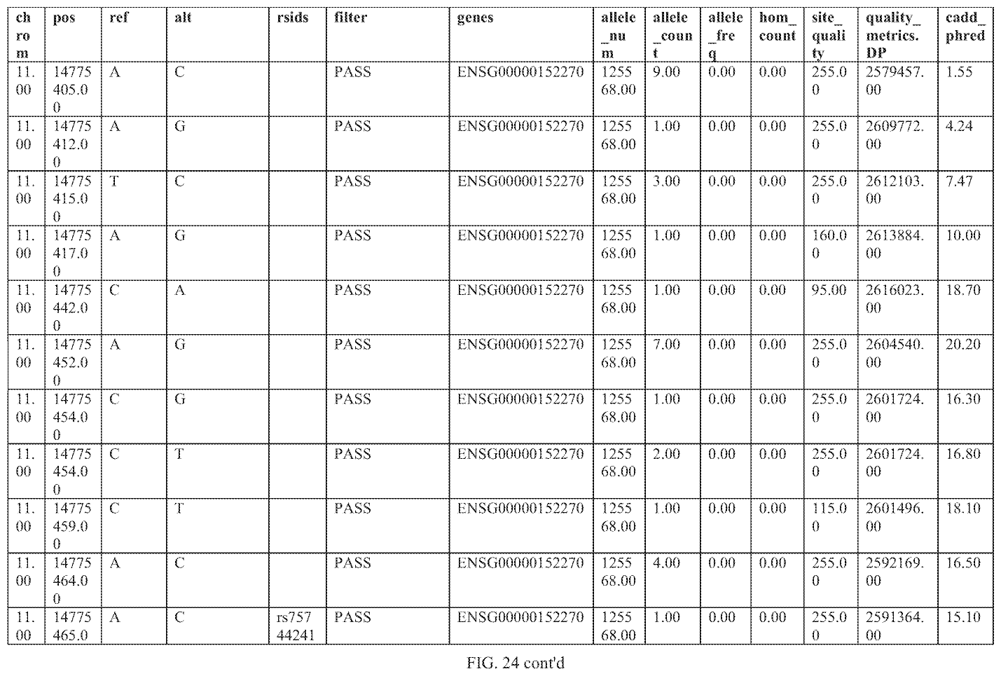

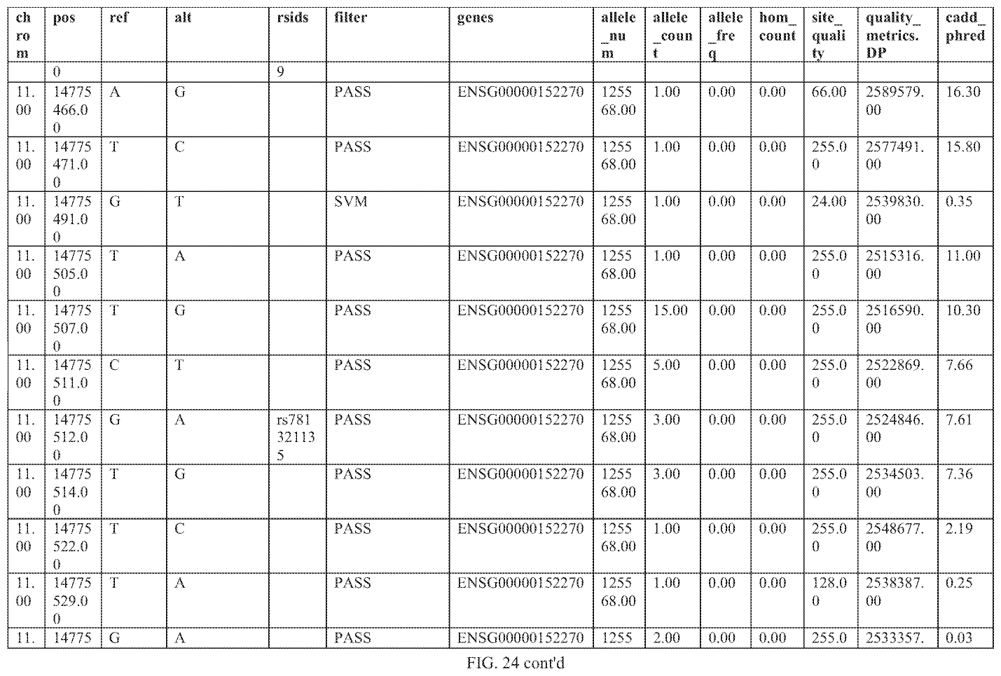

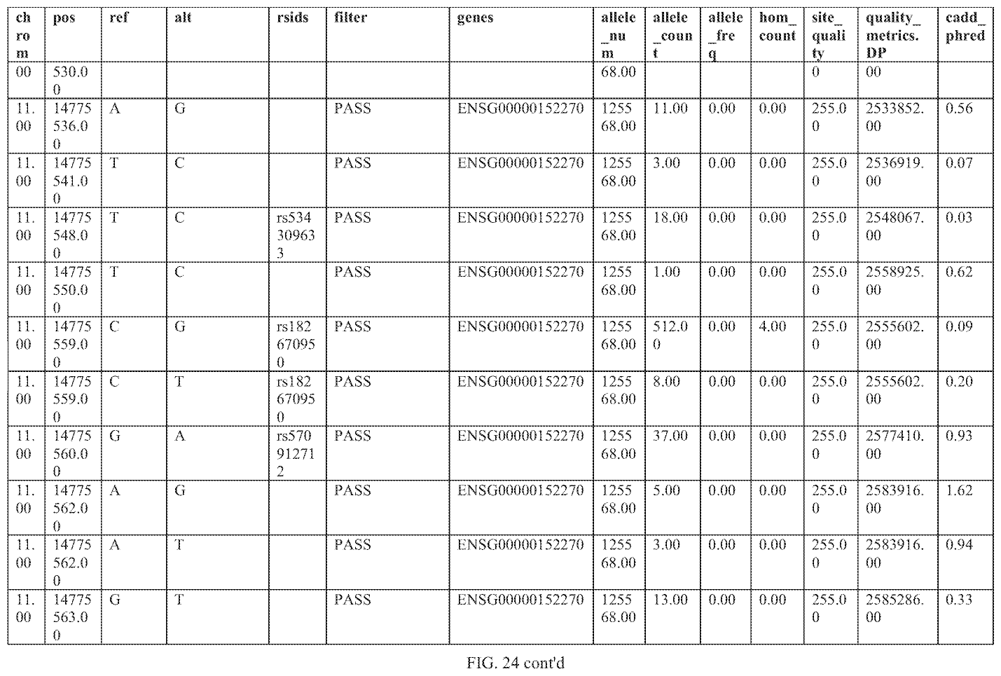

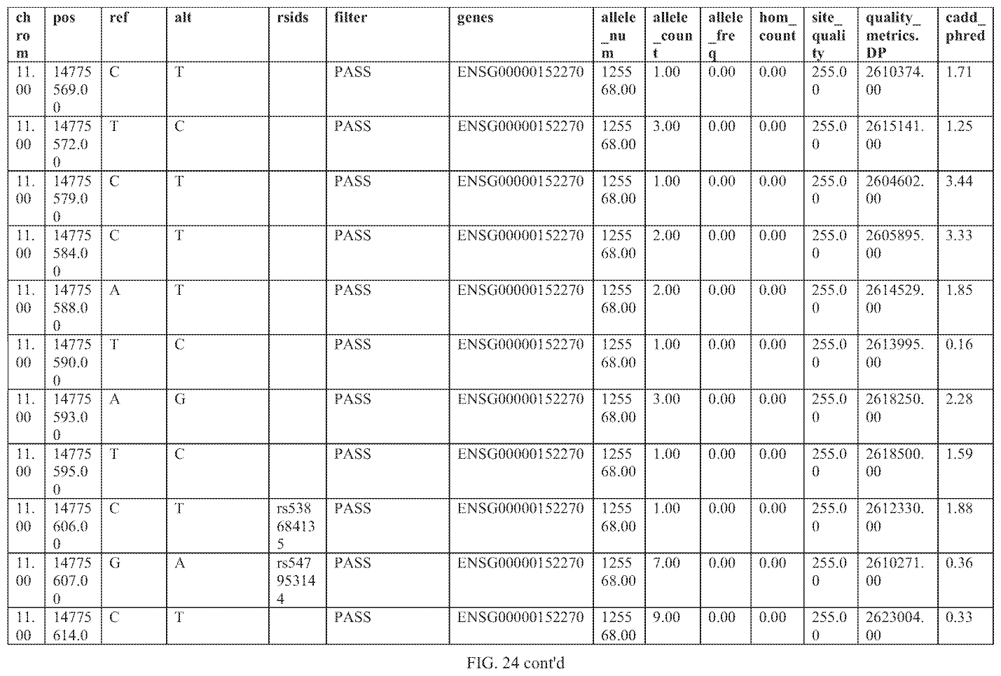

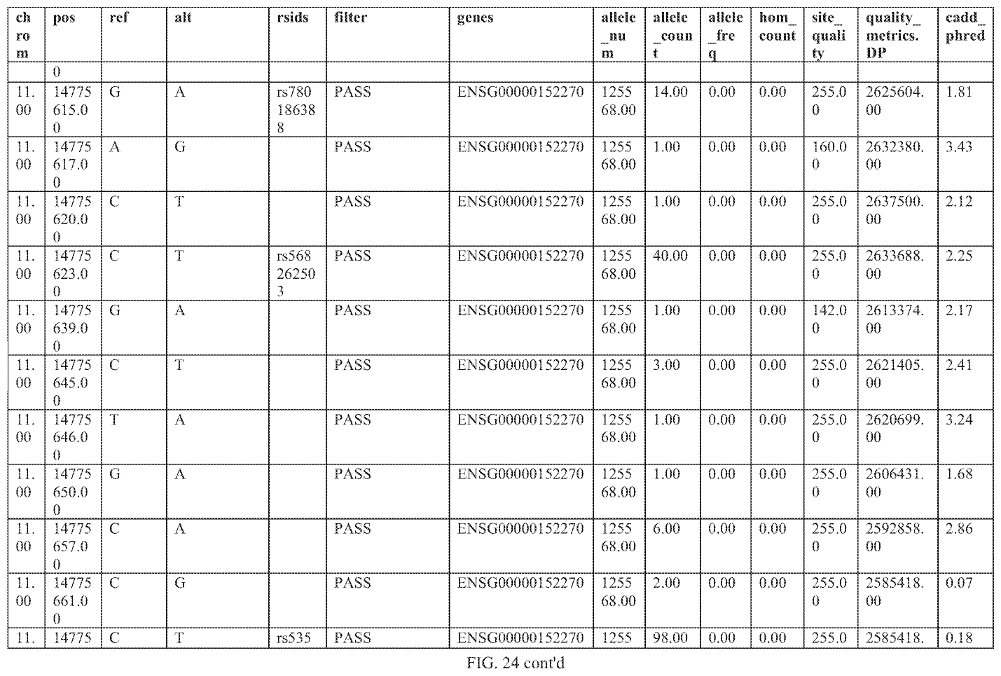

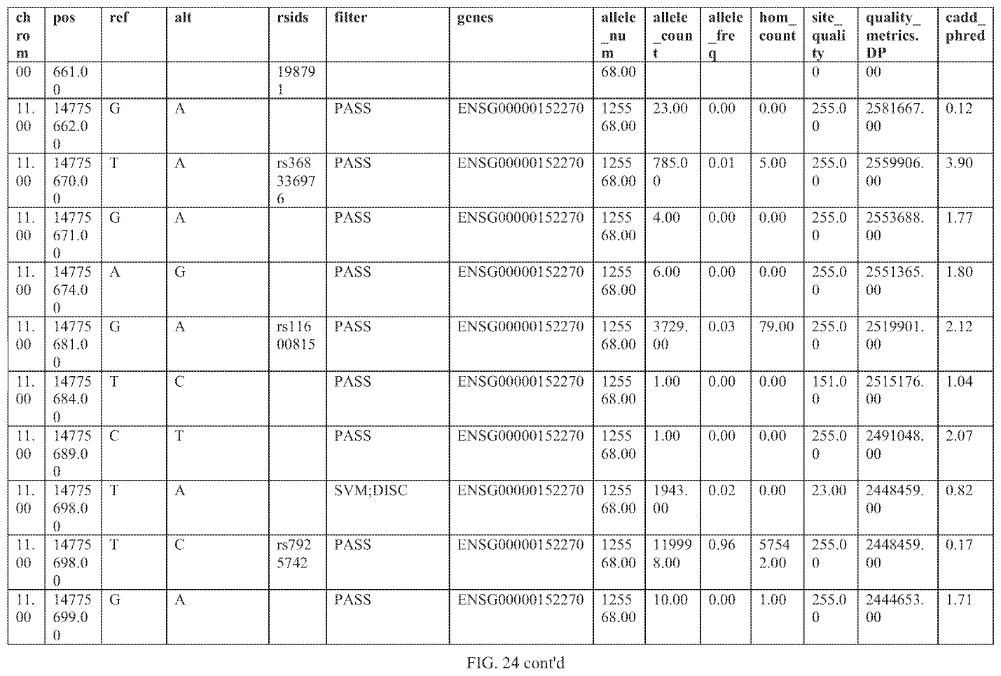

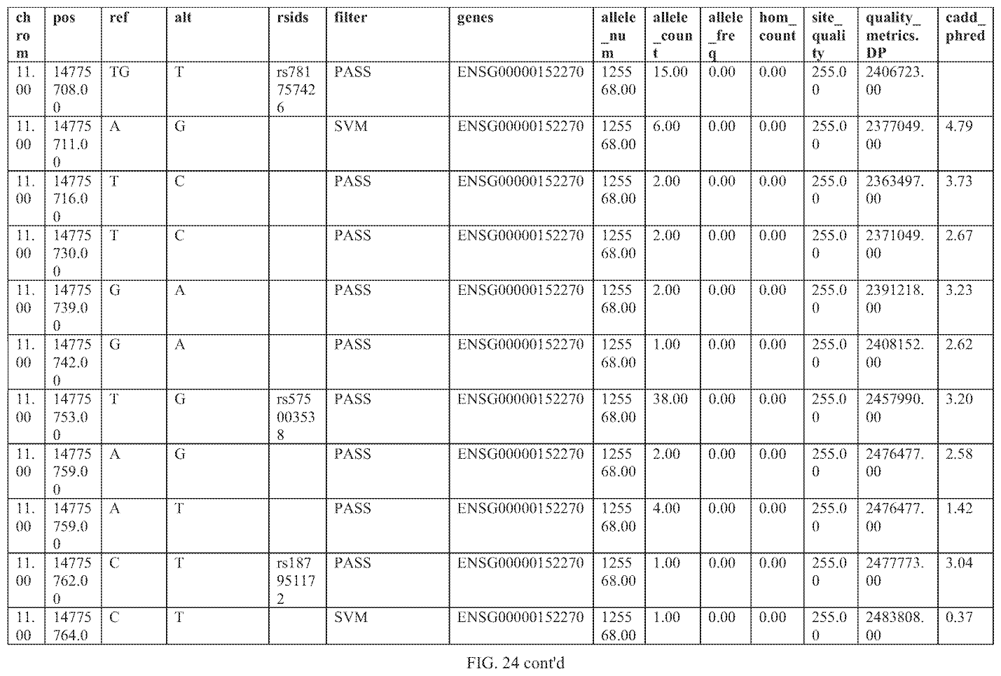

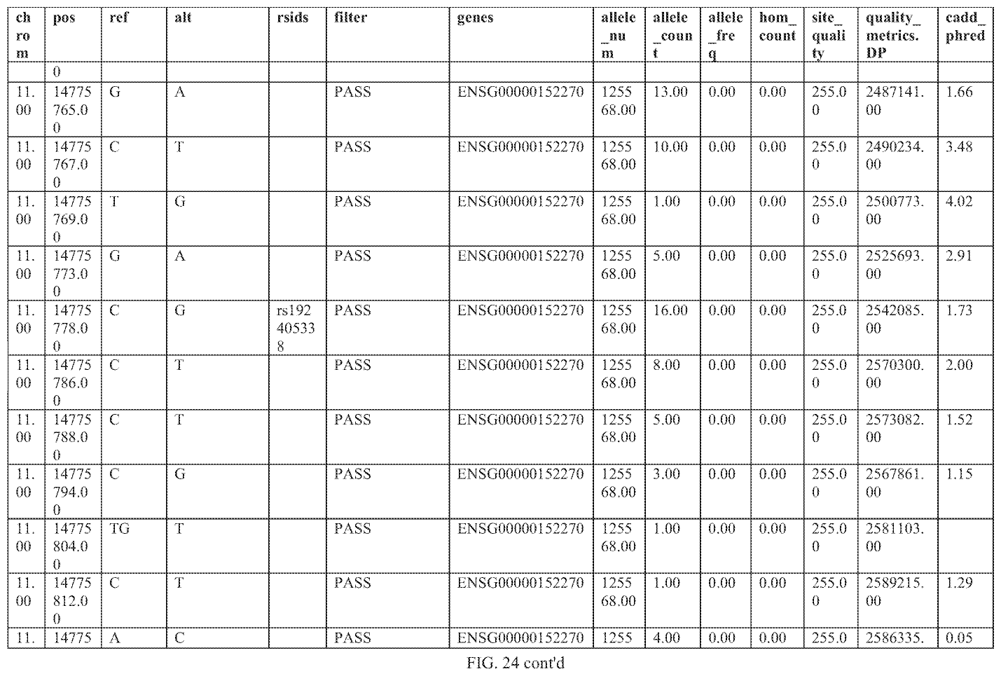

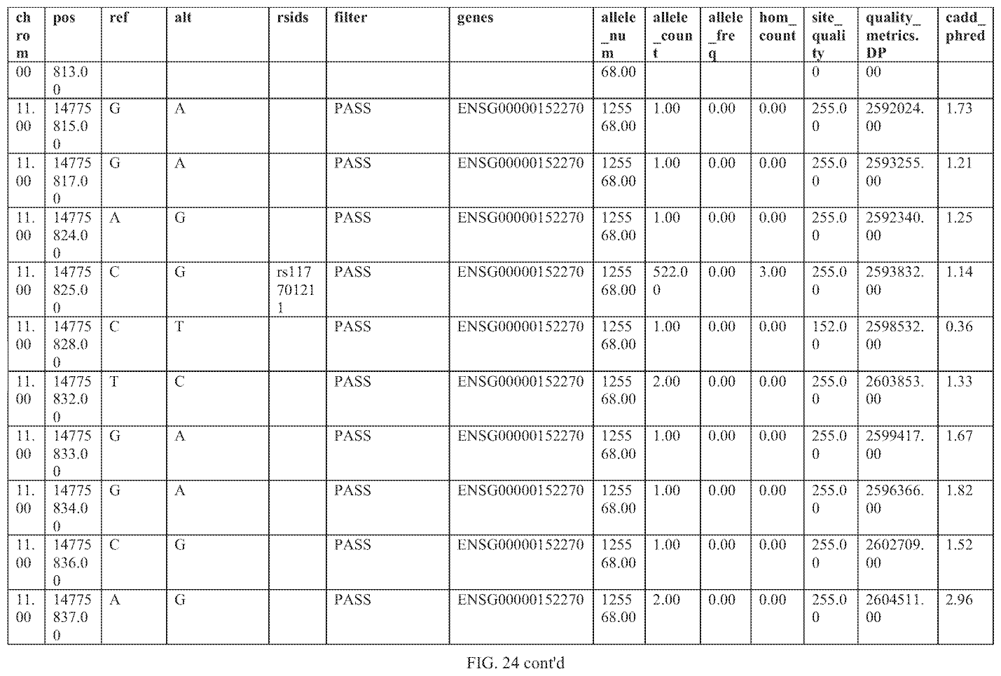

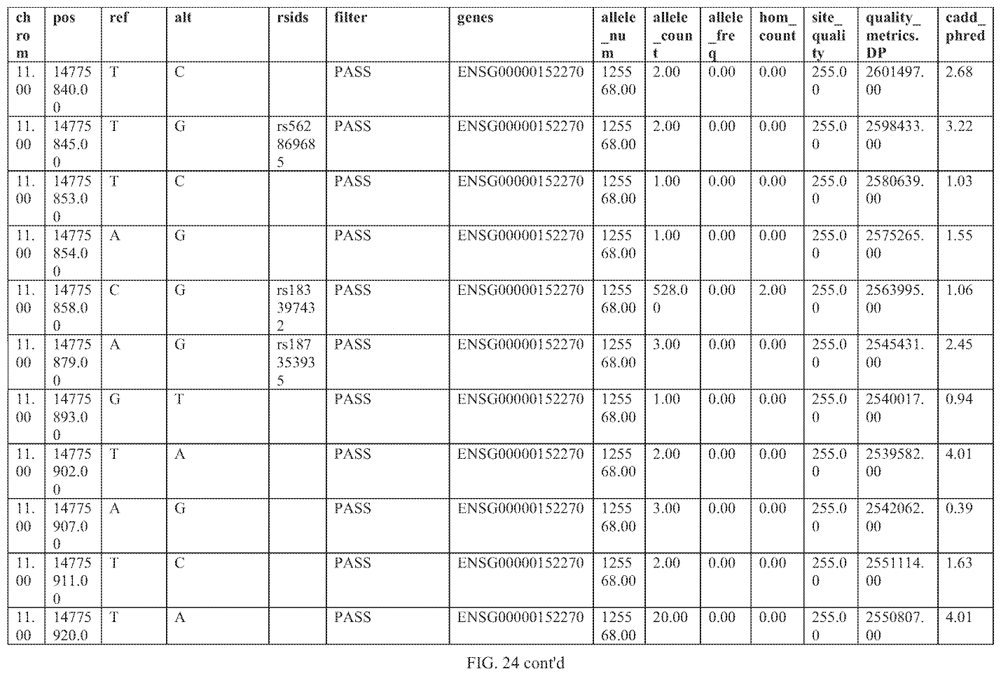

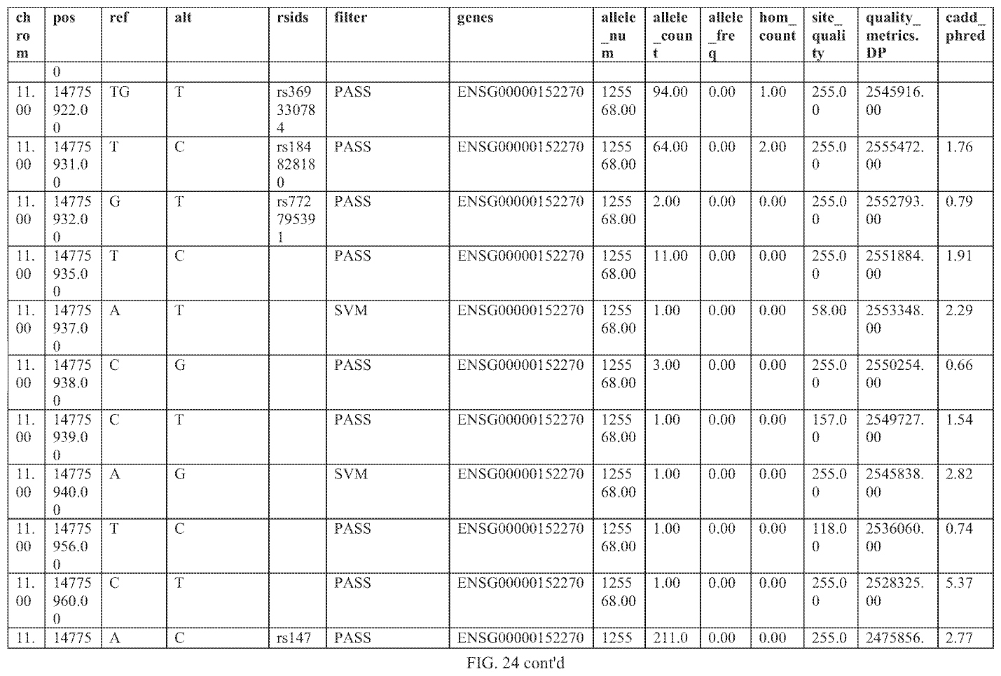

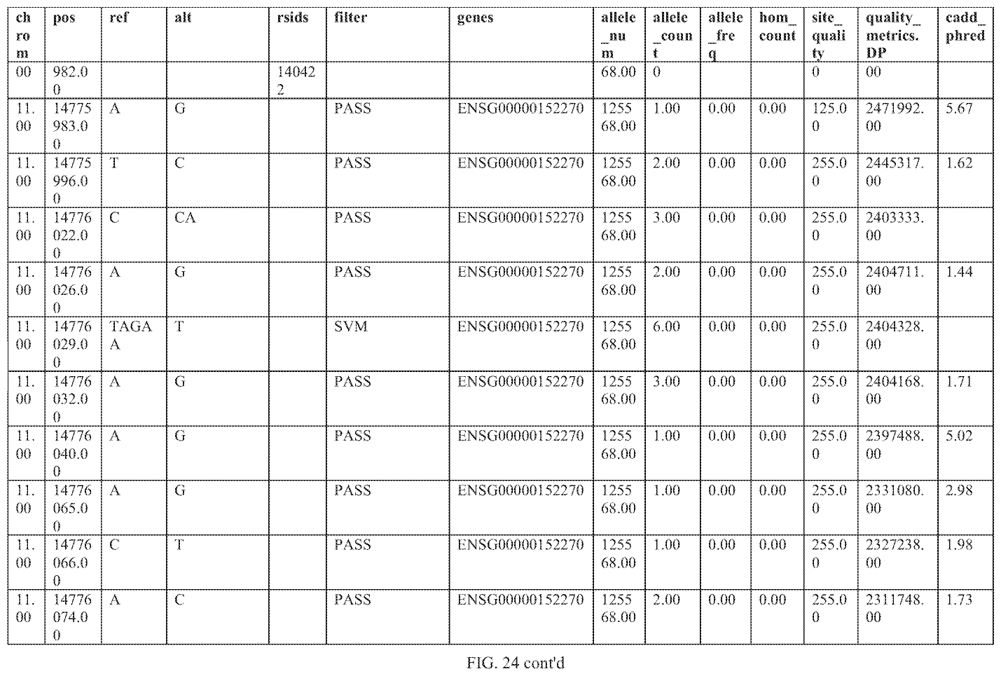

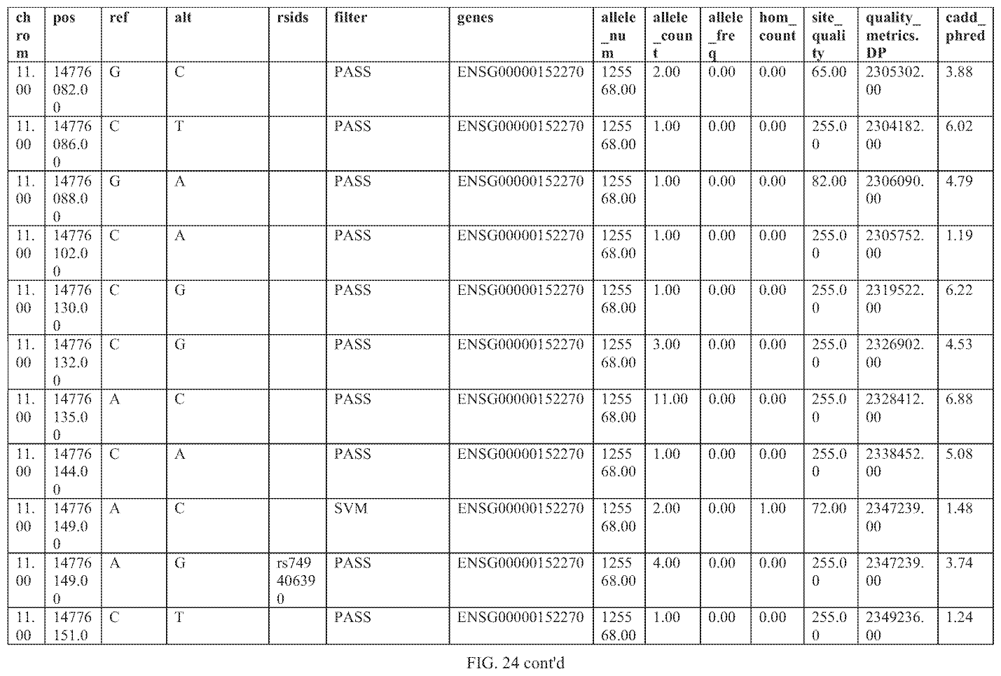

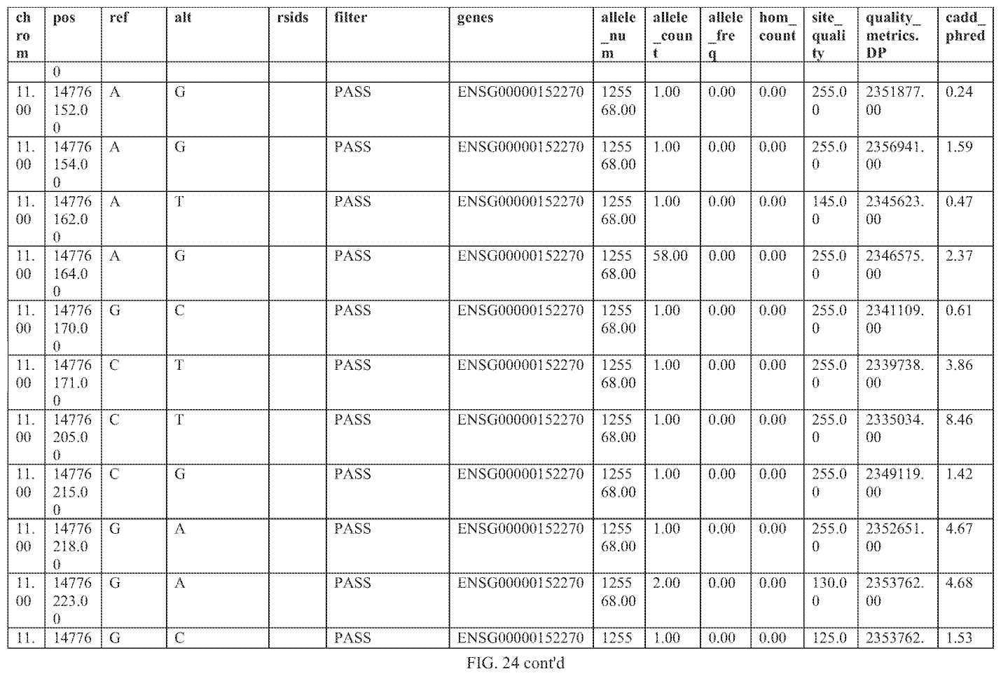

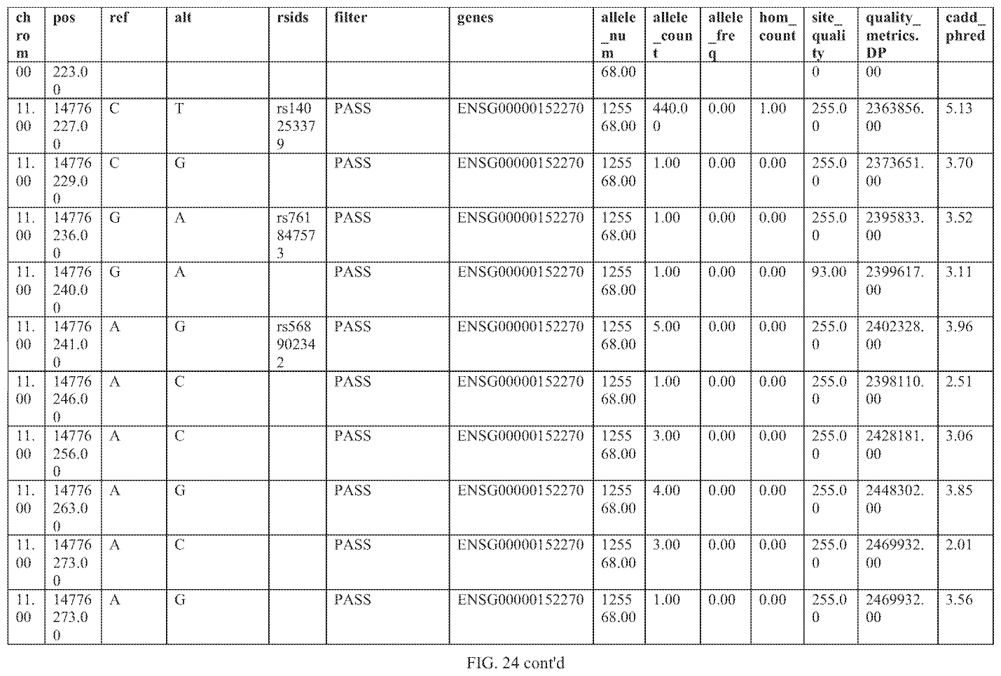

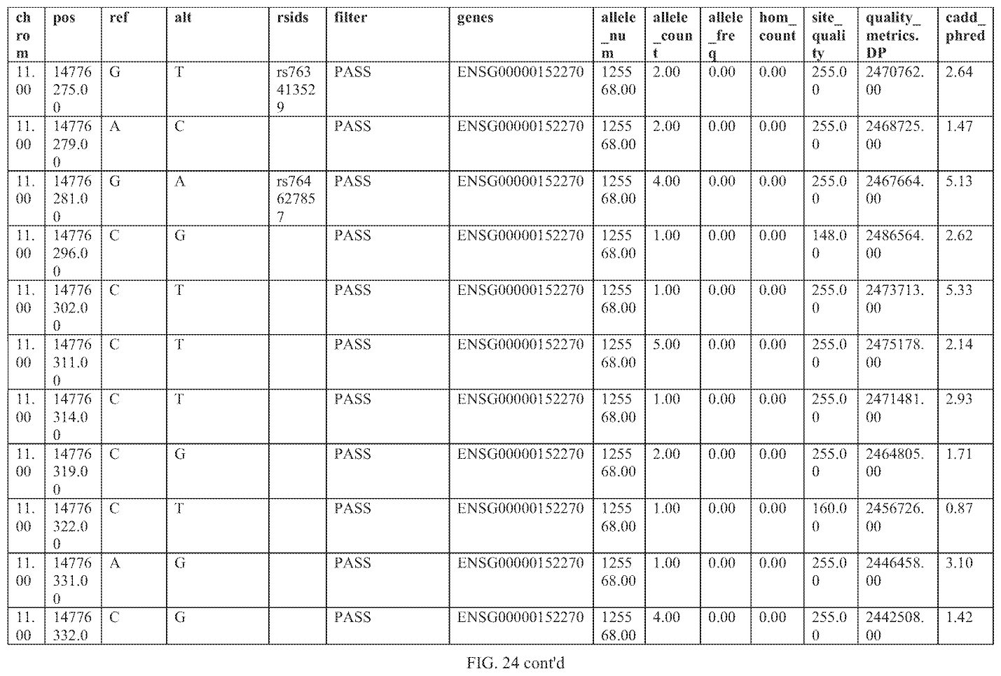

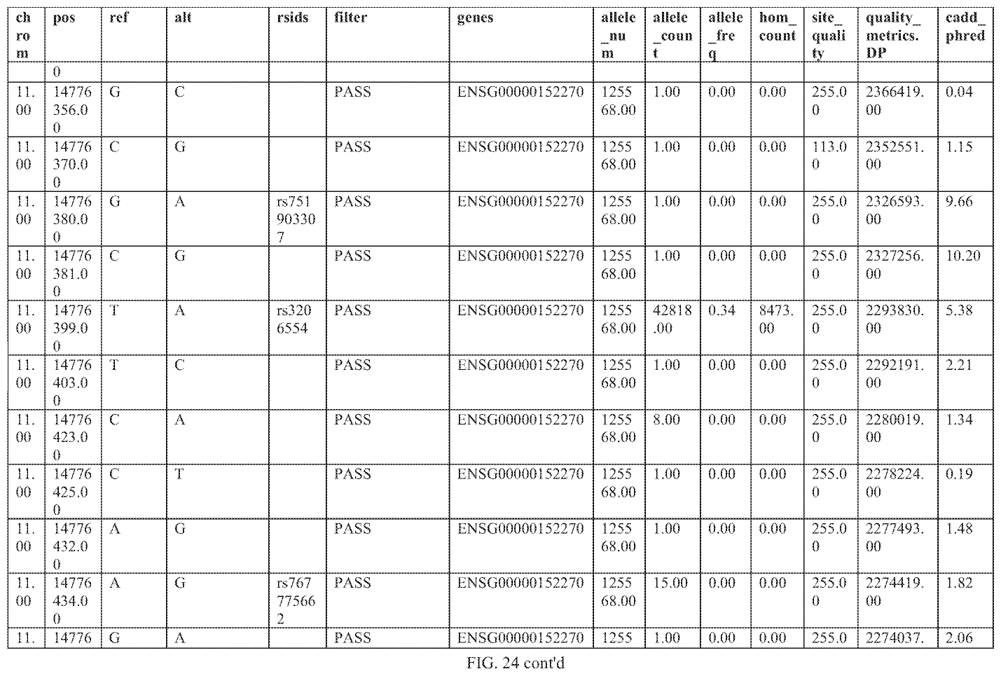

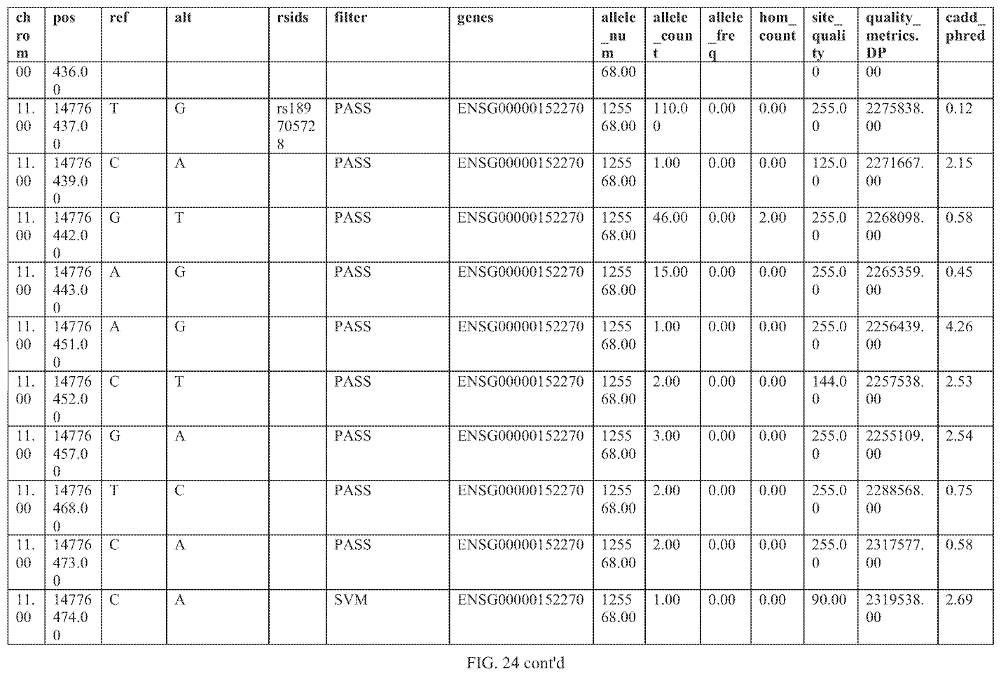

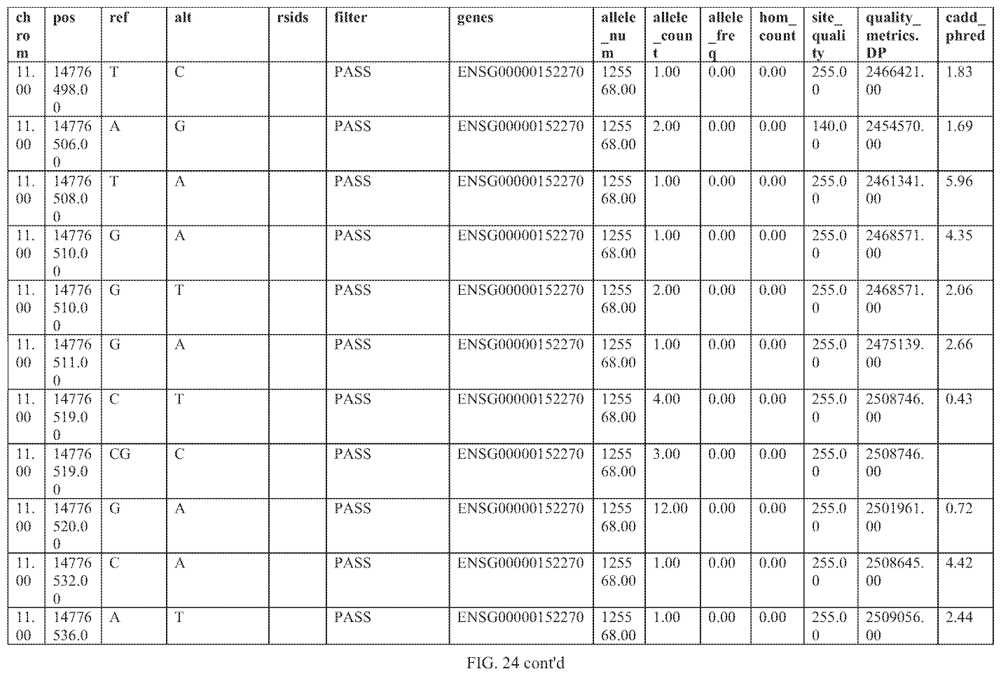

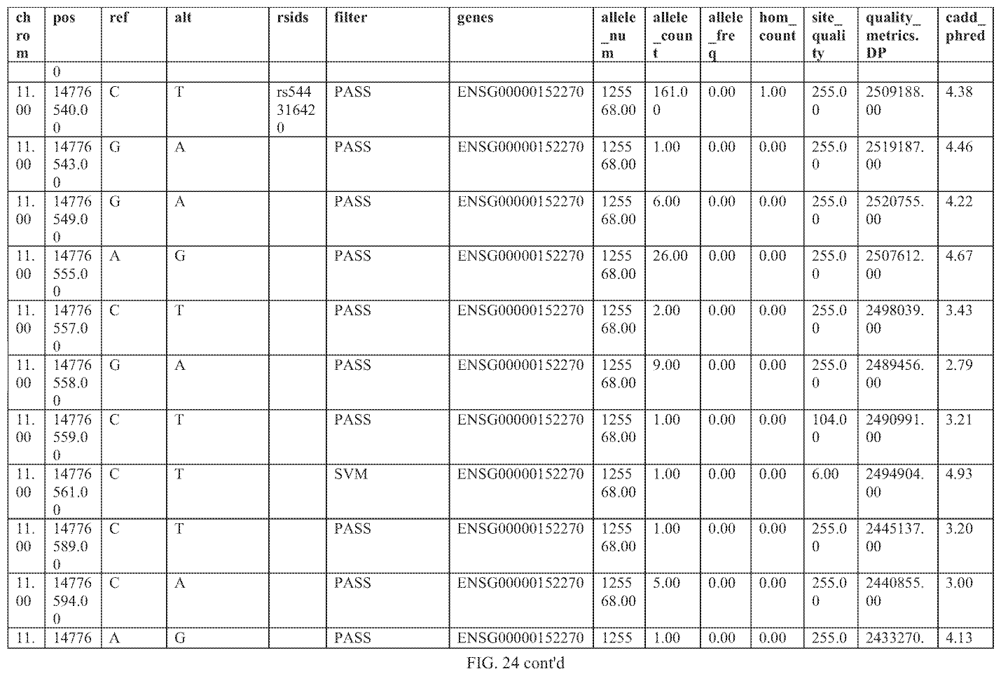

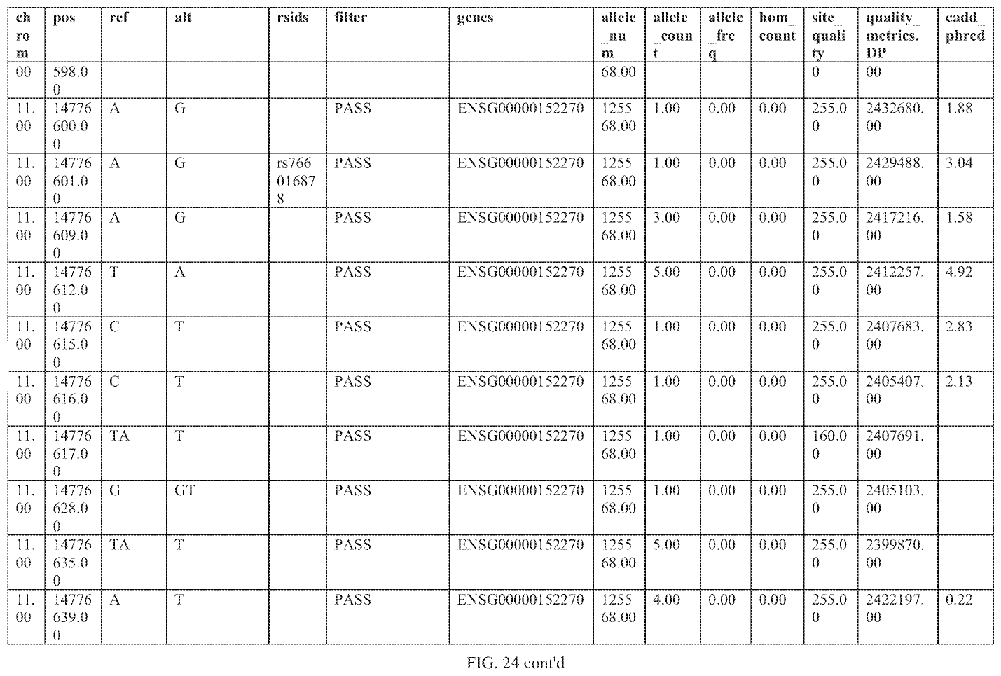

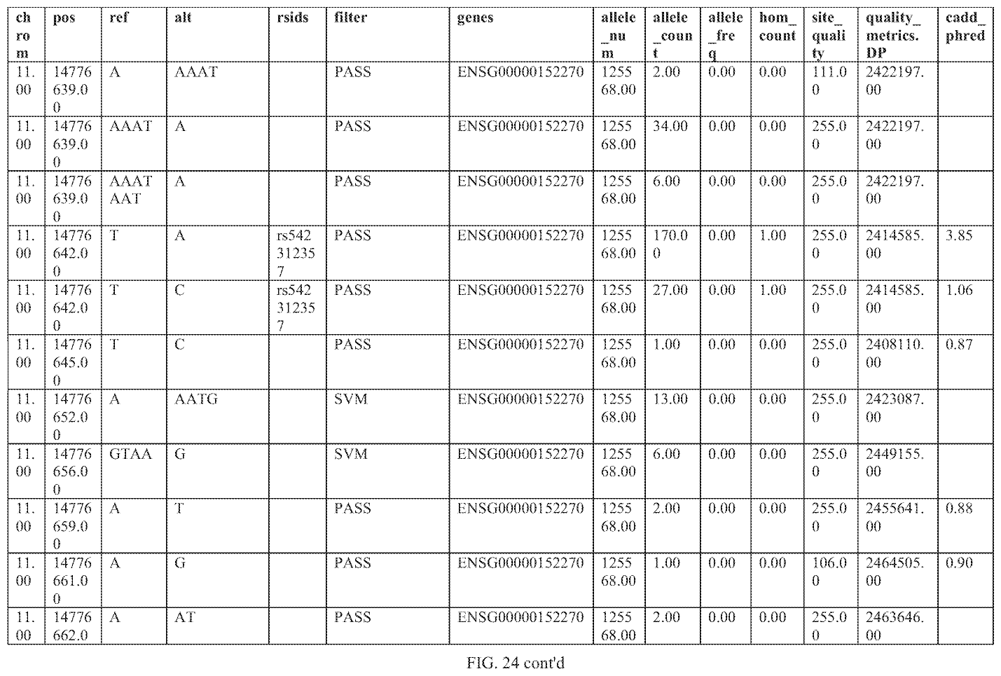

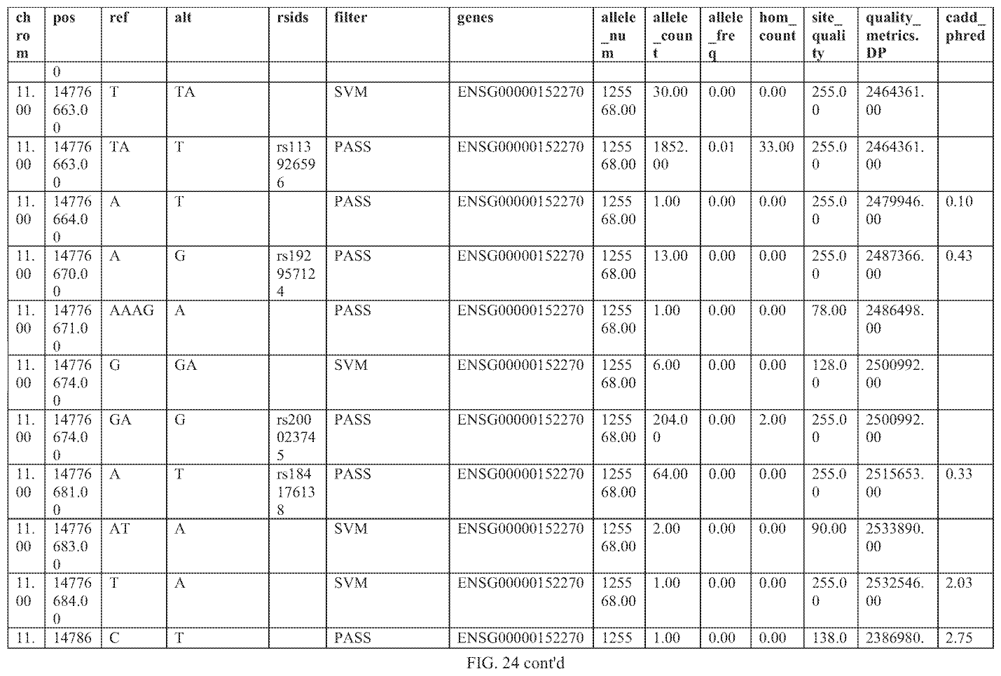

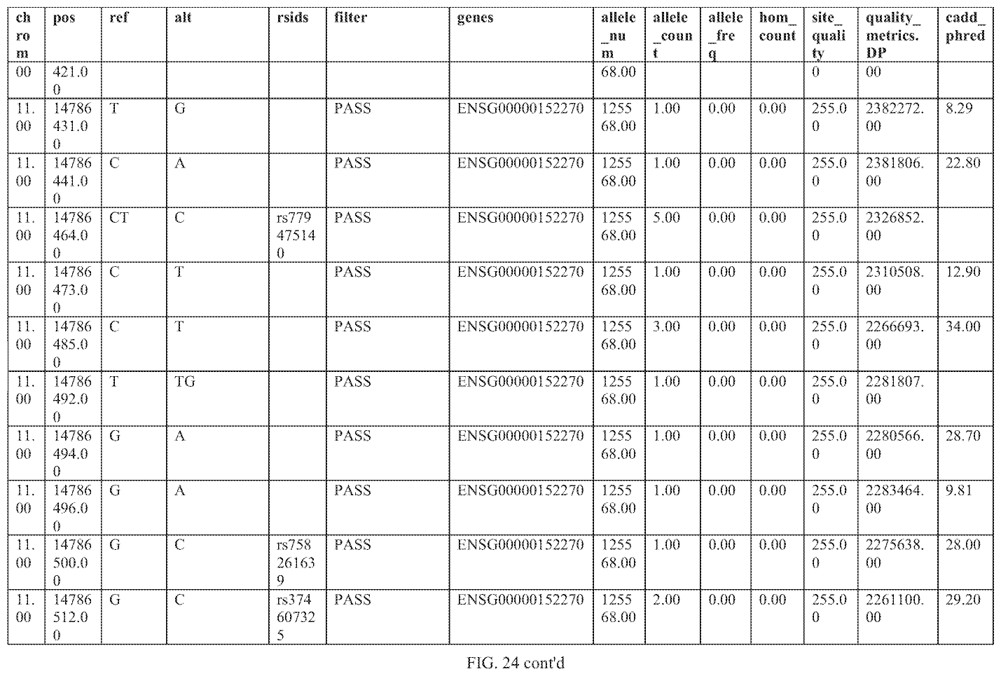

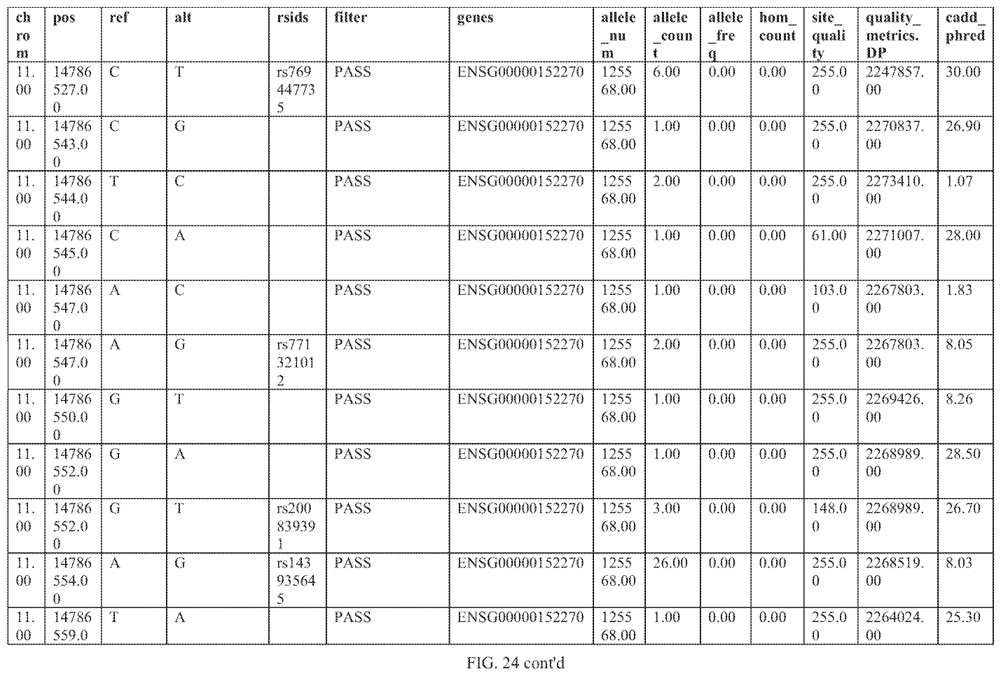

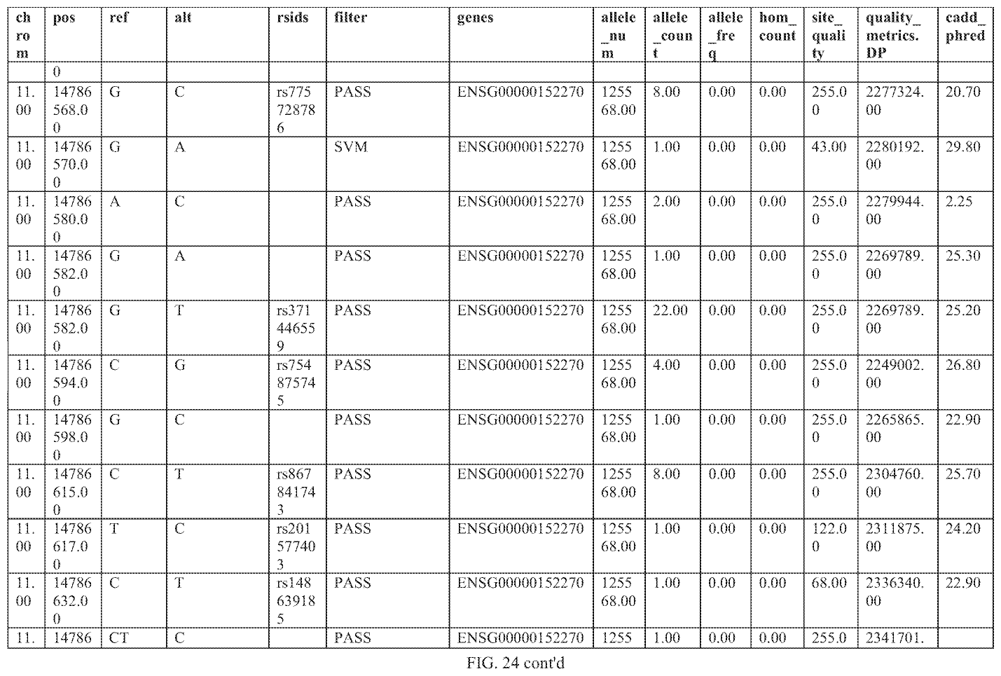

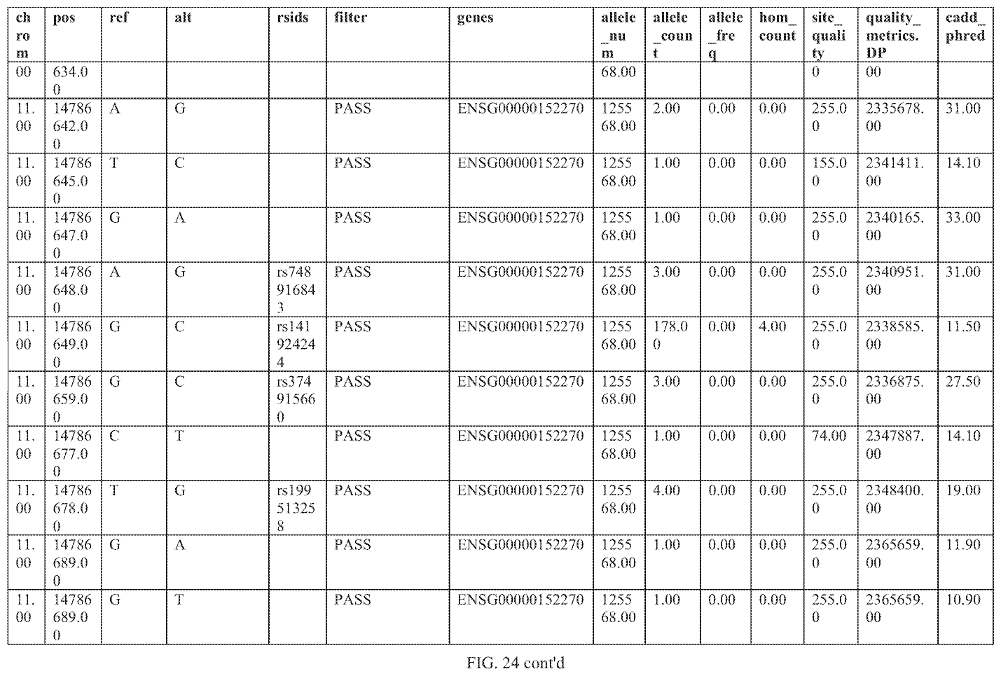

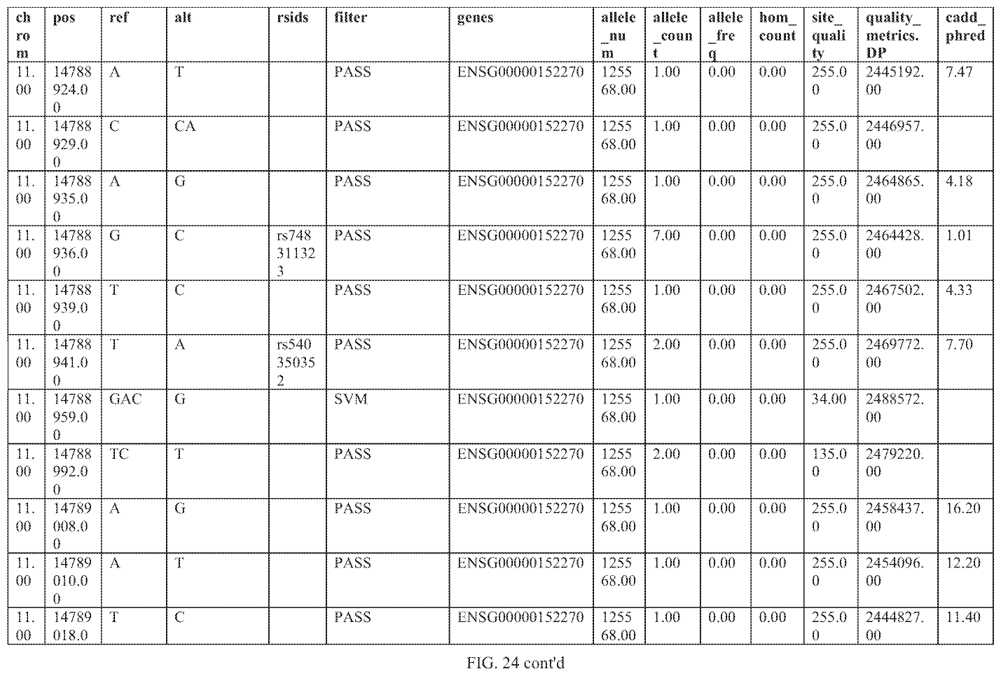

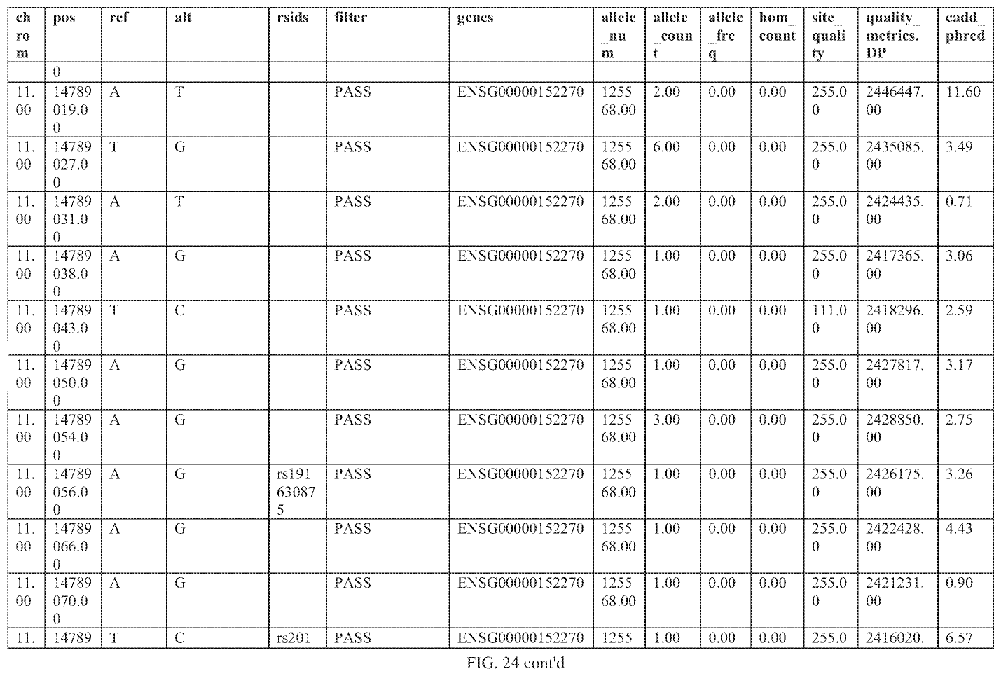

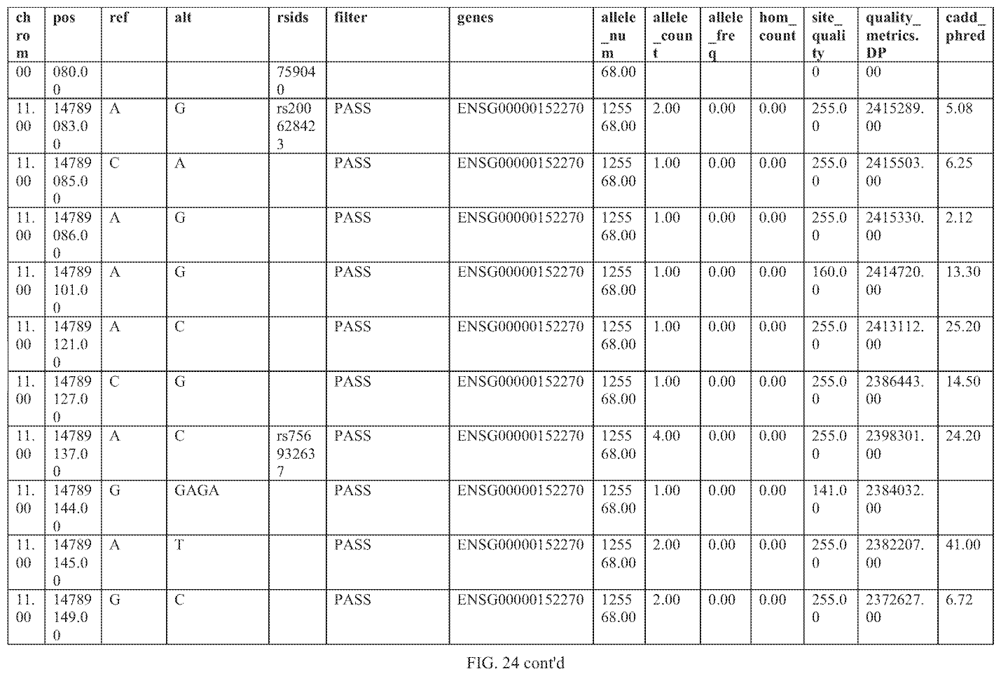

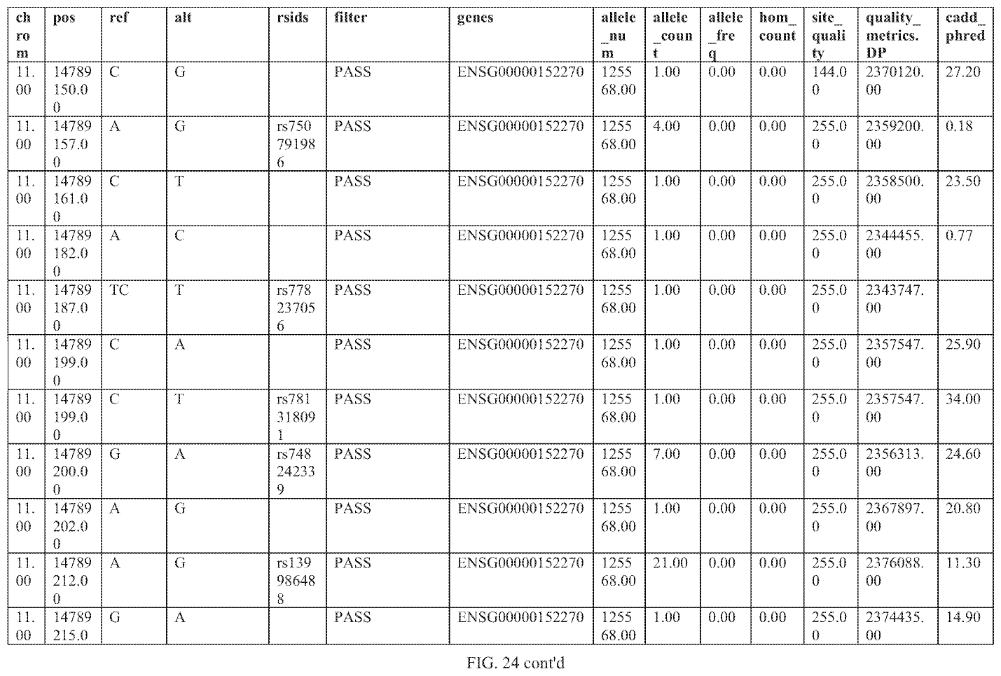

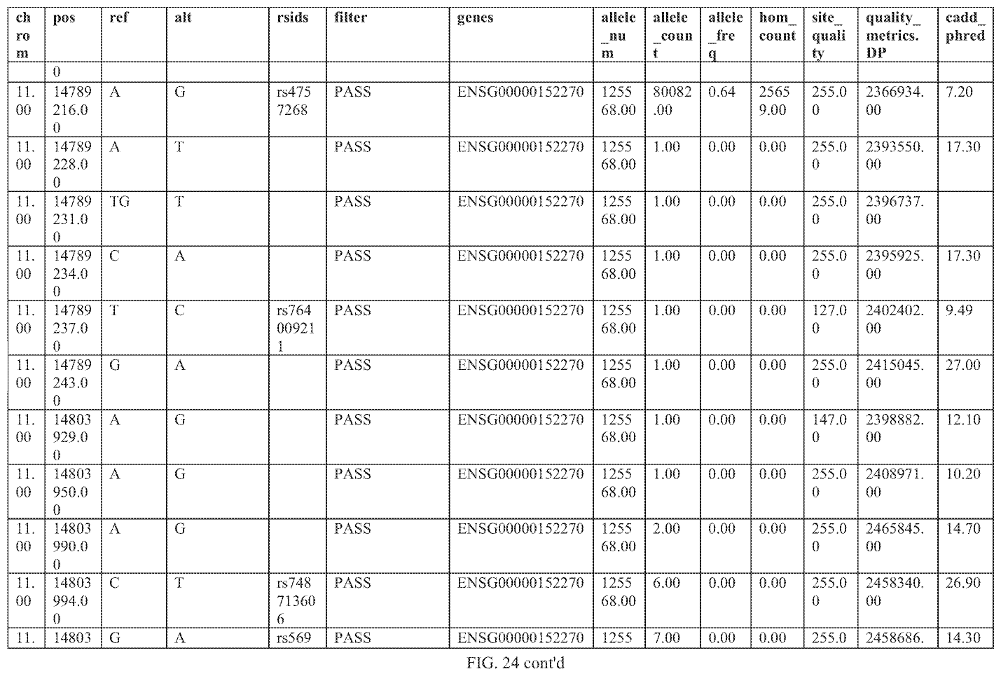

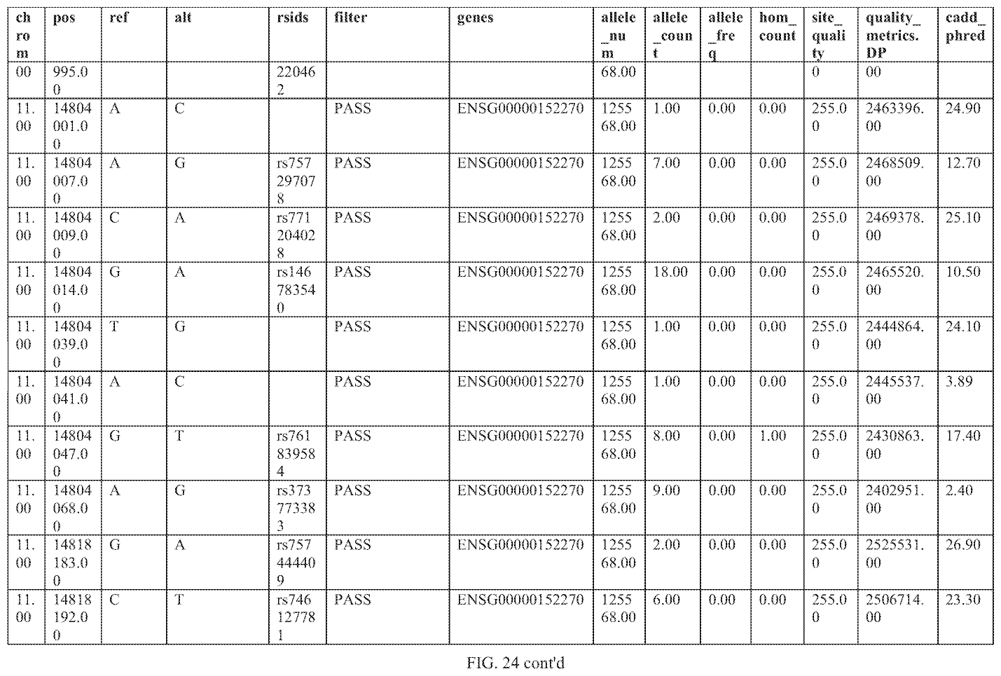

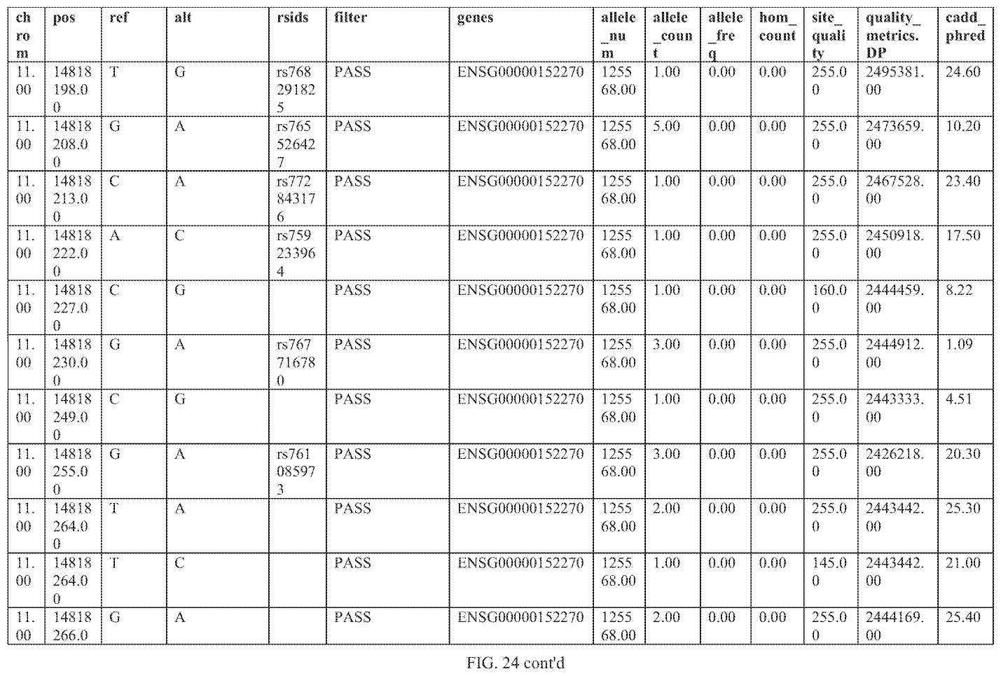

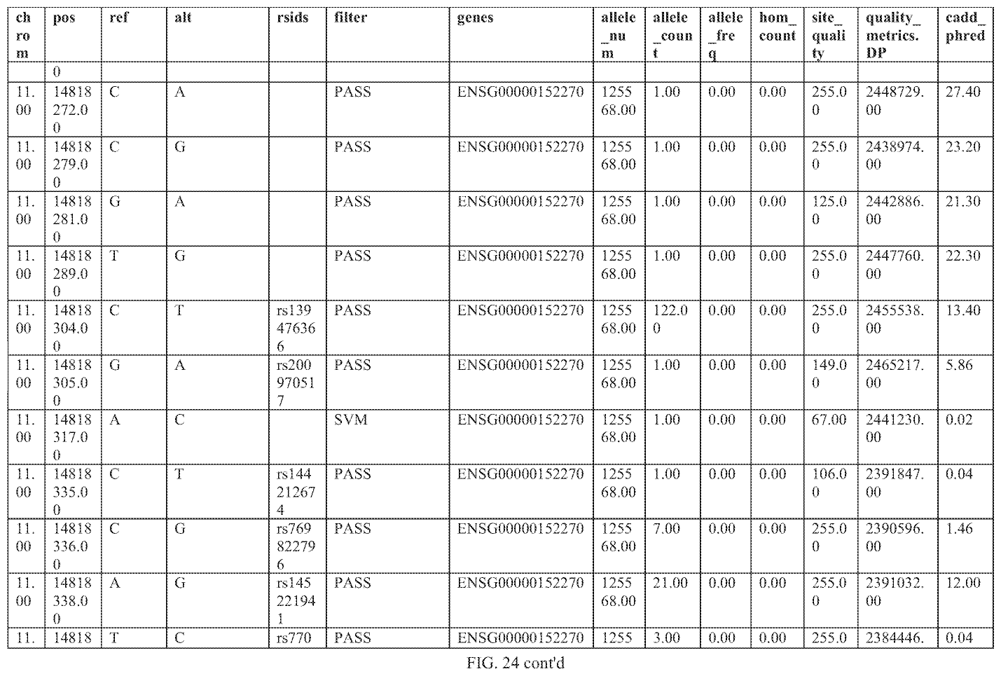

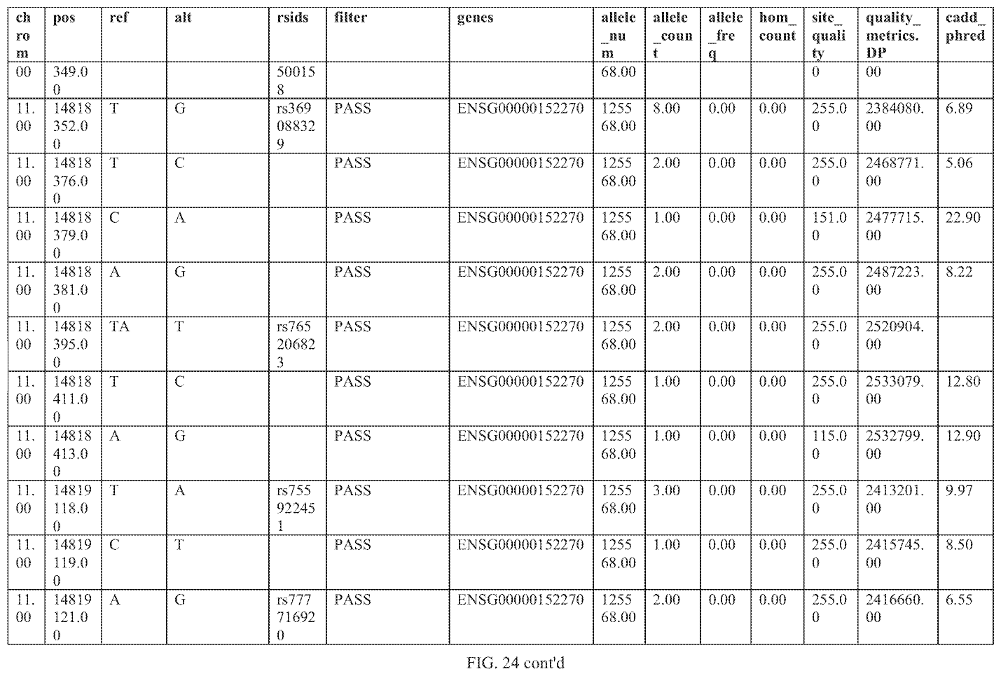

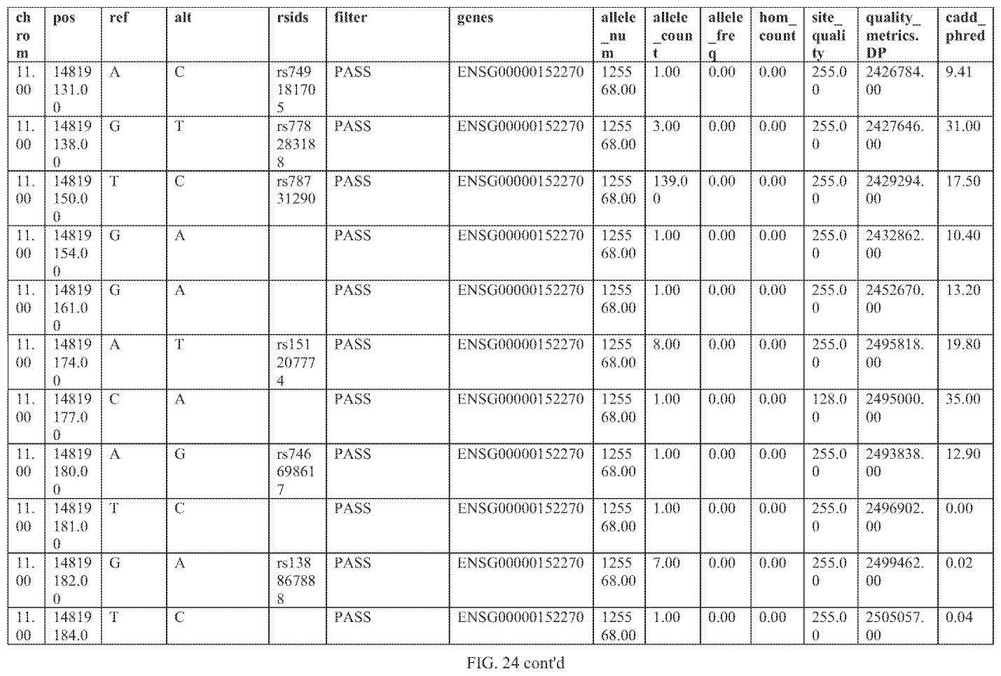

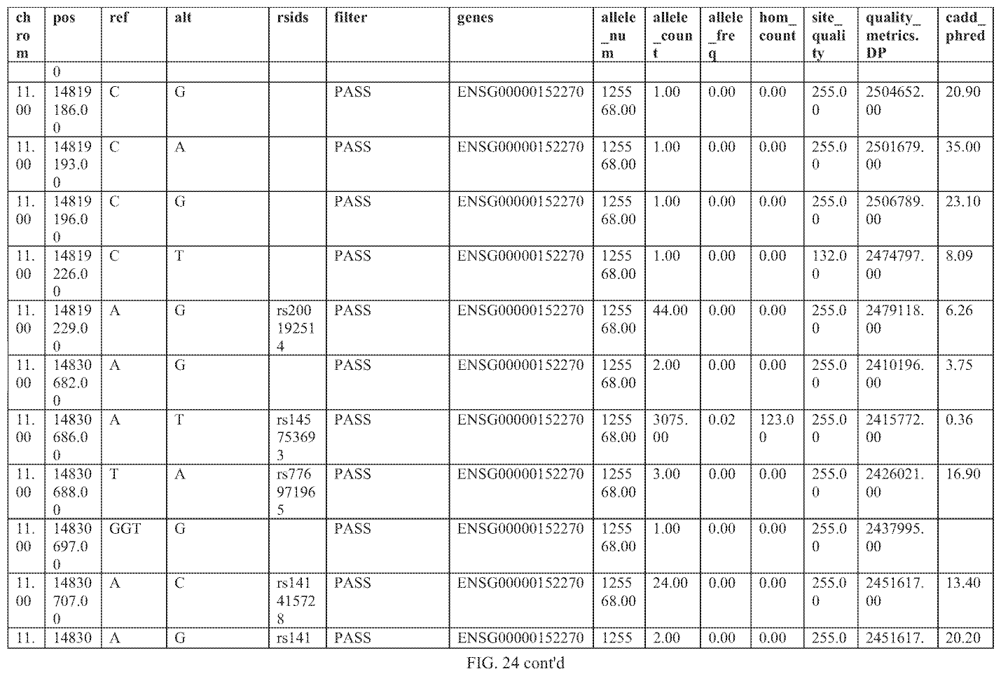

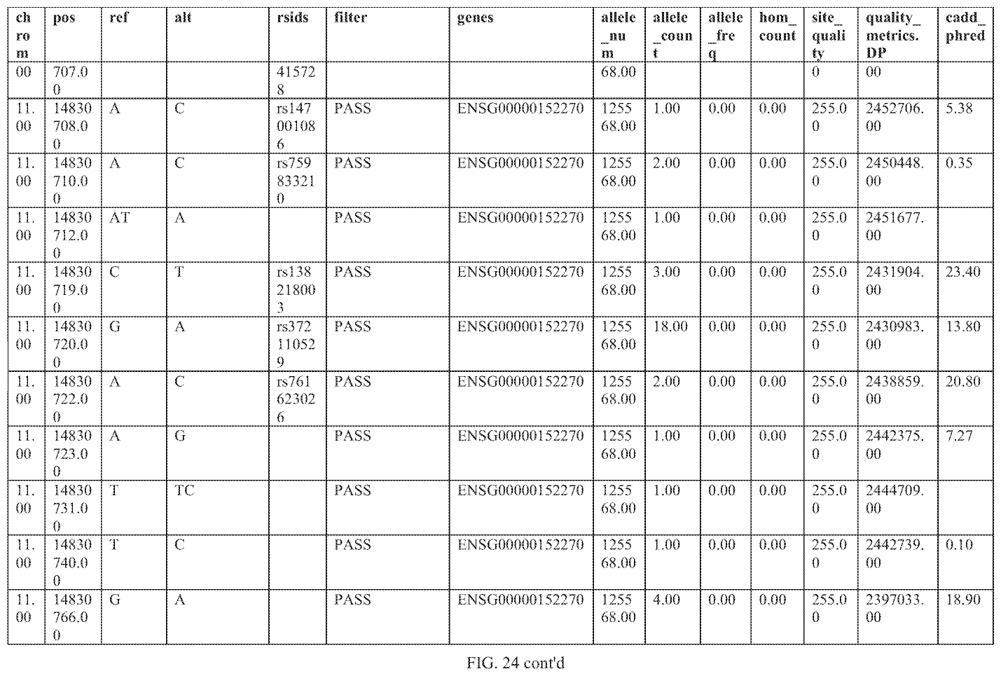

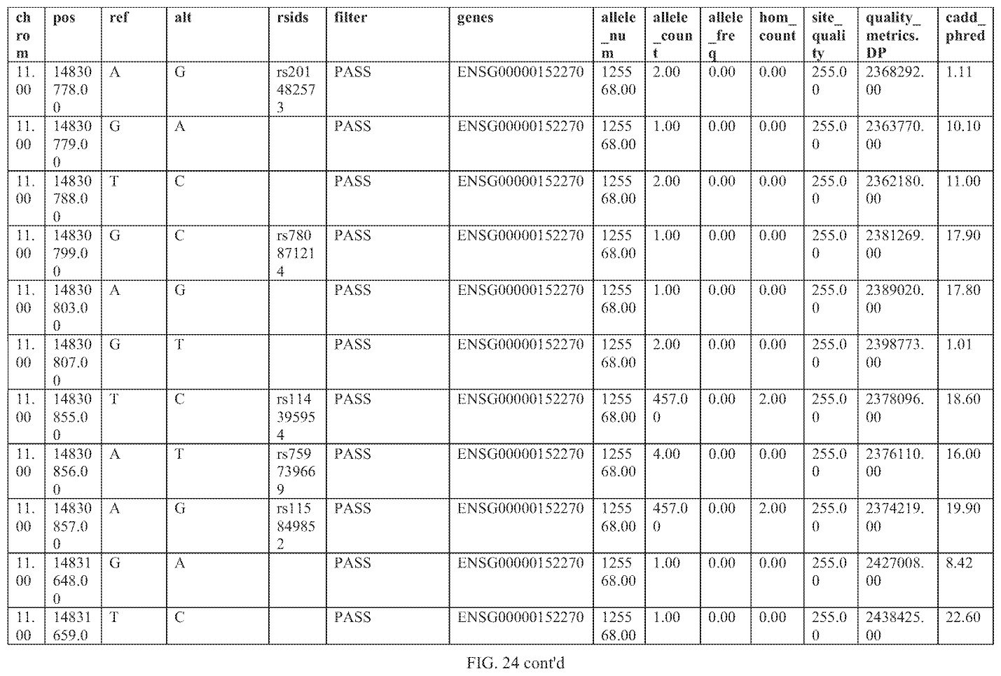

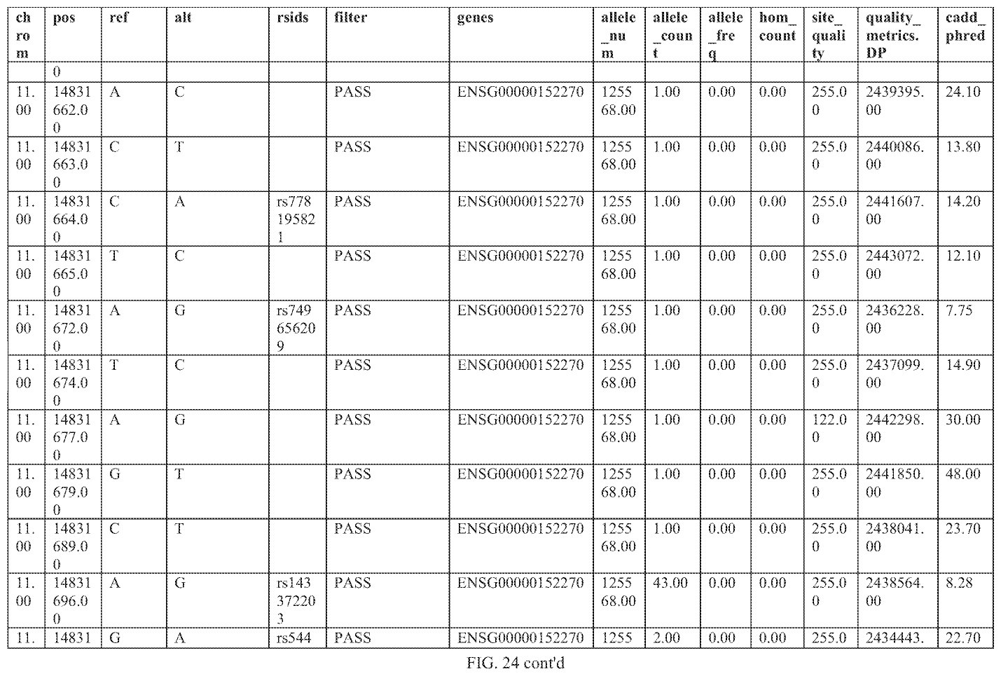

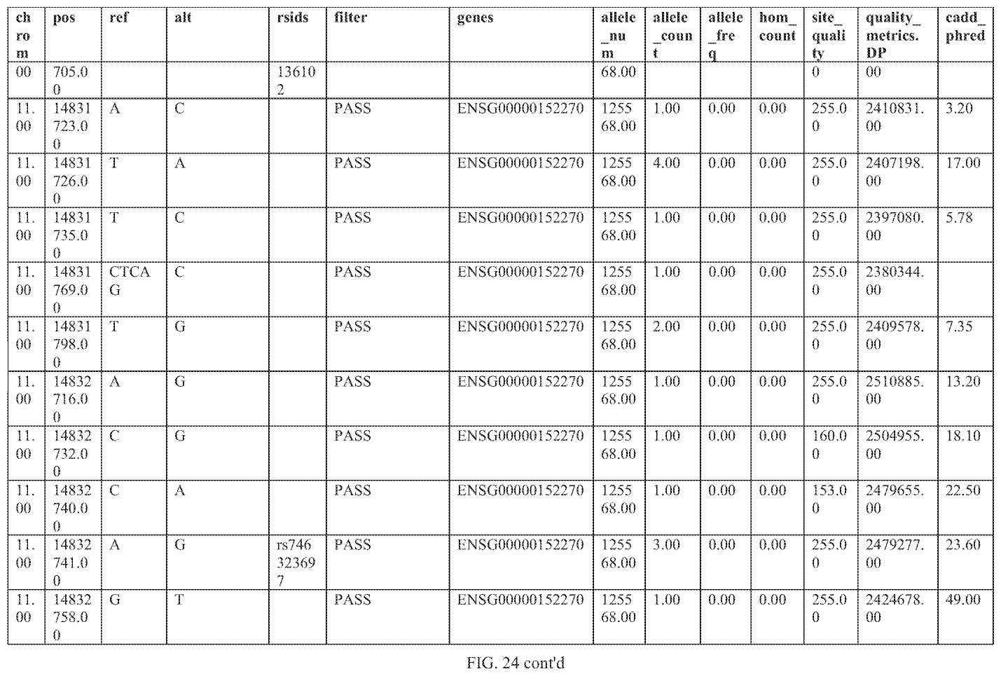

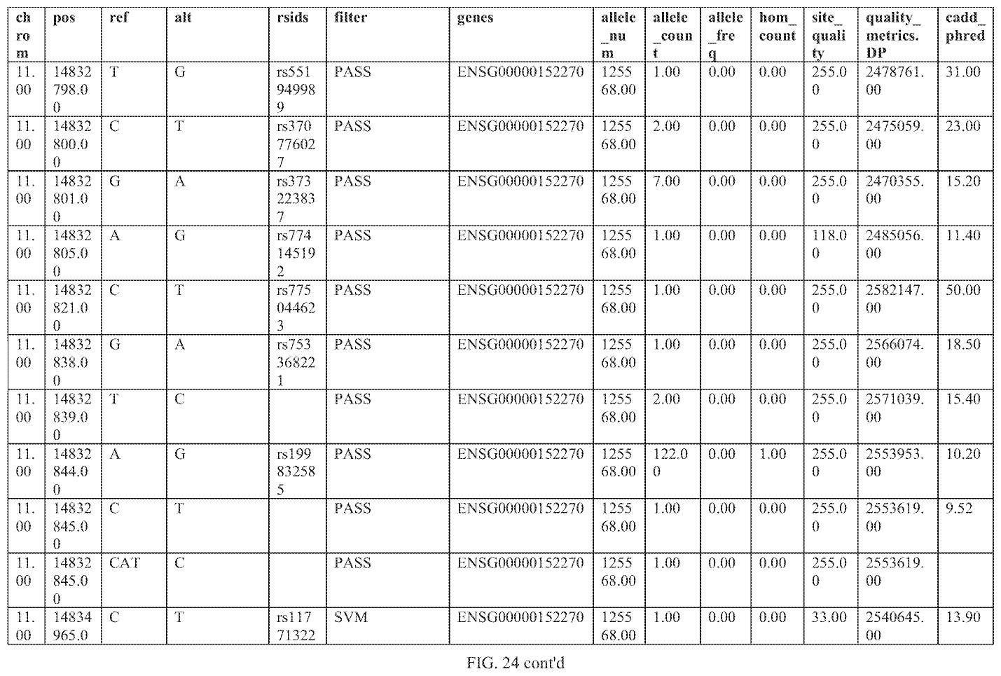

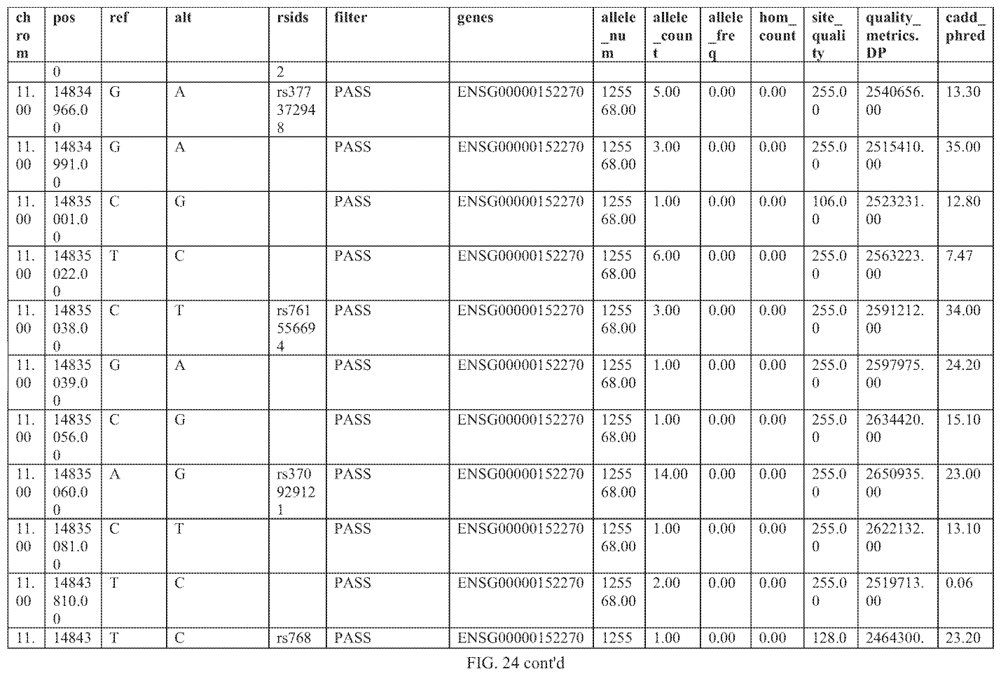

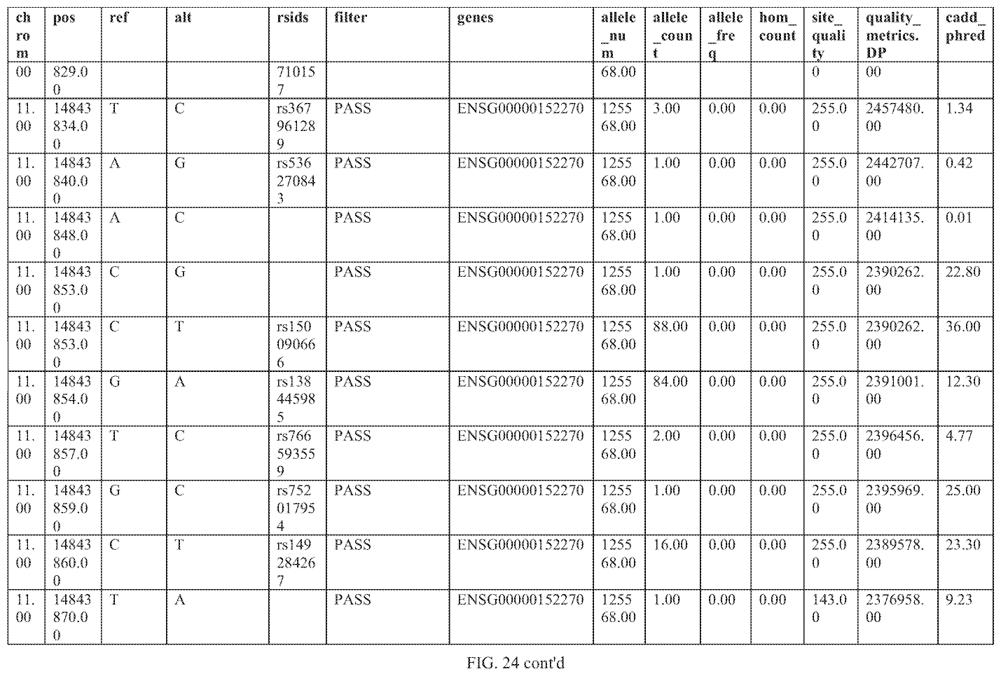

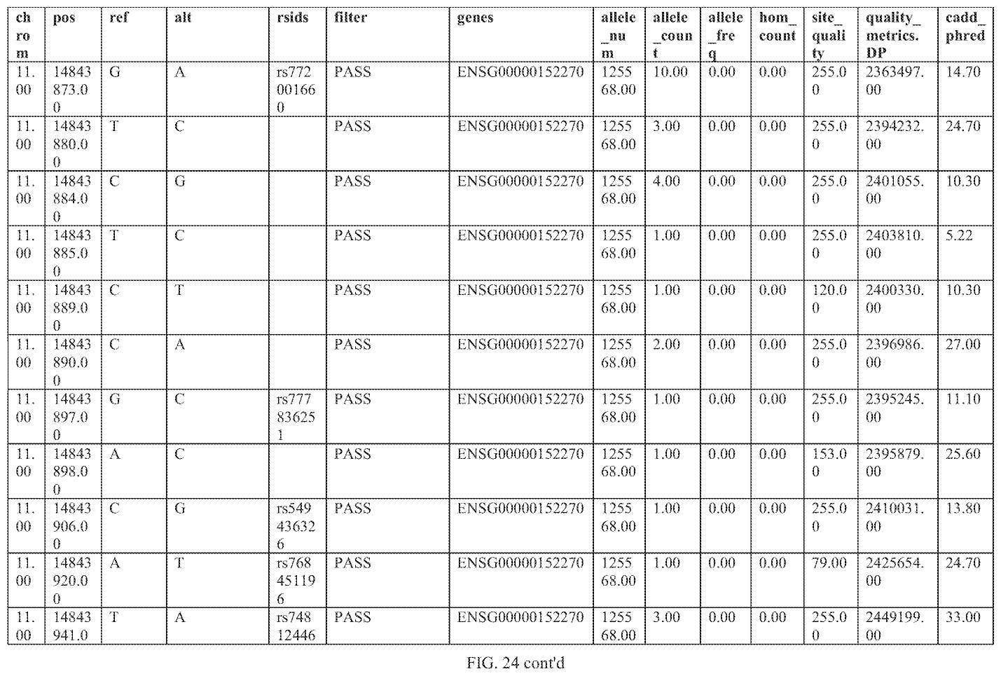

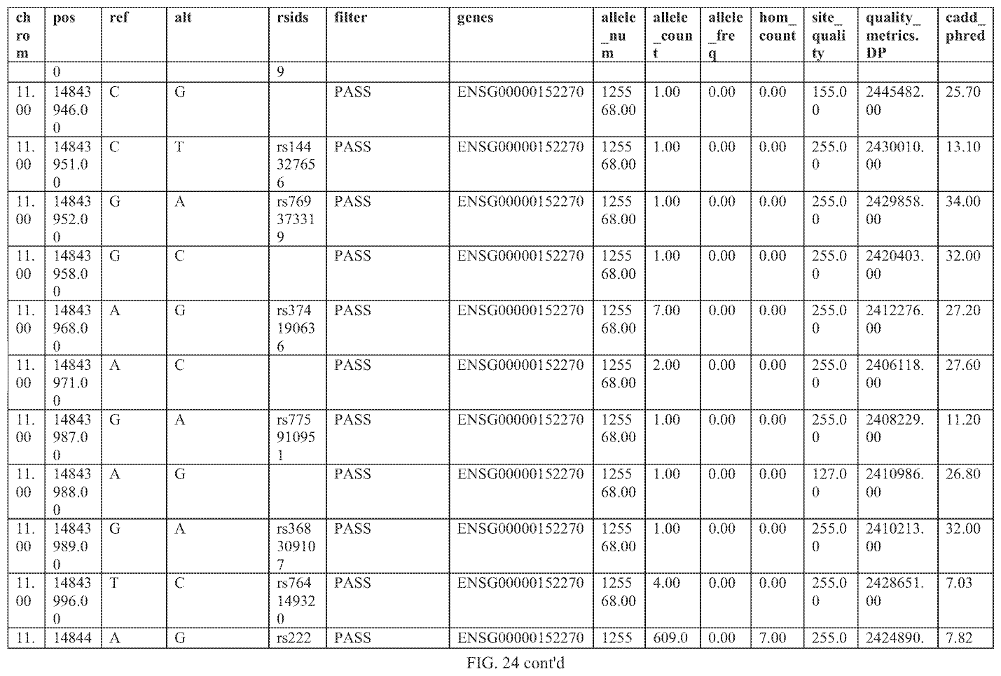

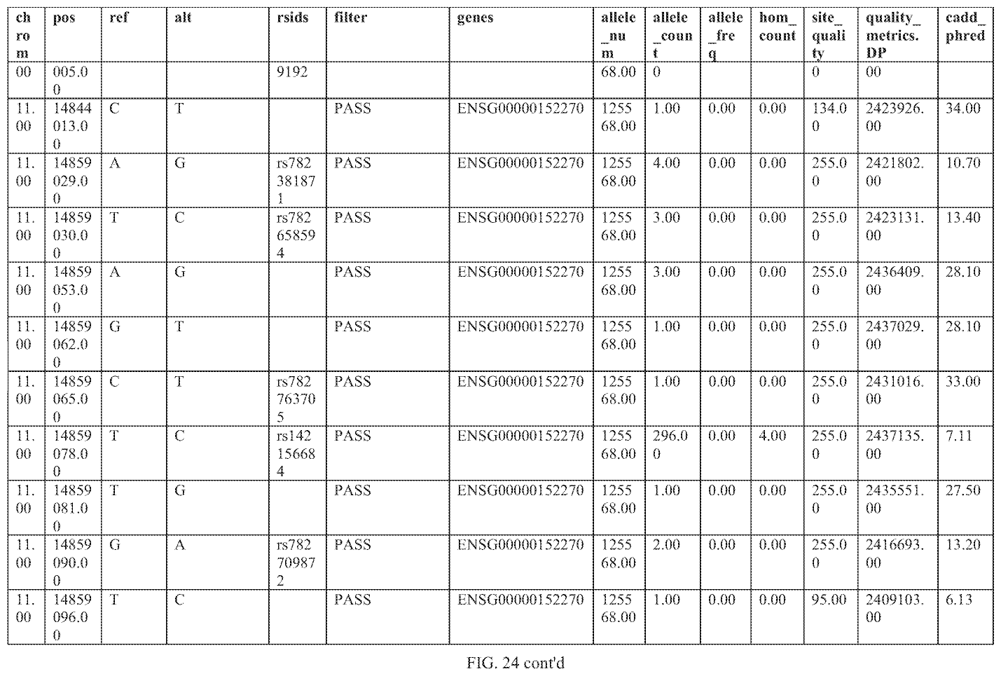

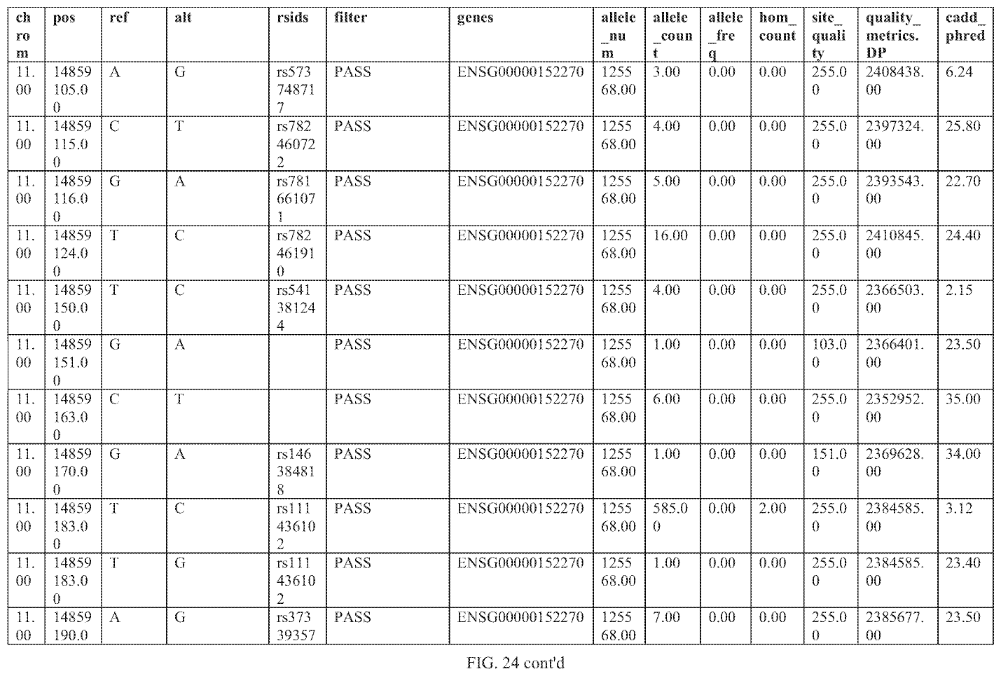

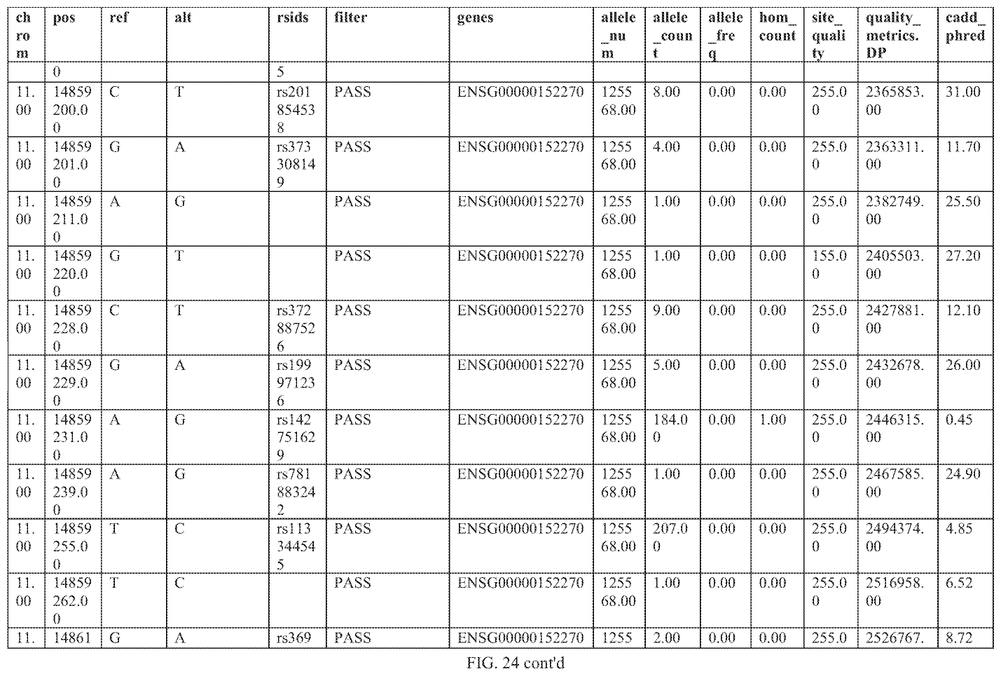

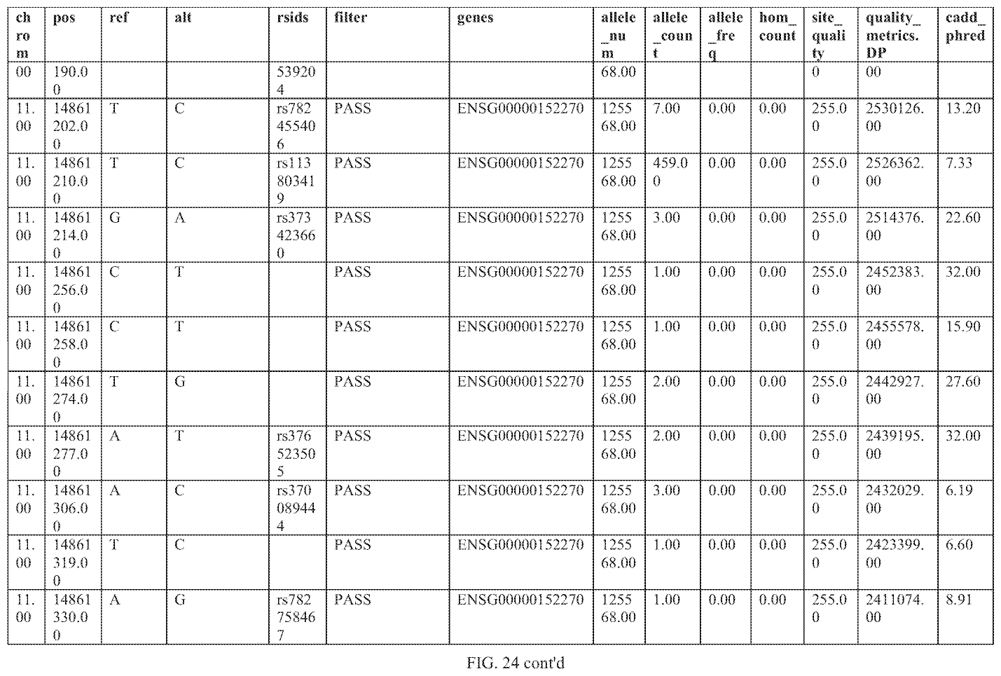

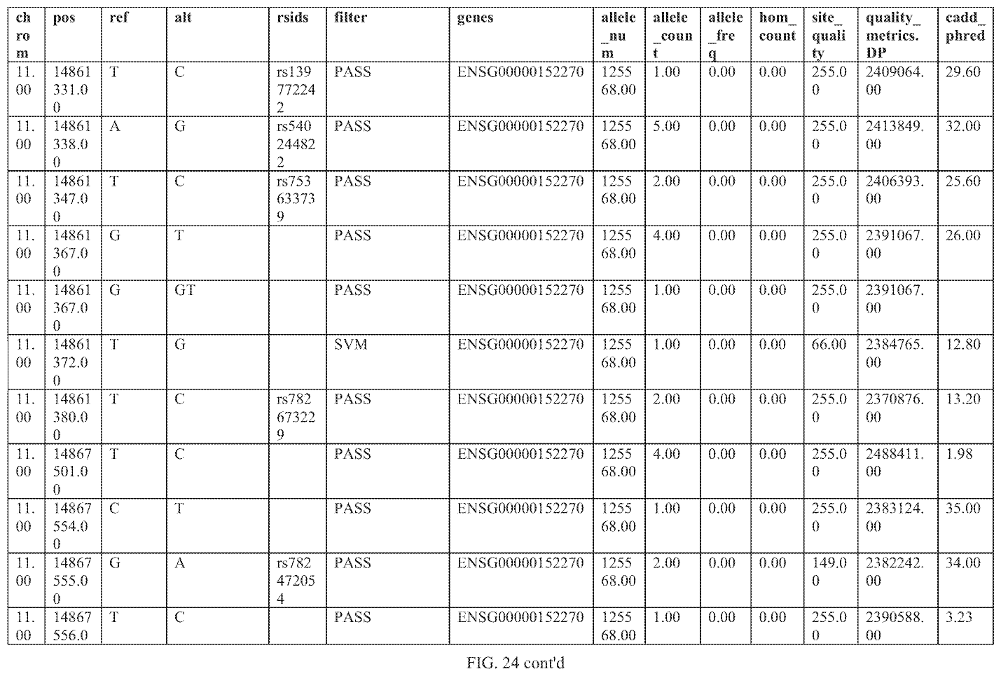

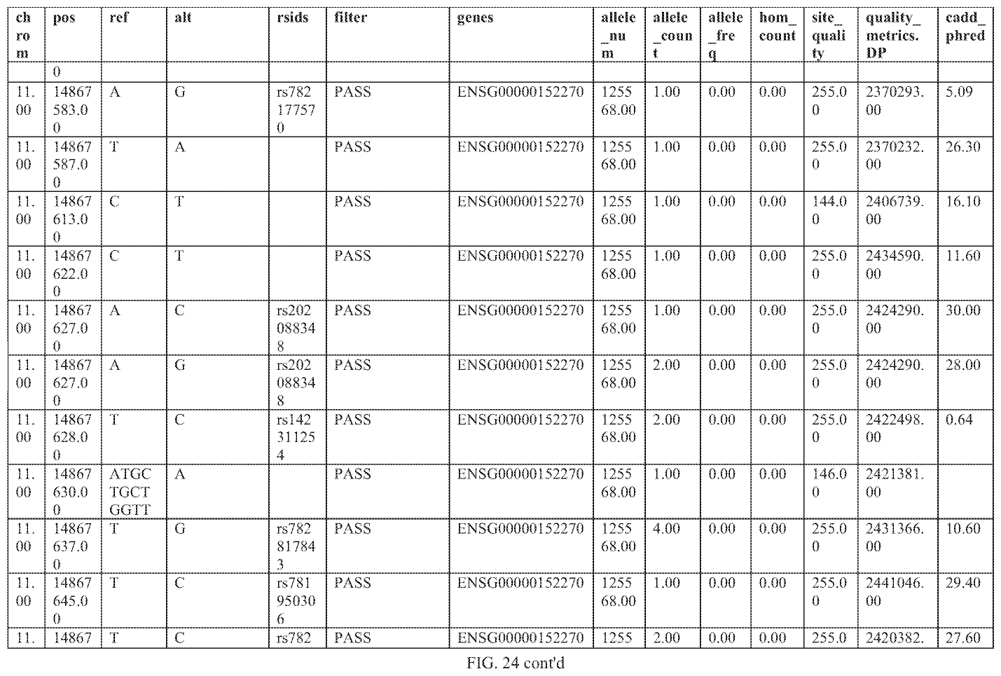

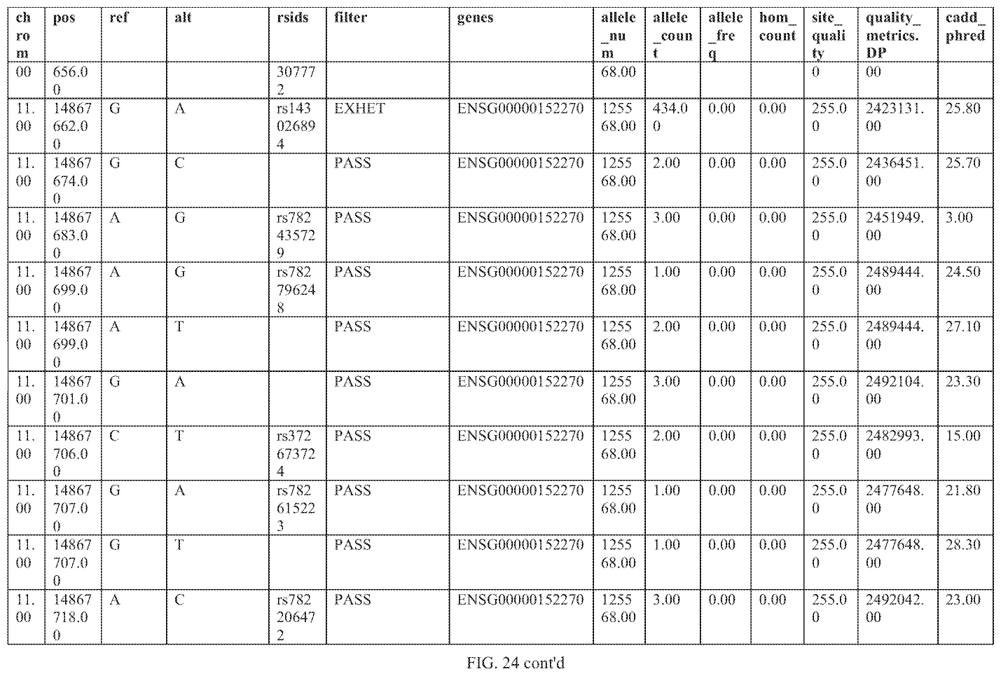

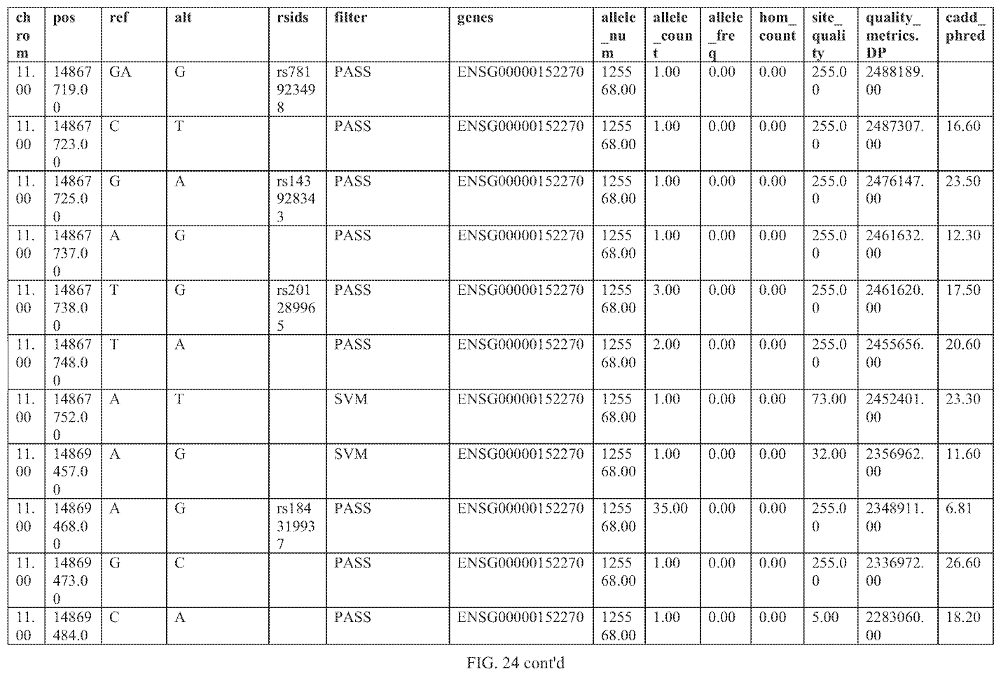

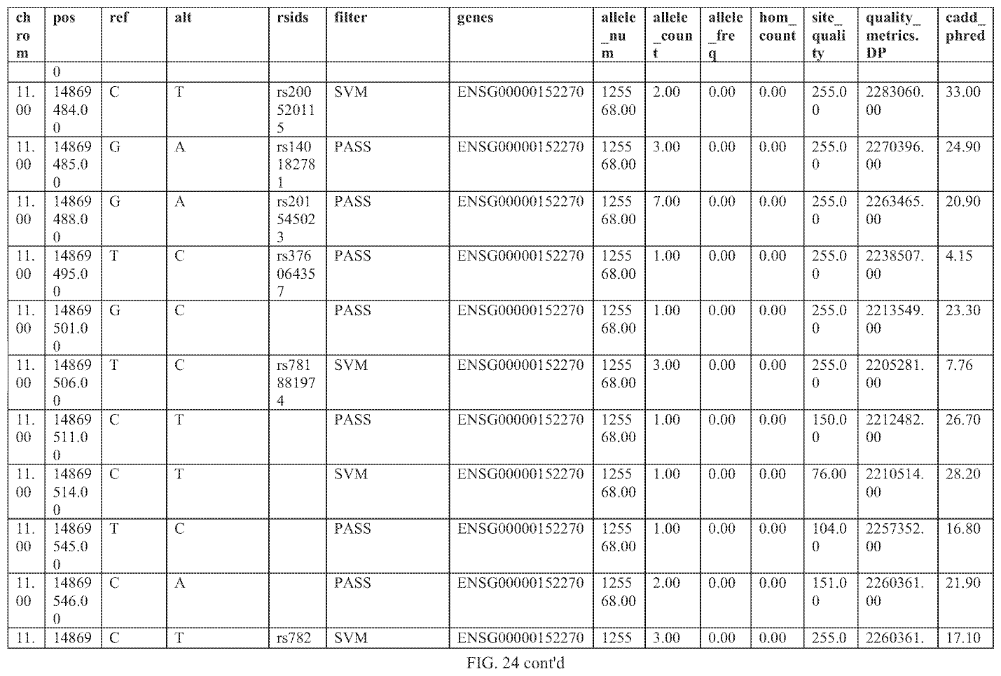

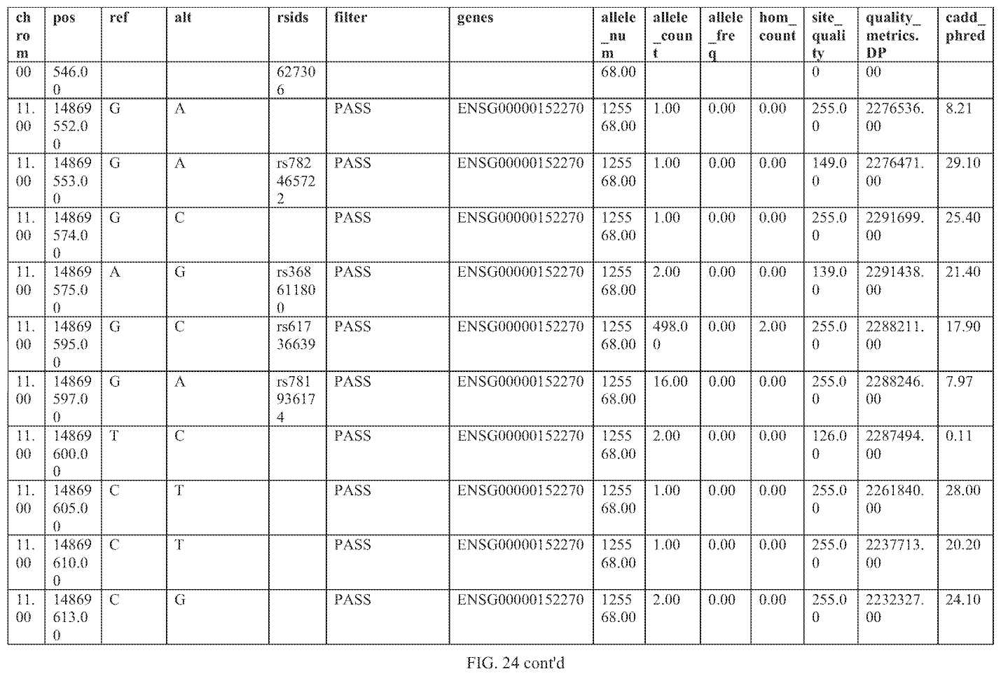

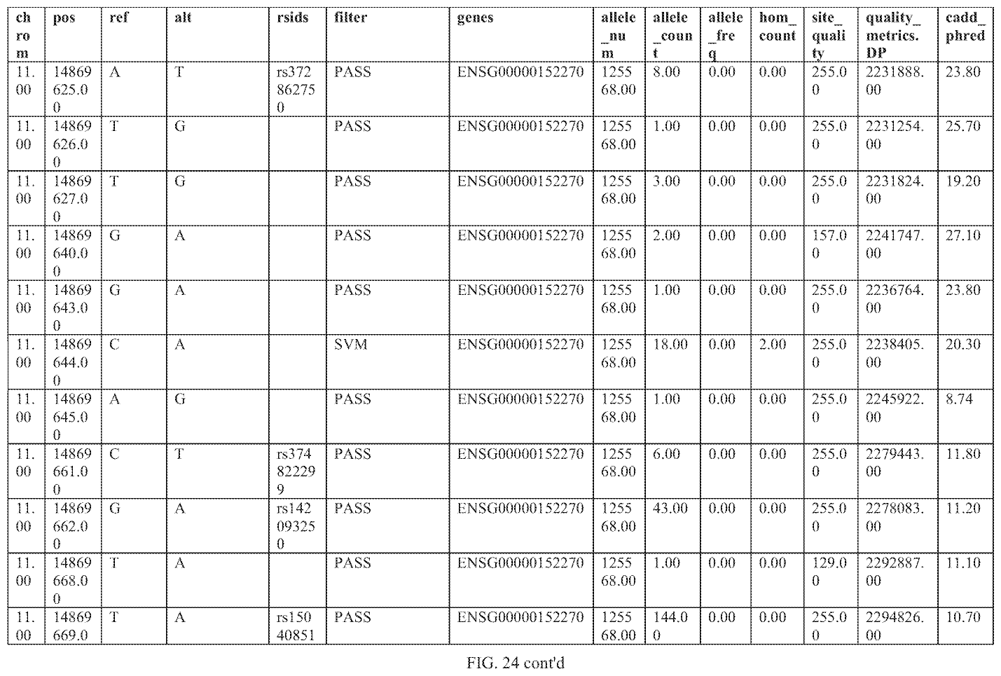

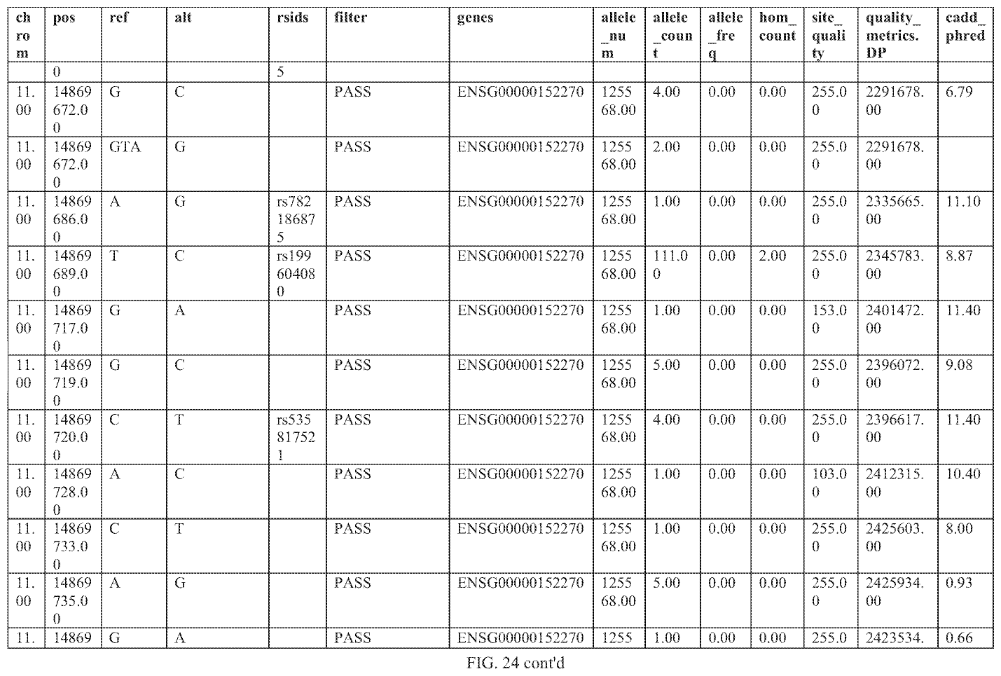

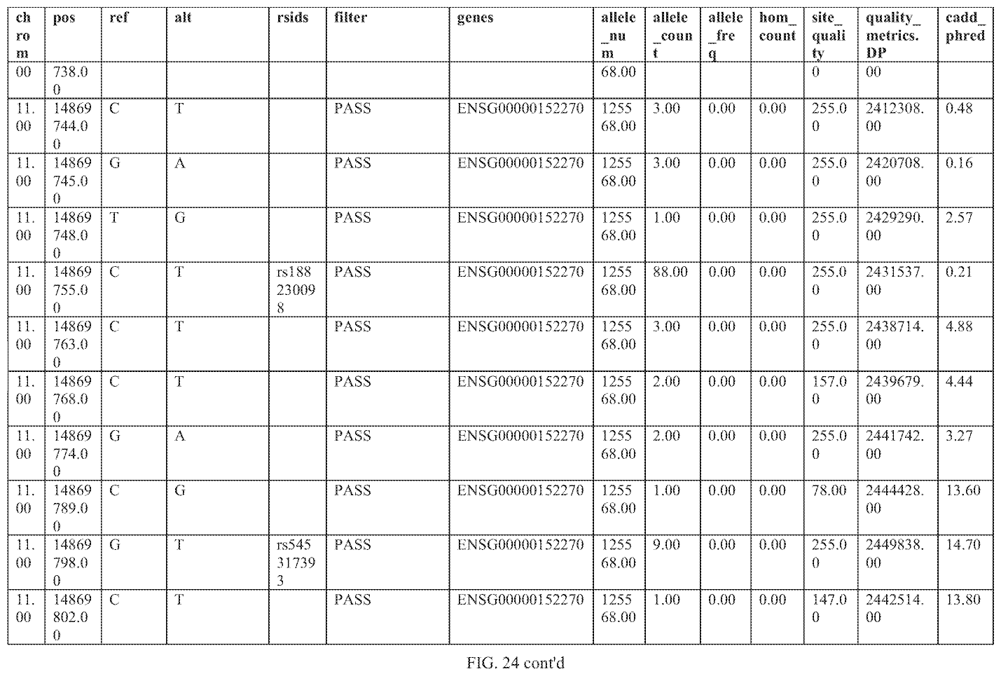

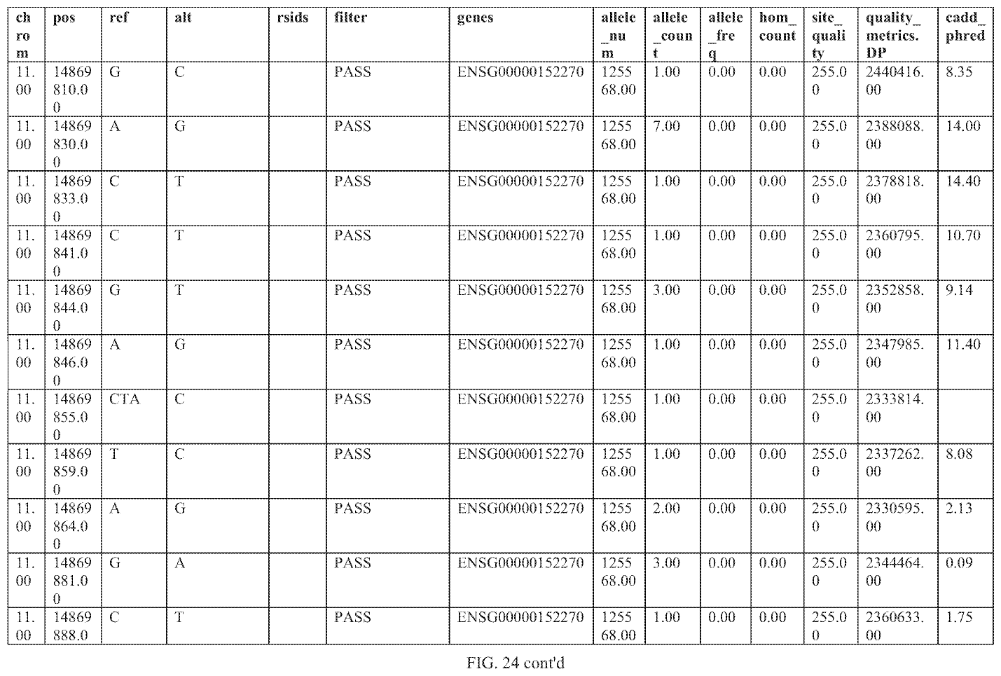

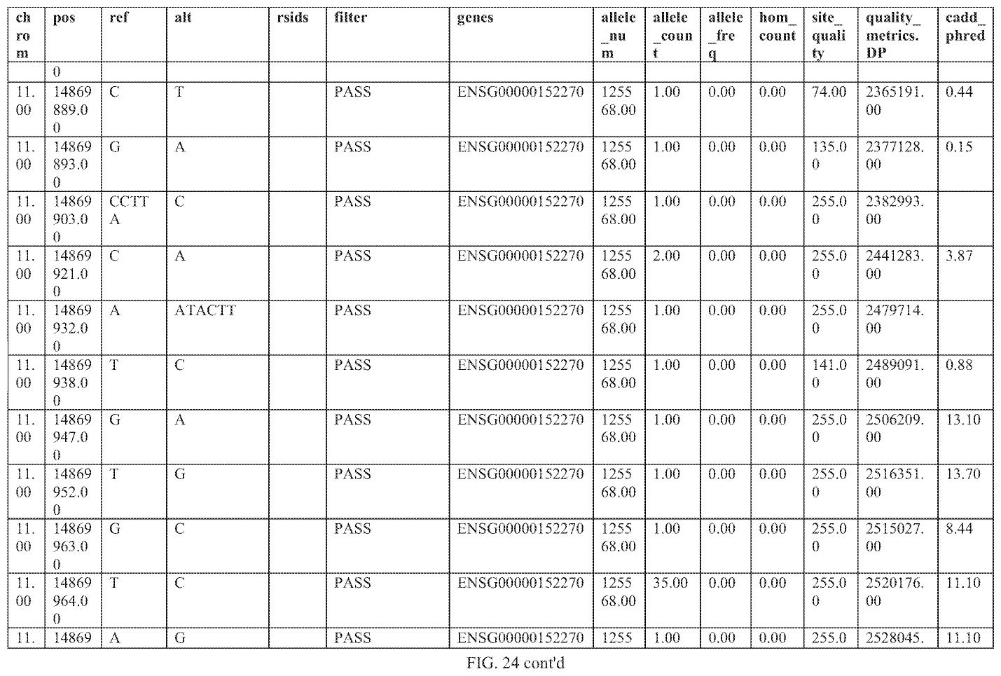

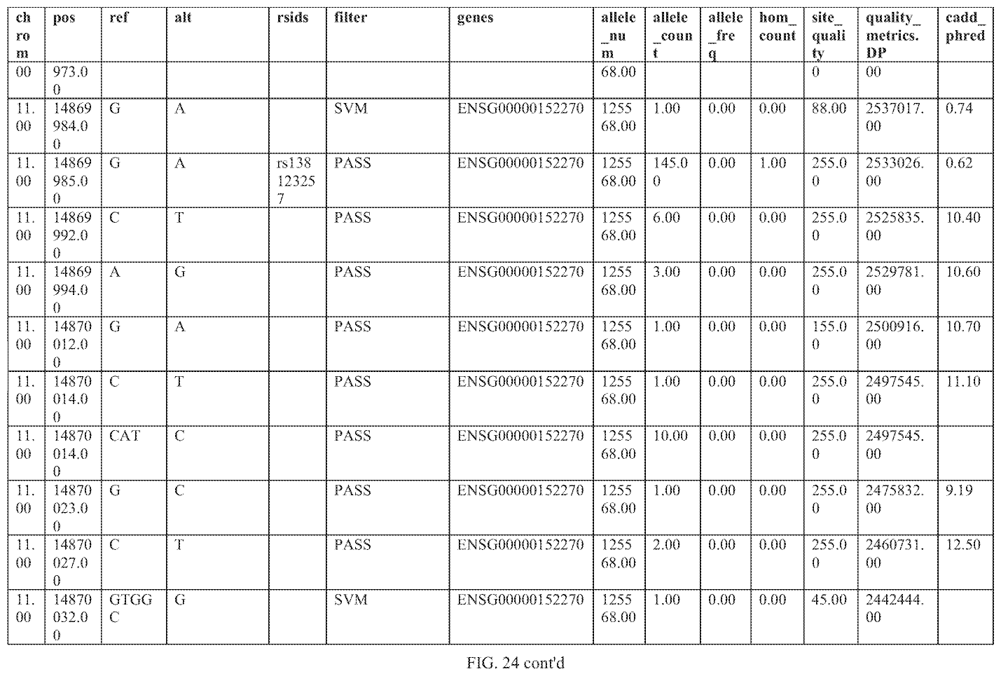

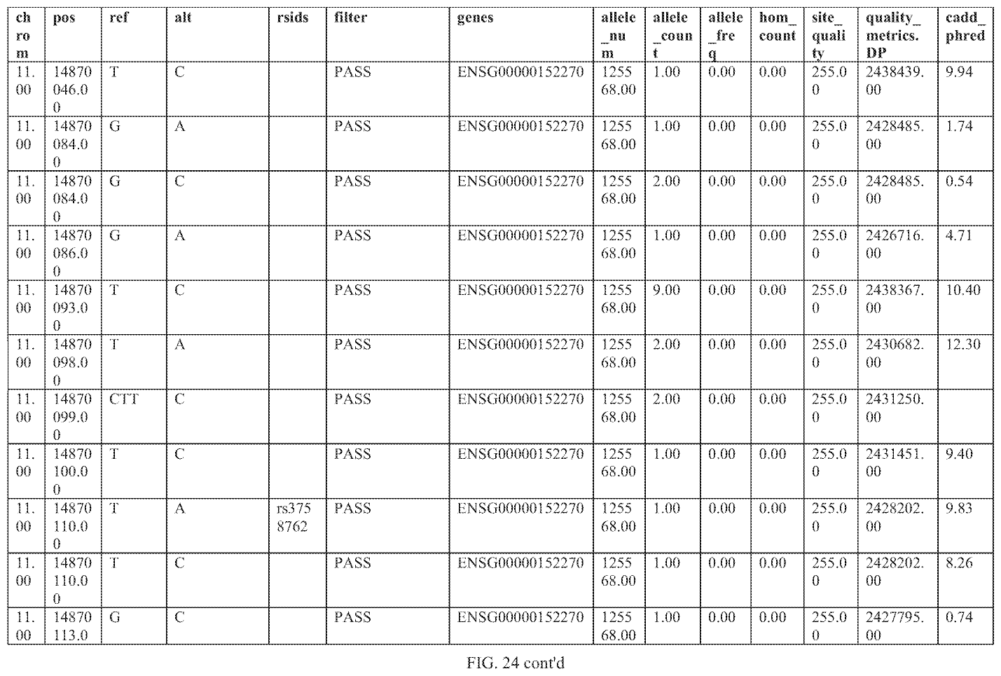

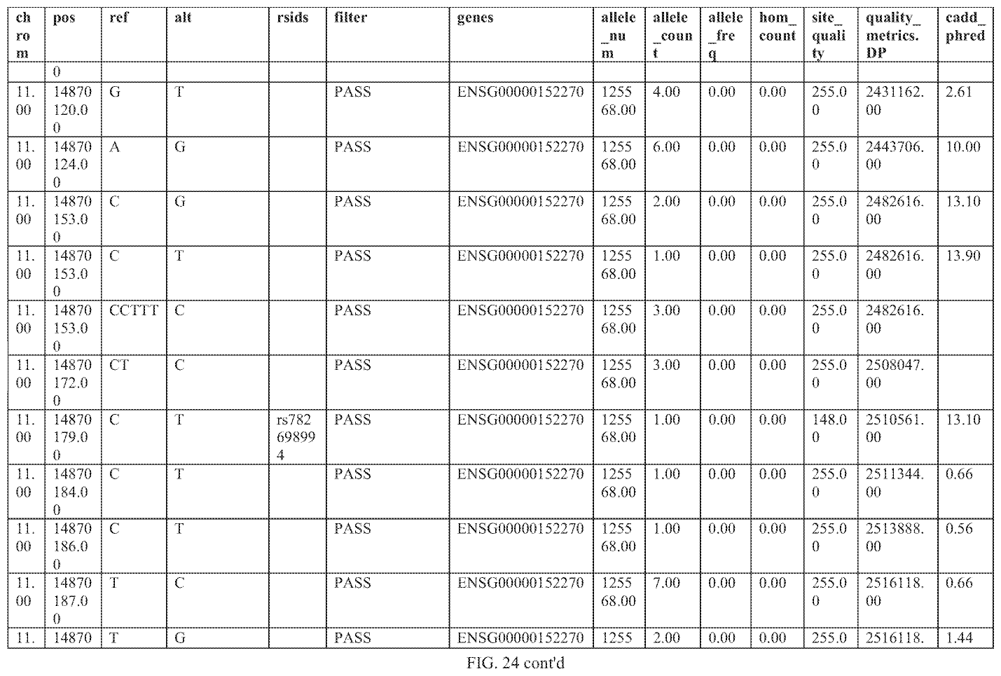

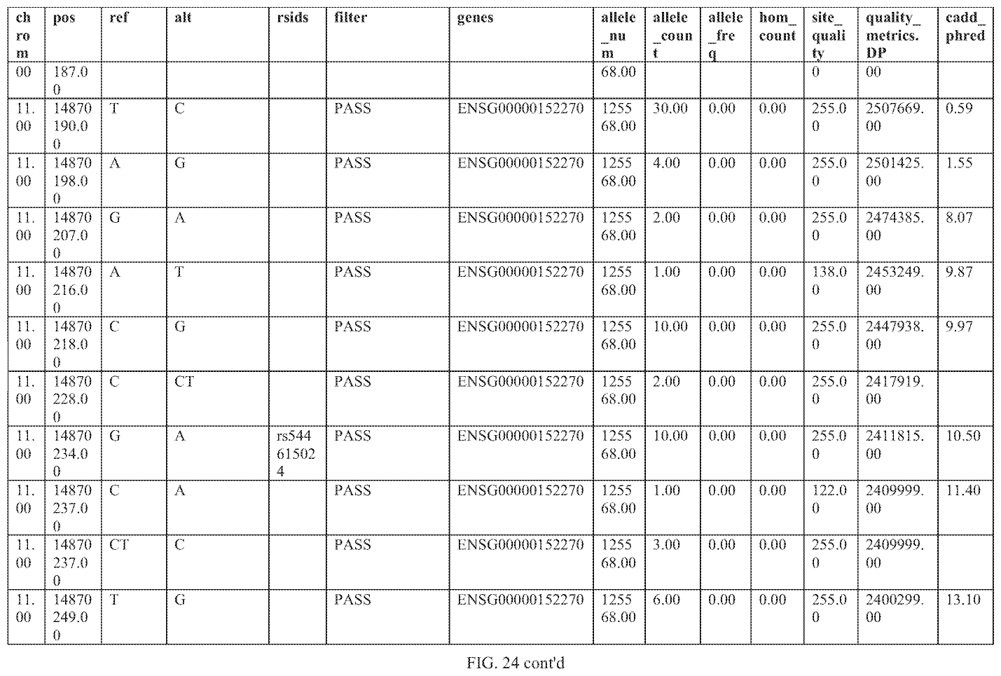

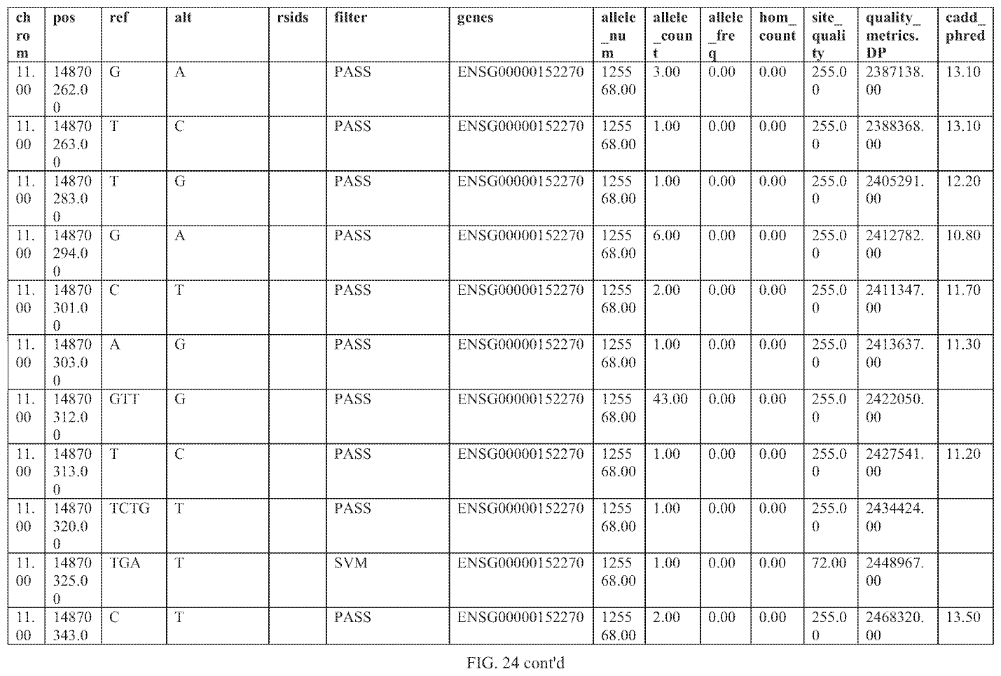

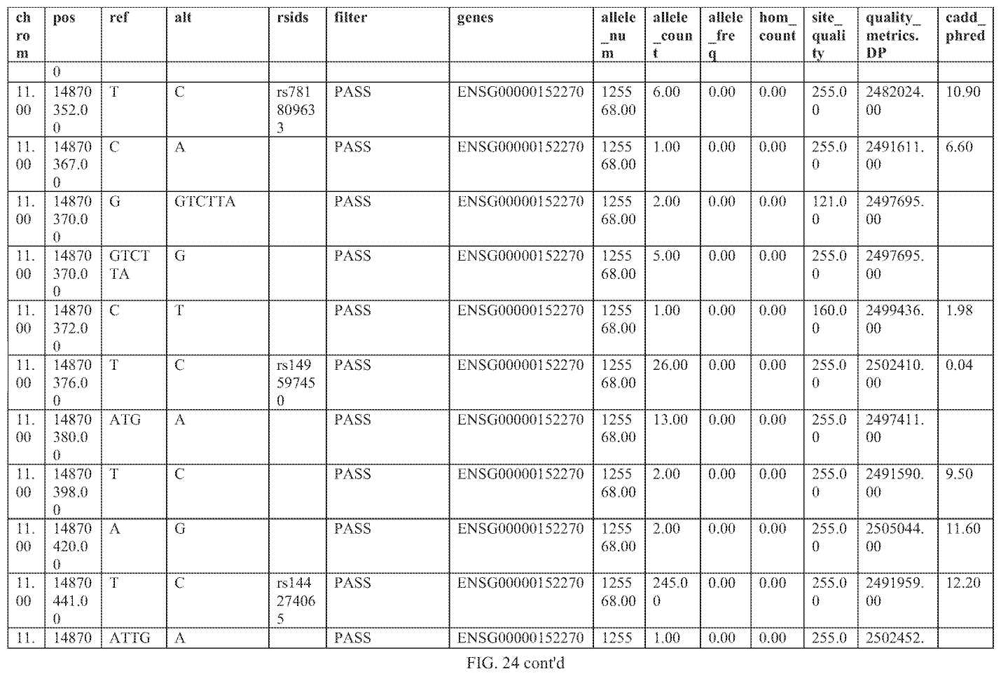

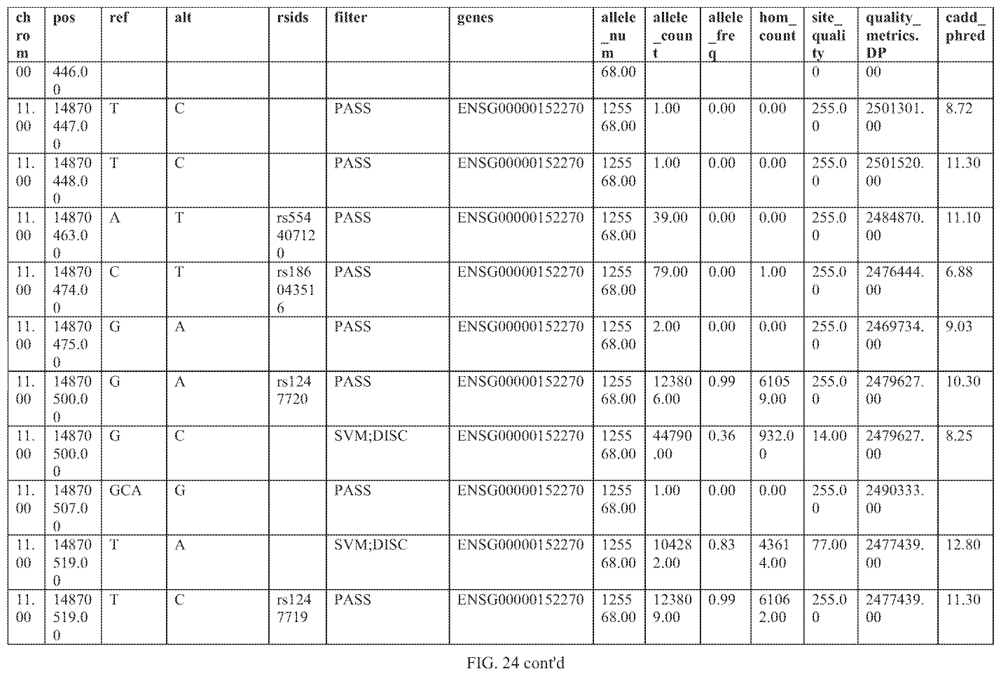

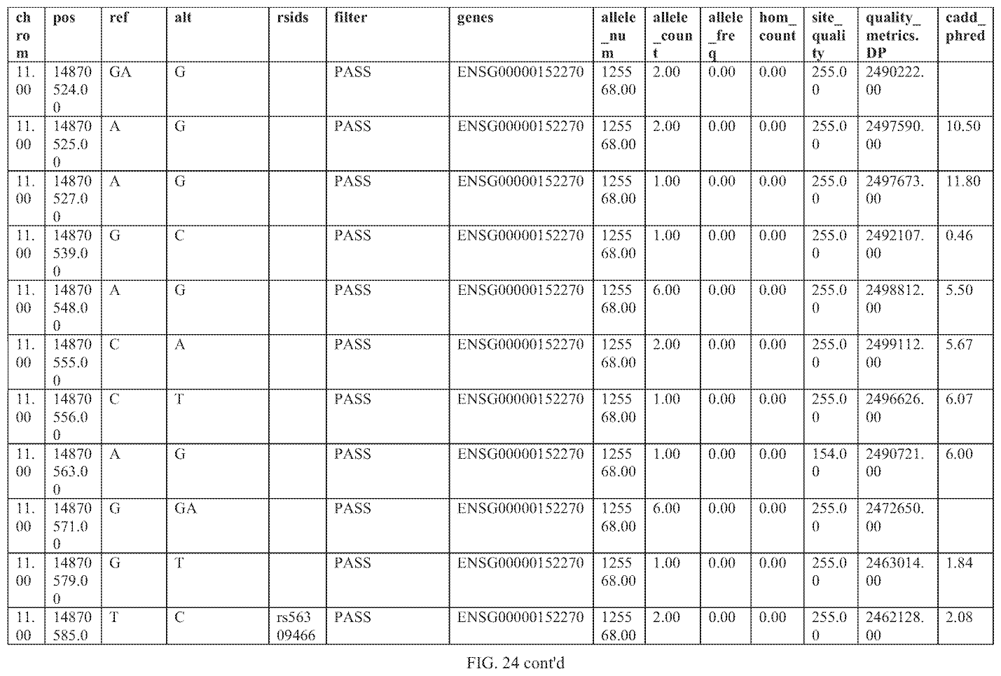

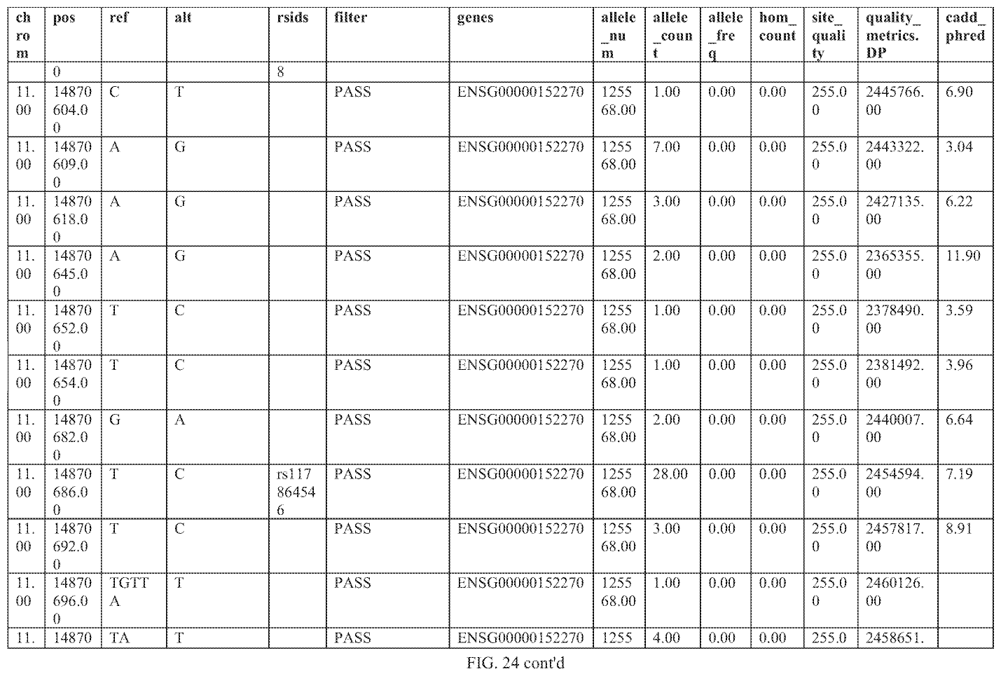

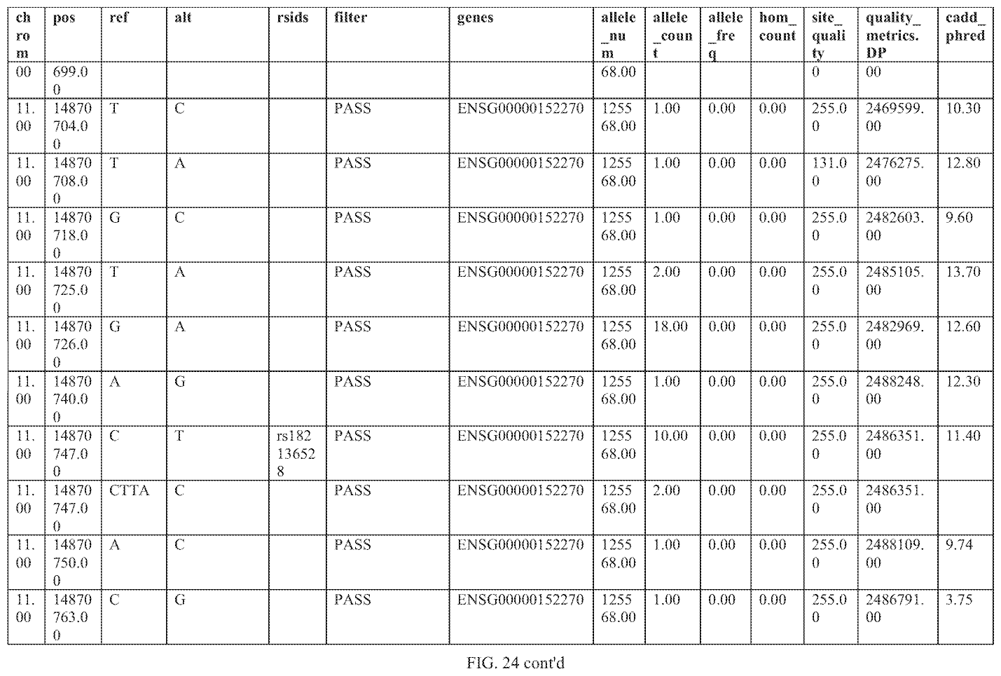

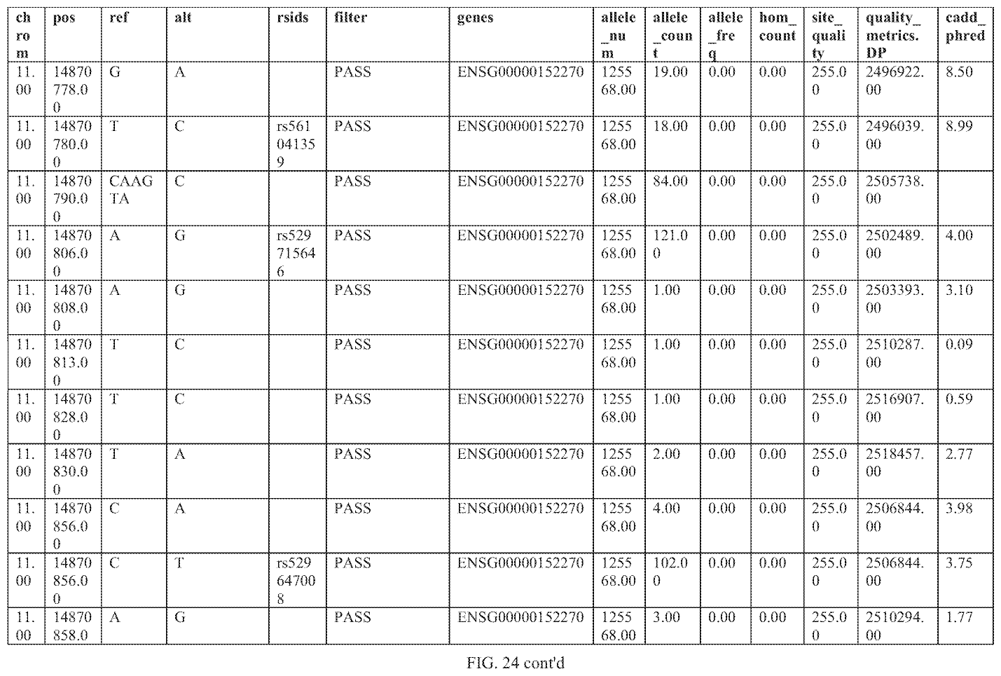

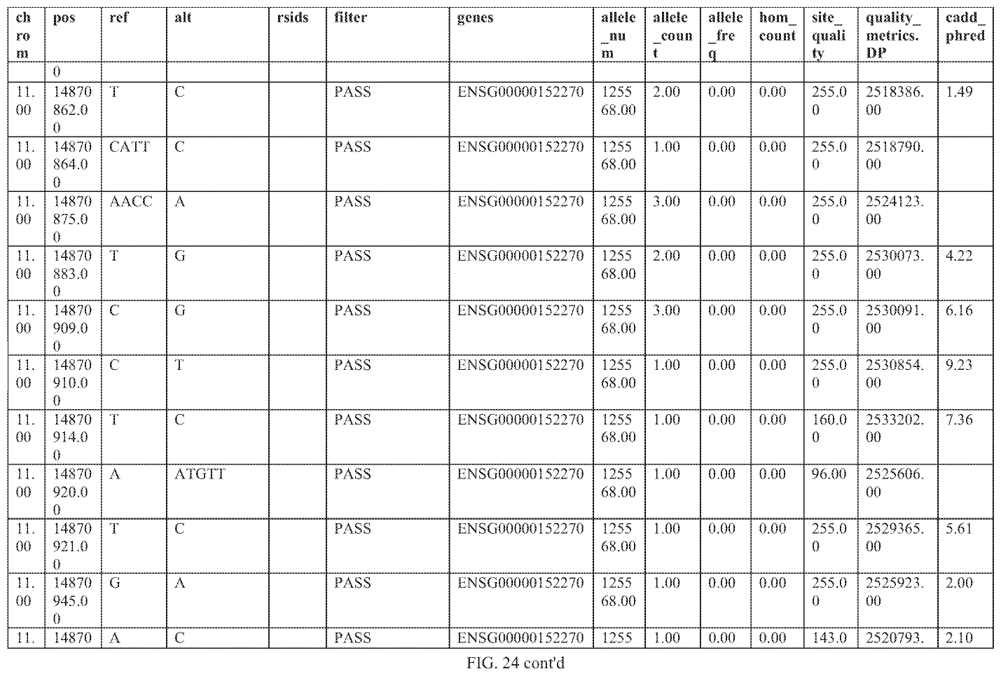

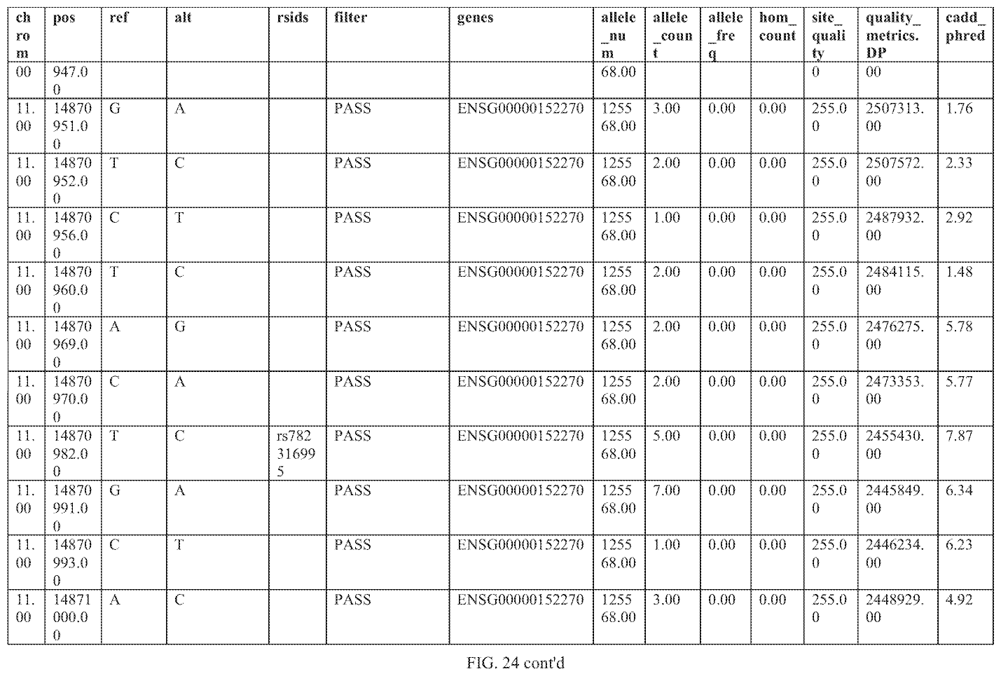

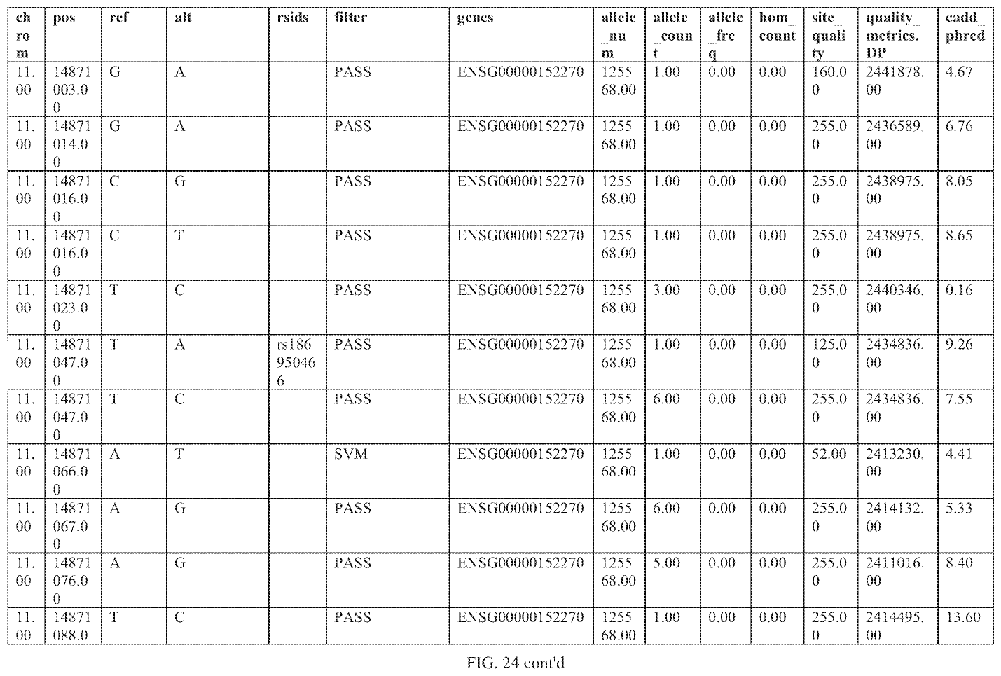

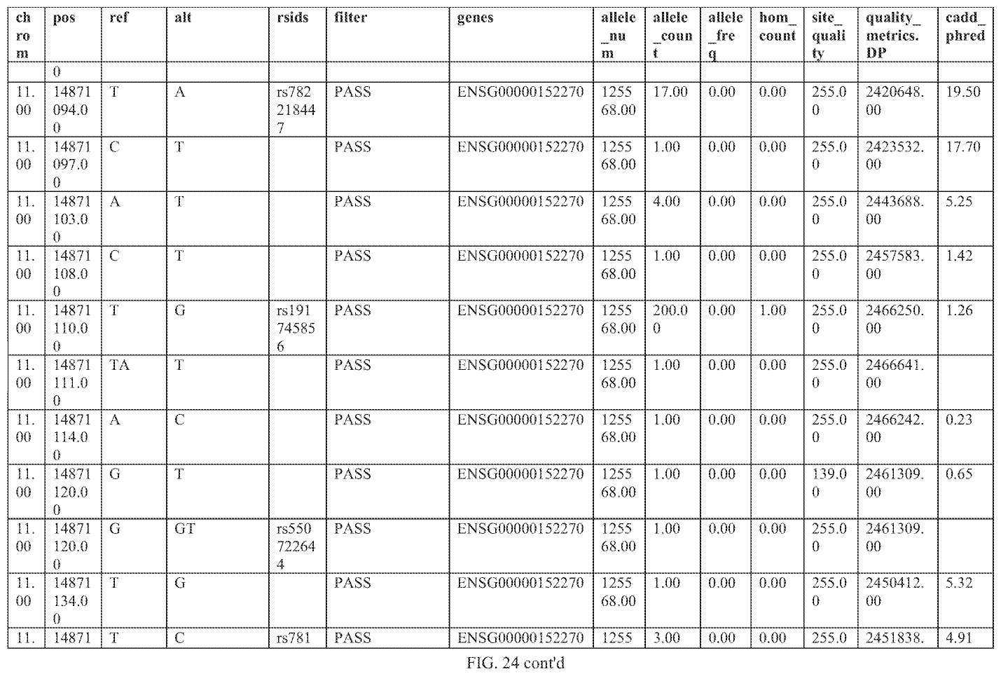

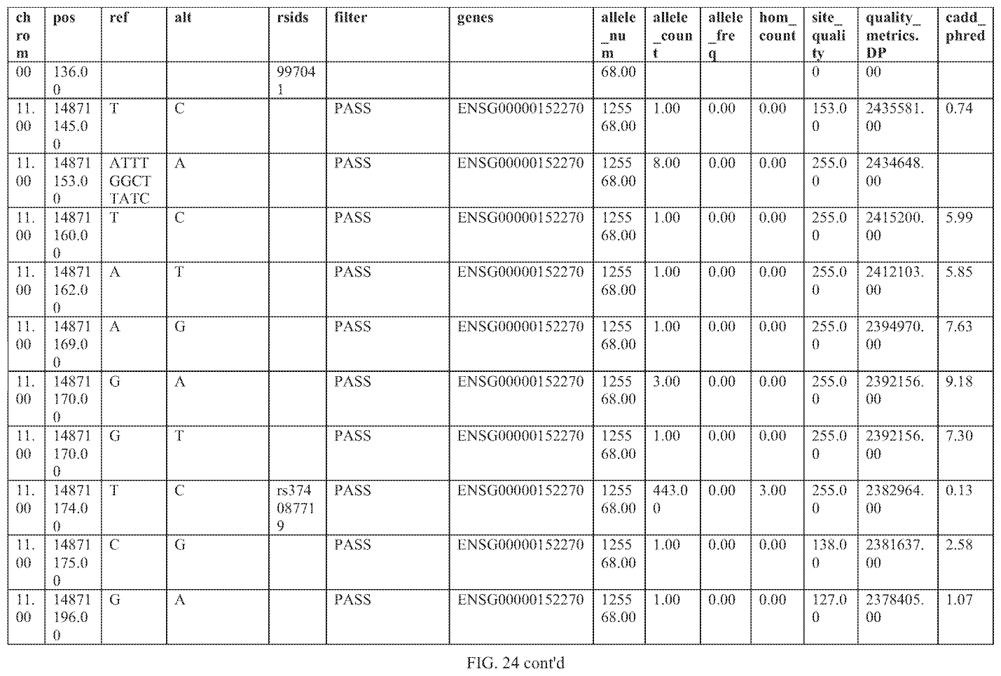

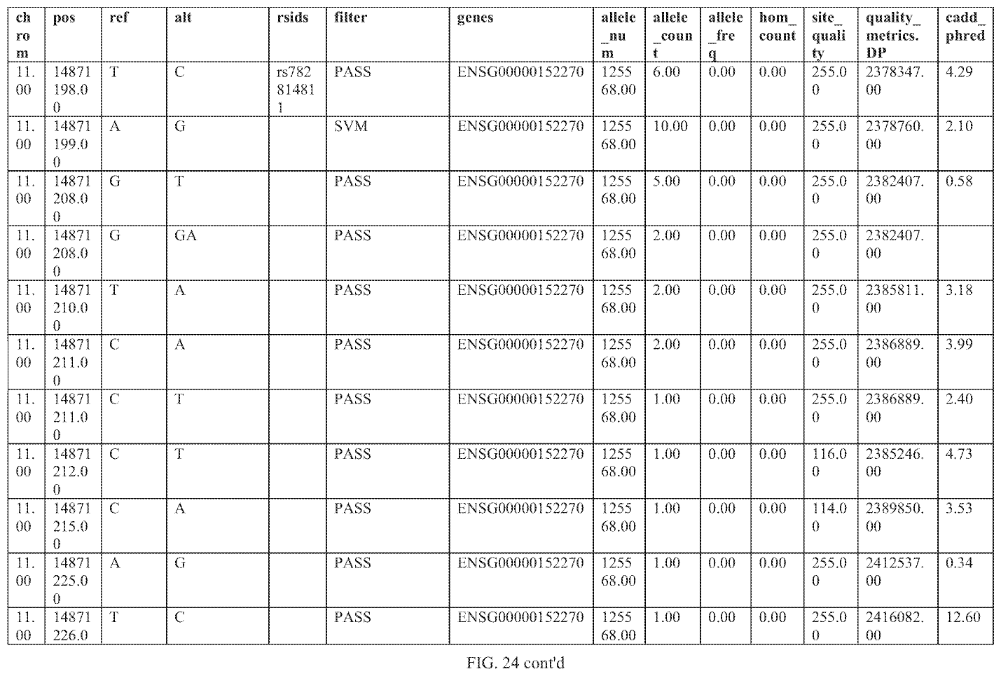

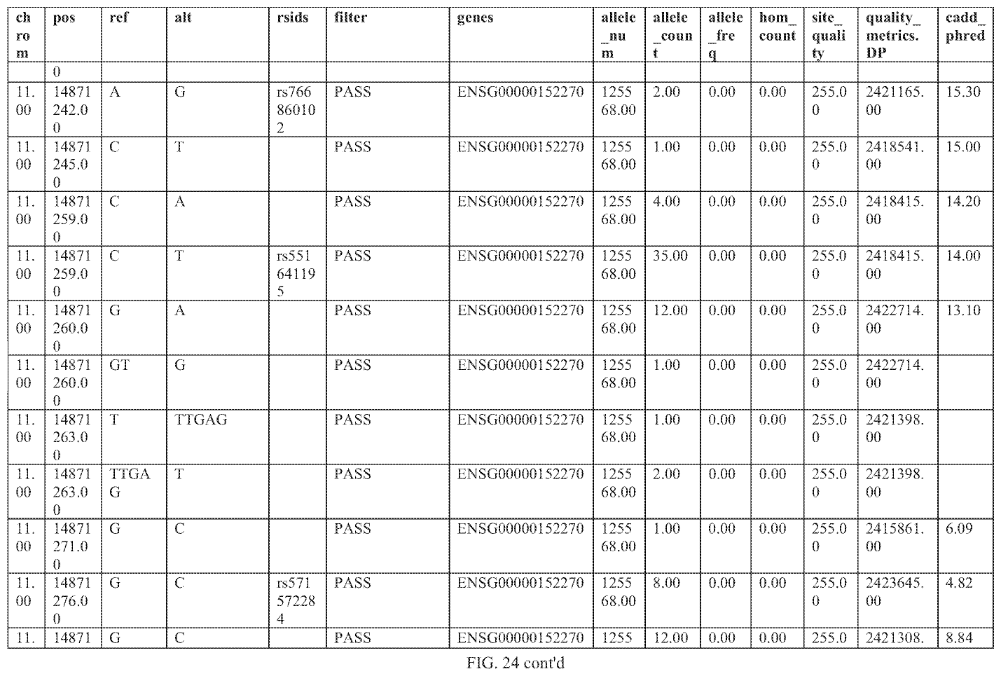

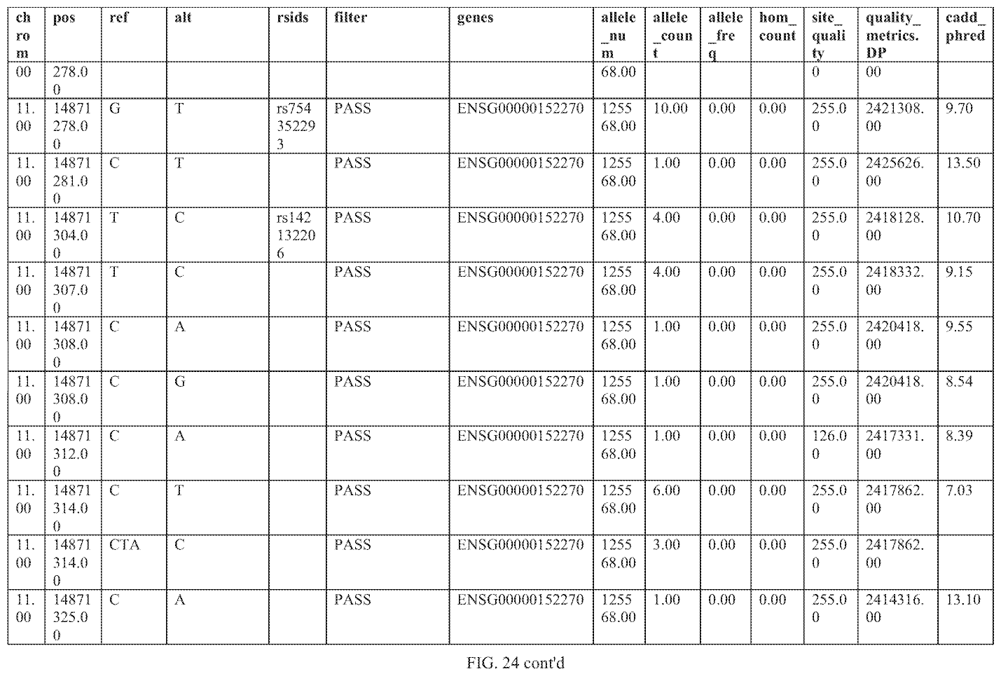

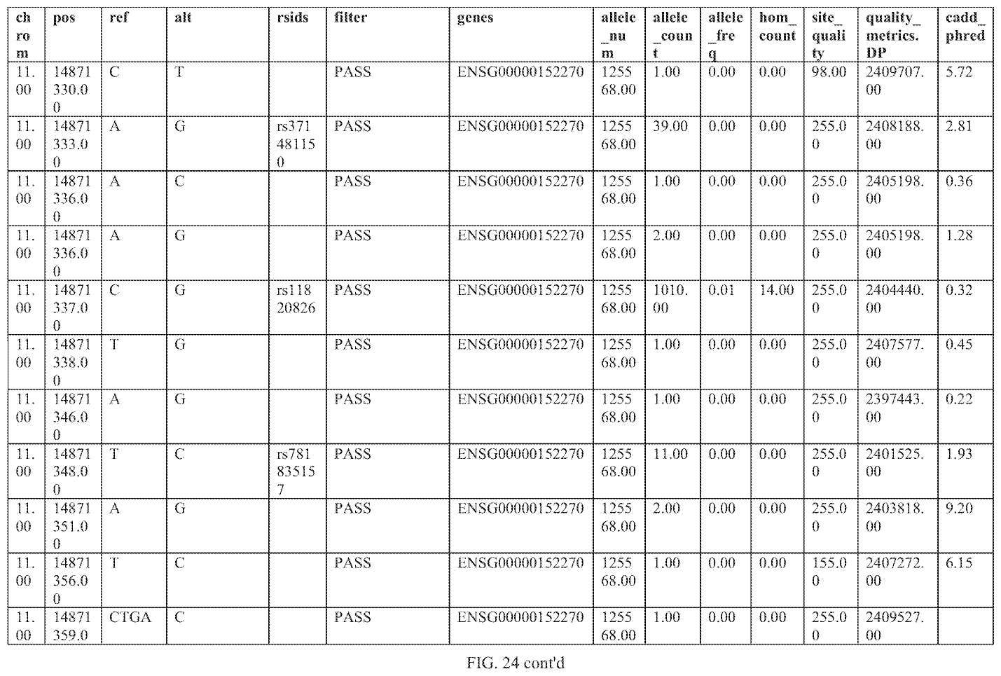

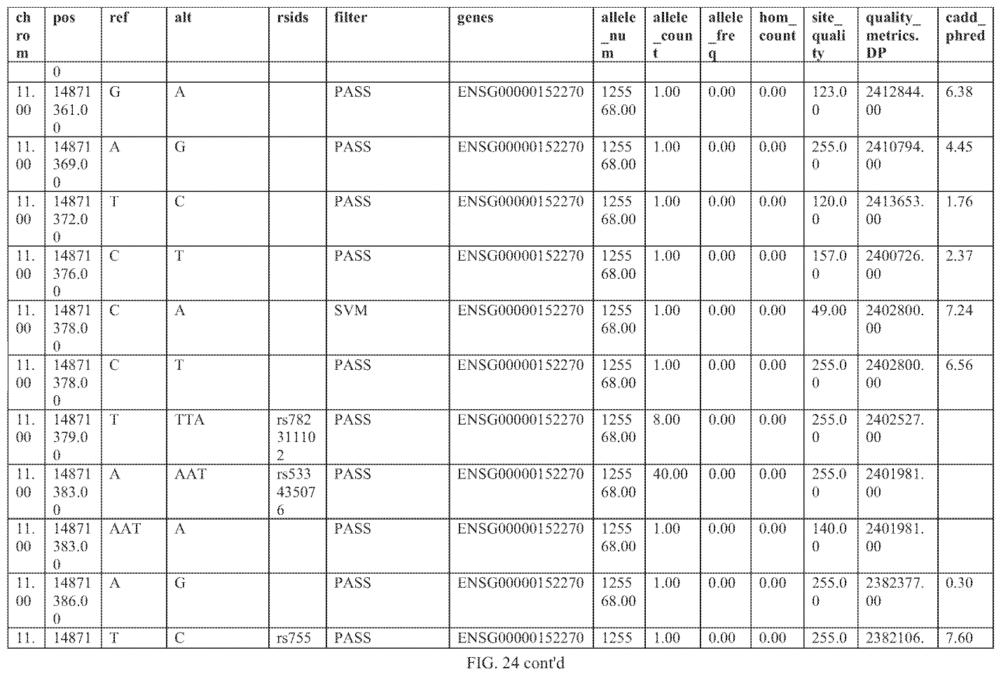

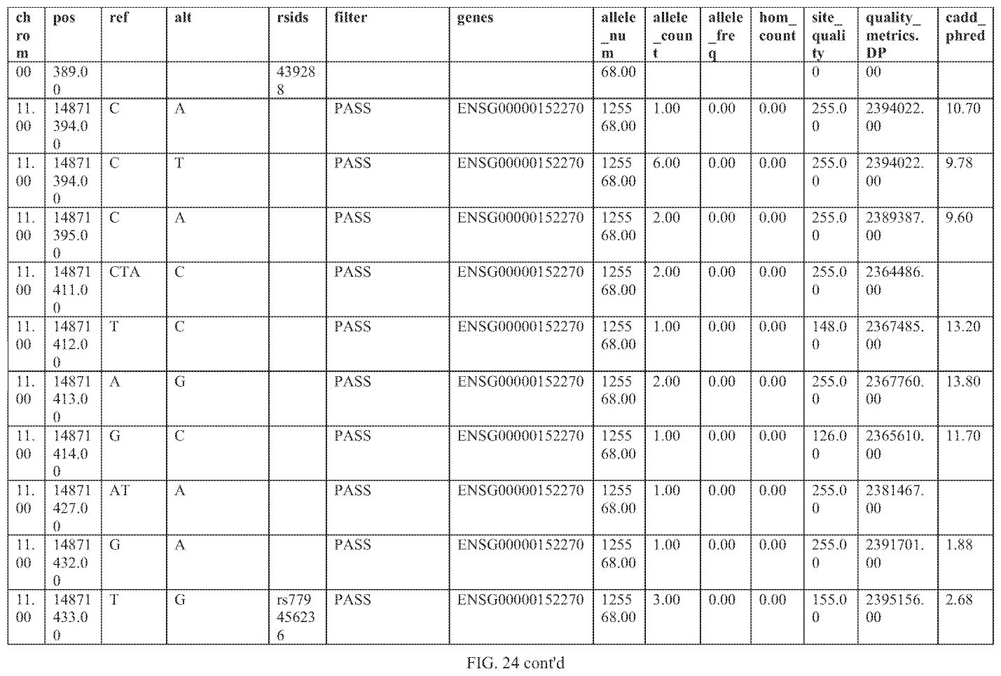

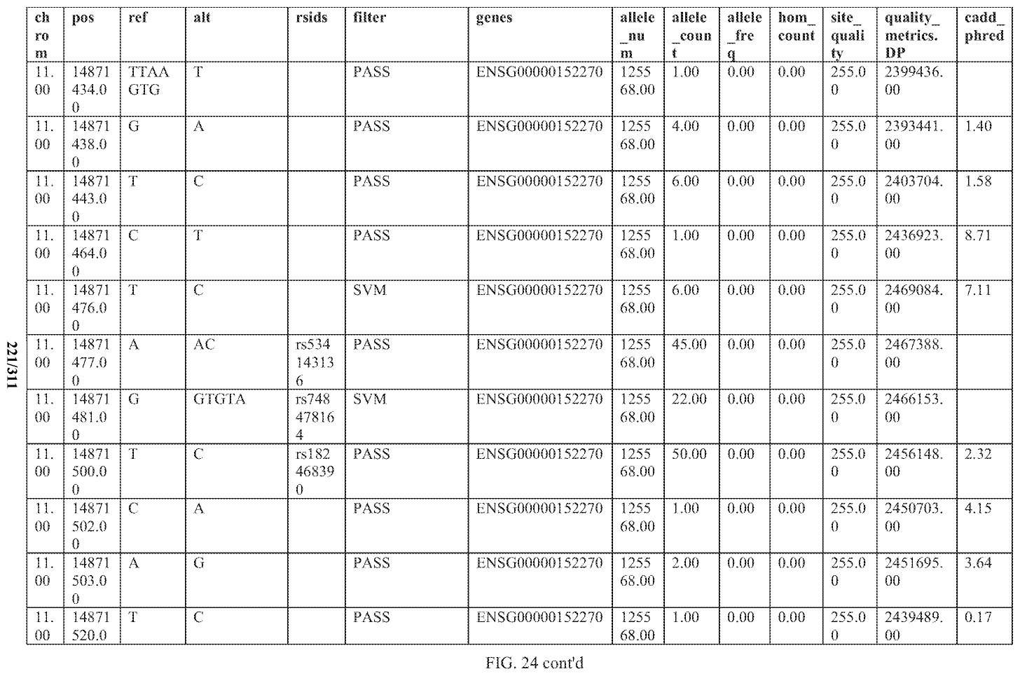

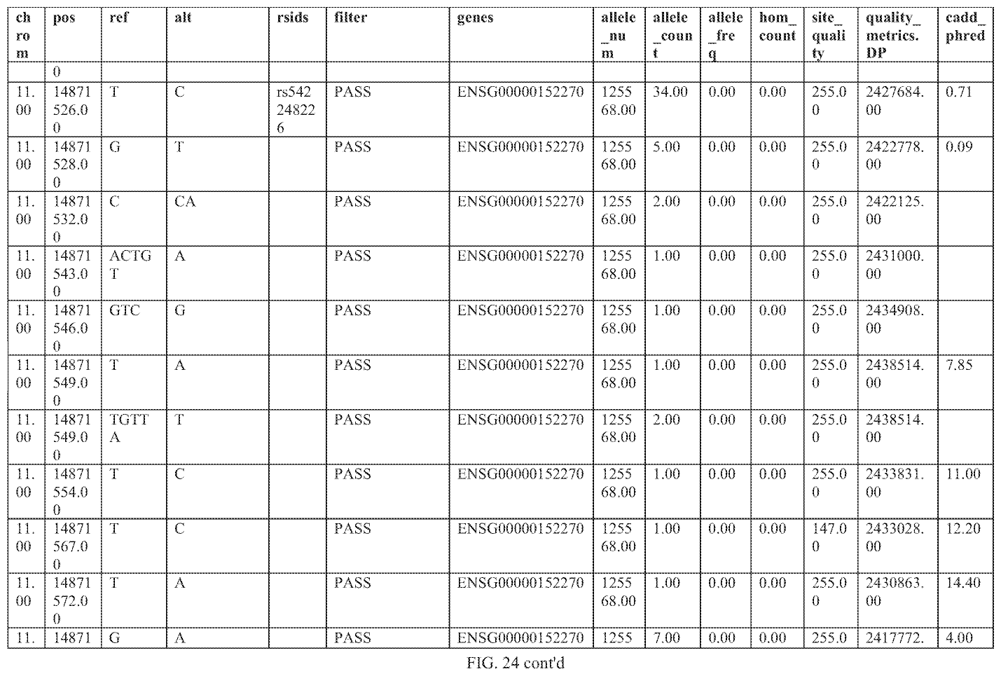

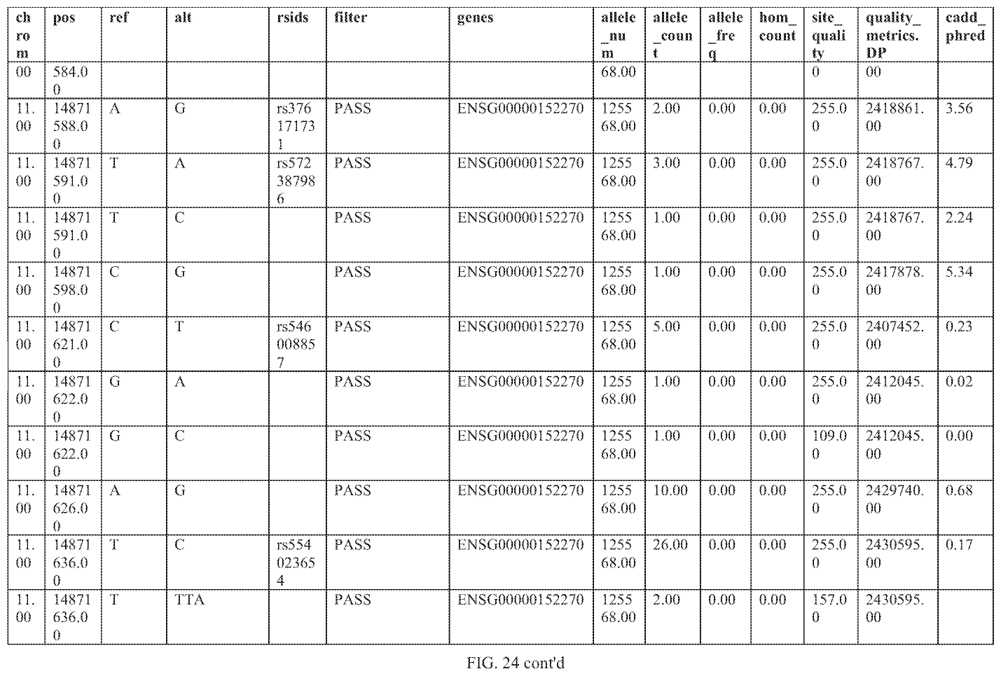

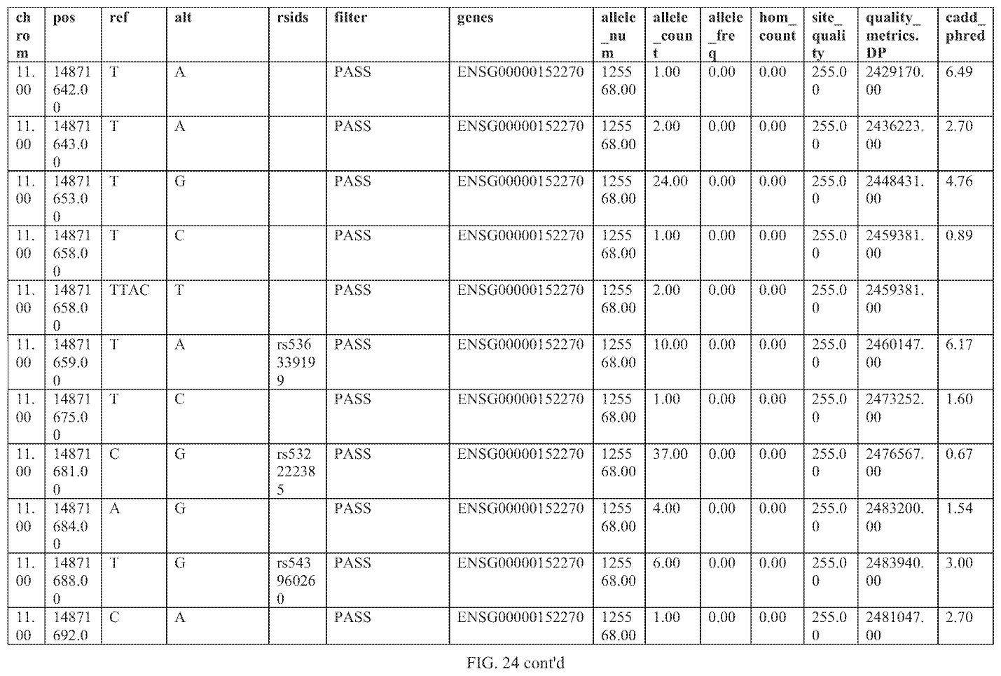

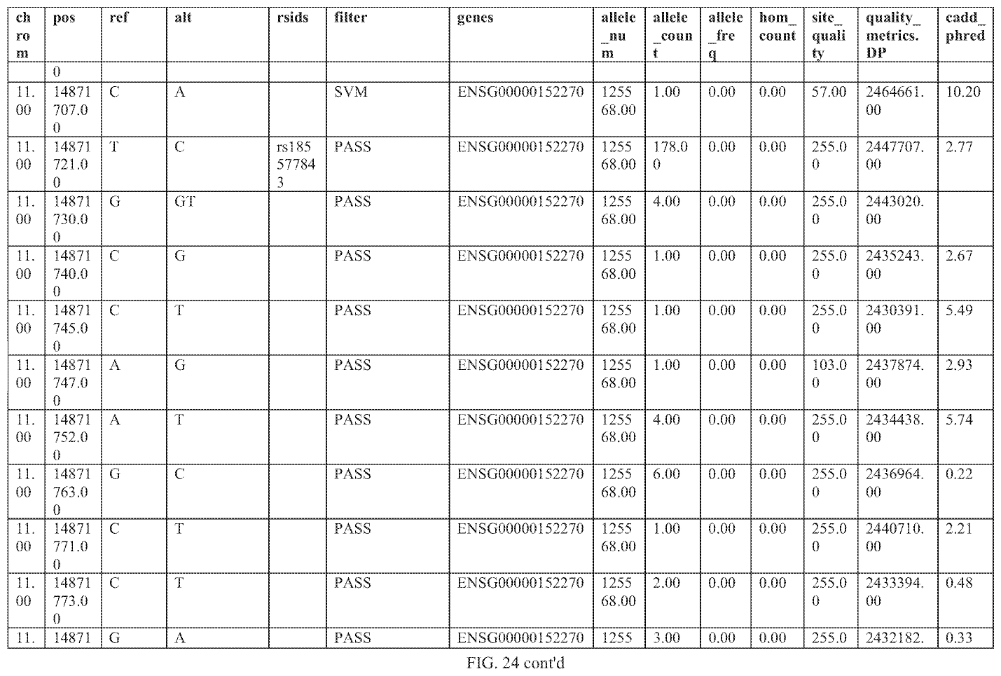

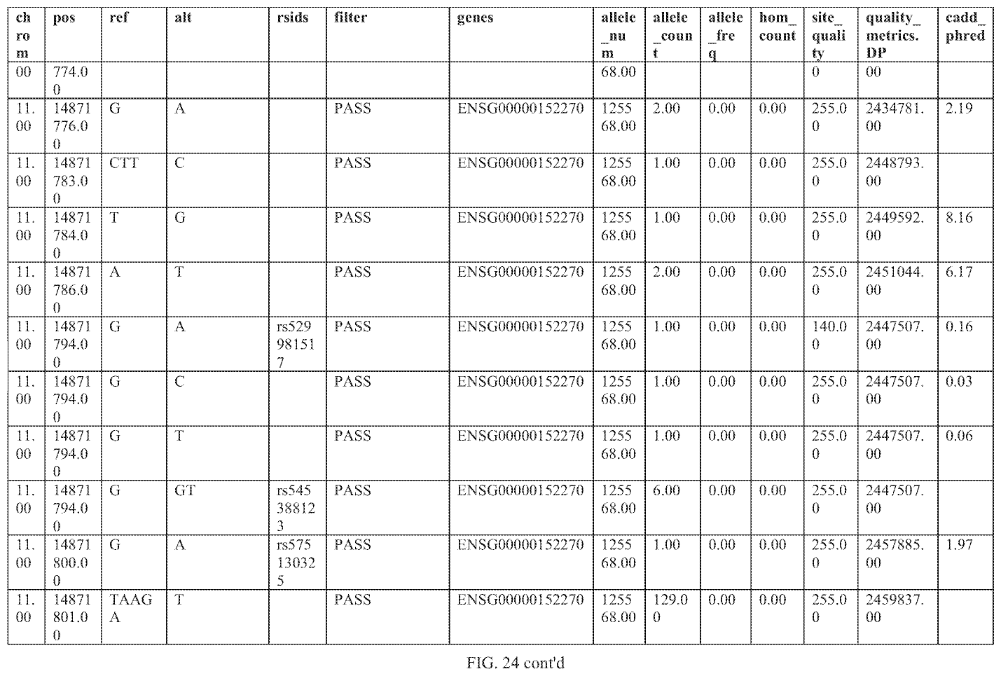

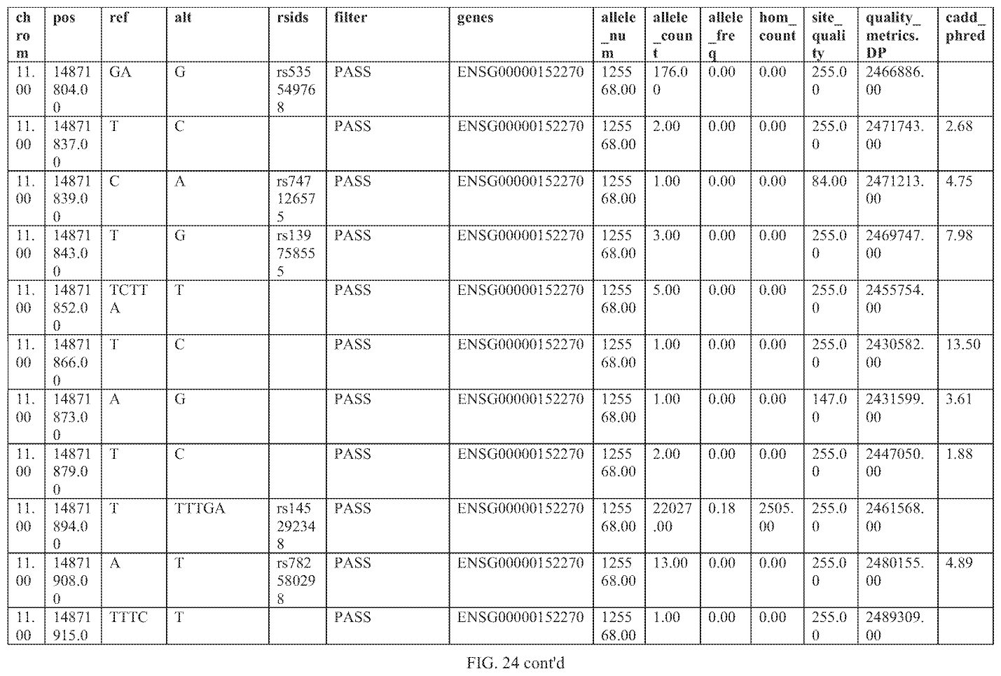

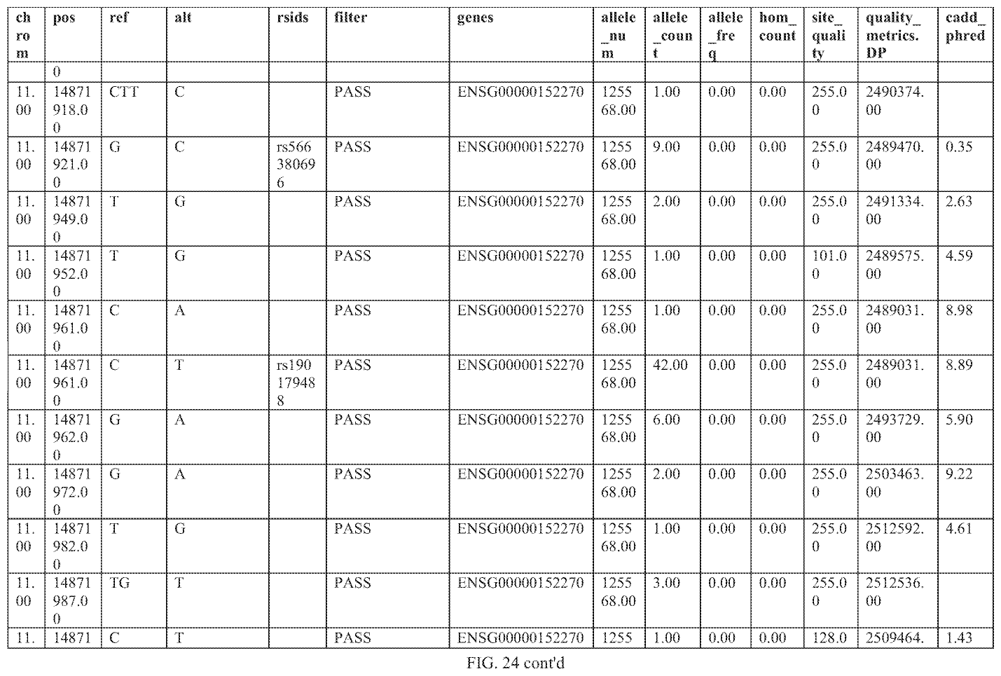

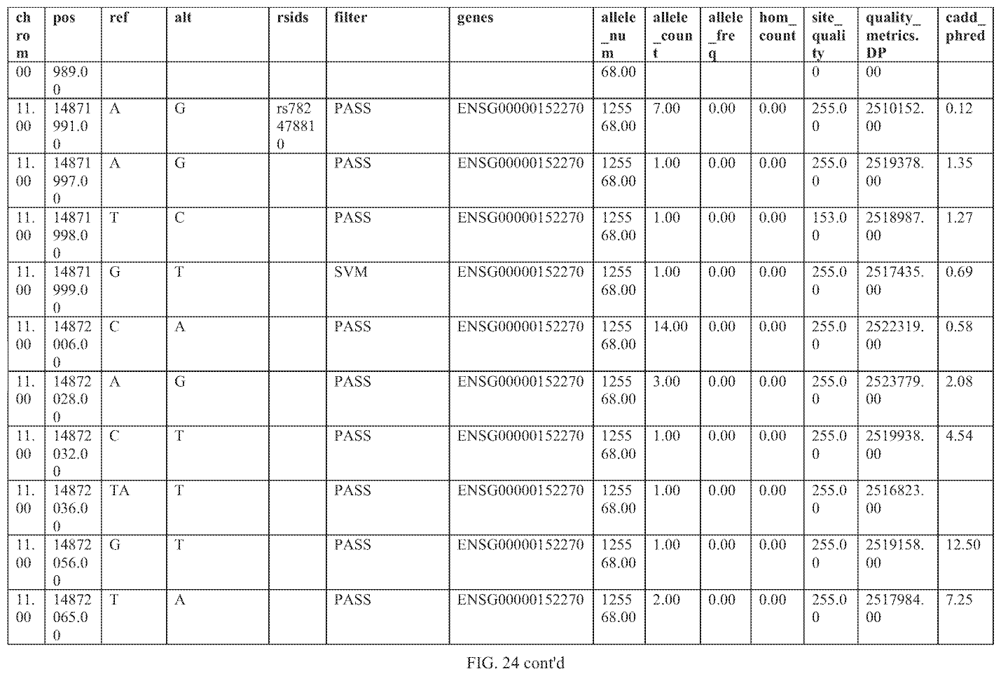

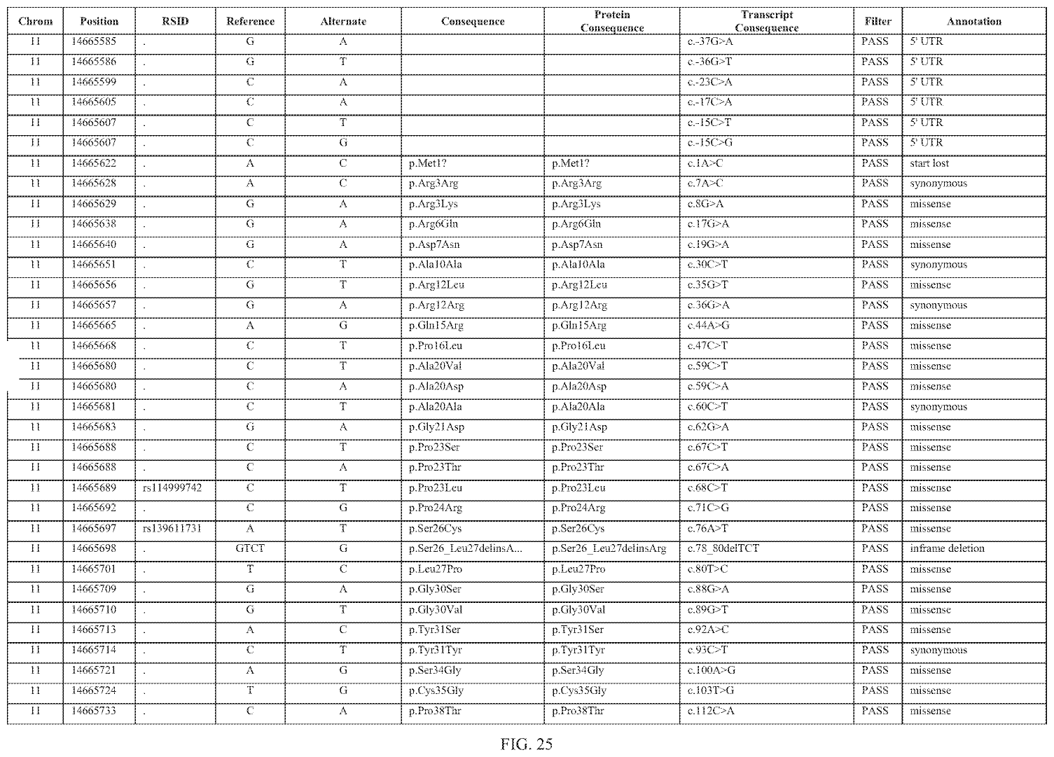

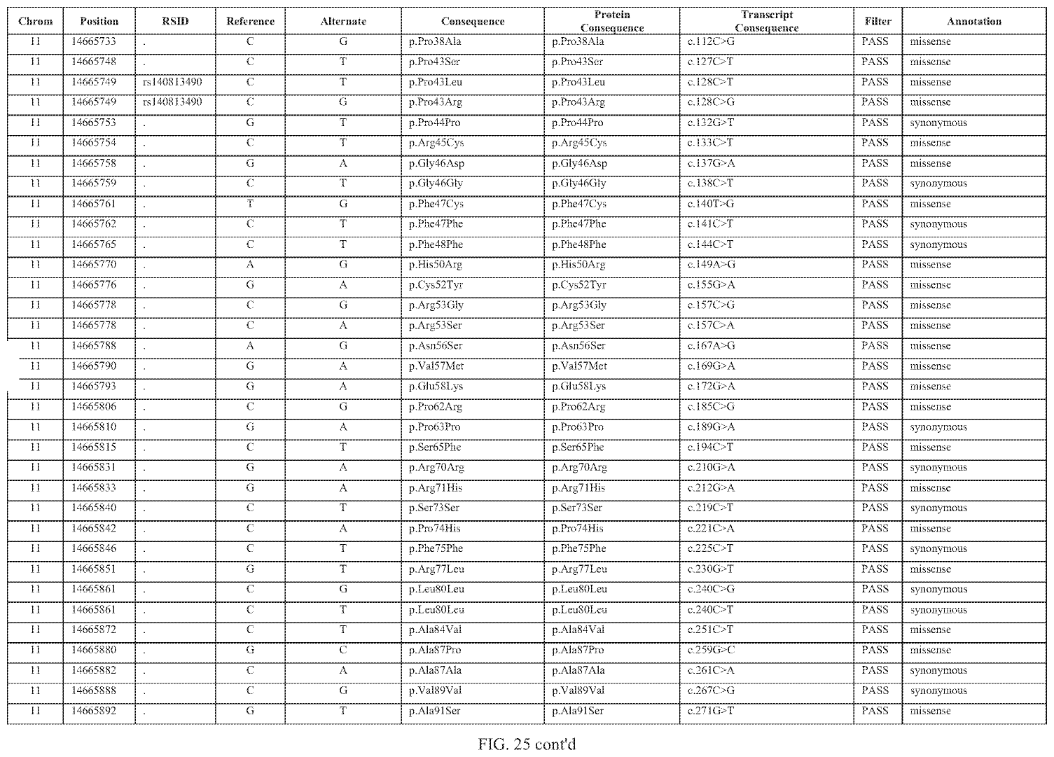

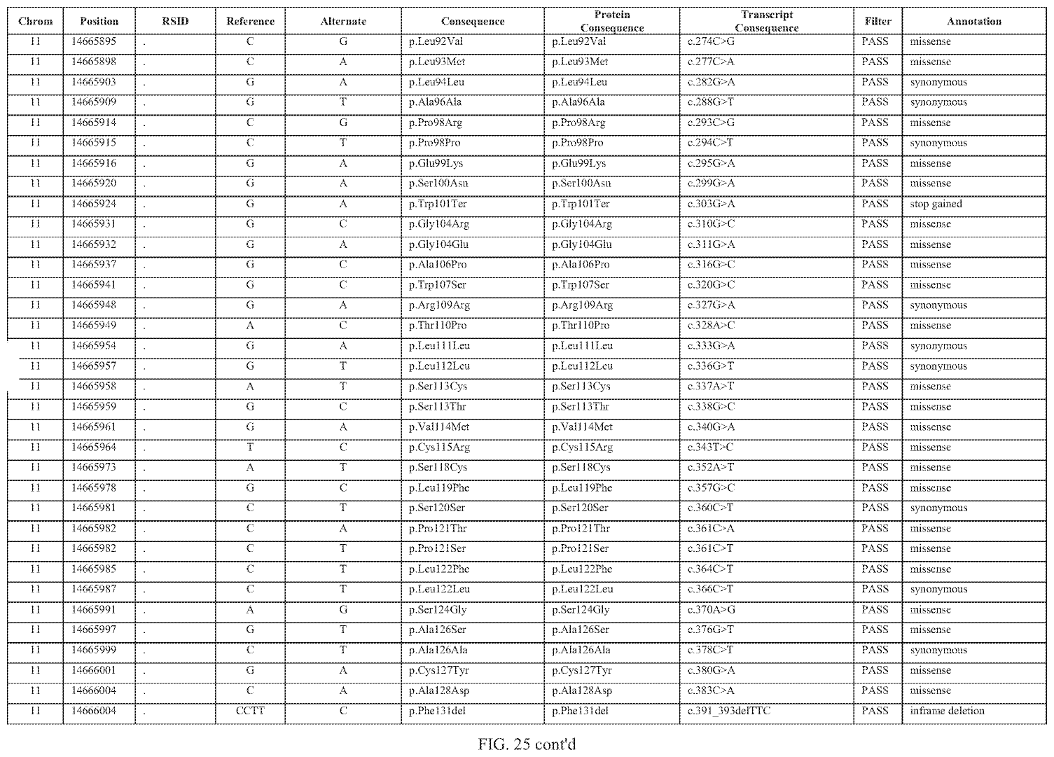

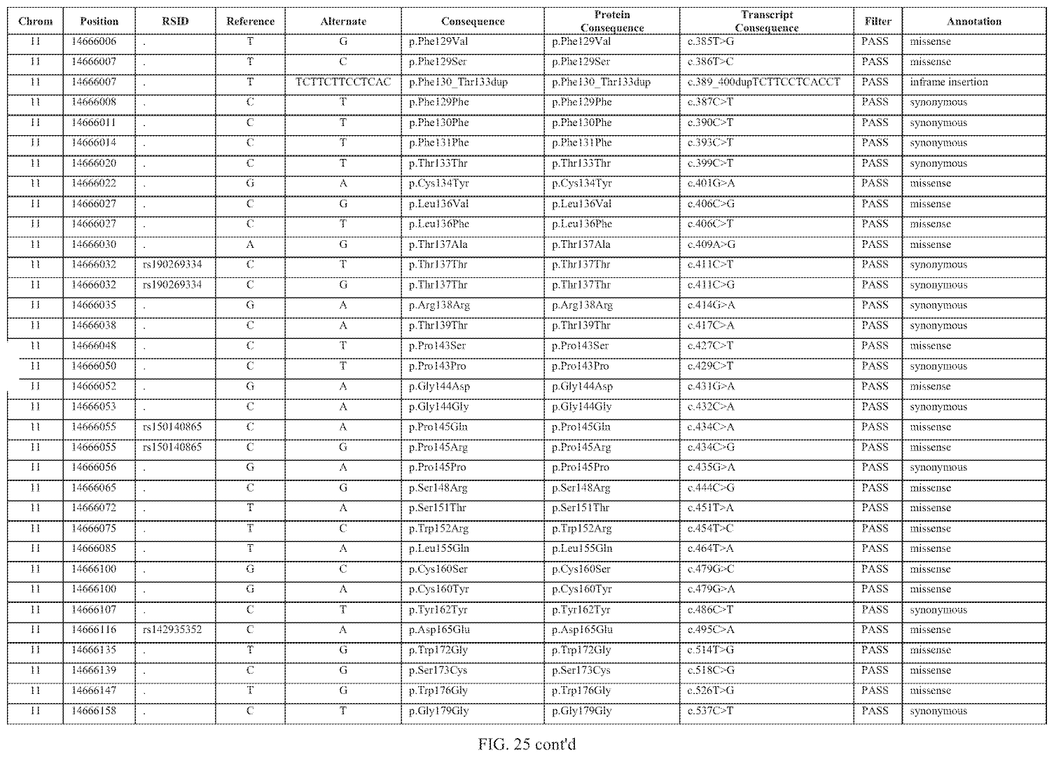

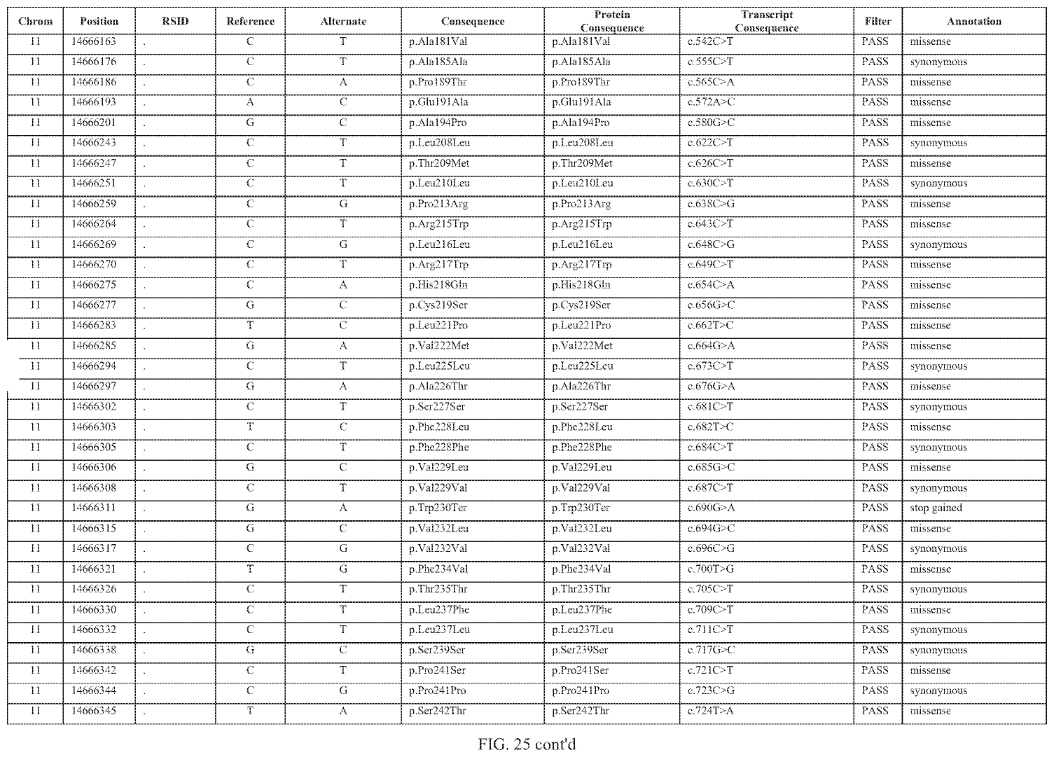

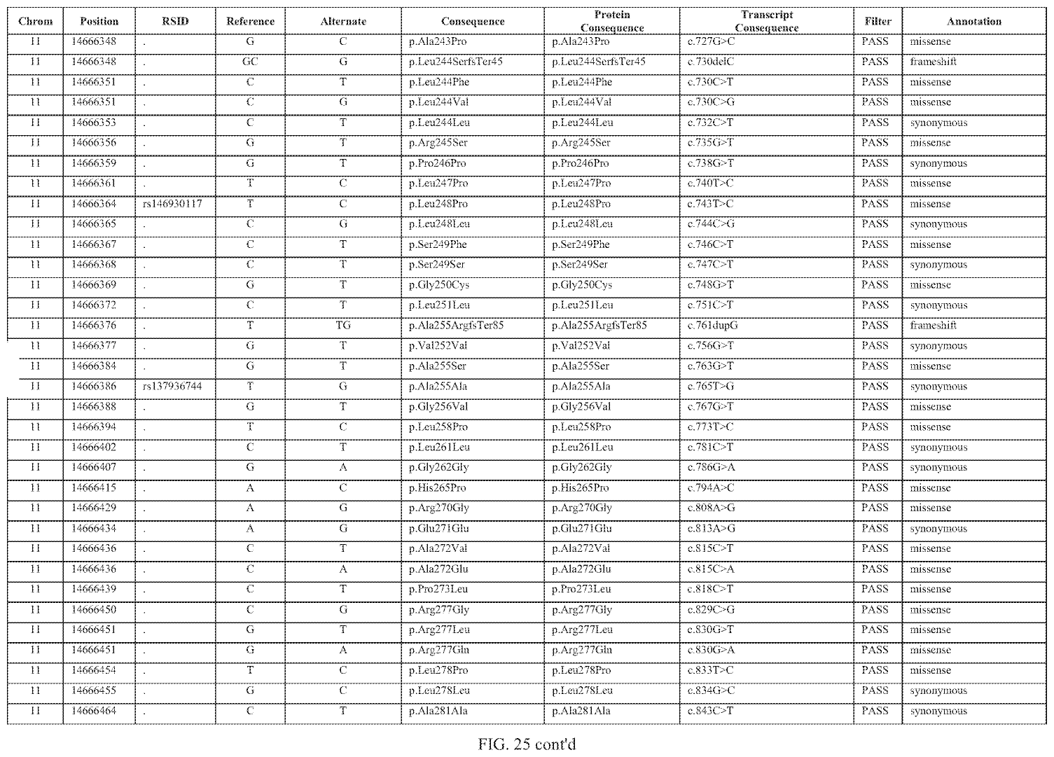

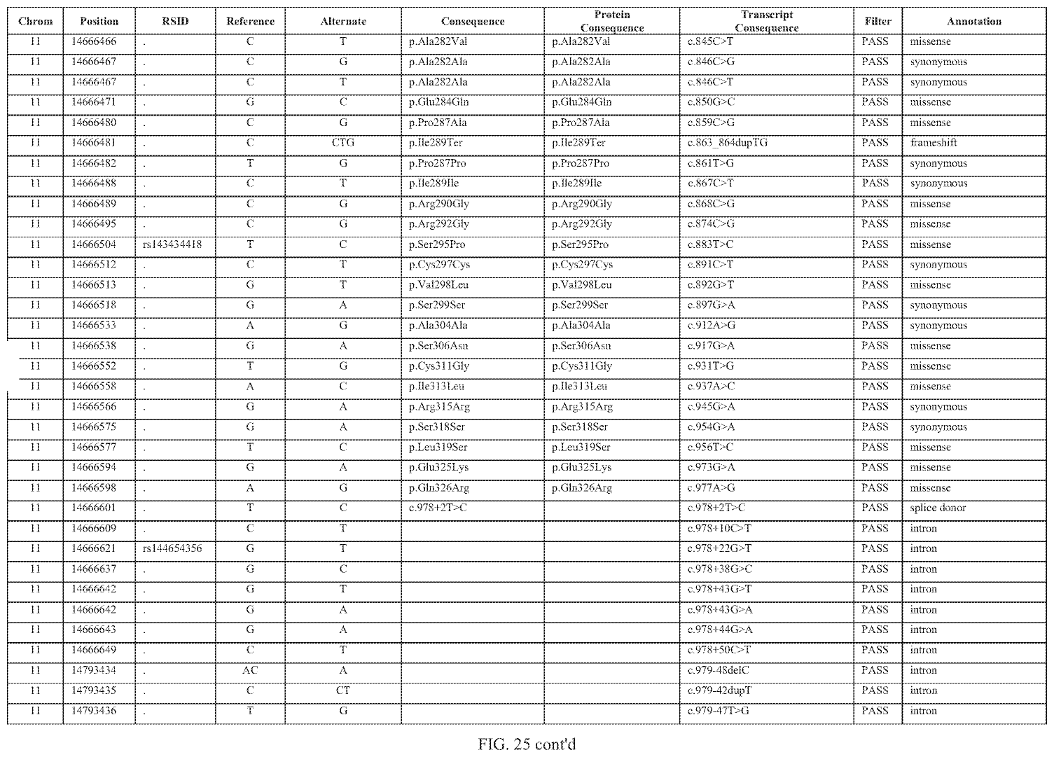

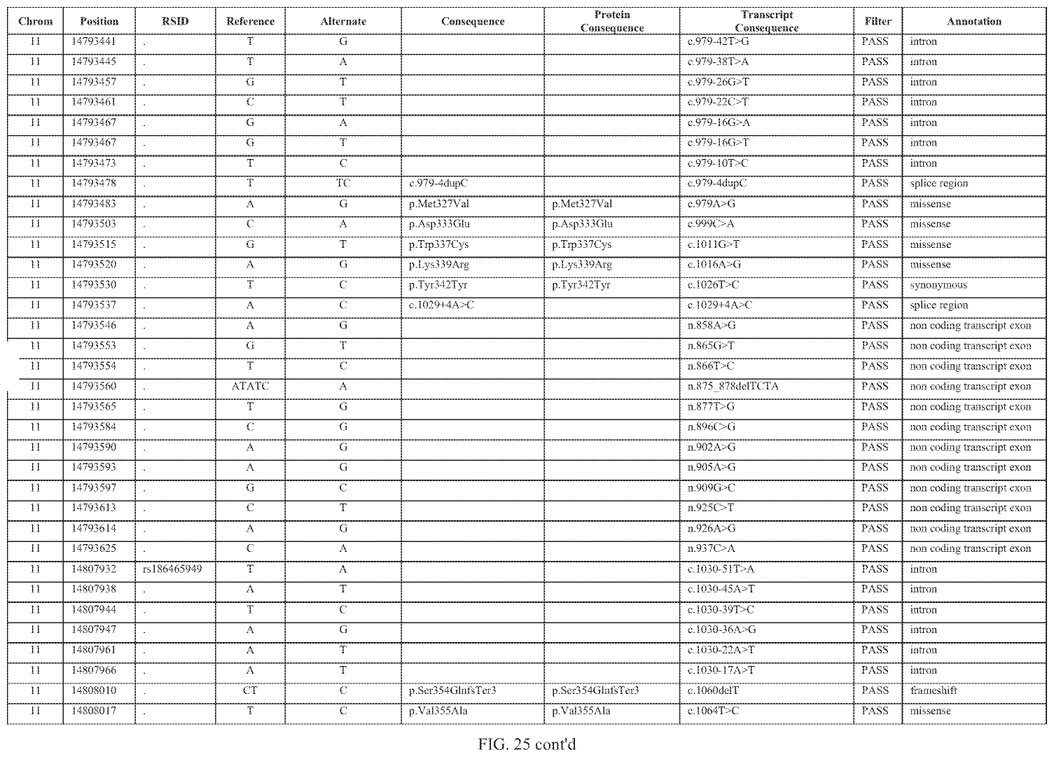

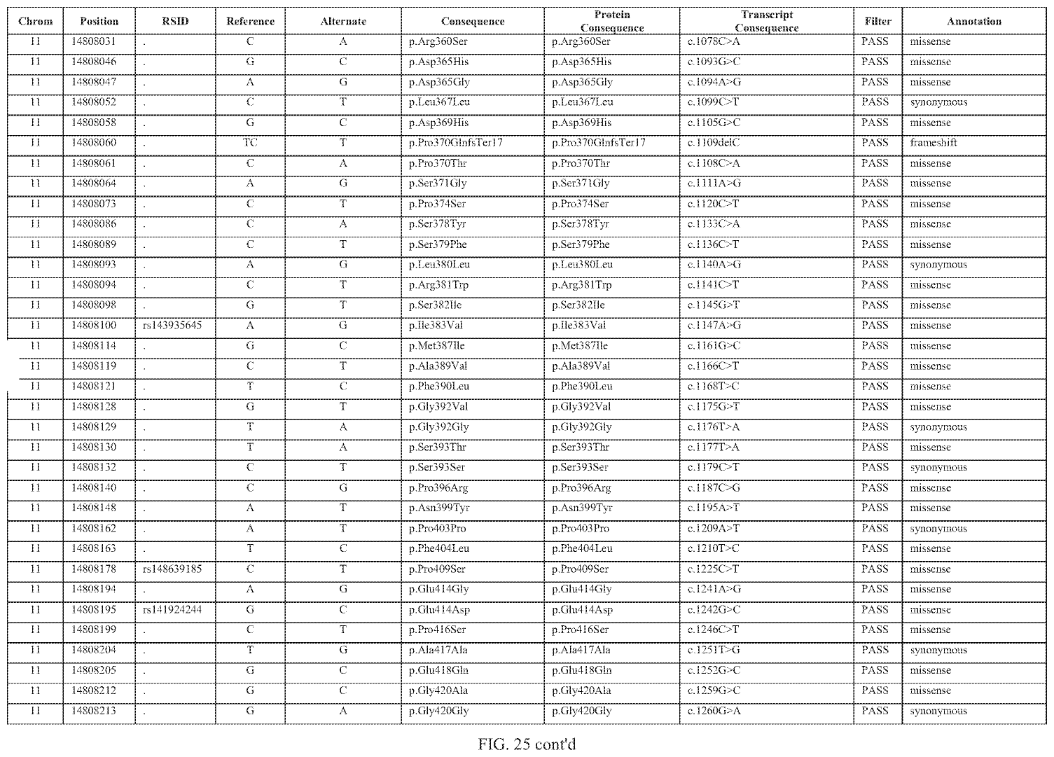

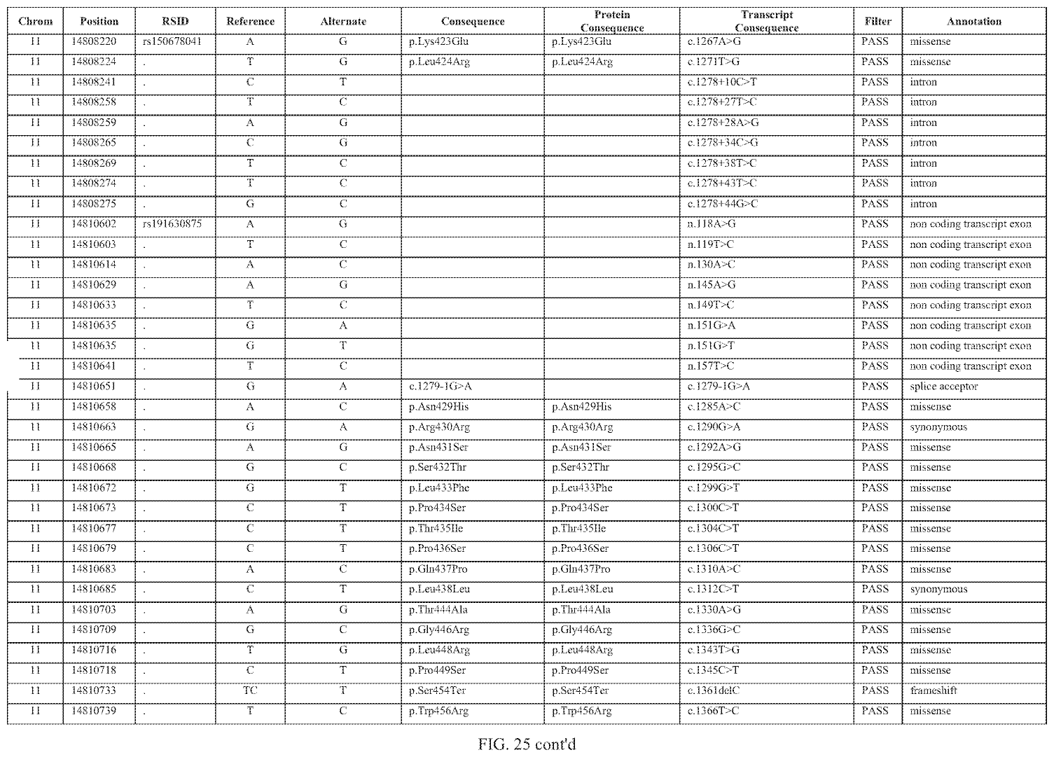

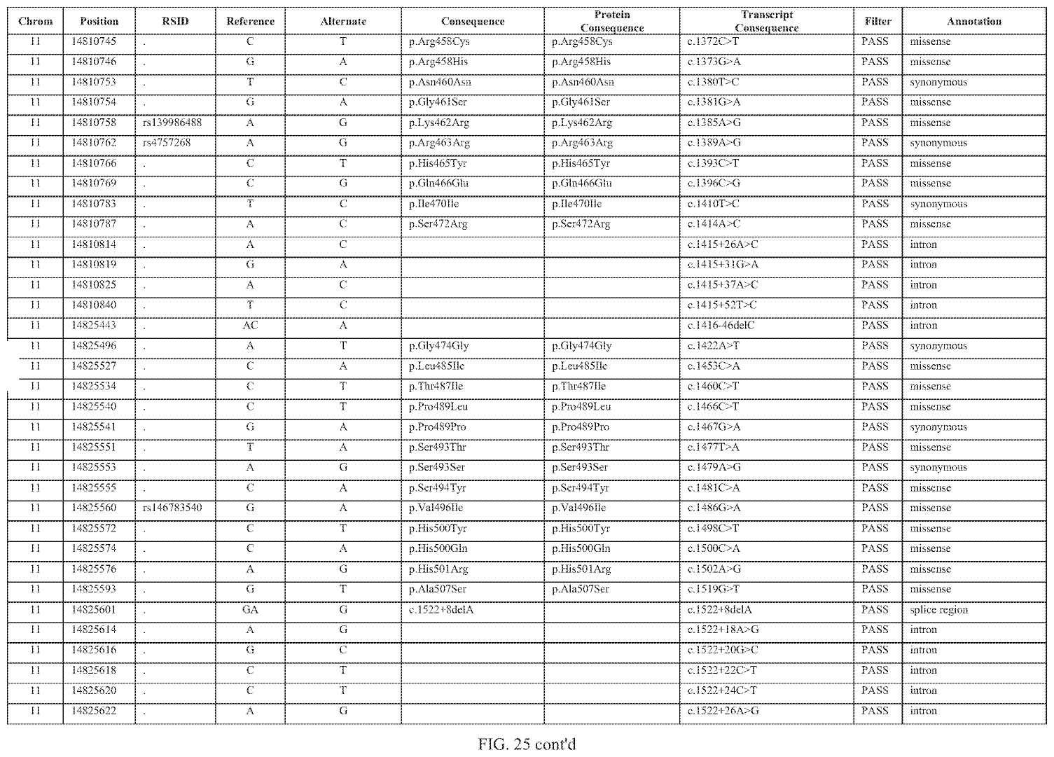

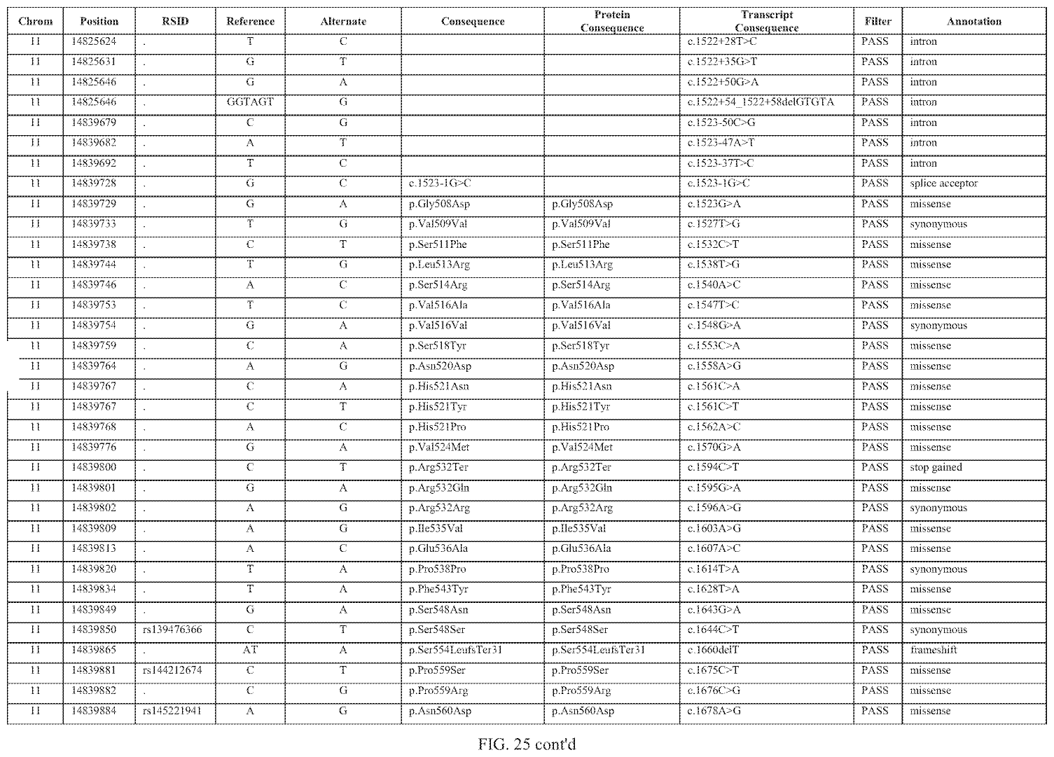

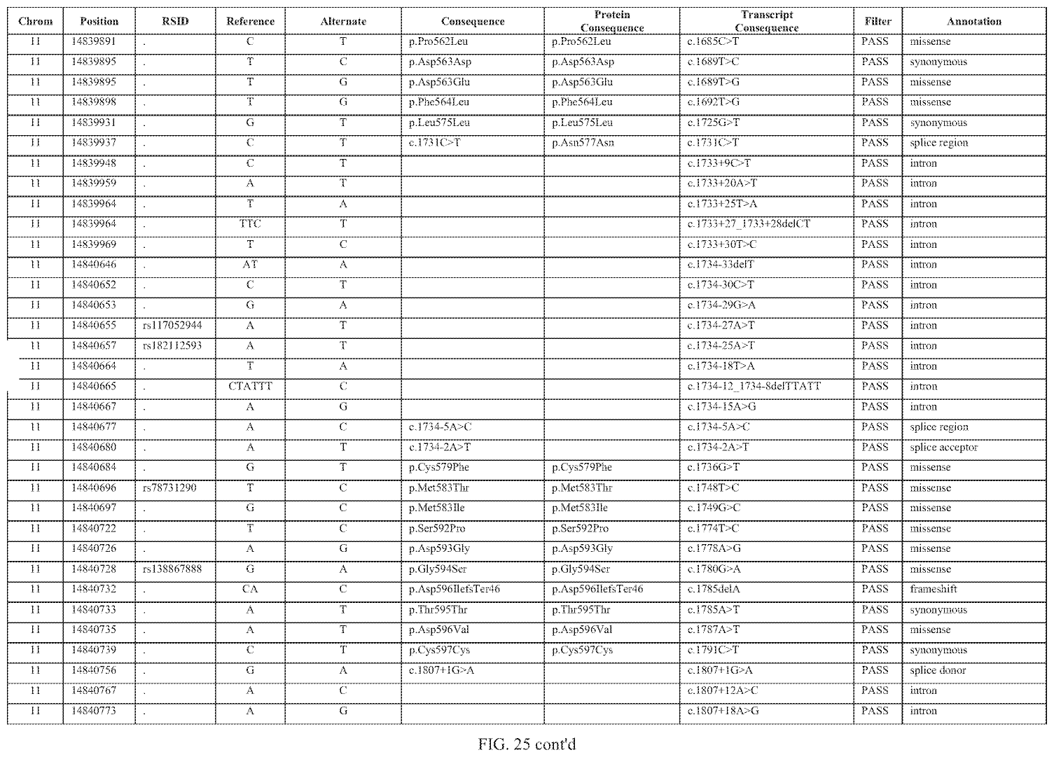

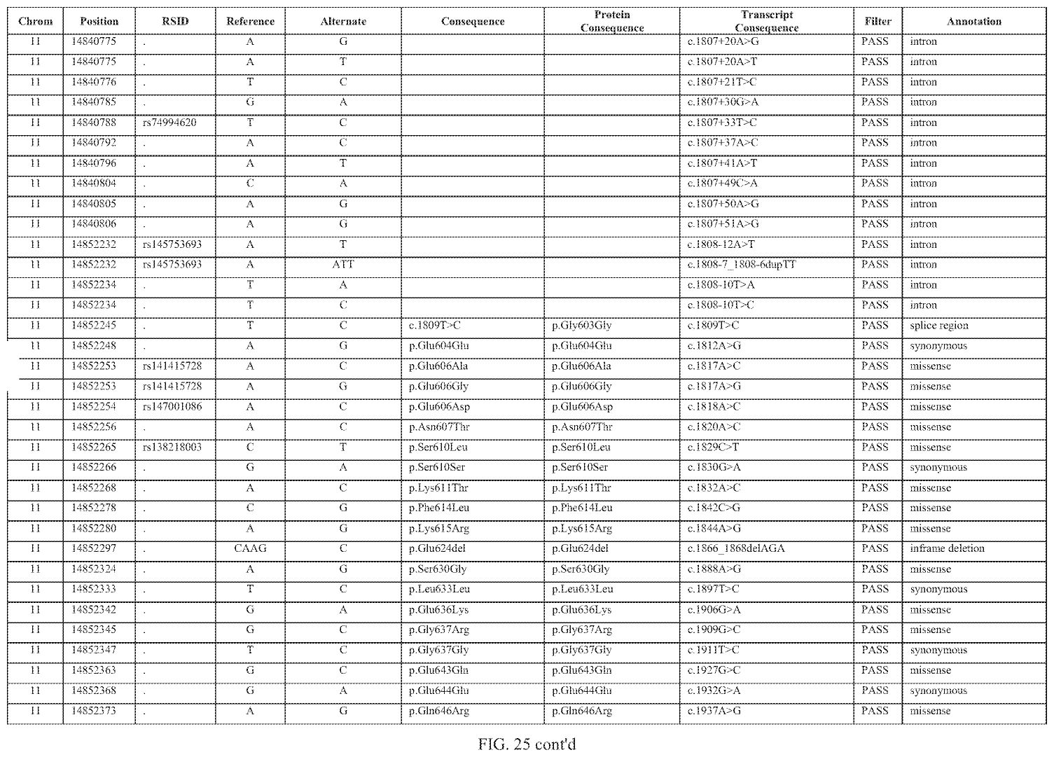

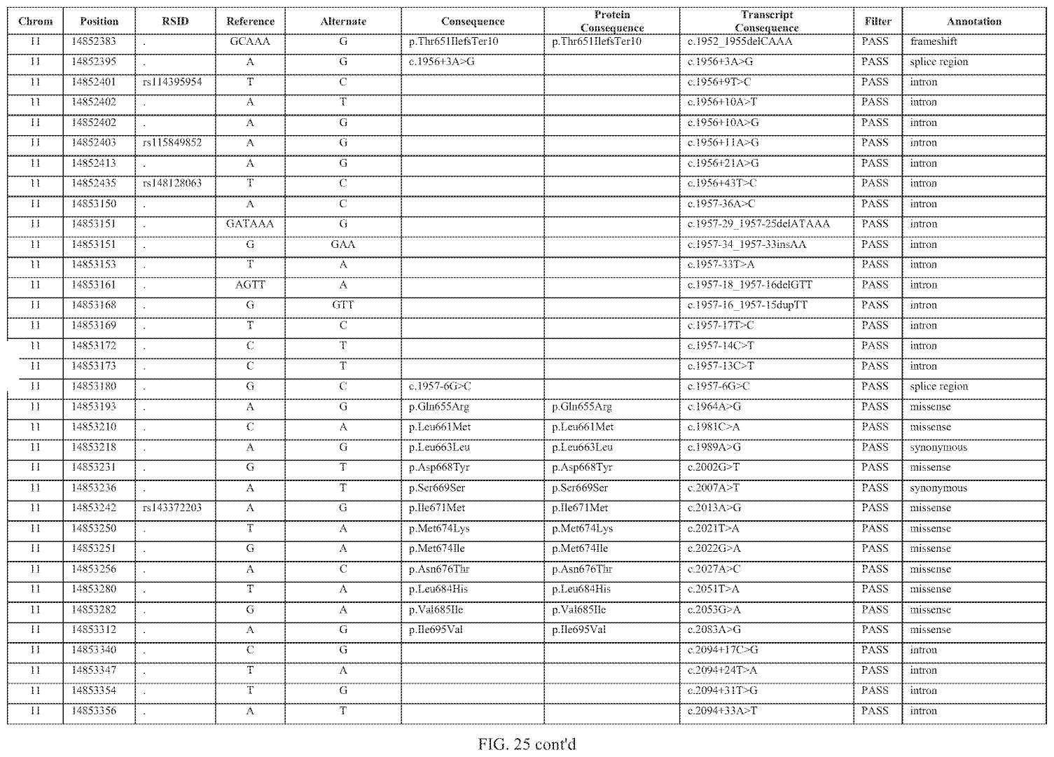

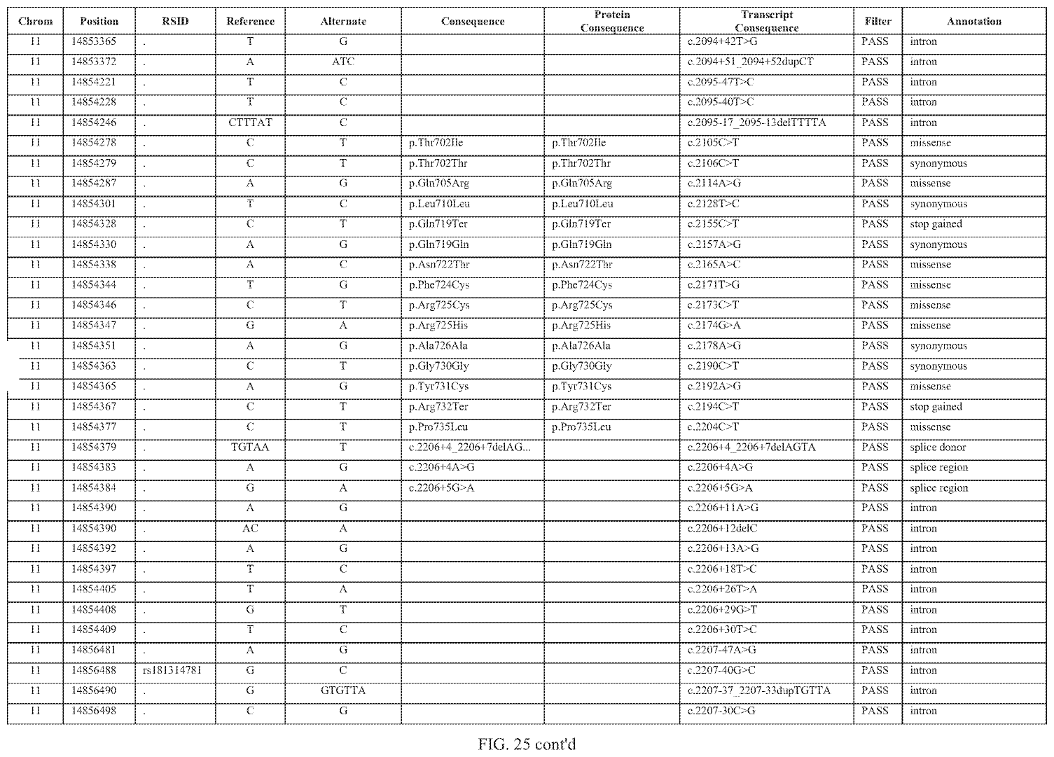

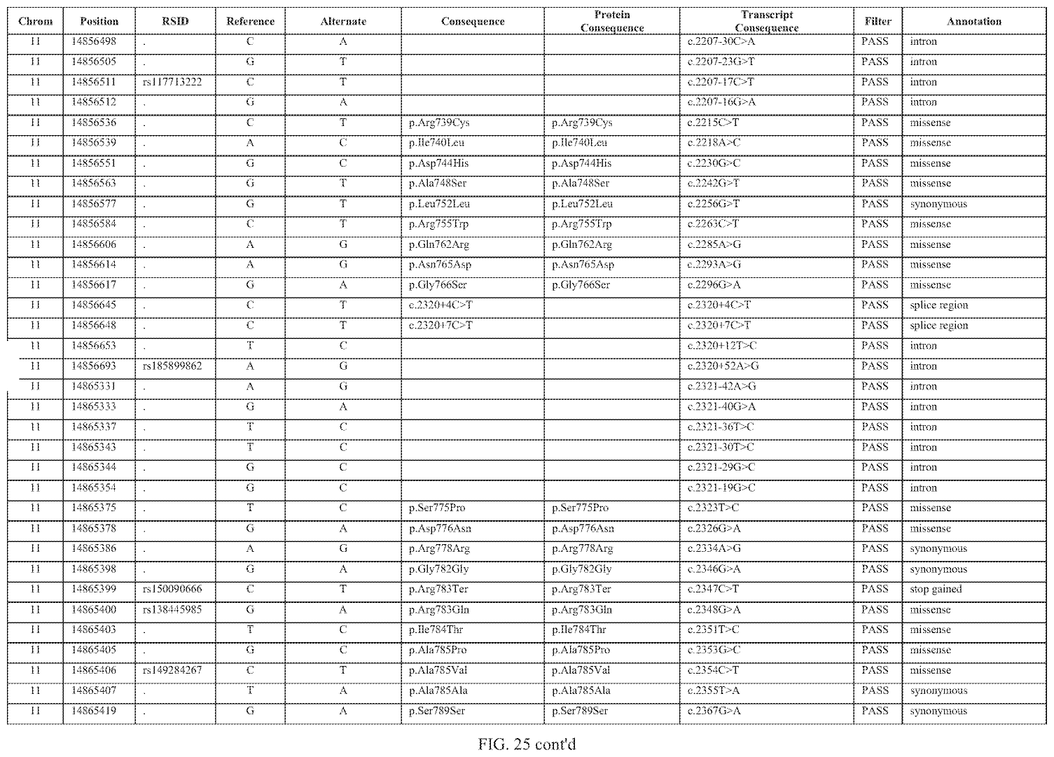

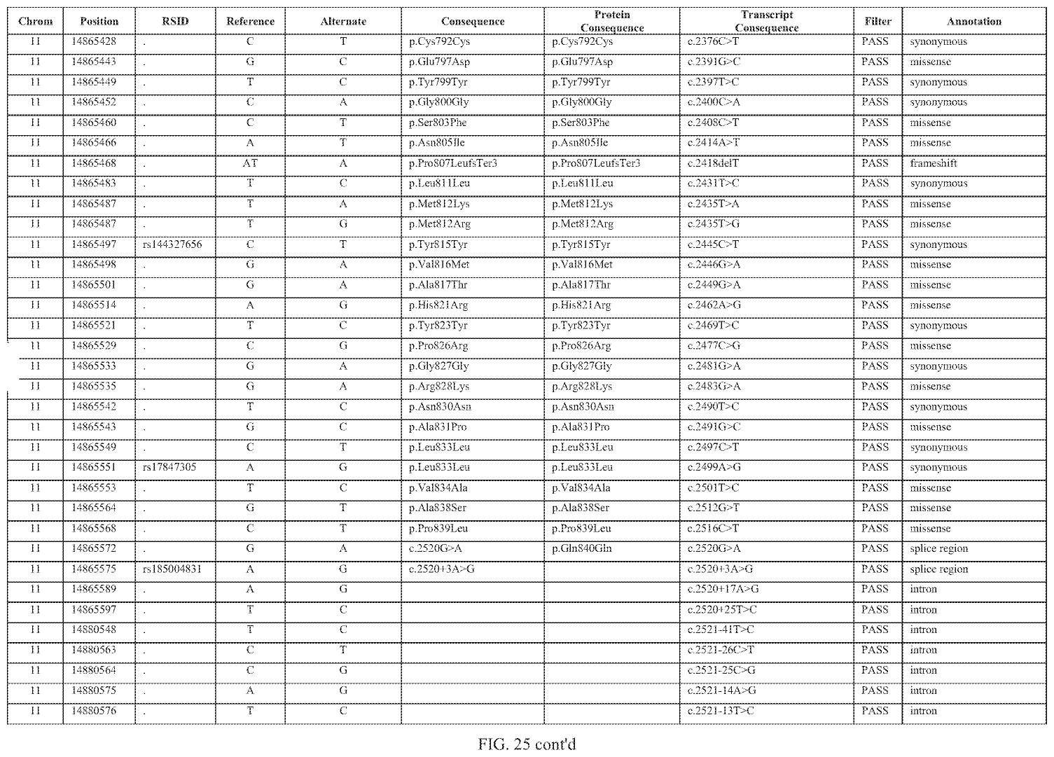

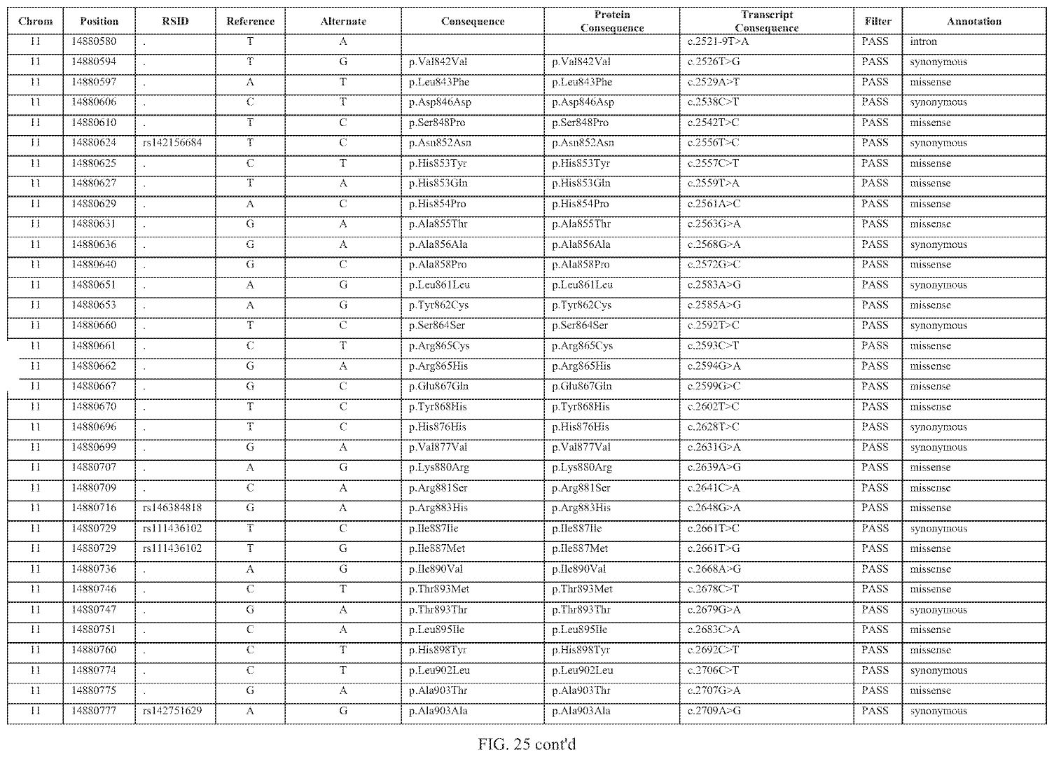

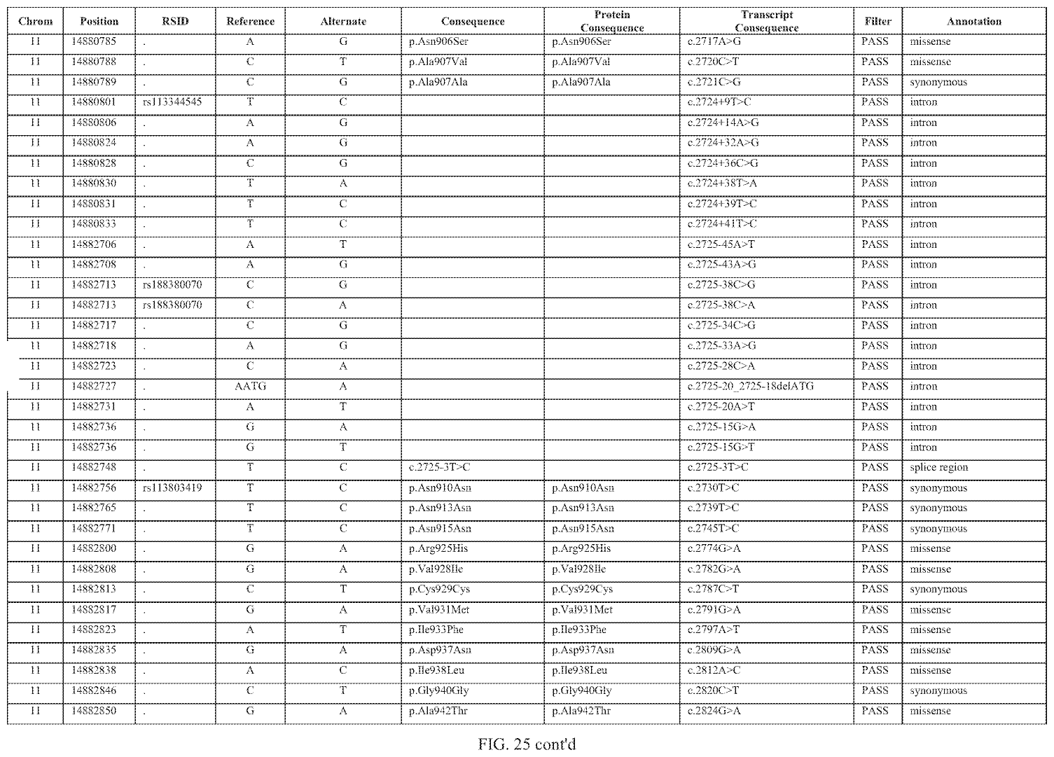

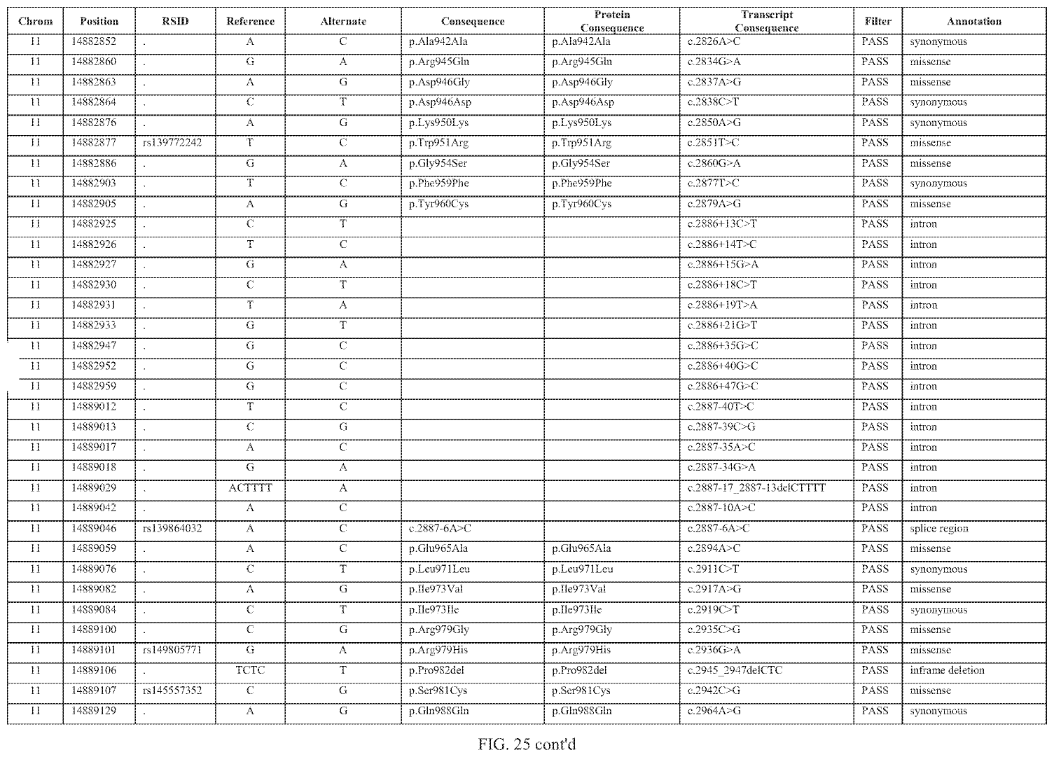

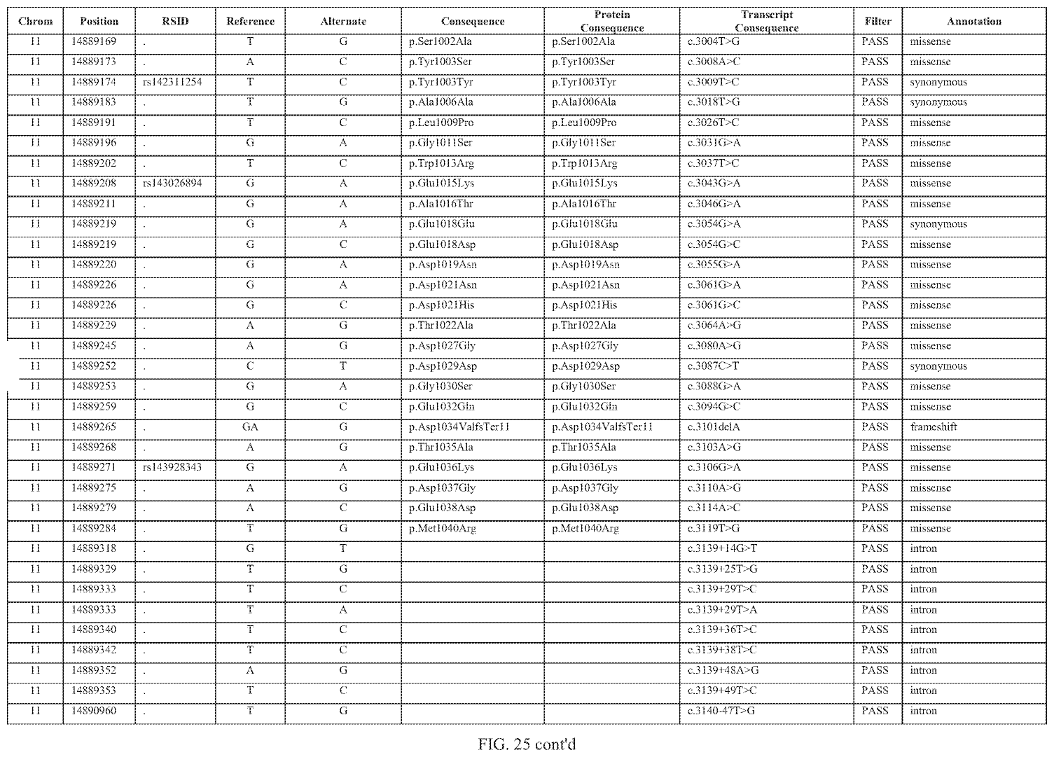

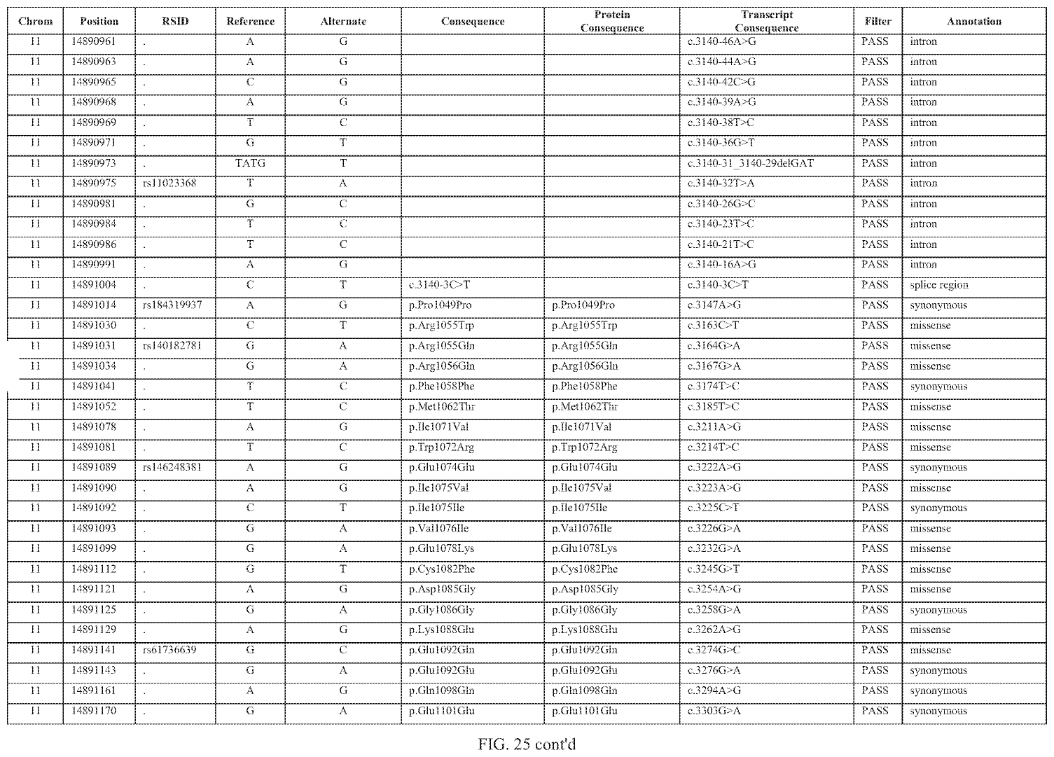

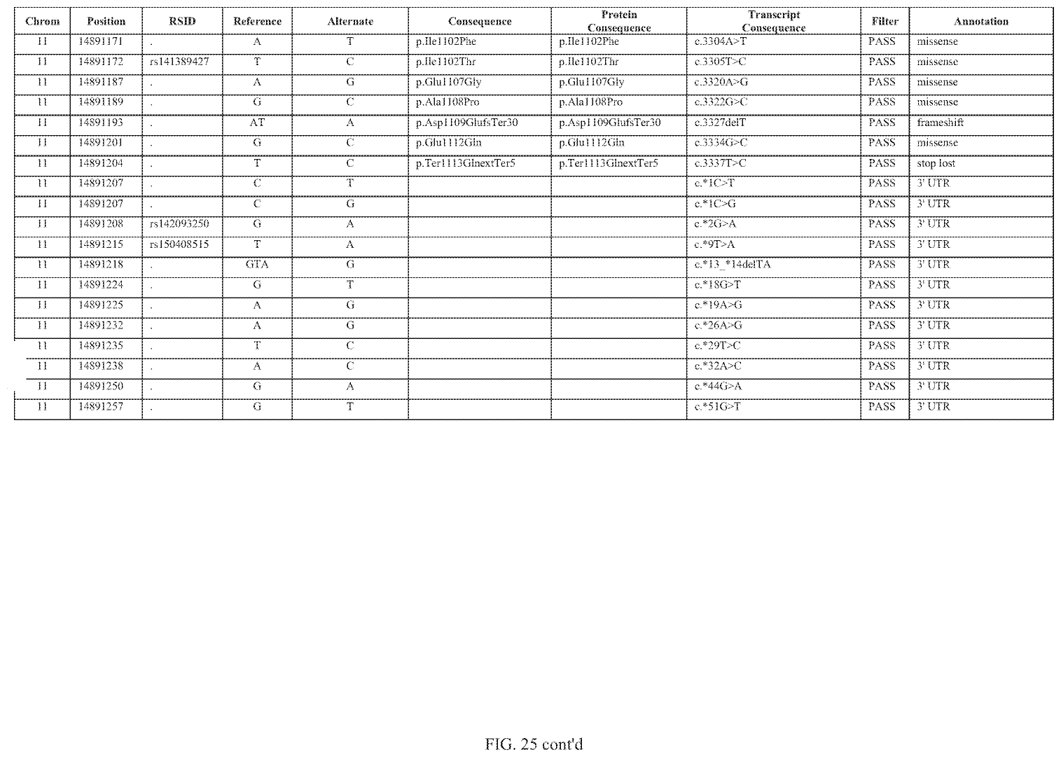

In some aspects, the PDE3B loss of function or damaging variant can be any of those found in FIGS. 24 and 25. In some aspects the PDE3B loss of function or damaging variant can be any of those variants provided in the ExAc Browser (Beta) Exome Agregation Consortium as found at http://exac.broadinstitute.org/.

In some aspects, the PDE3B loss of function or damaging variant results in a PDE3B protein having the mutation Arg783Ter or rs150090666.

In some aspects, the PDE3B loss of function or damaging variant is determined from a sample obtained from the subject. The sample obtained from the subject can be, for example, blood, plasma, serum, cells, urine, mucus, spinal fluid, or sweat.

In some aspects, the PDE3B loss of function or damaging variant is determined by amplifying or sequencing a nucleic acid sample obtained from the subject. In some aspects, the amplifying can be performed using polymerase chain reaction (PCR). In some aspects, the amplifying or sequencing comprises using primers having sequences complementary to PDE3B DNA or RNA sequences. For example, disclosed are primers and probes having sequences complementary to a portion of the PDE3B nucleic acid sequence found in accession number NM_000922.3.

### C. Methods of Detecting PDE3B Loss of Function or Damaging Variants

Disclosed are methods of detecting one or more PDE3B loss of function or damaging variants in a subject, said method comprising: obtaining a biological sample from a subject; detecting whether one or more PDE3B loss of function or damaging variants are present in the biological sample by contacting the biological sample with an anti-PDE3B loss of function or damaging variant antibody or antigen binding fragment thereof and detecting binding between the one or more PDE3B loss of function or damaging variants and the antibody, or fragment thereof.

Disclosed are methods of detecting one or more PDE3B loss of function or damaging variants in a subject, said method comprising: obtaining a biological sample from a subject; detecting whether one or more PDE3B loss of function or damaging variants are present in the biological sample by performing whole genome or whole exome sequencing. After detecting the presence of a variant the effect of these variant on function of the protein or expression of the protein can be predicted. pLOFs can lead to truncation of a protein, splice site problems, or frameshifts.

Disclosed are methods comprising: obtaining a sample from a subject; detecting whether one or more PDE3B loss of function or damaging variants are present in the sample; diagnosing the subject as having a greater likelihood of responding to PDE3B inhibitors when there is an absence of the one or more PDE3B loss of function or damaging variants; and administering an effective amount of a PDE3B inhibitor to the subject. In some aspects, the sample can be, but is not limited to, blood, plasma, serum, cells, urine, mucus, spinal fluid, or sweat. In some aspects, the sample can be DNA or protein.

In some aspects of the disclosed methods, the PDE3B loss of function or damaging variant can be any of those found in FIGS. 24 and 25. In some aspects, a PDE3B loss of function or damaging variant can have the mutation Arg783Ter.

In some aspects, the PDE3B inhibitor can be a compound, protein, DNA, RNAi, CRISPR, or siRNA.

### D. Methods of Treating

Disclosed are methods of treating a subject comprising administering a composition that inhibits the function of PDE3B to a subject, wherein the subject has been determined to lack one or more loss of function or damaging mutations in PDE3B. In some aspects, a PDE3B loss of function or damaging variant results in a PDE3B protein having the mutation Arg783Ter. Thus, a subject lacking the loss of function mutation in PDE3B can be a subject that does not contain the Arg783Ter mutation. In some aspects, a subject lacking the loss of fuction mutation in PDE3B can be a subject that does not contain any of the mutations in FIGS. 24 and 25.

In some aspects, the composition administered to the subject can be a compound, protein, DNA, RNAi, CRISPR, or siRNA.

Disclosed are methods for identifying a subject in need of treatment for coronary artery disease comprising determining in the subject the presence of a PDE3B loss of function or damaging variant, wherein the presence of a PDE3B loss of function or damaging variant indicates that the subject is not in need of treatment for coronary artery disease. Thus, also disclosed are methods for identifying a subject in need of treatment for coronary artery disease comprising determining in the subject the lack of a PDE3B loss of function or damaging variant, wherein the lack of a PDE3B loss of function or damaging variant indicates that the subject is in need of treatment for coronary artery disease. In some aspects, the PDE3B loss of function or damaging variant results in a PDE3B protein having the mutation Arg783Ter. In some aspects, the PDE3B loss of function or damaging variant results in a PDE3B protein having the mutation of any of those in FIGS. 24 and 25.

### E. Methods of Screening

Disclosed are methods of screening for test compositions that cause a loss of function or damaging mutation in PDE3B comprising: contacting a PDE3B gene with a test composition; detecting the presence of one or more mutations in the PDE3B gene; and determining if the one or more mutations is a loss of function or damaging mutation, wherein the presence of one or more loss of function or damaging mutations in PDE3B indicates a test composition that causes a loss of function or damaging in PDE3B. In some aspects, prior to contacting a PDE3B gene with a test composition, the presence of a loss of function or damaging mutation is first analyzed in the PDE3B gene. If no loss of function or damaging mutation is detected then the PDE3B gene can be contacted with a test composition.

In some aspects, the PDE3B loss of function or damaging variant results in a PDE3B protein having the mutation of any of those in FIGS. 24 and 25. In some aspects, the loss of function or damaging mutation in PDE3B results in a PDE3B protein having the mutation Arg783Ter.

Disclosed are methods of screening for therapeutic candidates for treating coronary artery disease compositions comprising: contacting a cell lacking a loss of function or damaging mutation in PDE3B with a test composition; and determining if the test composition inhibits PDE3B in the cell, wherein if the test composition inhibits PDE3B then it is a therapeutic candidate for treating coronary artery disease.

Disclosed are methods of identifying a subject in need of screening for the development of coronary artery disease comprising determining in the subject the absence of a PDE3B loss of function or damaging variant, wherein the absence of a a PDE3B loss of function or damaging variant indicates a subject in need of screening for the development of coronary artery disease. In some aspects, the loss of function or damaging mutation in PDE3B results in a PDE3B protein having the mutation Arg783Ter.

### F. Methods of Inducing Loss of Function or Damaging PDE3B Variants

Disclosed are methods of inducing a loss of function or damaging mutation in PDE3B comprising administering a test composition determined from the disclosed methods of screening for test compositions that cause a loss of function or damaging mutation in PDE3B. In some aspects, the loss of function or damaging mutation in PDE3B results in a PDE3B protein having the mutation Arg783Ter.

### G. Vectors

Disclosed are vectors comprising a loss of function or damaging PDE3B variant, wherein the loss of function or damaging mutation in PDE3B results in a PDE3B protein having the mutation Arg783Ter.

In some aspects, the vectors can be viral or non-viral vectors. The term “vector”, as used herein, refers to a composition capable of transporting a nucleic acid. In some cases, a vector can be a plasmid, i.e., a circular double stranded piece of DNA into which additional DNA segments can be ligated. In some cases, a vector can be a viral vector, wherein additional DNA segments can be ligated into the viral genome. In some cases, a vector can autonomously replicate in a host cell into which they are introduced (e.g., bacterial vectors having a bacterial origin of replication and episomal mammalian vectors). In other cases, vectors (e.g., non-episomal mammalian vectors) can be integrated into the genome of a host cell upon introduction into the host cell, and thereby are replicated along with the host genome. Moreover, certain vectors can direct the expression of genes to which they are operatively linked. Such vectors can be referred to as “recombinant expression vectors” (or simply, “expression vectors”).

In some aspects, the proteins encoded by the PDE3B variants are expressed by inserting DNAs encoding the PDE3B variants into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. In some instances nucleic acids comprising the PDE3B variants can be ligated into a vector such that transcriptional and translational control sequences within the vector serve their intended function of regulating the transcription and translation of the PDE3B variant. The expression vector and expression control sequences are chosen to be compatible with the expression host cell used. Nucleic acid sequences comprising the PDE3B variants can be inserted into separate vectors or into the same expression vector. A nucleic acid sequence comprising the PDE3B variants can be inserted into the expression vector by standard methods (e.g., ligation of complementary restriction sites on the nucleic acid comprising the PDE3B variants and vector, or blunt end ligation if no restriction sites are present).

In addition to a nucleic acid sequence comprising the PDE3B variants, the recombinant expression vectors can carry regulatory sequences that control the expression of the genetic variant in a host cell. It will be appreciated by those skilled in the art that the design of the expression vector, including the selection of regulatory sequences can depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615. Methods of expressing polypeptides in bacterial cells or fungal cells, e.g., yeast cells, are also well known in the art.

In addition to a nucleic acid sequence comprising the PDE3B variants and regulatory sequences, the recombinant expression vectors can carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, incorporated herein by reference). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification), the neo gene (for G418 selection), and the glutamate synthetase (GS) gene.

### H. Cells

Disclosed are cells comprising the disclosed vectors. In some instances, a cell can be transfected with a nucleic acid comprising the PDE3B variants. In some instances, a cell comprising one or more of the PCKS9 variants can express the protein encoded by the one or more disclosed genetic variants and therefore, also disclosed are cells comprising a protein encoded by one or more PDE3B variants.

### I. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits that can comprise an assay or assays for detecting one or more PDE3B variants in a sample of a subject.

### J. Engineered CRISPR-CAS System

Disclosed are engineered, non-naturally occurring CRISPR-CAS system comprising: a guide RNA that hybridizes with a target sequence, wherein the target sequence comprises a PDE3B loss of function or damaging variant, and a Cas protein or gene encoding a Cas protein. In some aspects, the Cas protein can be a Type-II Cas9 protein or a gene encoding a Type-II Cas9 protein. In some aspects, the Cas9 protein and the guide RNA do not naturally occur together

In some aspects of the engineered, non-naturally occurring CRISPR-CAS system, the PDE3B loss of function or damaging variant comprises the mutation Arg783Ter in the PDE3B protein.

In some aspects, the guide RNA sequence can comprise a sequence that binds to a portion of the PDE3B nucleic acid sequence found in accession number NM_000922.3.

### K. Methods of Altering Expression of a Gene Product

Disclosed are methods of altering expression of at least one gene product, wherein the at least one gene product is a gene product from a PDE3B loss of function or damaging variant, wherein the method comprises administering a guide RNA that hybridizes with a target sequence, wherein the target sequence comprises the PDE3B loss of function or damaging variant, and a Cas protein or gene encoding a Cas protein, whereby the guide RNA targets the target sequence and the Cas9 protein cleaves the nucleic acid molecule which comprises the PDE3B loss of function or damaging variant, whereby expression of the at least one gene product is altered. In some aspects, the PDE3B loss of function or damaging variant comprises the mutation Arg783Ter in the PDE3B protein.

Disclosed are methods of altering expression of at least one gene product, wherein the at least one gene product is a gene product from a PDE3B loss of function or damaging variant, wherein the method comprises administering a vector that comprises a first regulatory element operable in a eukaryotic cell operably linked to at least one nucleotide sequence encoding a CRISPR-Cas system guide RNA that hybridizes with a target sequence, wherein the target sequence comprises the PDE3B loss of function or damaging variant; and a second regulatory element operable in a eukaryotic cell operably linked to a nucleotide sequence encoding a Cas9 protein, whereby the guide RNA targets the target sequence and the Cas9 protein cleaves the target sequence, whereby expression of the at least one gene product is altered. In some aspects, the PDE3B loss of function or damaging variant comprises the mutation Arg783Ter in the PDE3B protein.

In some aspects, the guide RNA sequence comprises the sequence of can comprise a sequence that binds to a portion of the PDE3B nucleic acid sequence found in accession number NM_000922.3.

### L. Methods of Silencing/Inhibiting Expression of PDE3B

Disclosed are methods of silencing or inhibiting expression of wild type PDE3B in a cell comprising providing at least one silencing agent to the cell, wherein said silencing agent silences or inhibits expression of the wild type PDE3B in the cell.

In some aspects, the cell is inside a subject and thus the method occurs in vivo. In some aspects, the silencing or inhibiting expression of PDE3B in a cell occurs in vitro.

In some aspects, the silencing agent can be RNAi, CRISPR, or siRNA.

Disclosed are methods of silencing or inhibiting expression of wild type PDE3B in a cell comprising providing at least one RNA to the cell in an amount sufficient to inhibit the expression of PDE3B, wherein the RNA comprises or forms a double-stranded structure containing a first strand comprising a ribonucleotide sequence which corresponds to a nucleotide sequence of PDE3B and a second strand comprising a ribonucleotide sequence which is complementary to the nucleotide sequence of PDE3B, wherein the first and the second ribonucleotide sequences are separate complementary sequences that hybridize to each other to form said double-stranded structure, and the RNA comprising the double-stranded structure inhibits expression of PDE3B.

In some aspects, the first strand comprises a sequence which corresponds to a portion of the PDE3B nucleic acid sequence found in accession number NM_000922.3. In some aspects, the second strand comprises a sequence that can bind to, or is complementary to, a portion of the PDE3B nucleic acid sequence found in accession number NM_000922.3.

Disclosed are methods of inhibiting expression of PDE3B in a cell comprising: isolating the cell; contacting the cell with a RNA comprising a double-stranded structure comprising a first strand comprising a ribonucleotide sequence which corresponds to a nucleotide sequence of PDE3B and a second strand comprising a ribonucleotide sequence which is complementary to the nucleotide sequence of PDE3B, wherein the first and the second ribonucleotide sequences are separate sequences that hybridize to each other to form said double-stranded structure, and subsequently introducing the cell into a host, wherein said RNA comprising the double-stranded structure inhibits expression of the target gene in the cell in the host.

“Silencing” or “inhibiting,” as it is used herein, is a term generally used to refer to suppression, full or partial, of expression of a gene.

### M. RNA

Disclosed are RNAs comprising a double-stranded structure containing a first strand comprising a ribonucleotide sequence which corresponds to a nucleotide sequence of PDE3B and a second strand comprising a ribonucleotide sequence which is complementary to the nucleotide sequence of PDE3B, wherein the first and the second ribonucleotide sequences are separate complementary sequences that hybridize to each other to form said double-stranded structure.

In some aspects, the first strand comprises a sequence corresponding to a portion of the PDE3B nucleic acid sequence found in accession number NM_000922.3. In some aspects, the second strand comprises a sequence that can bind to, or is complementary to, a portion of the PDE3B nucleic acid sequence found in accession number NM_000922.3.

### N. Animal Models

The disclosed nucleic acids that encode the PDE3B variants or their modified forms can also be used to generate either transgenic animals or “knock out” animals which, in turn, are useful in the development and screening of therapeutically useful reagents as well as studying the mechanism of action of the genetic variant. A transgenic animal (e.g., a mouse or rat) is an animal having cells that contain a transgene, which transgene was introduced into the animal or an ancestor of the animal at a prenatal, e.g., an embryonic stage. A transgene is a DNA that is integrated into the genome of a cell from which a transgenic animal develops. In some instances, cDNA encoding one or more of the PDE3B variants can be used to clone genomic DNA encoding the one or more of the disclosed genetic variants in accordance with established techniques and the genomic sequences used to generate transgenic animals that contain cells that express DNA encoding one or more of the PDE3B variants.

### Examples

### A. Genetics of Blood Lipids Among 300,000 Multi-Ethnic Participants of the Million Veteran Program

Large-scale biobanks offer the potential to link genes to health traits documented in electronic health records (EHR) with unprecedented power. In turn, these discoveries are expected to improve the understanding of the etiology of common and complex diseases as well as the ability to treat and prevent these conditions. To this end, the Million Veteran Program (MVP) was established by the U.S. Veterans Health Administration in 2011 as a nationwide research program within the Veteran Administration (VA) healthcare system. The overarching goal of MVP is to reveal new biologic insights and clinical associations broadly relevant to human health and enhance the care of veterans through precision medicine.

Blood concentrations of total cholesterol (TC), low-density lipoprotein (LDL) cholesterol, high-density lipoprotein (HDL) cholesterol, and triglycerides (TG) are heritable risk factors for cardiovascular disease, a highly prevalent condition among U.S. veterans. Genome-wide association studies (GWAS) to date have identified at least 268 loci that influence these levels, many of which are under investigation as potential therapeutic targets. However, off-target effects have dampened enthusiasm for some of these molecules, and understanding the full spectrum of clinical consequences of a given DNA sequence variant through phenome-wide association scanning (“PheWAS”) can shed light on potential unintended effects as well as novel therapeutic indications.

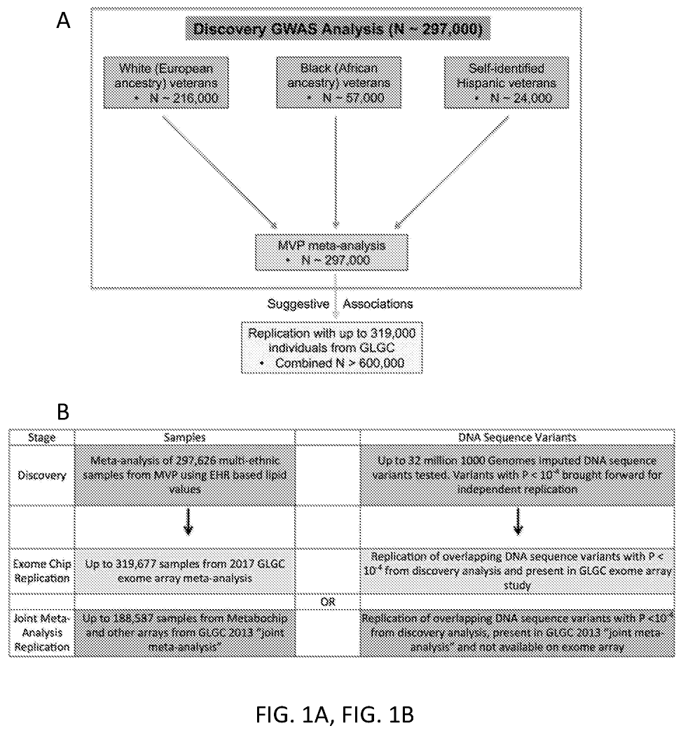

A GWAS was performed, including a discovery phase in MVP and a replication phase in the Global Lipids Genetics Consortium (GLGC) (FIG. 1). In the discovery phase, association testing was performed among 297,626 white (European ancestry), black (African ancestry), and Hispanic MVP participants with lipids stratified by ethnicity followed by a meta-analysis of results across all three groups. Replication of MVP findings was conducted in one of two independent studies from the GLGC. Novel, genome-wide lipid-associated, low-frequency missense variants unique to black and Hispanic individuals were then examined. Results for predicted loss of gene function (pLoF) mutations were focused on, as these as associations have revealed target pathways for pharmacologic inactivation and modulation of cardiovascular risk. Finally, a PheWAS was performed for a set of DNA sequence variants within genes that have already emerged as therapeutic targets for lipid modulation, leveraging the full catalog of ICD-9 diagnosis codes in the VA EHR to better understand the potential consequences of pharmacologic modulation of these genes or their products.

A transcriptome-wide association study (TWAS) and a competitive gene set pathway analysis was then performed. Novel, genome-wide lipid-associated, low-frequency missense variants unique to black and Hispanic individuals were examined. Results for predicted loss of gene function (pLoF) mutations were focused on, as these associations have revealed target pathways for pharmacologic inactivation and modulation of cardiovascular risk. A PheWAS was performed for a set of DNA sequence variants within genes that have already emerged as therapeutic targets for lipid modulation, leveraging the full catalog of ICD-9 diagnosis codes in the VA EHR to better understand the consequences of pharmacologic modulation of these genes or their products. Lastly, the causal relationship of lipids on abdominal aortic aneurysm (AAA) development were explored through a multivariate Mendelian randomization analysis.

1. Results

i. Demographic and Clinical Characteristics of Genotyped Participants in the Million Veteran Program

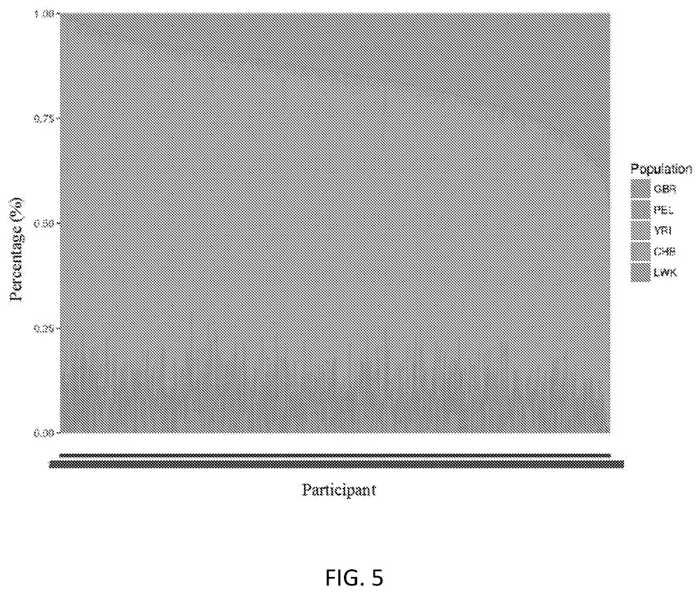

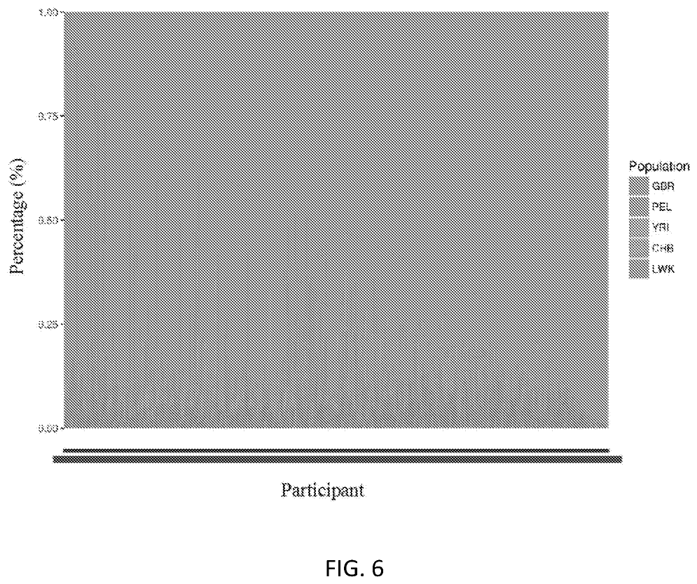

A total of 353,323 veterans had genetic data available in MVP, with clinical phenotypes recorded in the VA EHR over 3,088,030 patient-years prior to enrollment (median of 10.0 years per participant) and 61,747,974 distinct clinical encounters (median of 99 per participant). Veterans were categorized into three mutually exclusive ancestral groups for association analysis: 1) non-Hispanic whites, 2) non-Hispanic blacks, and 3) Hispanics. Admixture plots depicting the genetic background of the black and Hispanic groups are shown in FIGS. 5 and 6. Demographics and participant counts for a number of cardiometabolic traits for the 312,571 white, black, and Hispanic MVP participants that passed our quality control are depicted in Table 1.

TABLE 1
Demographic and clinical characteristics of black,
white, and Hispanic individuals passing quality
control in the Million Veteran Program

| Basic Demographics | Genotyped Veterans | |
|---|---|---|
| N | 312,571 | |
| Age at Enrollment ± SD, years | 62.4 ± 13.5 | |
| Male, n (%) | 287,441 | (92.0%) |
| Body Mass Index ± SD, kg/m² | 30.3 ± 6.0 | |
| Current Smoker, n (%) | 59,385 | (19.0%) |
| Former Smoker, n (%) | 159,459 | (51.0%) |
| N with ≥1 Measurement of | 297,626 | (95.2%) |
| Plasma Lipids, (%) | | |
| Number of Lipid Measurements, | 15,456,328 | (12) |
| (Median Per Lipid Fraction) | | |

Race/Ethnicity

| | | |
|---|---|---|
| Black, n (%) | 59,007 | (18.9%) |
| White, n (%) | 227,817 | (72.8%) |
| Hispanic, n (%) | 25,747 | (8.1%) |

Cardiometabolic Disease at Enrollment*

| | | |
|---|---|---|
| Coronary Artery Disease, n (%) | 67,912 | (21.7%) |
| Type 2 Diabetes, n (%) | 92,079 | (29.5%) |
| Peripheral Artery Disease, n (%) | 21,418 | (6.9%) |
| Abdominal Aortic Aneurysm, n (%) | 5,618 | (1.8%) |
| Deep Venous Thrombosis or | 7,009 | (2.2%) |
| Pulmonary Embolism, n (%) | | |

*Diseases are defined by International Classification of Disease, Ninth Edition (ICD-9) diagnosis codes.
Abbreviations:
SD, Standard Deviation

A subset of 297,626 participants passing quality control had at least 1 laboratory measurement of blood lipids in their EHR. These individuals collectively had a total of 15,456,328 lab entries for blood lipids, or a median of 12 measures per lipid fraction per participant. To minimize potential confounding from the use of lipid-altering agents with variable adherence, a participant's maximum LDL cholesterol, TG, and TC as well as his or her minimum HDL cholesterol were selected for genetic association analysis. Table 2 summarizes characteristics at enrollment and the distribution lipid levels for MVP participants included in the analysis. Participants were largely male, 72% white, and while 39-46% of participants in each ancestral group had statin therapy prescriptions at the time of enrollment, only 8-9% were prescribed statin therapy at the time of their maximum LDL or TC measurement used for GWAS analysis.

TABLE 2
Demographic and clinical characteristics for 297,626 veterans
in the Million Veteran Program lipids analysis

| | White | Black | Hispanic |
|---|---|---|---|
| Veterans, N (%) | 215,551 (72.4%) | 57,332 (19.3%) | 24,743 (8.3%) |
| Age at Enrollment ± SD, years | 64.2 ± 13 | 57.7 ± 11.8 | 56.3 ± 15.0 |
| Male, n (%) | 200,900 (93.2%) | 50,059 (87.3%) | 22,601 (91.3%) |
| Body Mass Index ± SD, kg/m² | 30.1 ± 5.9 | 30.4 ± 6.3 | 30.7 ± 5.8 |
| Statin Therapy Prescription at | 100,024 (46.4%) | 23,302 (40.6%) | 9,646 (39.0%) |
| Enrollment, n (%) | | | |
| Statin Therapy Prescription at | 18,818 (8.7%) | 5,024 (8.8%) | 2,262 (9.1%) |
| time of Max LDL Blood Draw, | | | |
| n (%) | | | |
| Statin Therapy Prescription at | 18,433 (8.6%) | 5,027 (8.8%) | 2,162 (8.7%) |
| time of Max TC Blood Draw, n | | | |
| (%) | | | |
| Mean Min HDL-C ± SD, | 36.2 ± 11.4 | 38.9 ± 12.8 | 36.4 ± 11.0 |
| mg/dL | | | |
| Mean Max LDL-C ± SD, | 139 ± 38.4 | 142.2 ± 40.7 | 141.3 ± 38.1 |
| mg/dL | | | |
| Median Max TG ± IQR, mg/dL | 211 ± 174 | 179 ± 149 | 221 ± 184 |
| Mean Max TC ± SD, mg/dL | 218.6 ± 46.7 | 220.8 ± 47.2 | 221.9 ± 48.0 |
| Variants Included in Analysis | 19,342,852 | 31,448,849 | 30,455,745 |

Abbreviations:
Min, Minimum;
Max, Maximum;
SD, Standard Deviation;
HDL-C, High-Density Lipoprotein Cholesterol;
LDL-C, Low-Density Lipoprotein Cholesterol;
TG, Triglycerides;
TC, Total Cholesterol

### Genetic Association Analysis of Lipids and Conditional Analysis

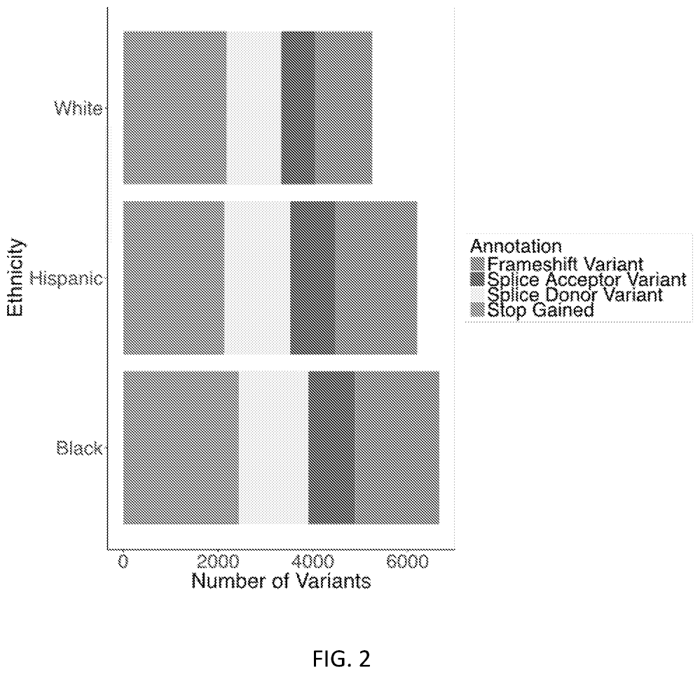

19.3, 31.4, and 30.4 million variants in white, black, and Hispanic veterans, respectively, were successfully imputed [INFO>0.3, minor allele frequency (MAF)>0.0003] using the 1000 Genomes Project reference panel (Table 2). Black and Hispanic participants had substantially more variants available for analysis, reflecting the known greater genetic diversity within these populations. We also identified 6,657 pLoF variants in 4,294 genes across the three ethnicities (FIG. 2).

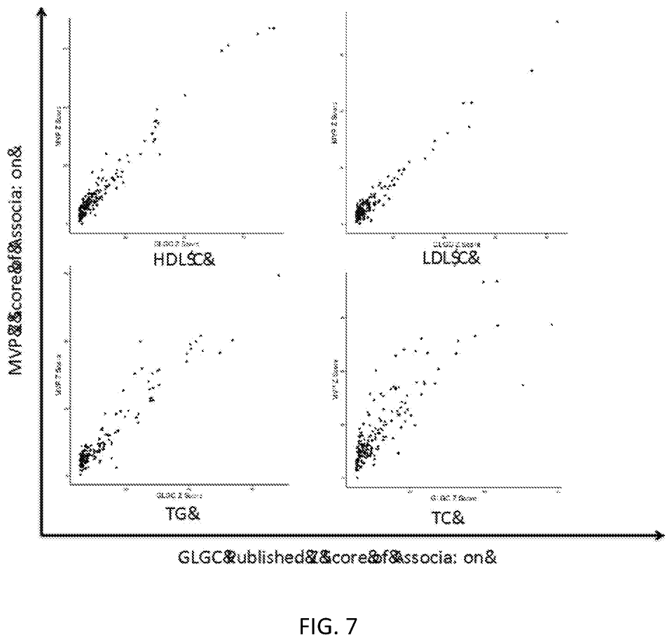

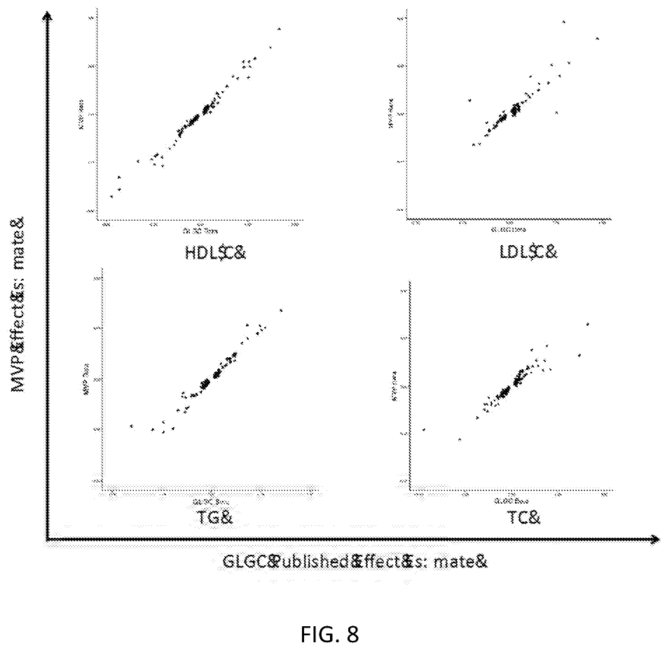

The Z scores and effect estimates from the published literature were compared with those observed in MVP for 444 previously reported exome-wide significant variants for lipids that were imputed using HapMap. A strong correlation of genetic associations was found across all four traits, validating the lipid phenotypes defined through EHR (FIG. 7,8).

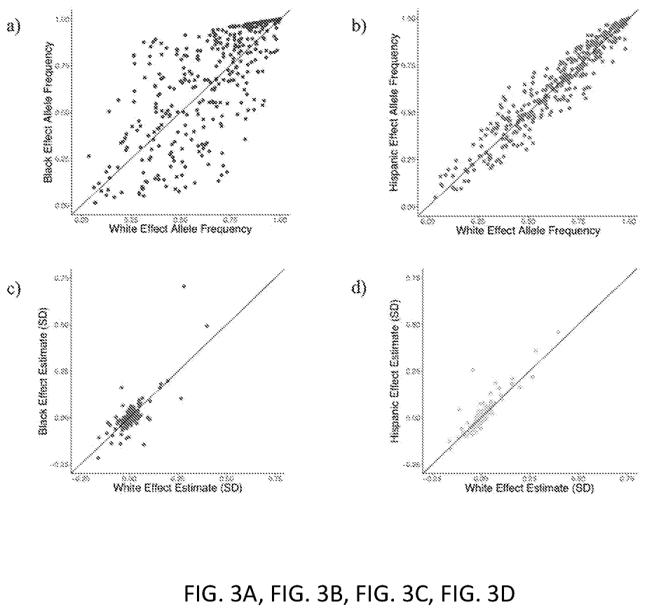

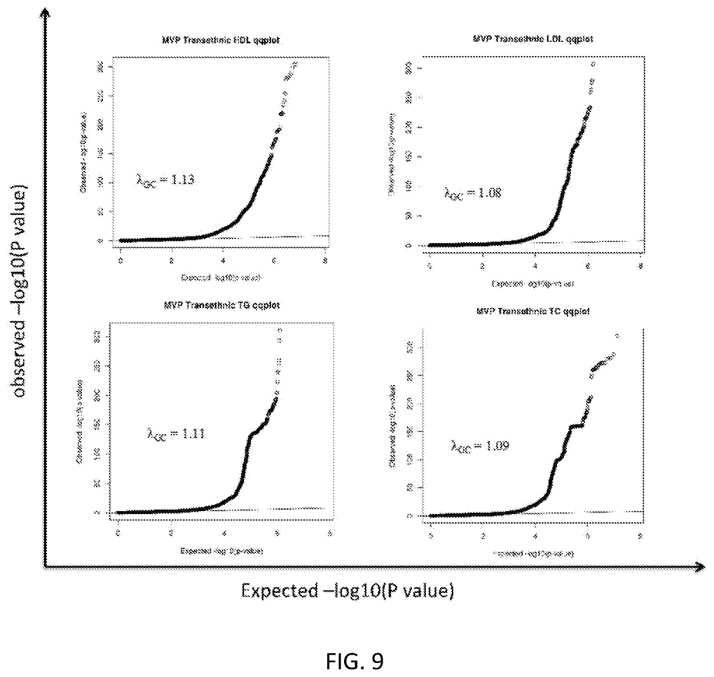

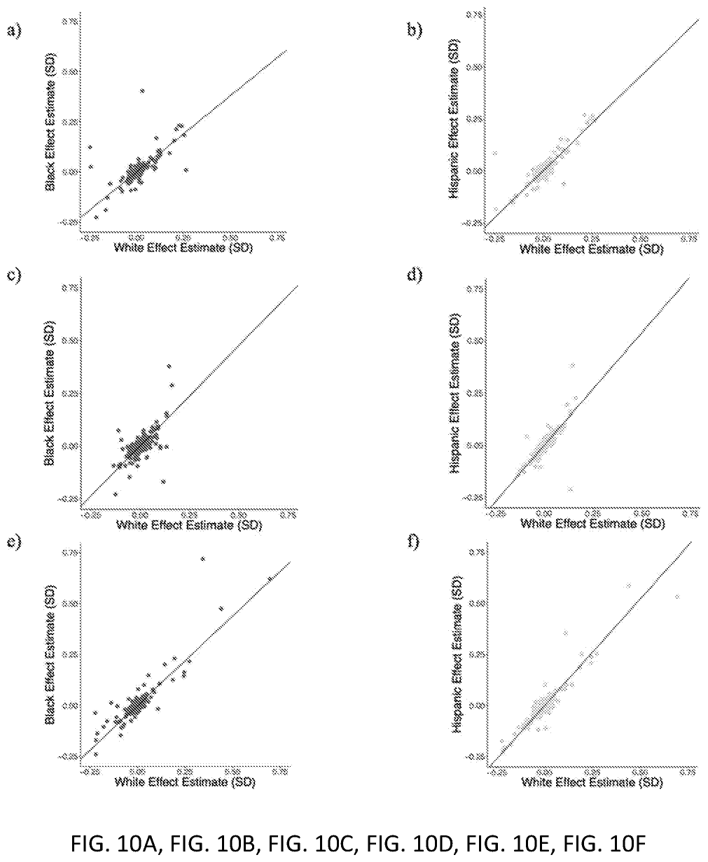

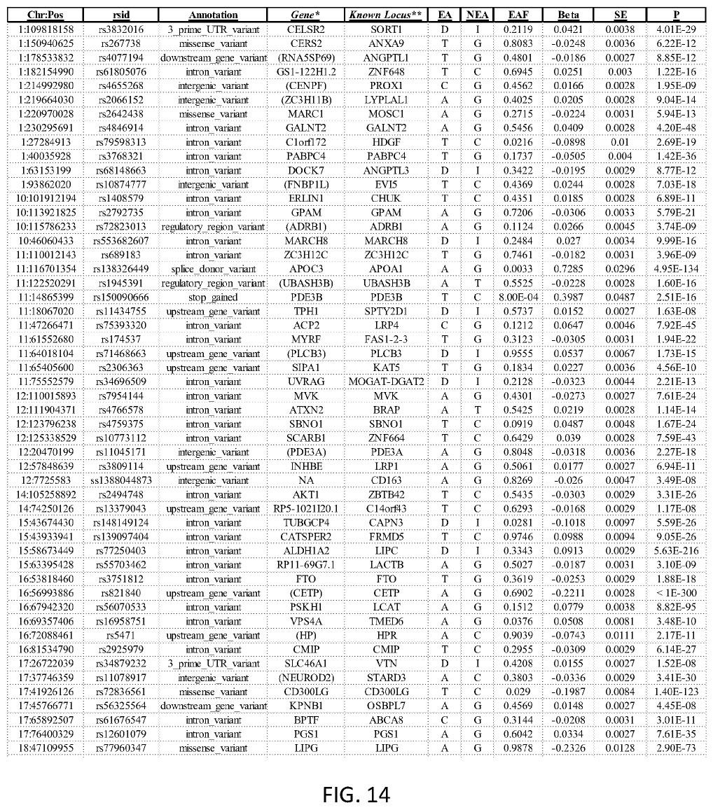

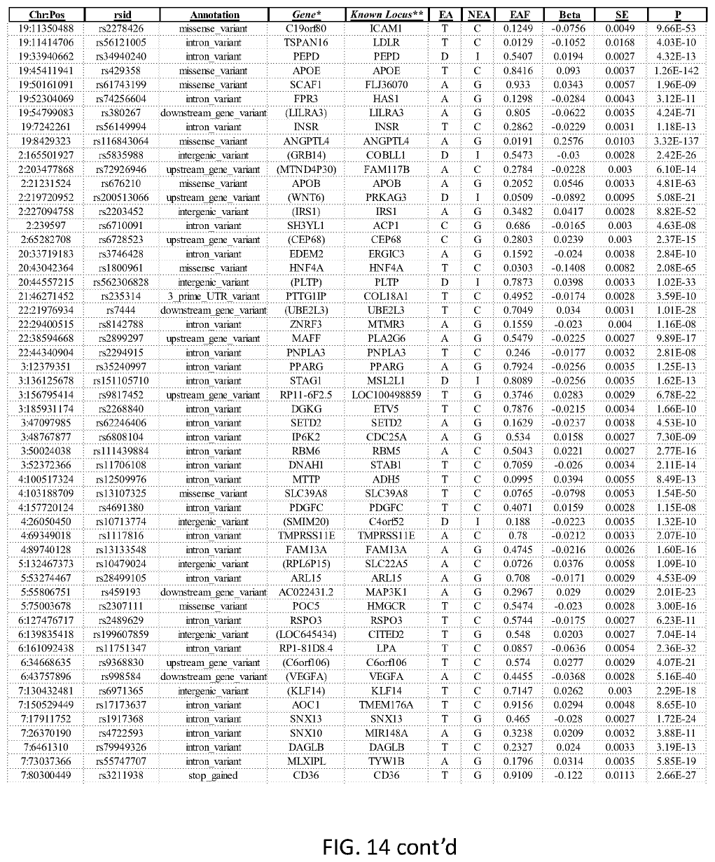

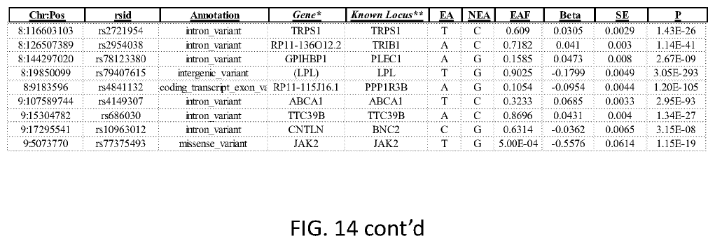

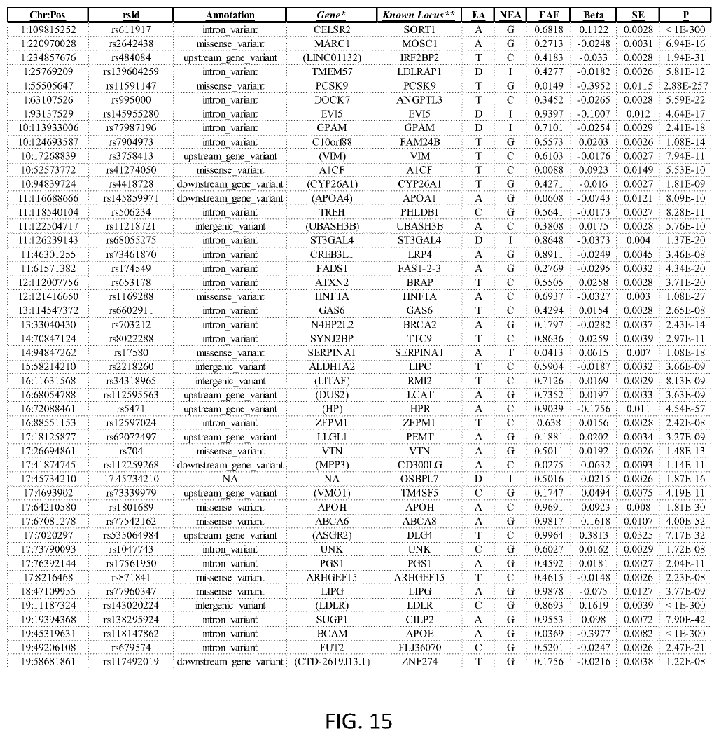

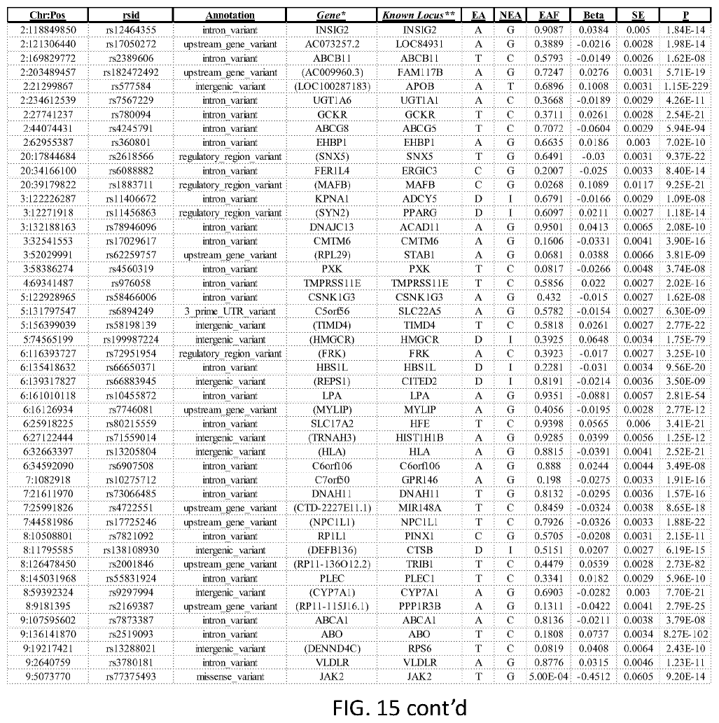

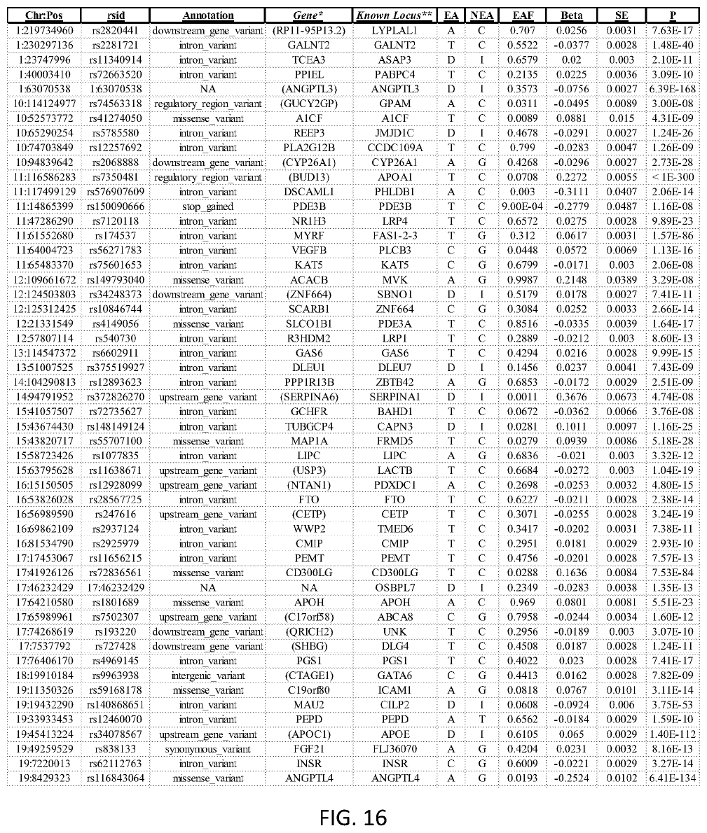

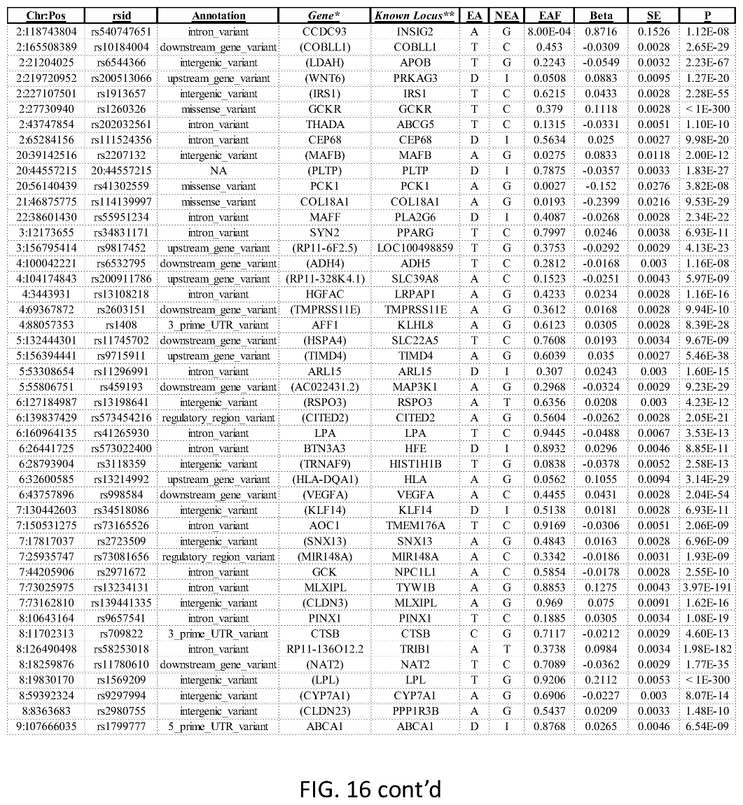

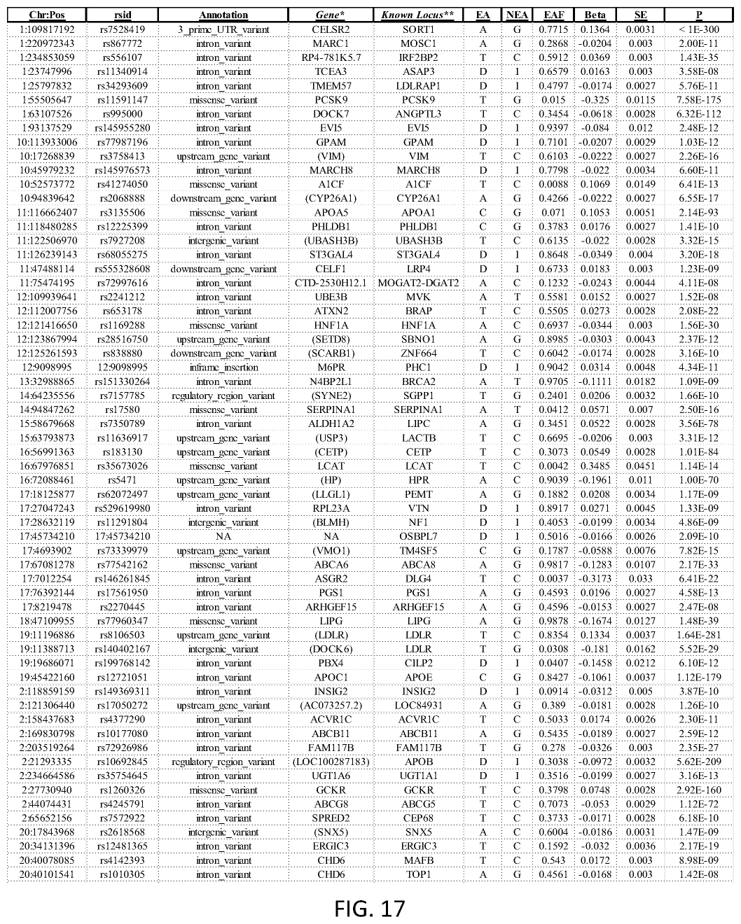

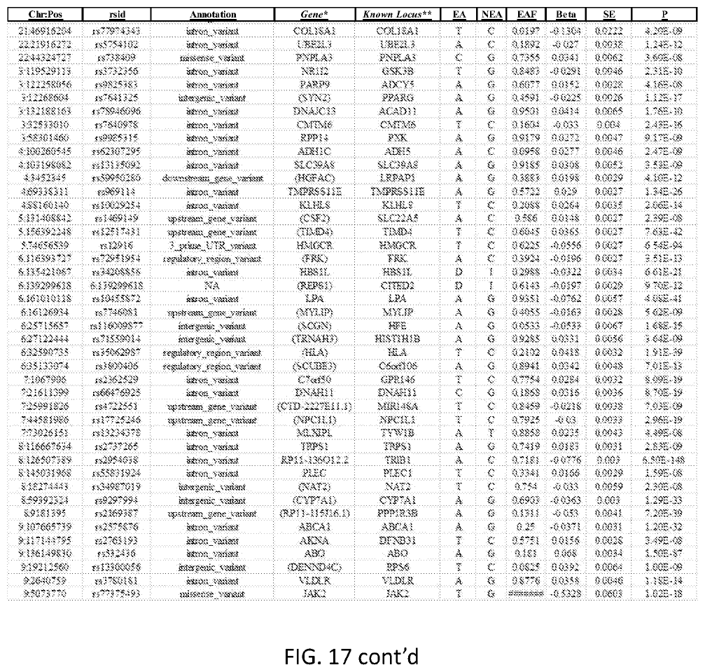

Association testing was performed separately among individuals of each of three ancestries (whites, blacks, and Hispanics) in the initial discovery analysis and then meta-analyzed results across ancestry groups using an inverse variance-weighted fixed effects method (FIG. 1a, FIG. 9). Following trans-ethnic meta-analysis in the discovery phase of the study, a total of 46,526 variants at 188 of the 268 known loci for lipids met the genome-wide significance threshold (P<5×10-8) (FIGS. 14-17). Pairwise comparisons of the allele frequencies and effect estimates were performed between whites and blacks as well as between whites and Hispanics for 354 of the 444 previously established independent genome-wide significant variants for lipids which were well imputed in all three ancestral groups in MVP (FIG. 3). A much stronger correlation between white and Hispanic effect allele frequencies (Pearson correlation coefficient R=0.96) than between whites and blacks (R=0.72) was noted, likely reflecting the greater European admixture in the MVP Hispanic participants. The correlation in effects estimates among the three ethnicities varied by lipid trait (FIG. 3, FIG. 10).

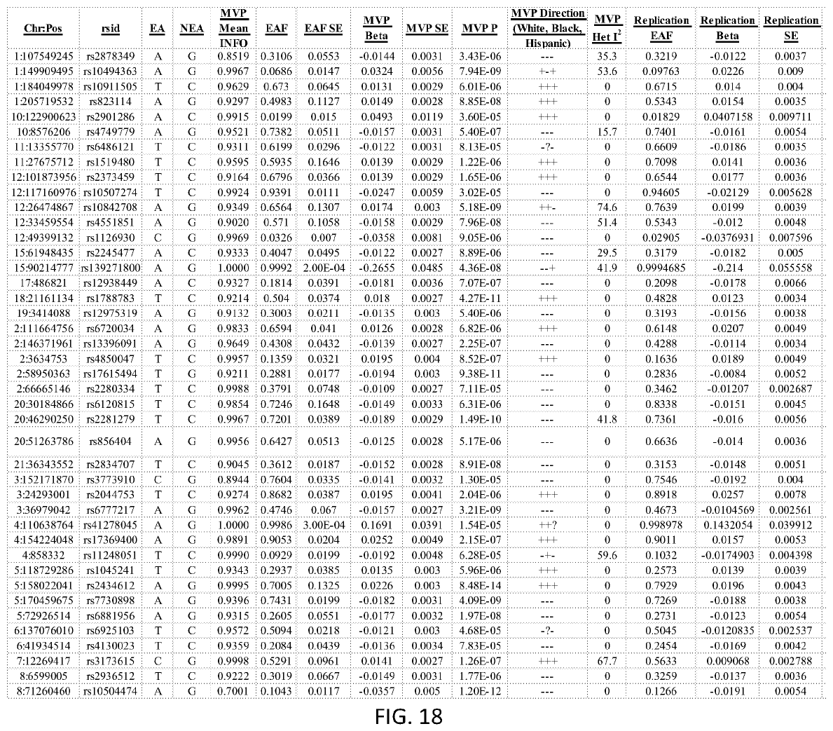

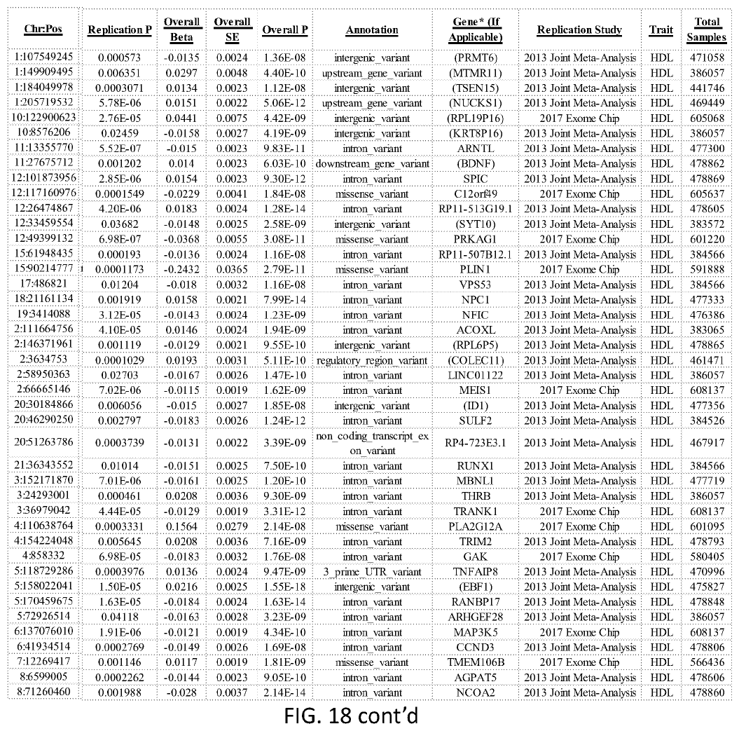

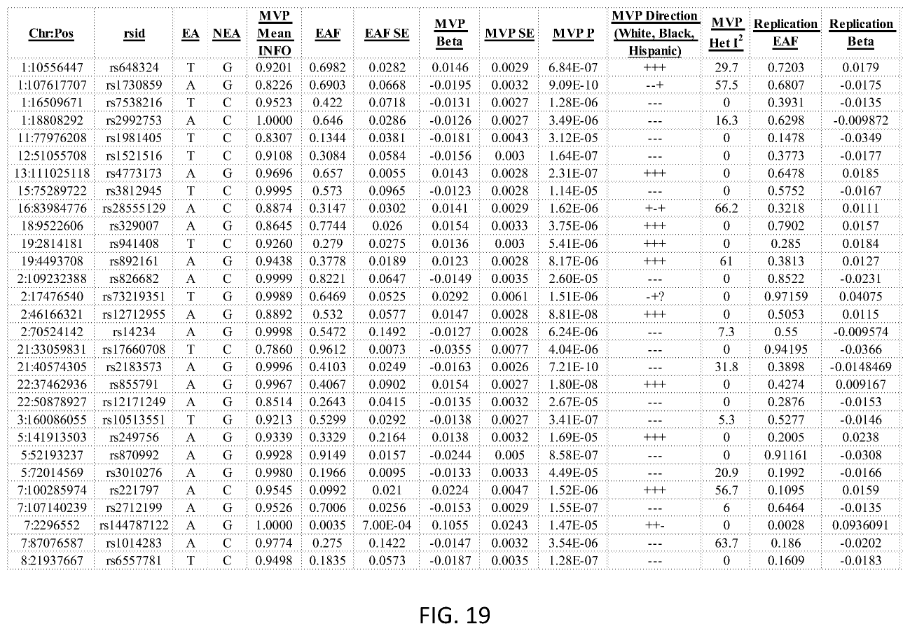

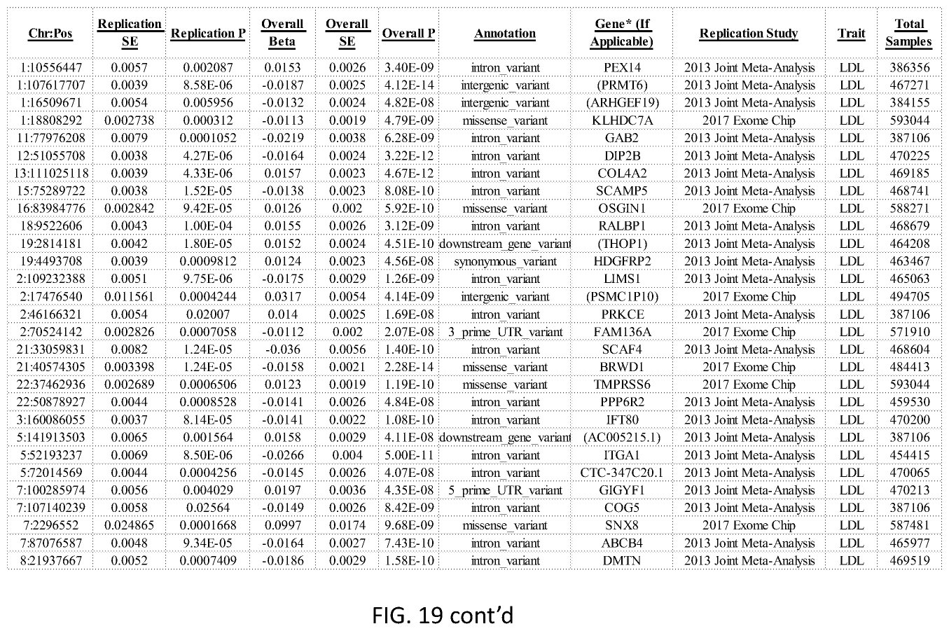

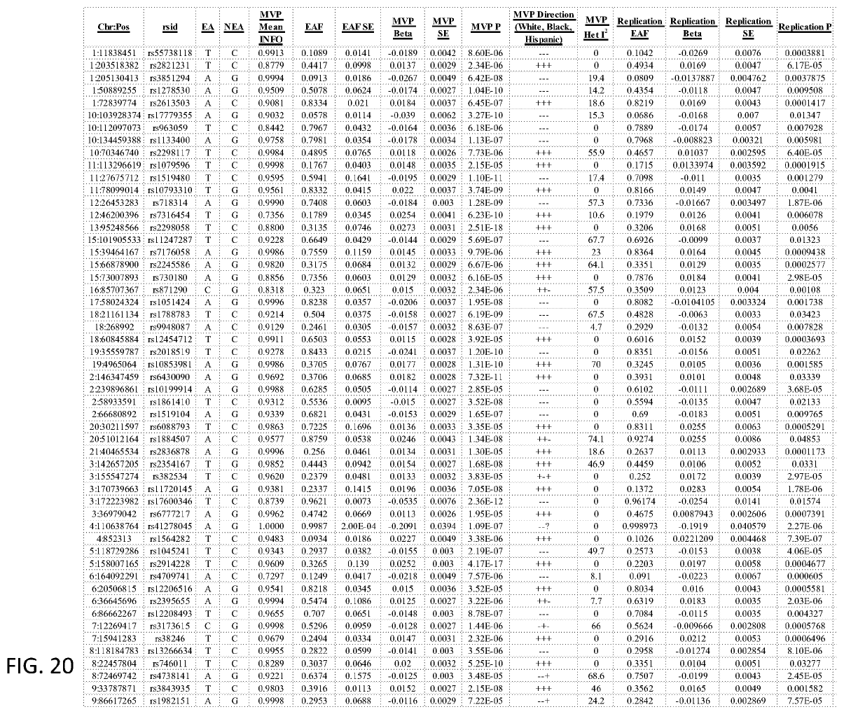

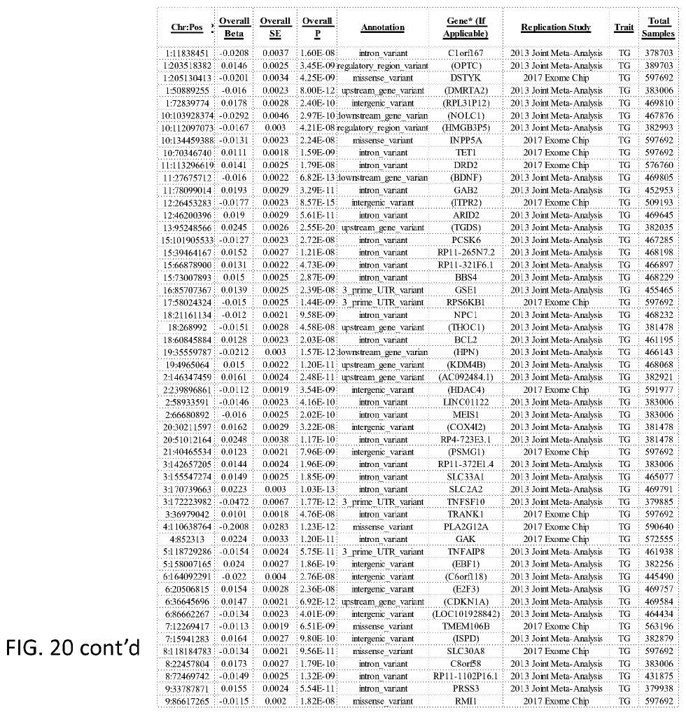

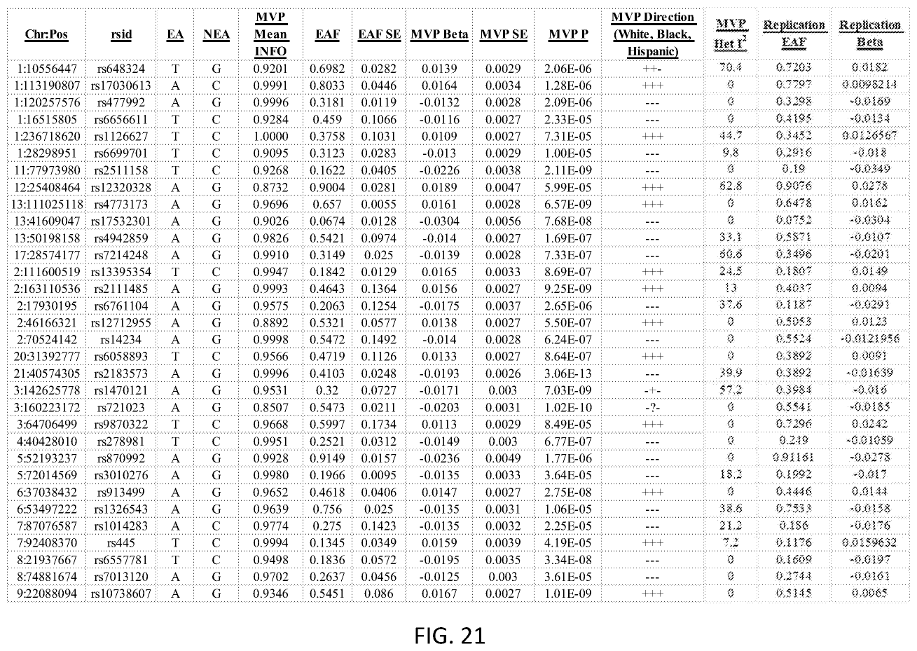

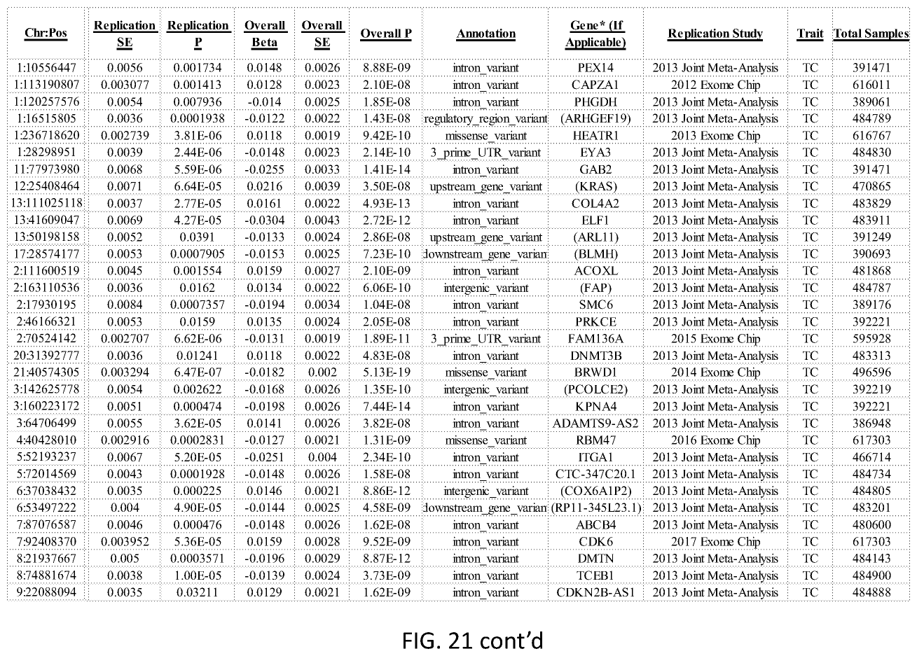

Replication for variants within MVP with suggestive associations (P<1×10-4) was sought in one of two independent studies (FIG. 1b). Replication was first performed using summary statistics from the 2017 GLGC exome array meta-analysis. If a DNA sequence variant was not available for replication in the above exome array-focused study, we sought replication of remaining variants from publicly available summary statistics from the 2013 GLGC “joint meta-analysis. A total of 170,925 variants demonstrated suggestive association (P<10-4) in the MVP discovery analysis. Among these variants, 39,663 were also available for in silico replication in at least one of the two GLGC studies involving up to 319,677 additional individuals. Significant novel associations were defined as those that were at least nominally significant in replication (P<0.05) and had an overall P<5×10-8 (genome-wide significance) in the discovery and replication cohorts combined. Following replication, 118 novel loci exceeded genome-wide significance (P<5×10-8, FIGS. 18-21). Minor allele frequencies (MAF) of lead variants ranged from 0.08% to 49.9%, with effect sizes ranging from 0.01 to 0.243 standard deviations. For example, carriers of a rare missense mutation in the gene encoding Sorting Nexin-8 [SNX8 p.Ile414Thr, (rs144787122) MAF=0.35% in MVP] demonstrated a 0.10 standard deviation (3.8 mg/dL) higher plasma LDL cholesterol after testing in 587,481 individuals.

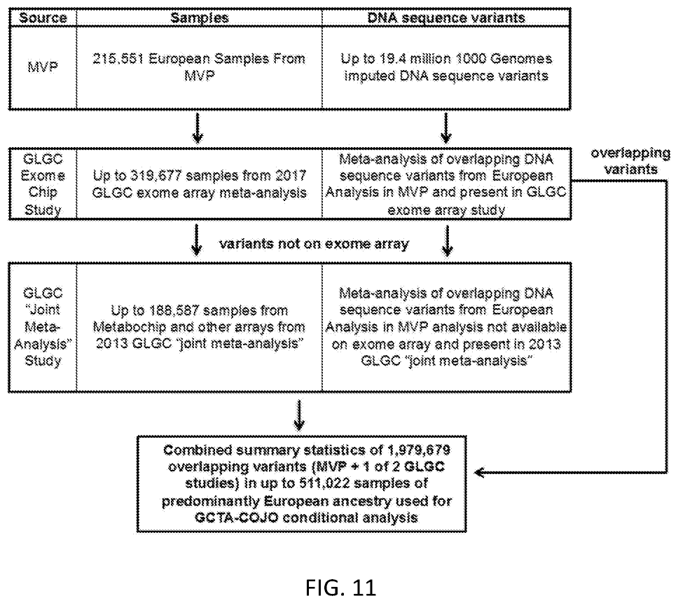

At any given genetic locus, more than one variant may independently affect plasma lipid levels. A conditional analysis was performed using combined summary statistics from MVP and publicly available data from GLGC for each lipid trait (FIG. 11). A total of 826 independently associated lipid variants were identified across 118 novel and 268 previously identified loci (data not shown).

ii. Variance Explained and Gain Using Multiple Lipid Measurements

The previously mapped 444 lipid variants explain about 7.5-10.5% of the phenotypic variance in lipid levels in the MVP population. The 118 novel loci explain an additional 0.38-0.74% in phenotypic variance, and the 826 independent variants identified in the conditional analysis increase the overall phenotypic variance explained to 8.8-12.3% (Table 3).

TABLE 3
Variance explained for 444 previously mapped independent
genome-wide variants, 118 novel loci identified in this
study, and 826 independent lipid genome-wide variants
identified on conditional analysis in this study

| | Variance Explained | | | |
|---|---|---|---|---|
| Variants | HDL | LDL | TG | TC |
| 444 Previously Mapped | 0.1055 | 0.0858 | 0.0921 | 0.0758 |
| Variants | | | | |
| 118 Novel Loci from | 0.006215 | 0.003862 | 0.007487 | 0.004655 |
| MVP Study | | | | |
| 826 Variants from MVP | 0.1231 | 0.0964 | 0.1095 | 0.0886 |
| Conditional Analysis | | | | |

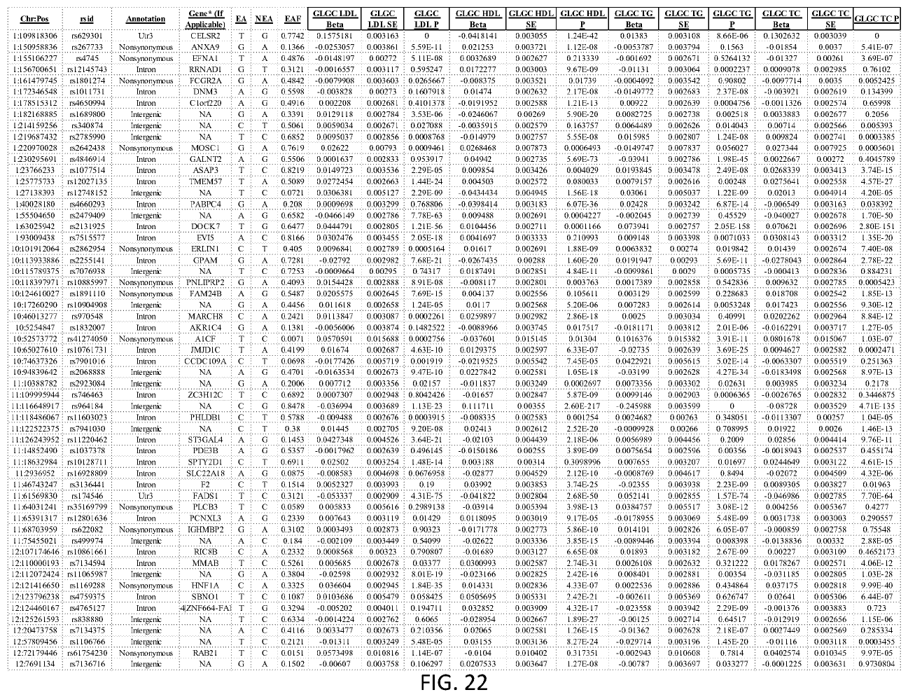

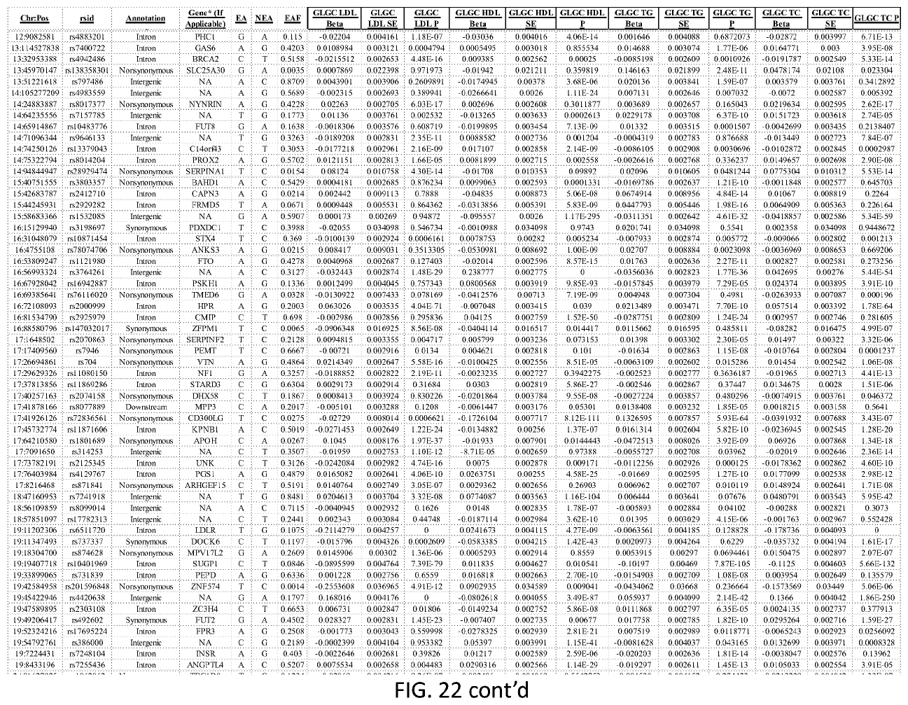

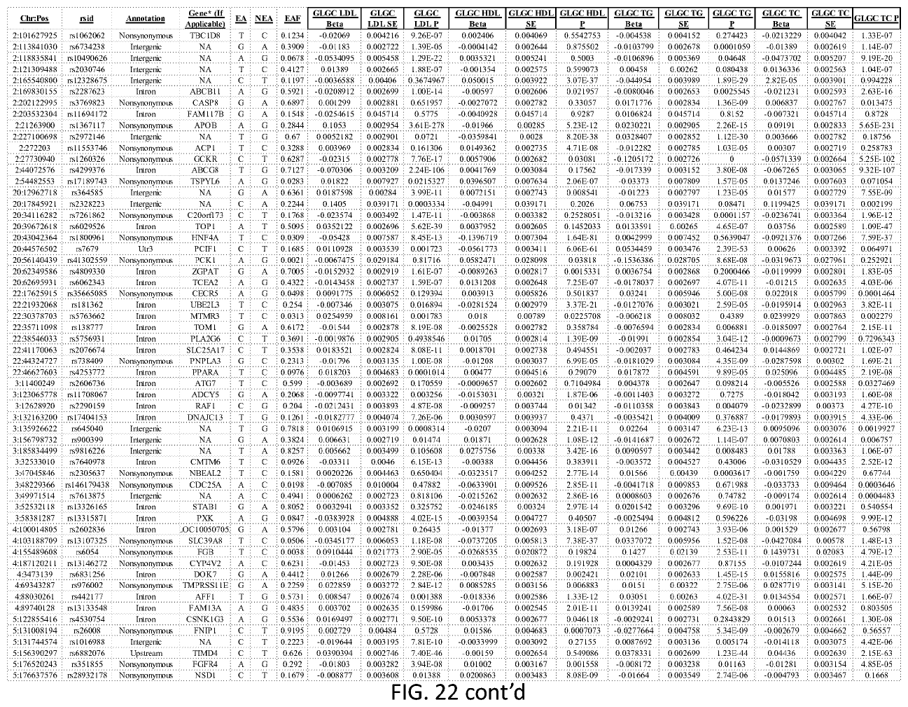

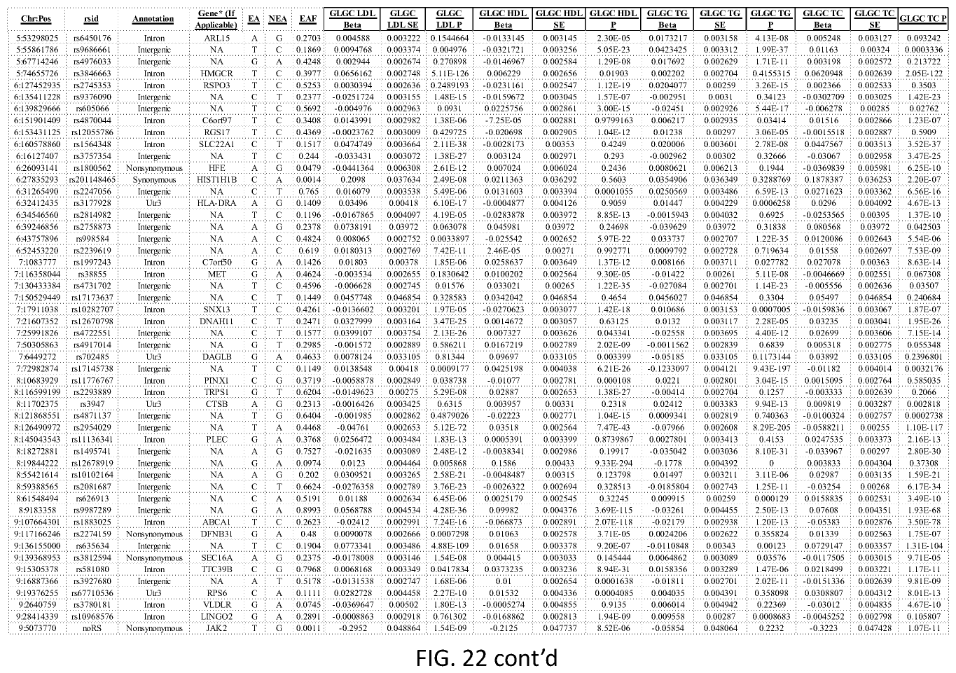

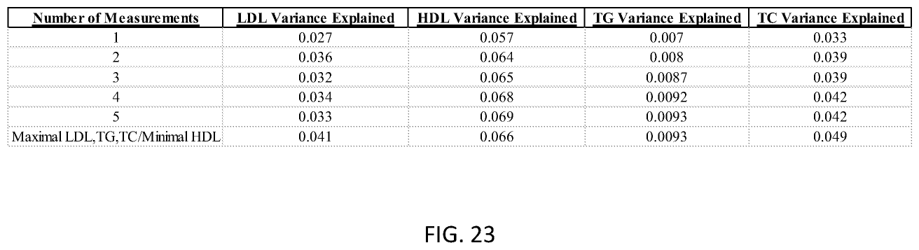

The impact of multiple lipid measurements was subsequently explored in an analysis restricted to 171,314 European MVP participants with >5 lipid measurements in their EHR. A weighted genetic risk score (GRS) of 223 variants was constructed across 268 of the previously mapped loci with effect estimates available in the 2017 GLGC exome array analysis summary statistics (FIG. 22). Generally across the four lipid traits, the GRS explained a larger proportion of the phenotypic variance with an increasing number of lipid measurements included in the analysis (FIG. 23). In addition, when the maximal/minimal lipid values were used as in GWAS, the GRS explained more total variance than when using up to 5 lipid measurements for the LDL-C, TG, and TC phenotypes.

iii. Transcriptome-wide Association Study

A TWAS23 was performed using: 1) pre-computed weights from expression array data measured in peripheral blood from 1,245 unrelated control individuals from the Netherlands Twin Registry (NTR), RNA-seq data measured in adipose tissue from 563 control individuals from the Metabolic Syndrome in Men study (METSIM), and RNA-seq data from post-mortem liver (97 individuals) and tibial artery (285 individuals) tissue from the Genotype-Tissue Expression project (GTEx V6), and 2) combined MVP and GLGC summary statistics for each of the four lipid traits (FIG. 11). Briefly, this approach integrates information from expression reference panels (variant-expression correlation), GWAS summary statistics (variant-trait correlation), and linkage disequilibrium (LD) reference panels (variant-variant correlation) to assess the association between the cis-genetic component of expression and phenotype. The results yield candidate causal genes from the GWAS results under the assumption that the causal mechanism of the tested genes involves changes in cis-expression.

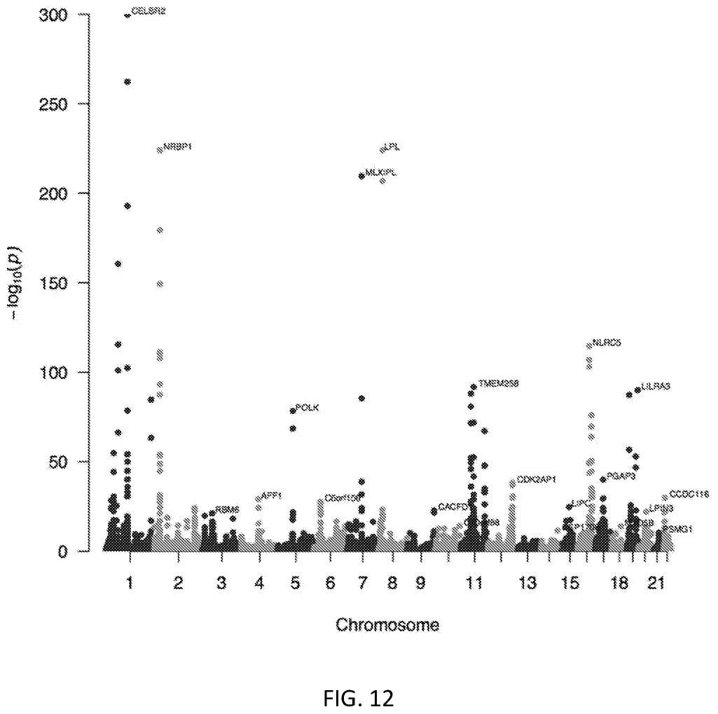

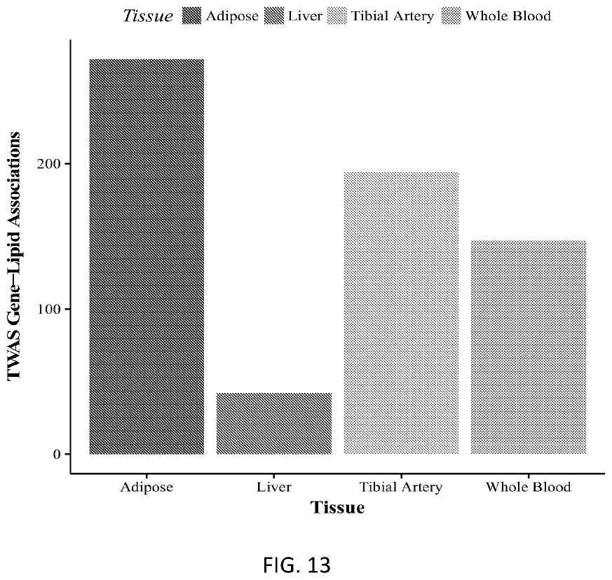

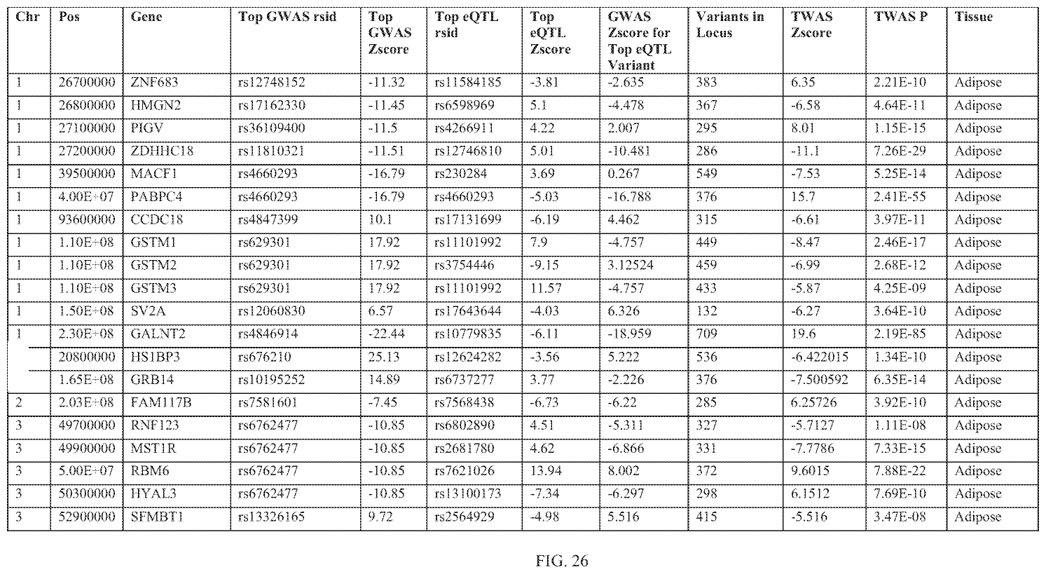

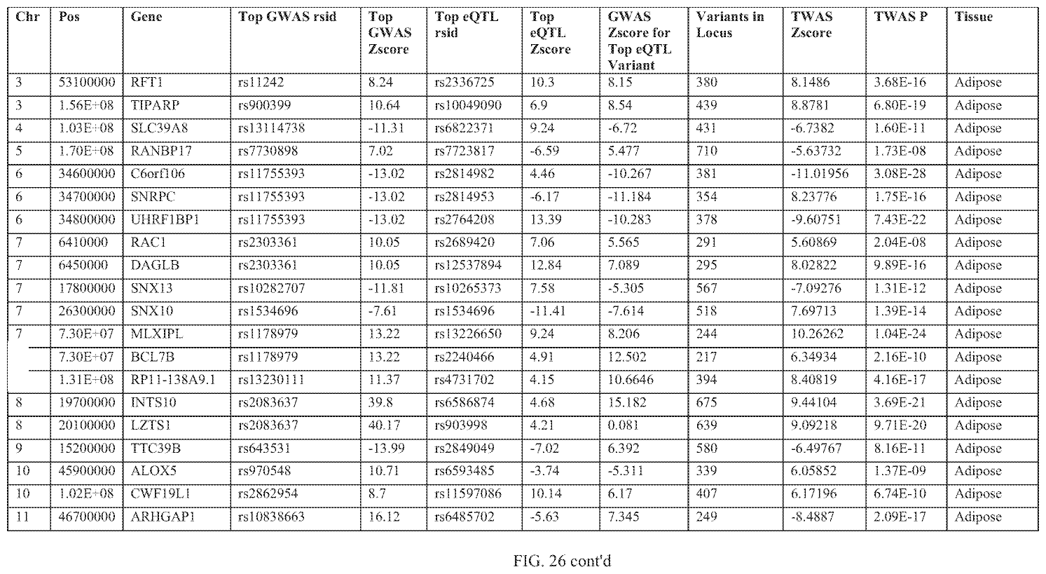

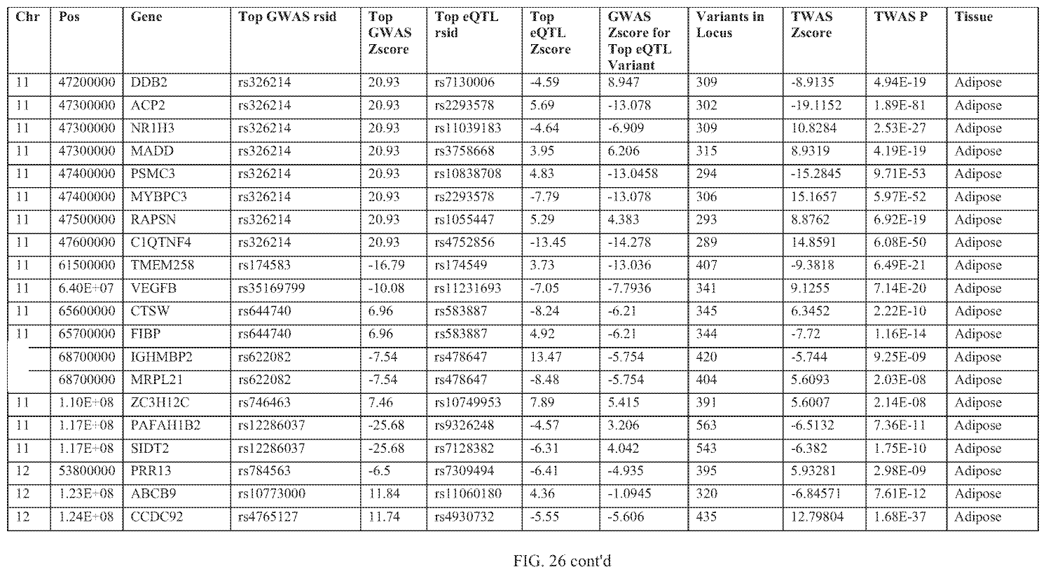

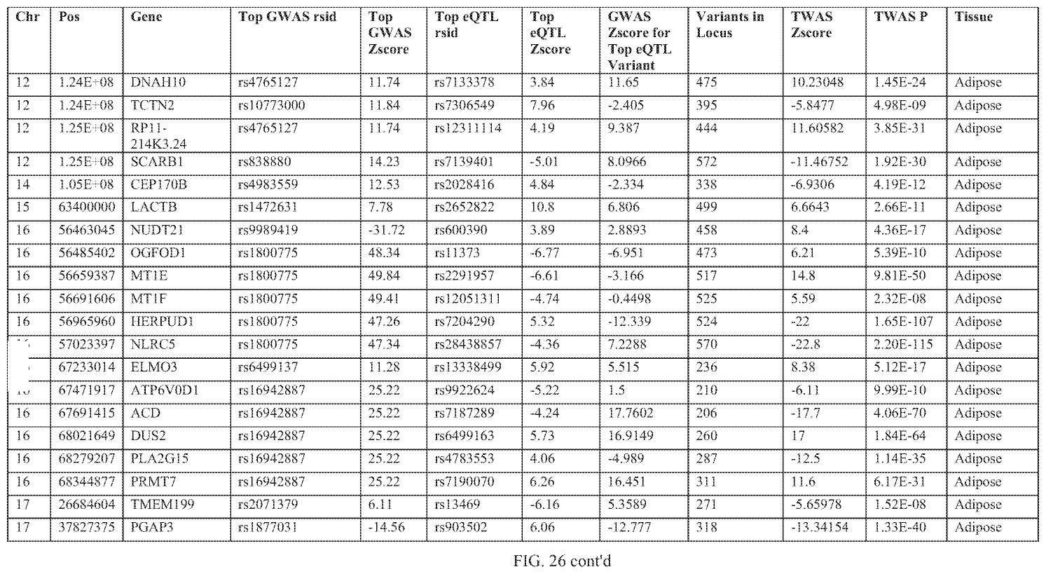

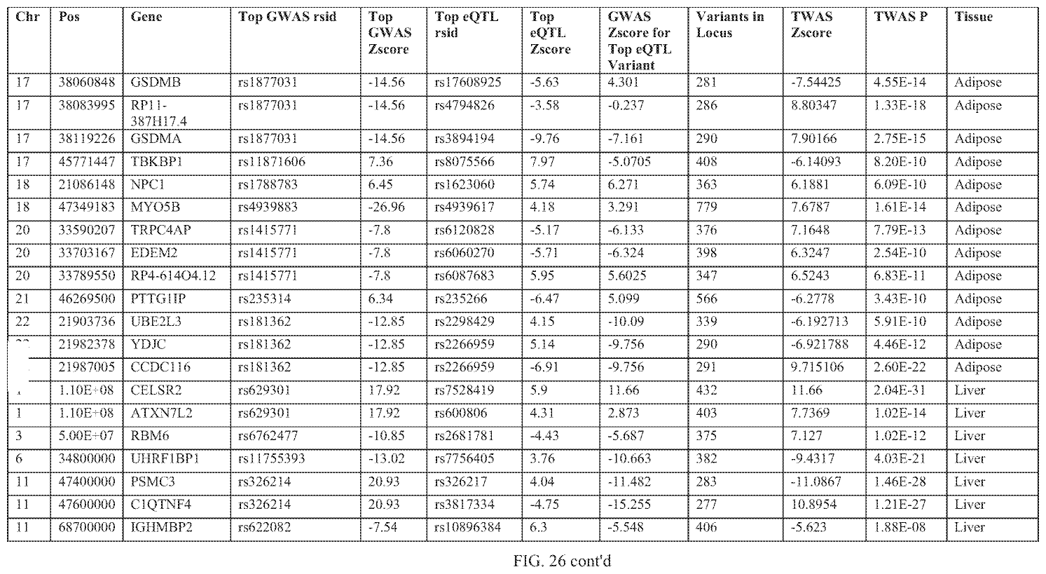

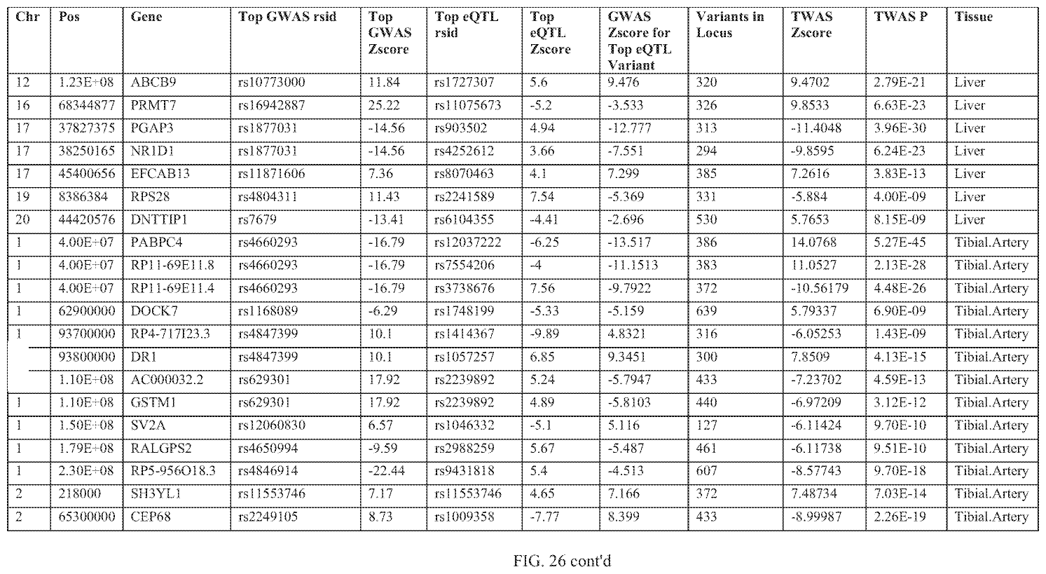

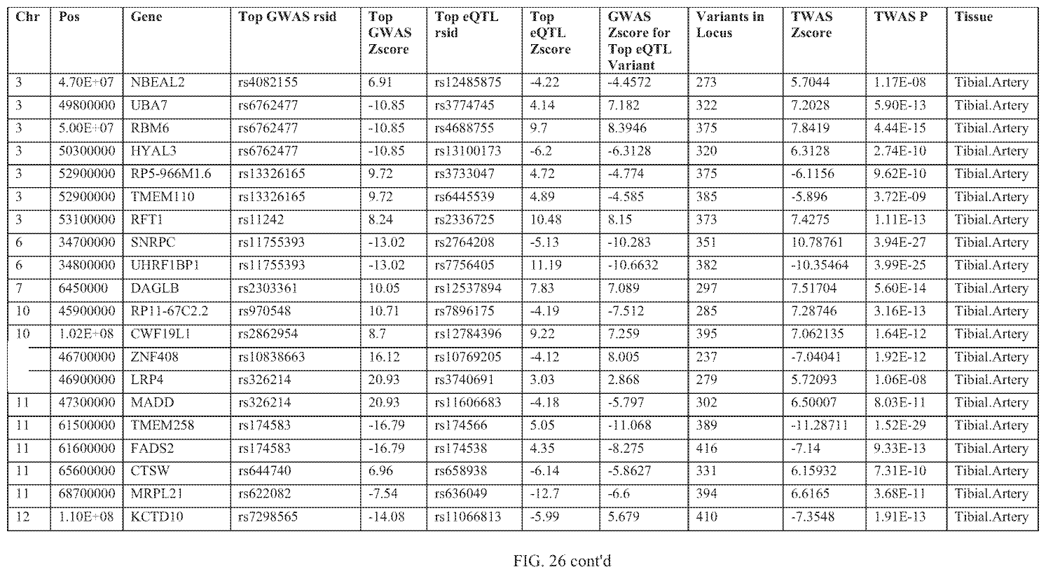

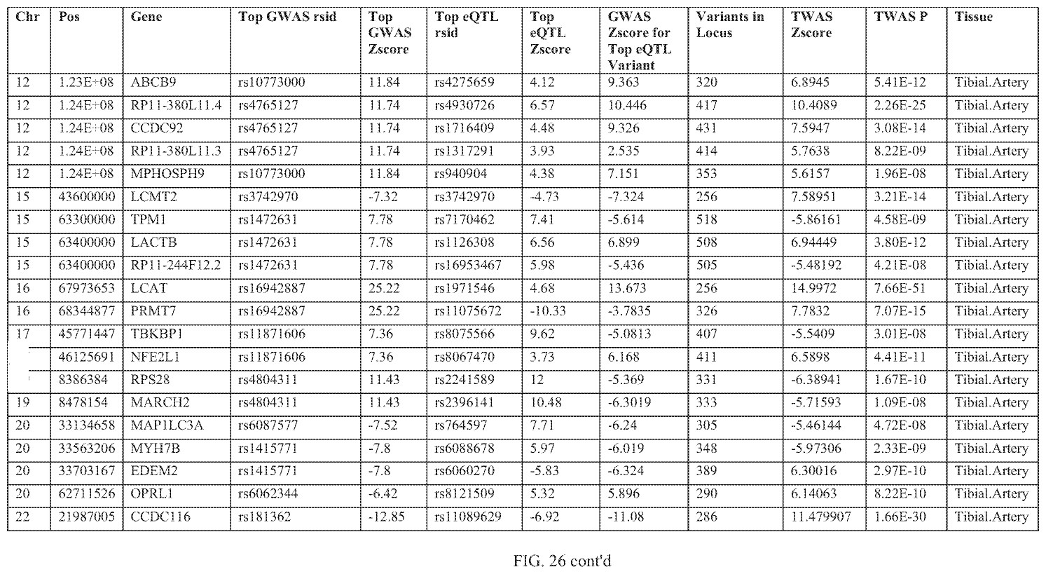

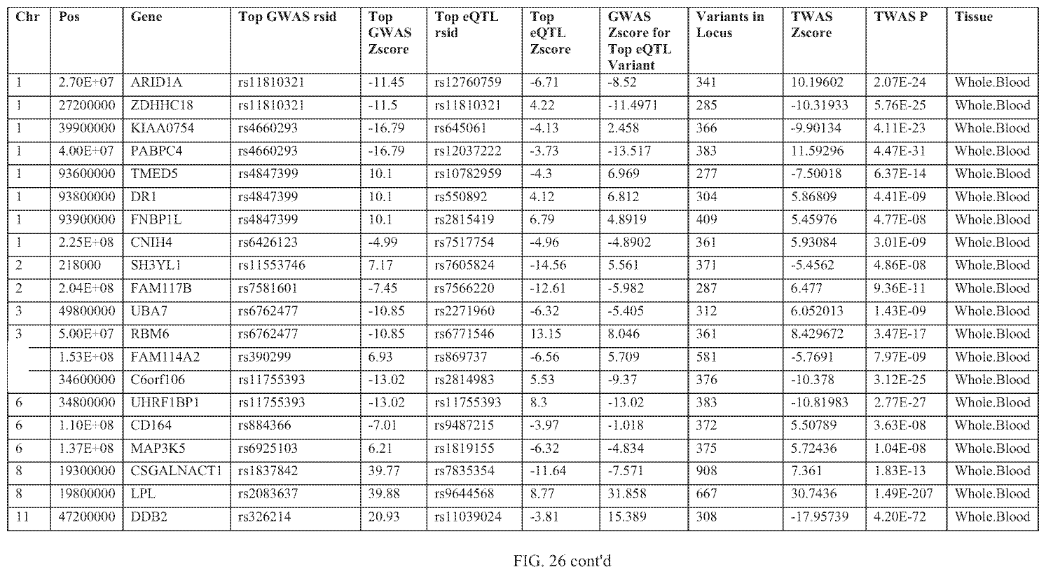

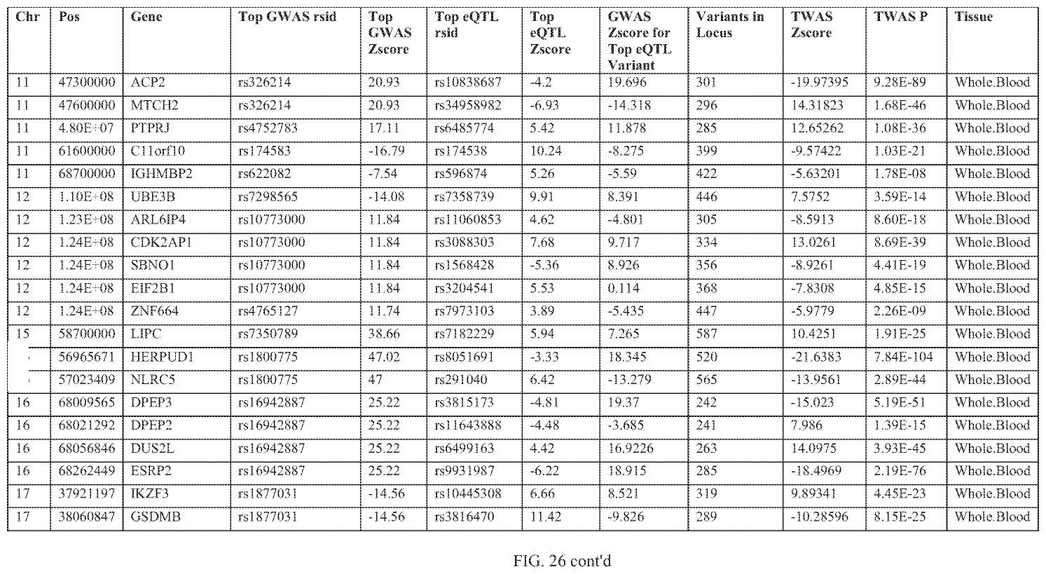

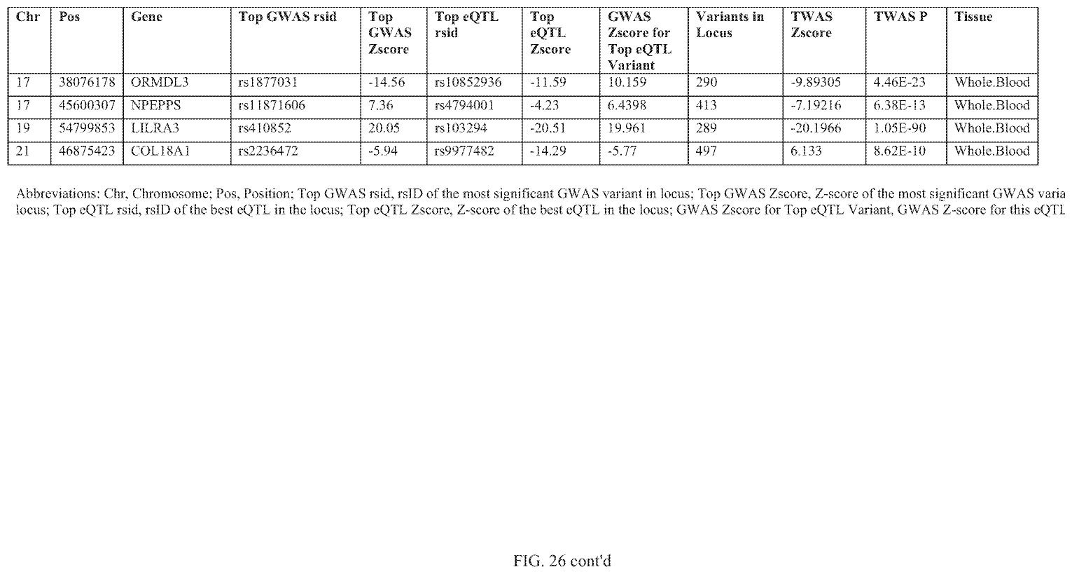

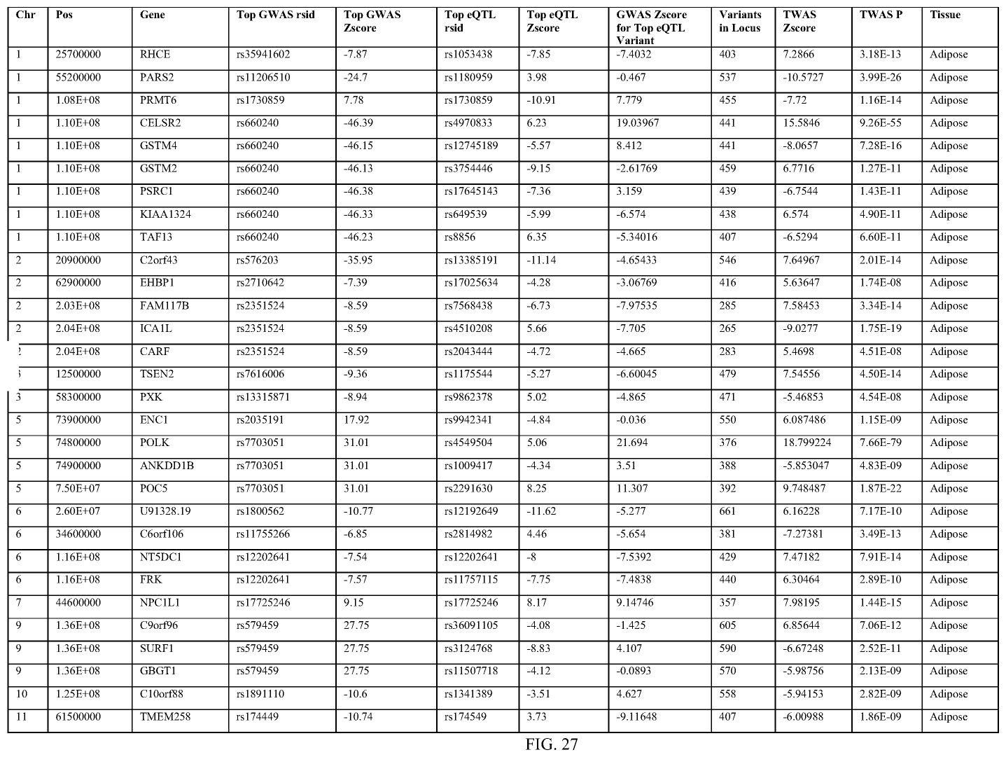

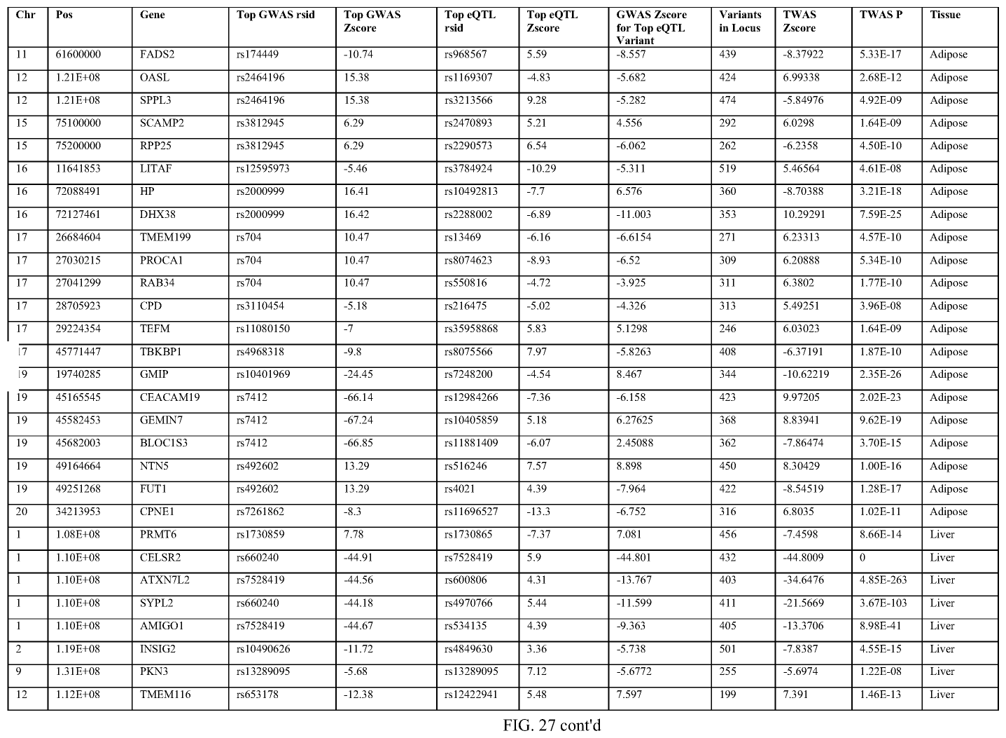

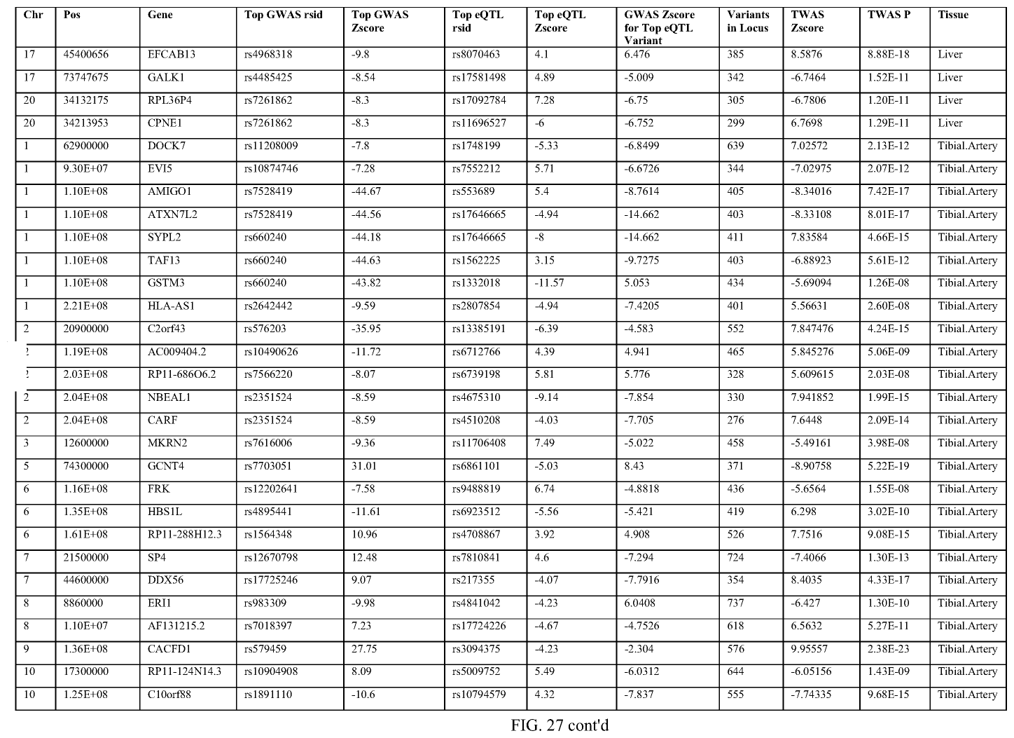

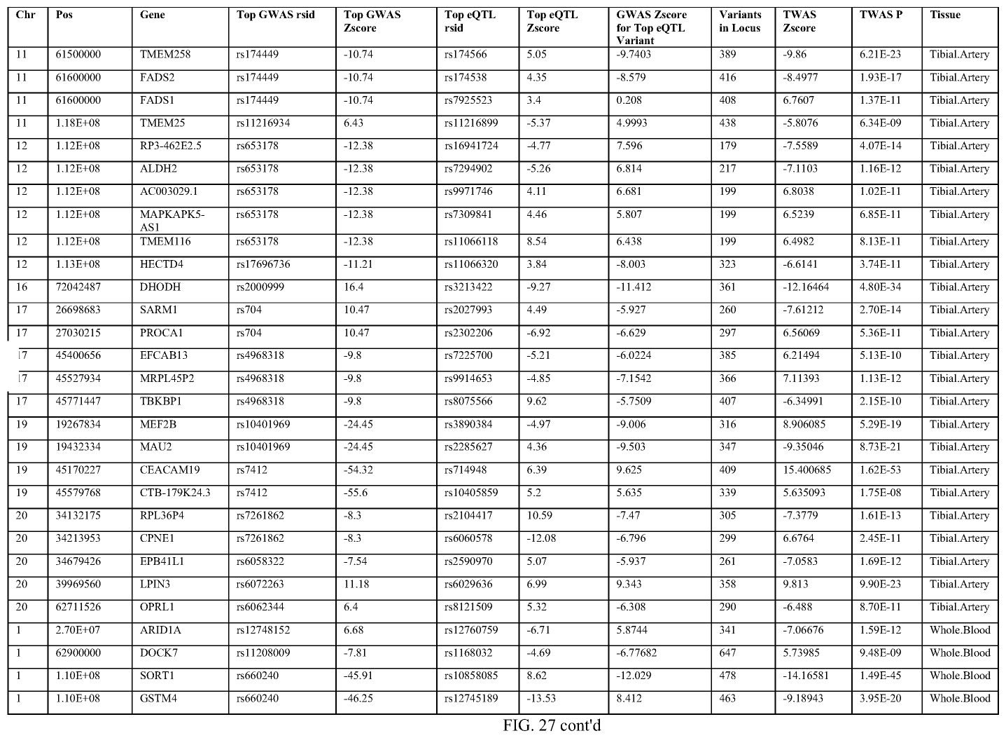

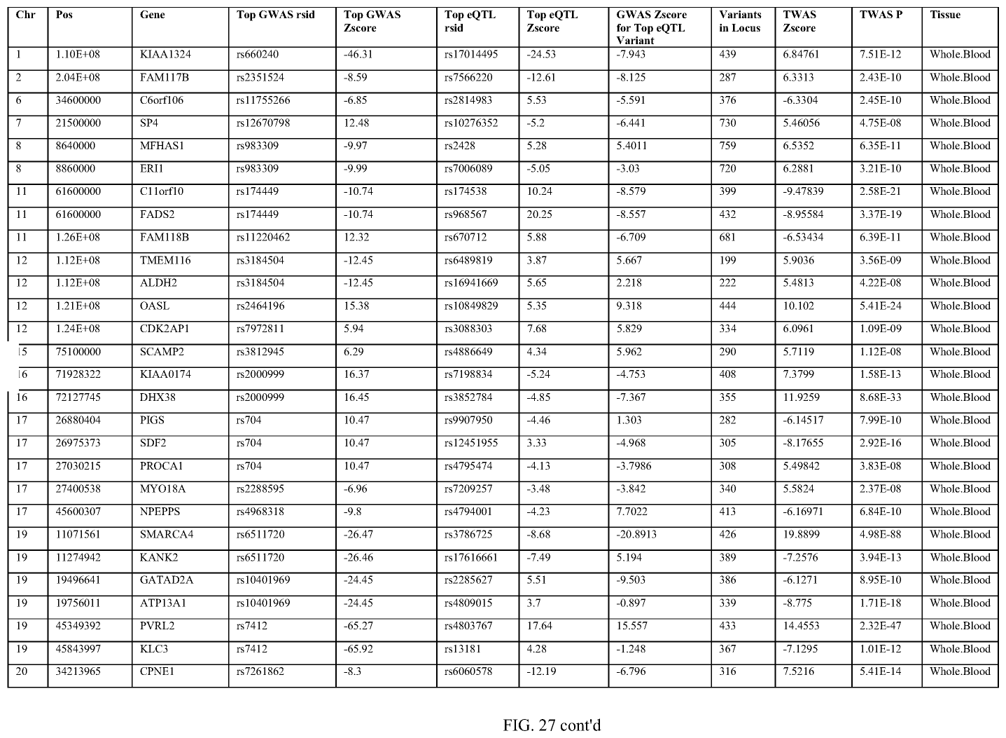

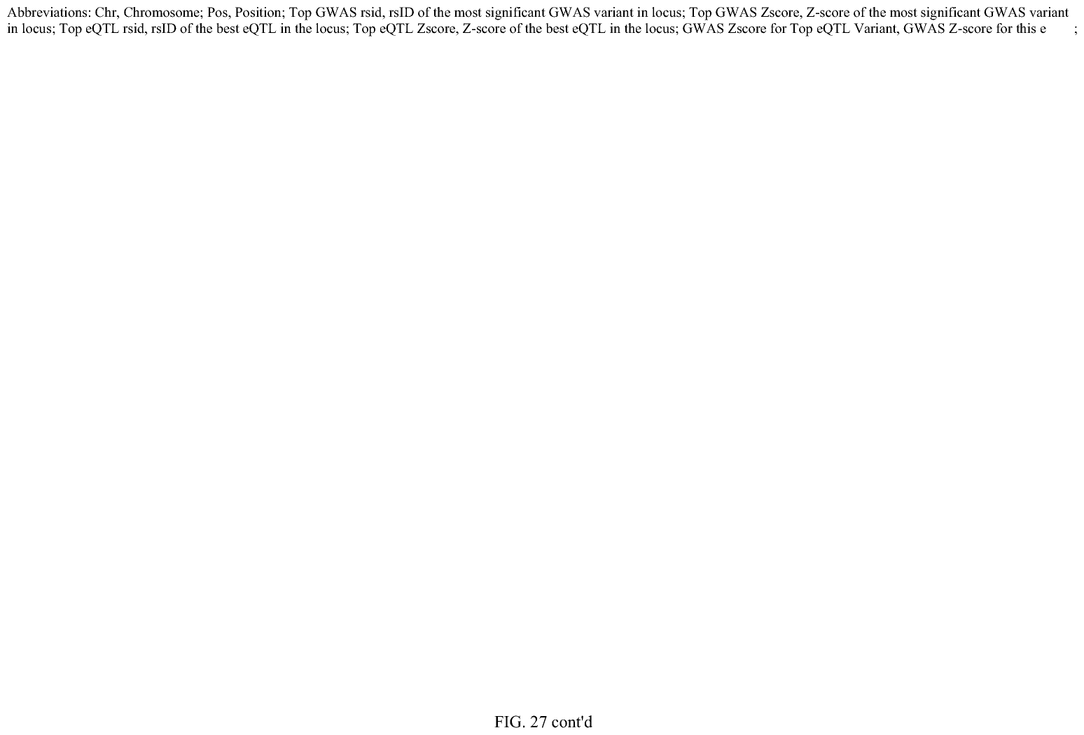

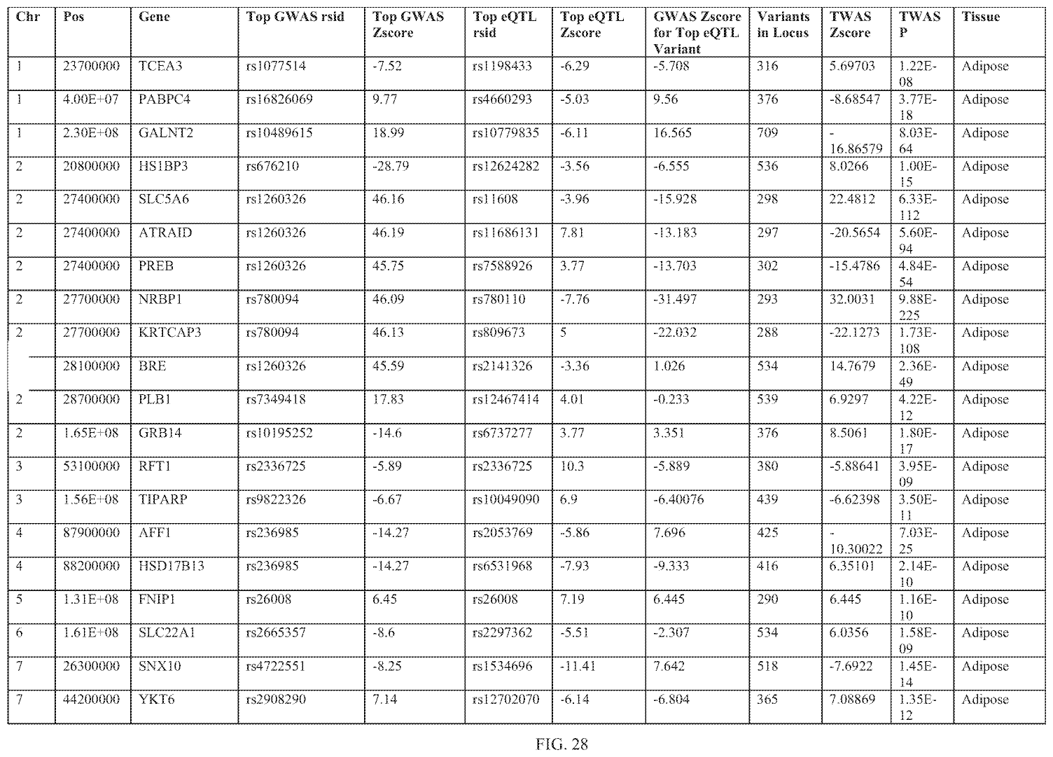

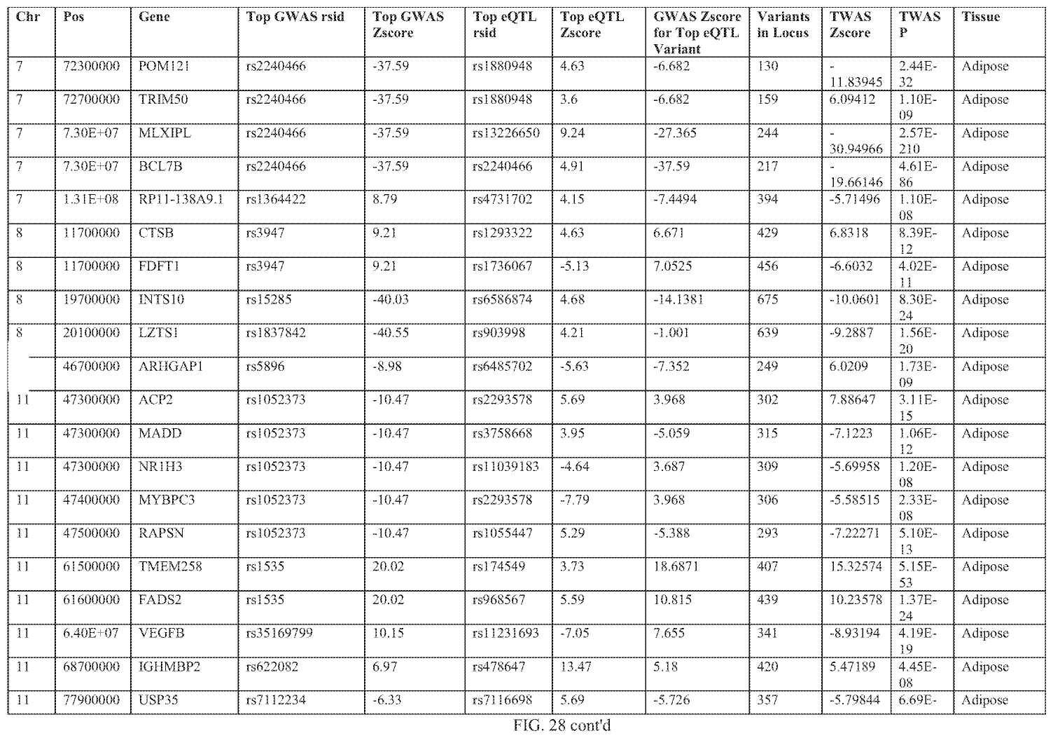

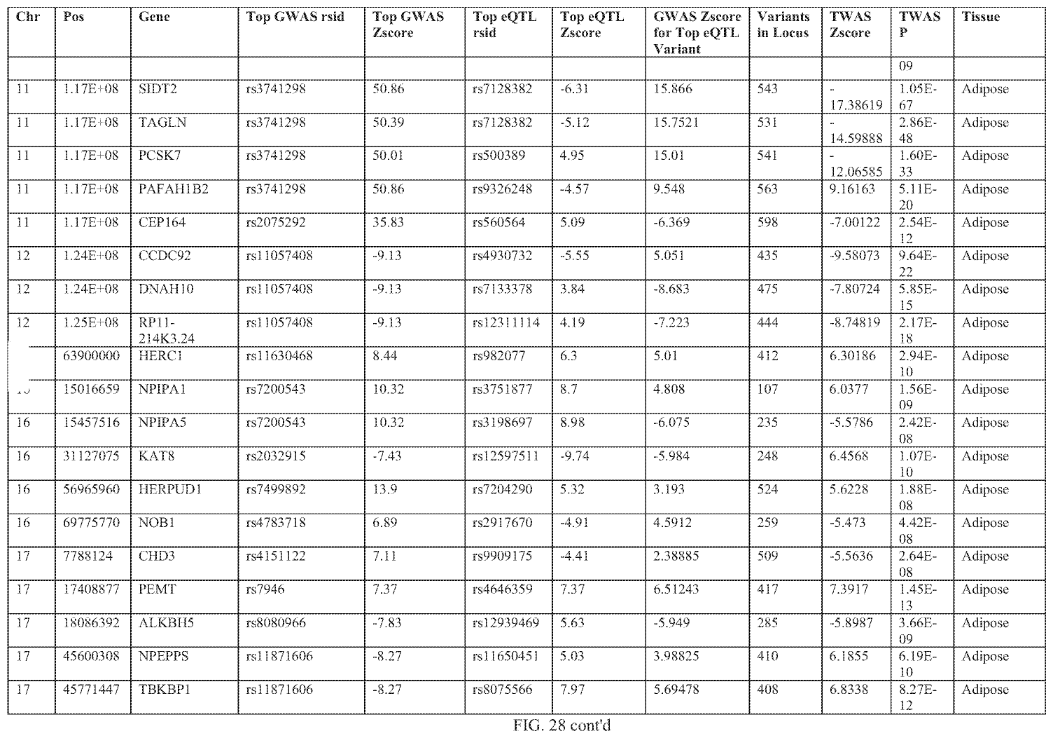

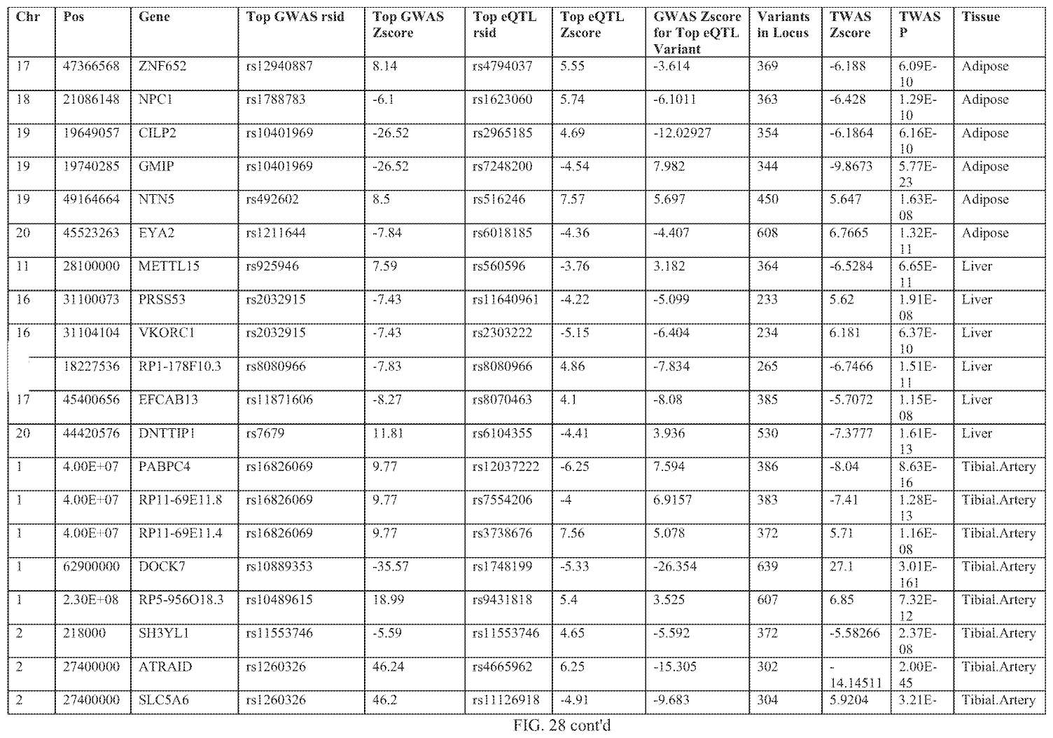

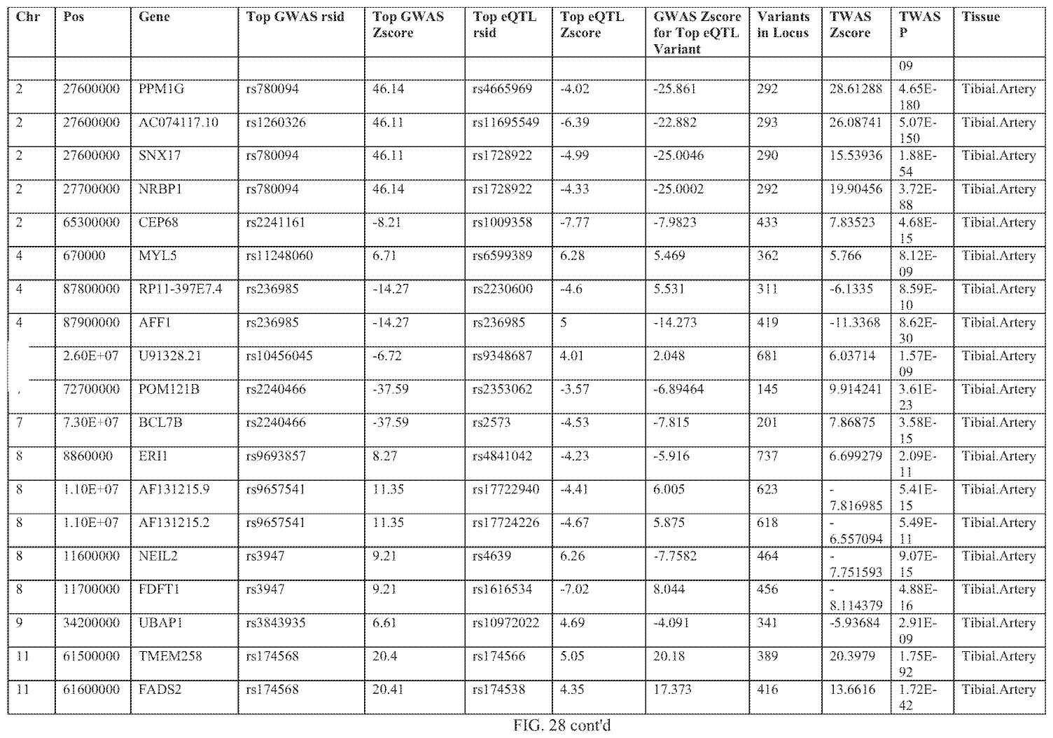

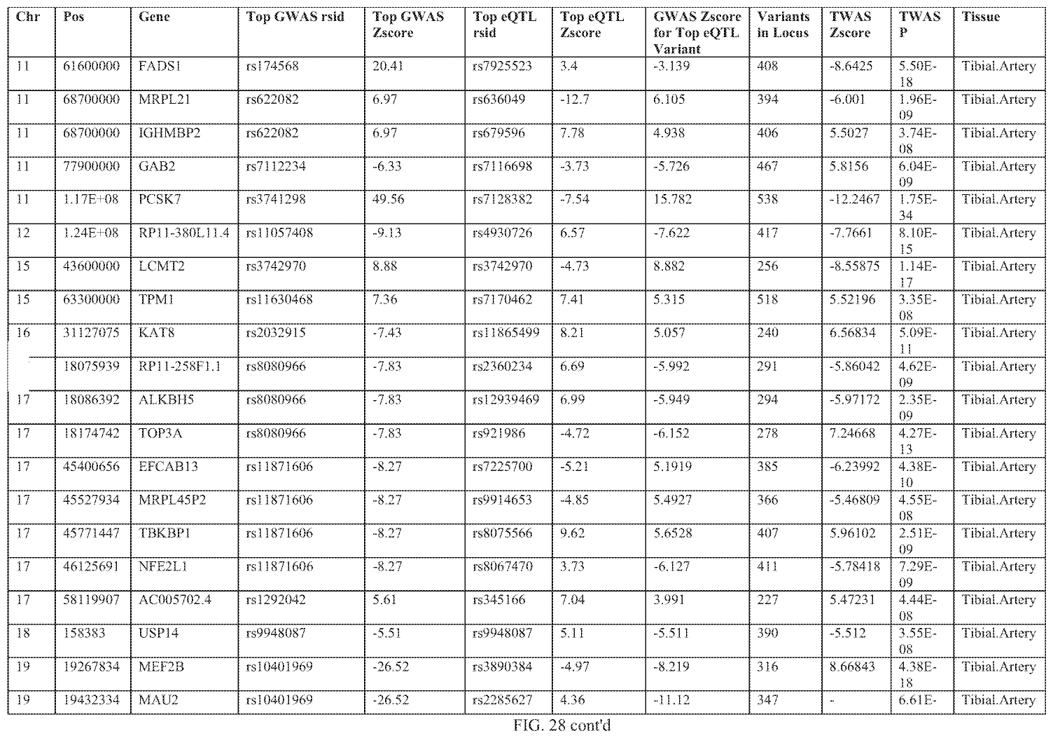

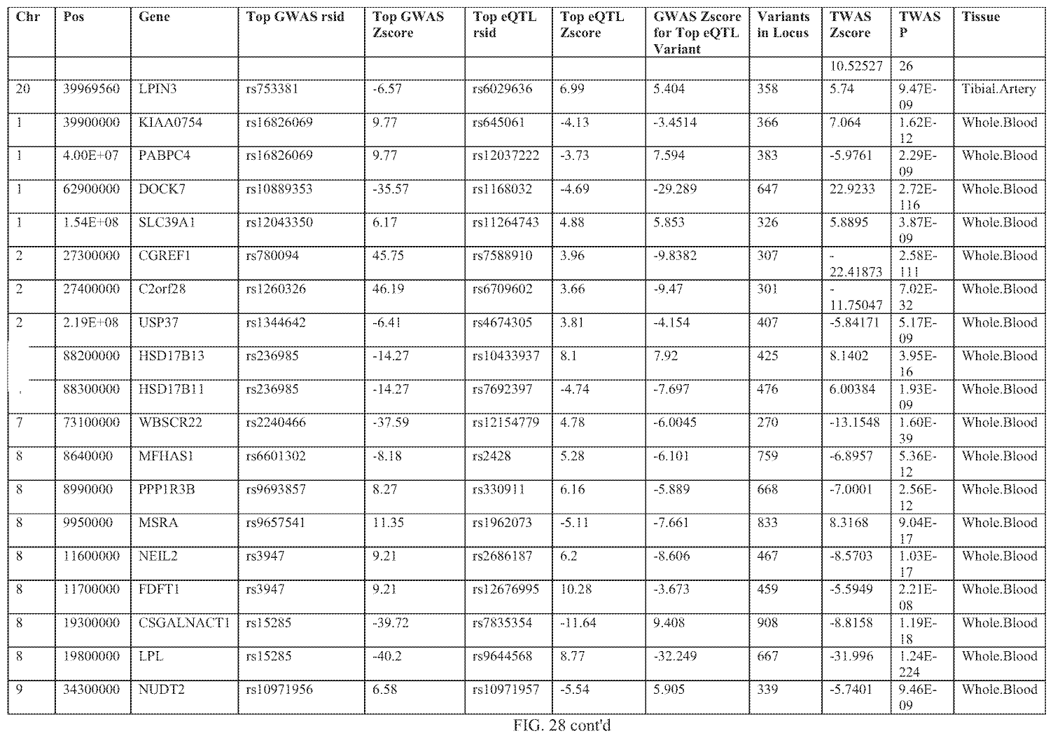

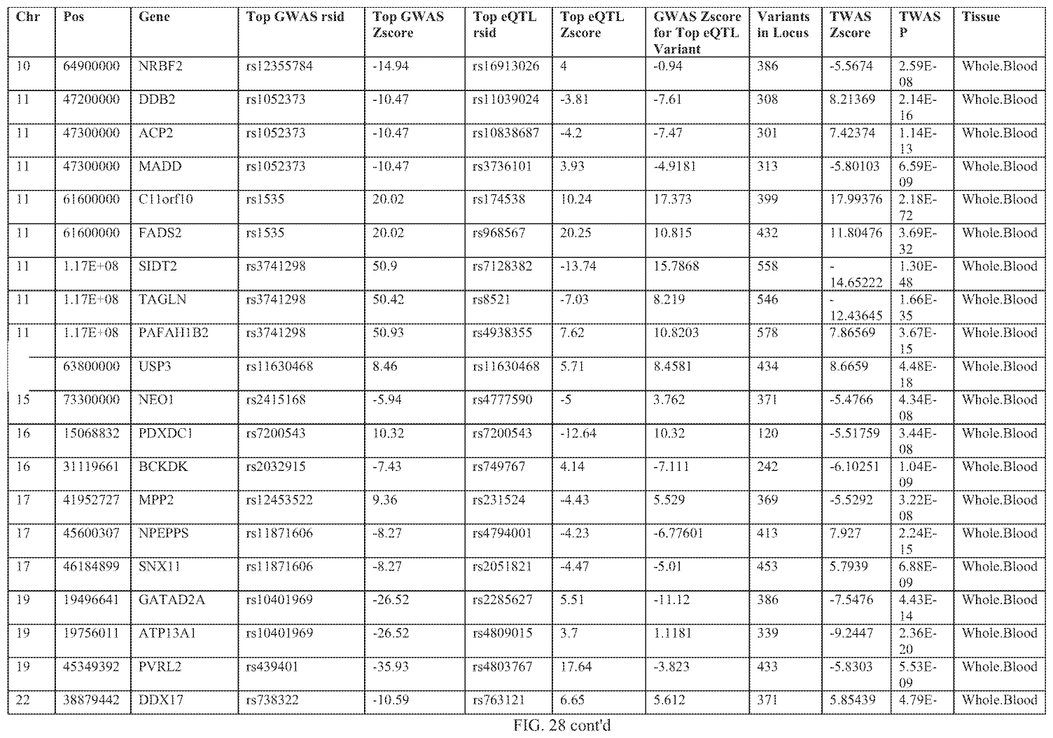

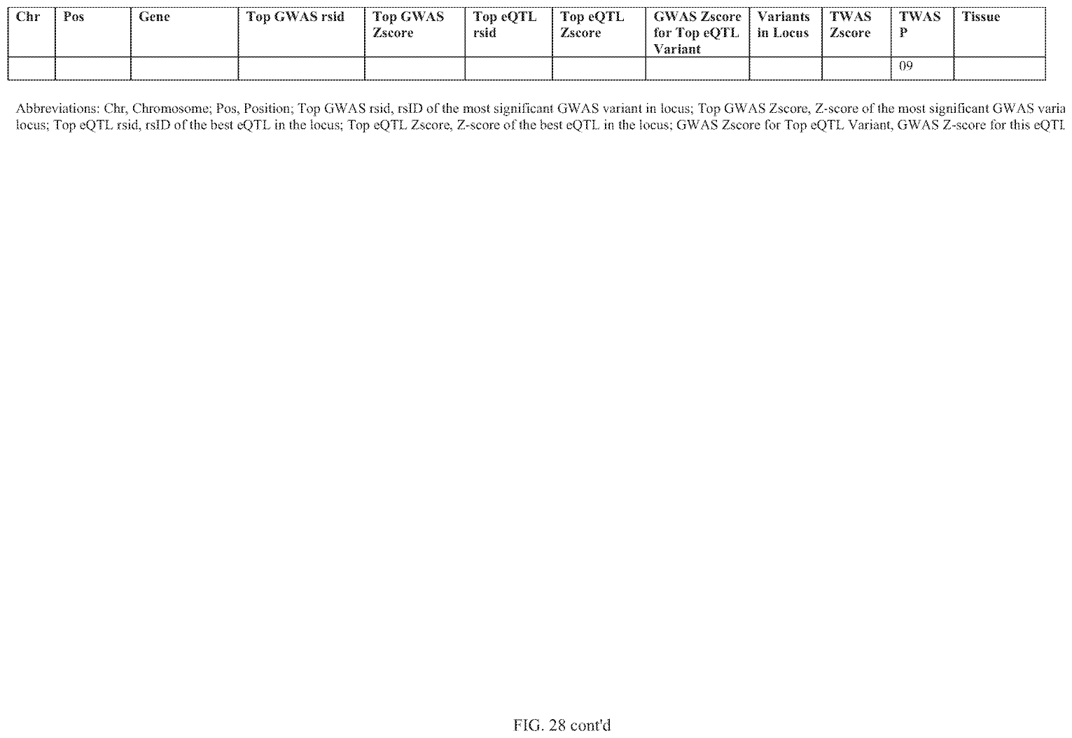

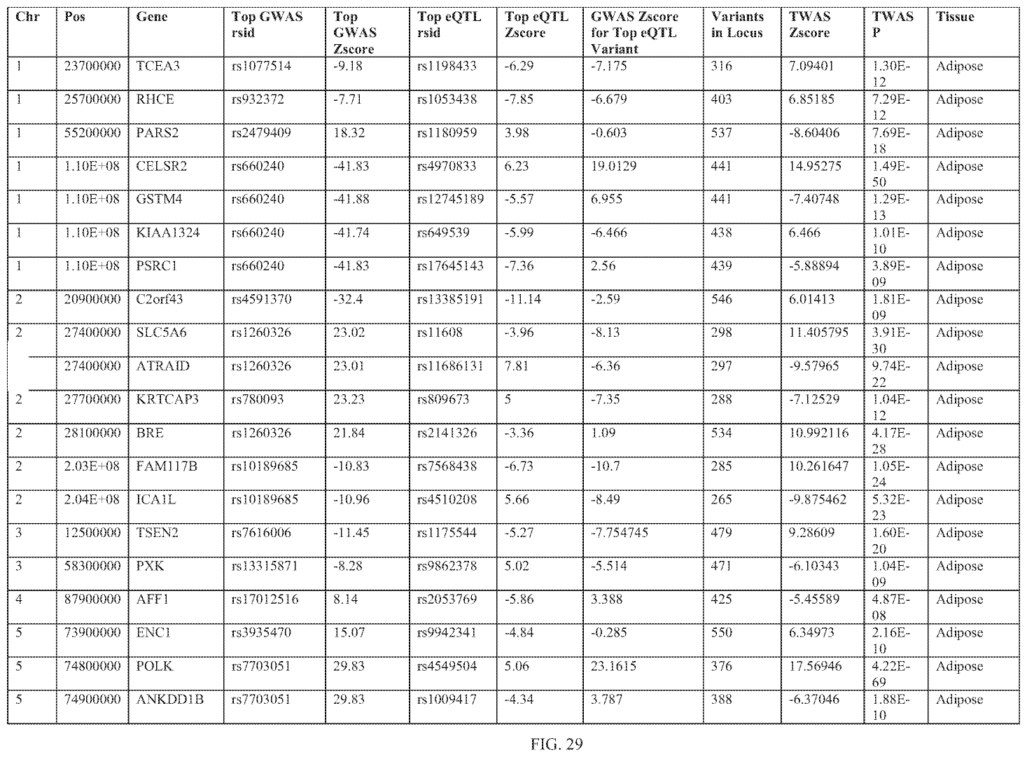

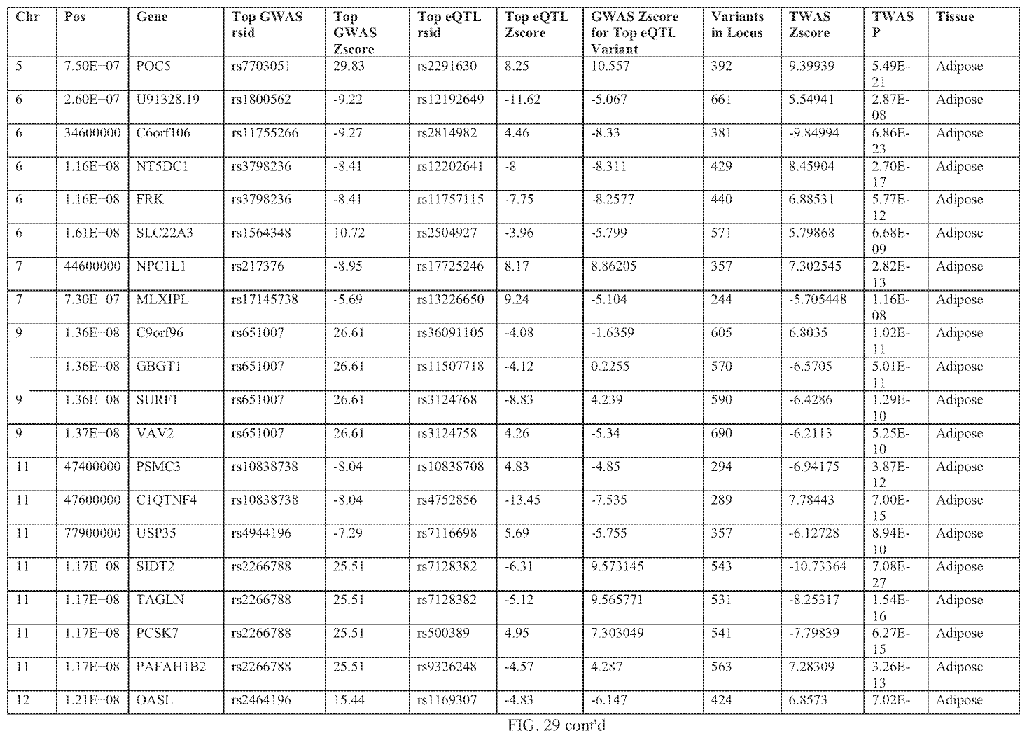

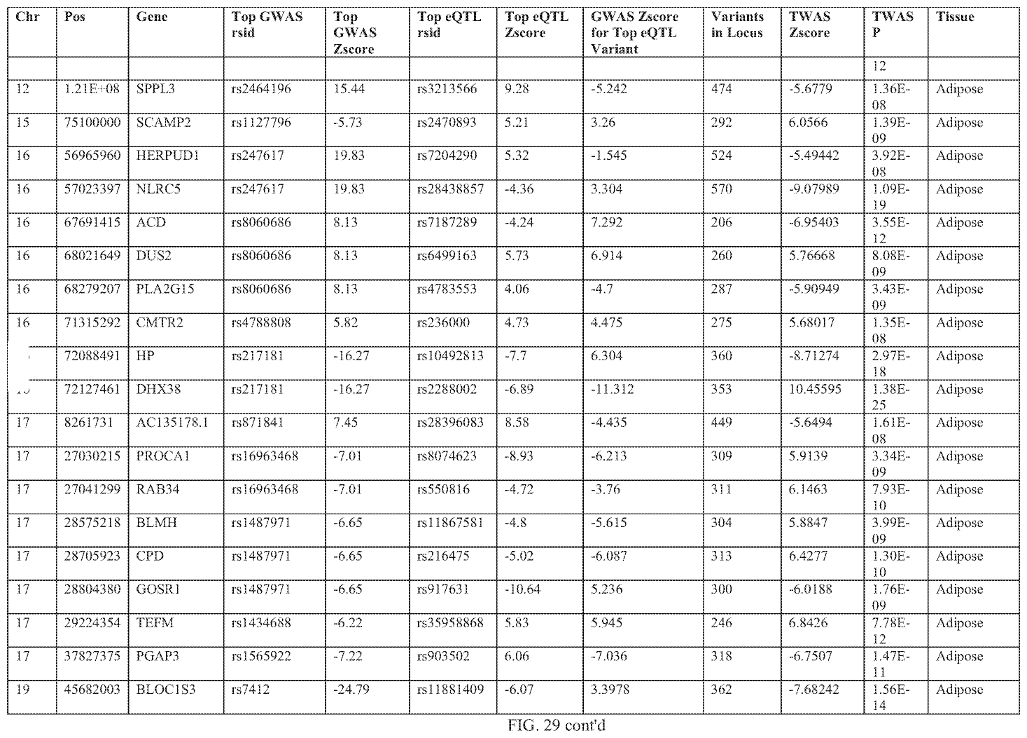

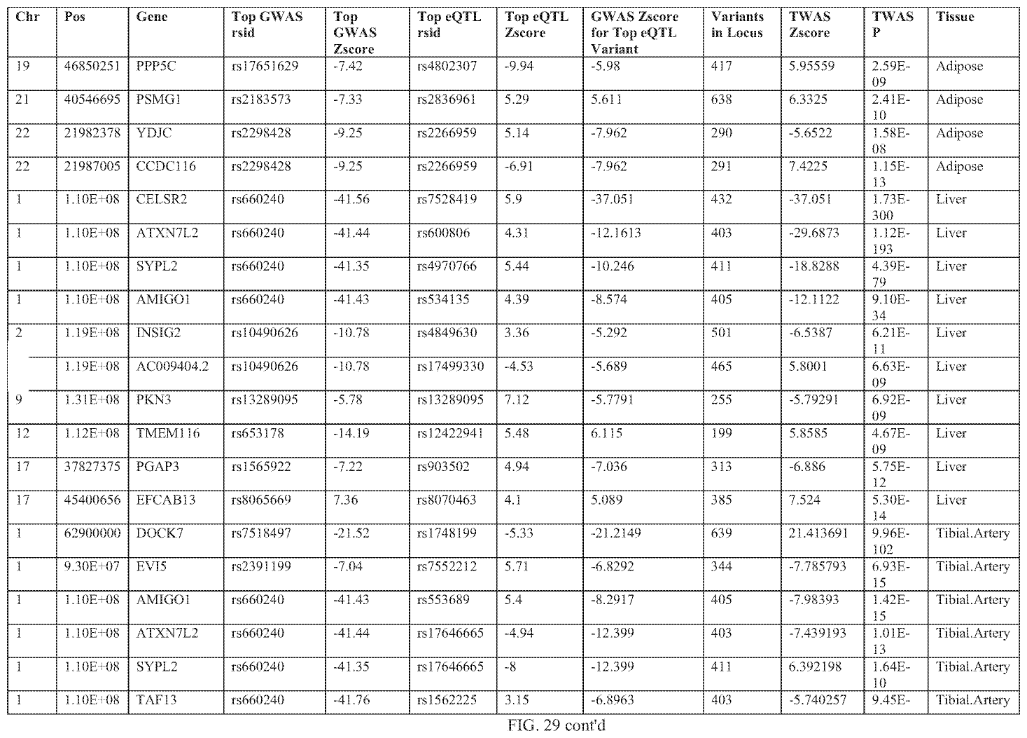

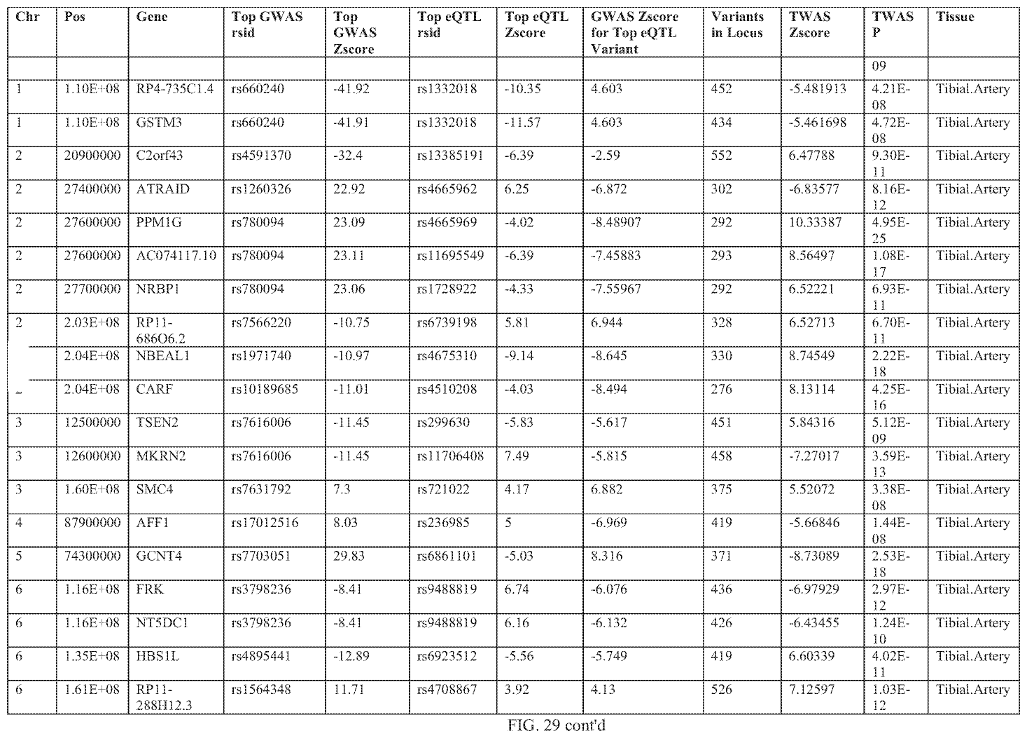

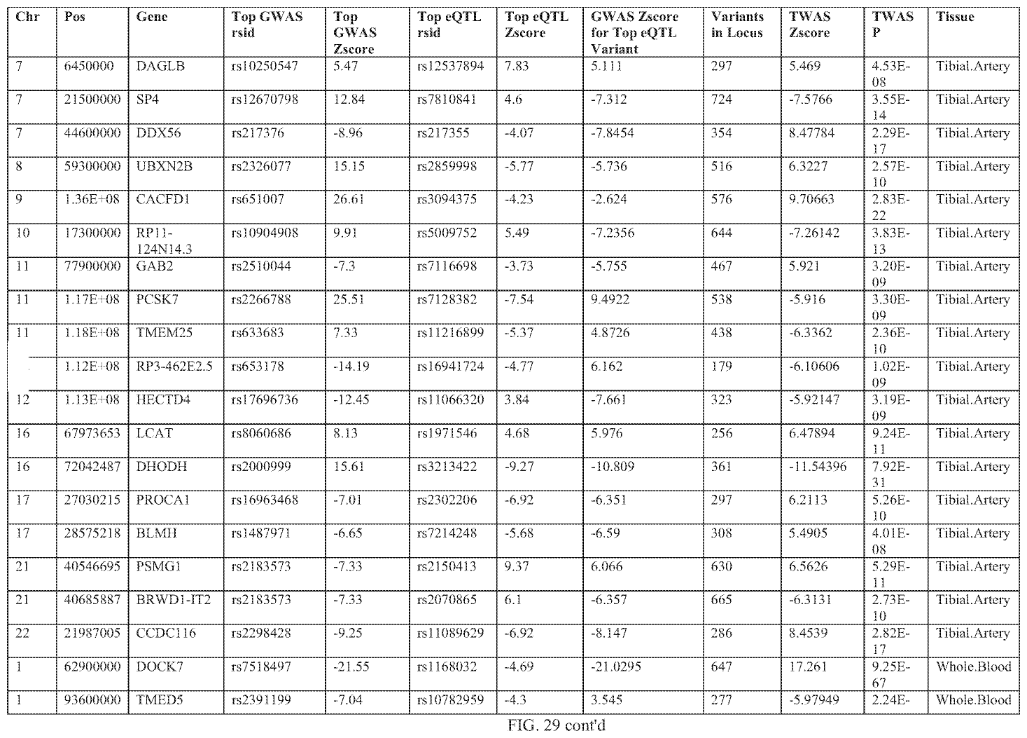

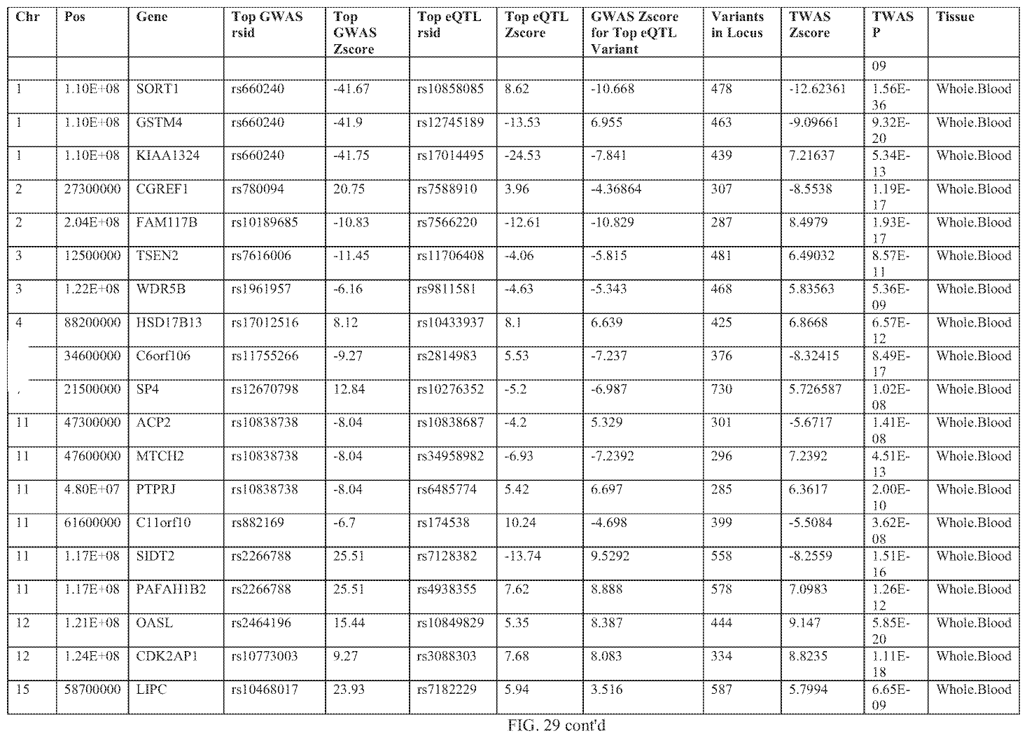

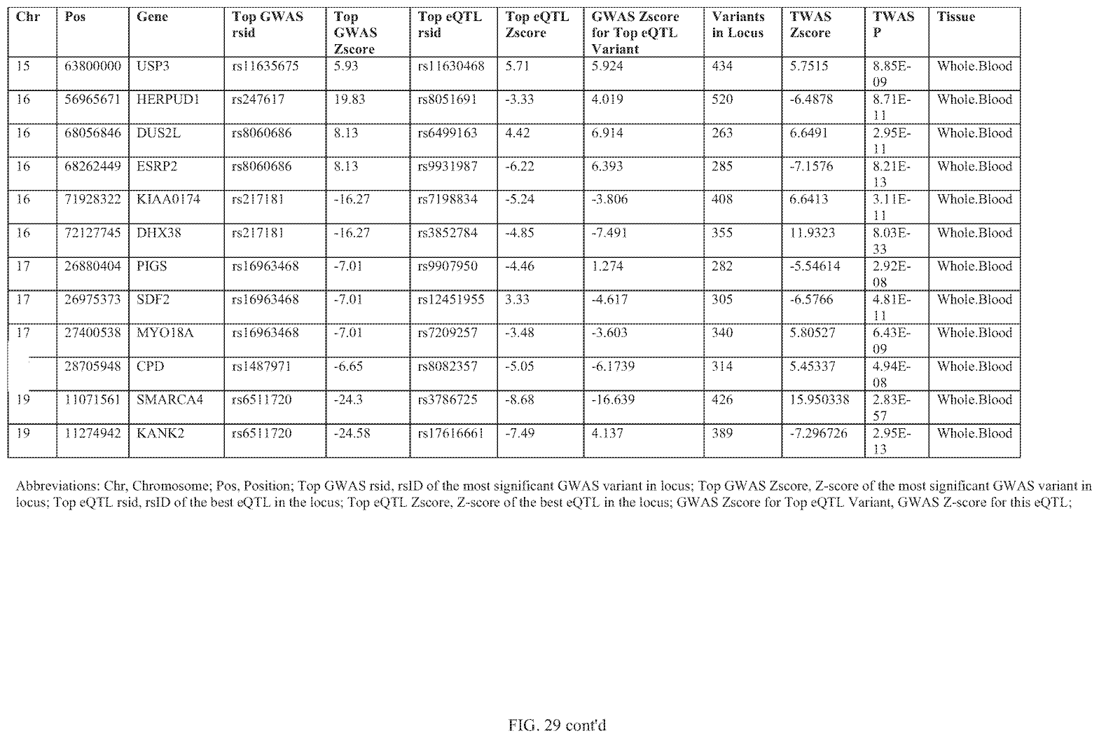

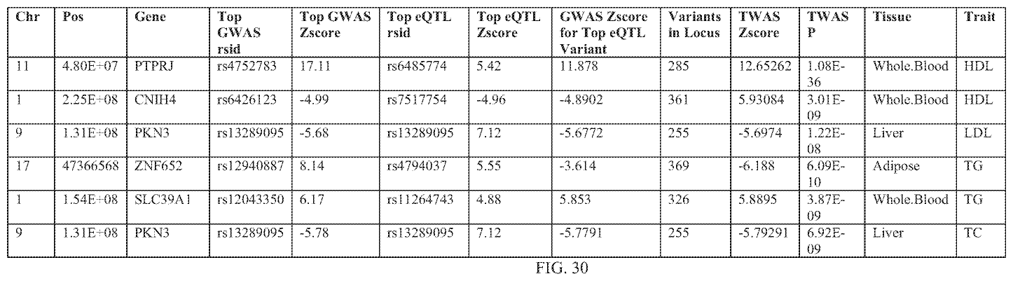

In total, the TWAS identified 655 genome-wide significant (P<5×10-8) gene-lipid associations (summed across expression reference panels) in a total of 333 distinct genes, including 194 that were significant in more than one tissue or lipid trait (FIGS. 12-13 and 26-29). The 333 distinct genes fell within 122 genomic loci, 117 of which were within a lipid GWAS region (±1 mB around a mapped sentinel GWAS variant) identified in a prior analysis or in the current study. However, 5 TWAS genes fell outside of a previously mapped GWAS region, representing novel lipid genomic loci (FIG. 30). Previous work has suggested that future lipid GWAS with larger sample sizes will likely confirm the novel lipid loci identified by TWAS.

iv. Tissue Expression Enrichment and Competitive Gene Set Pathway Analysis

Multi-marker Analysis of GenoMic Annotation (MAGMA) was used as implemented in the FUMA pipeline to perform a competitive gene set analysis of curated gene sets and GO terms (pathways) obtained from the Molecular Signature Database, as well as a gene-property analysis for gene expression of GTex²⁵ tissues for LDL-C, TG, and HDL. As expected, the pathway analysis revealed a significant enrichment for several biological processes related to lipoprotein metabolism including sterol homeostasis, acylglycerol homeostasis, chylomicron mediated transport,acyl reverse cholesterol transport, and regulation of lipoprotein lipase activity (P Bonferroni <0.05). MAGMA gene-property analysis revealed a significant enrichment of GWAS signal overlapping genes expressed in the liver, adrenal gland, and the ovary for LDL-C, subcutaneous and visceral adipose tissue, liver, adrenal gland, and pancreas for TG, and liver for HDL-C.

v. Predicted Loss of Gene Function Lipid Associations

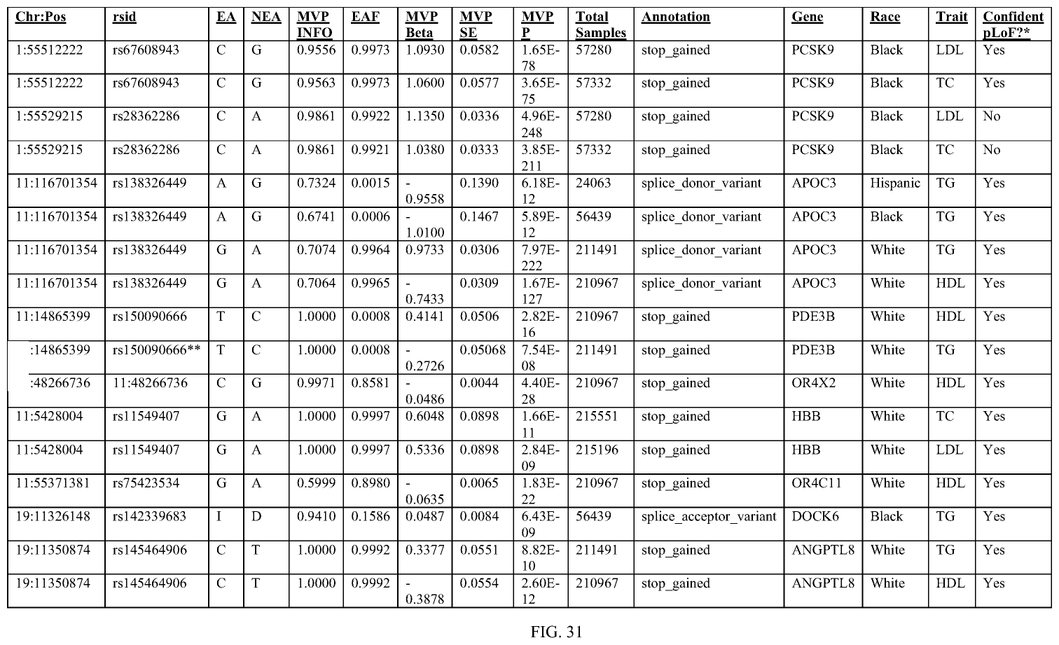

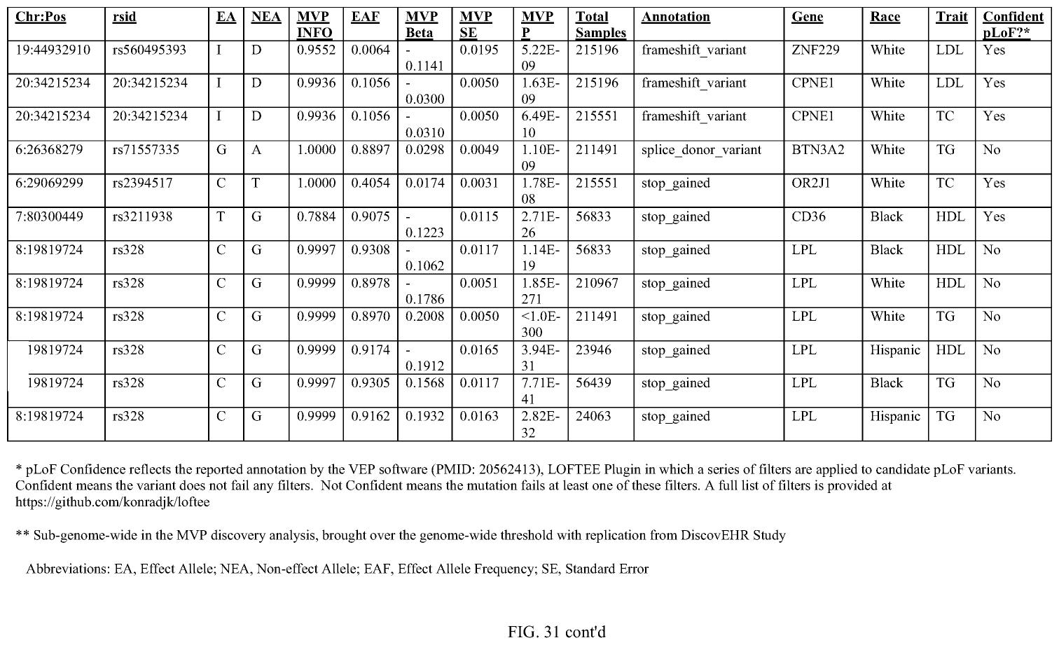

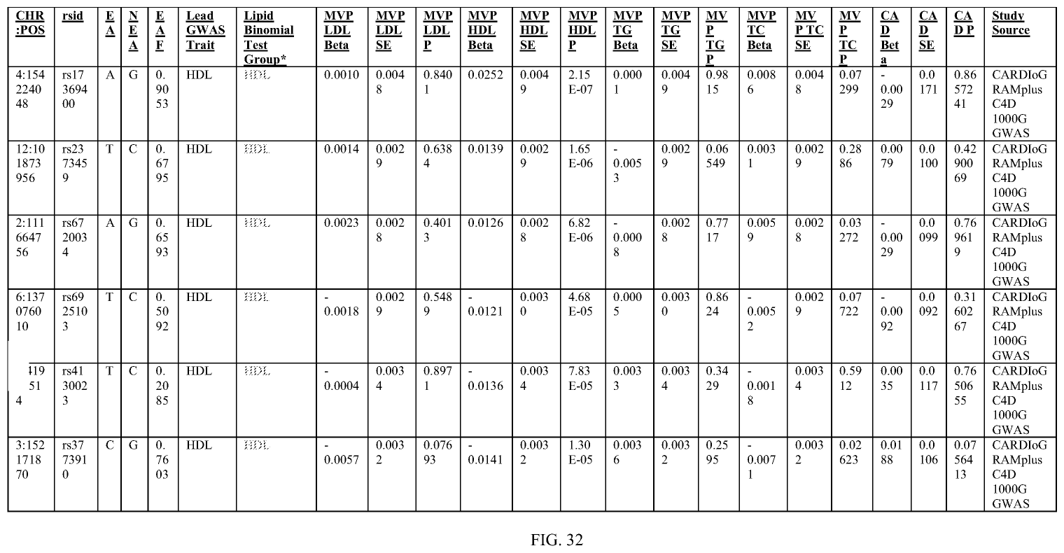

The subset of genotyped or imputed pLoF variants [variants annotated as: premature stop (nonsense), canonical splice-sites (splice-donor or splice-acceptor) or insertion/deletion variants that shifted frame (frameshift) by the Variant Effect Predictor software] was then studied. A total of 15 unique pLoF variants demonstrated genome-wide significant lipid associations across individuals of all three ethnic groups (FIG. 31). Known pLoF associations were replicated at PCSK9, APOC3, ANGPTL8, LPL, CD36, and HBB and genome-wide significant associations of comparable magnitude of effect in each of the three ethnic groups for 2 pLoF variants: APOC3 c.55+1G>A and LPL p. Ser747Ter were observed.

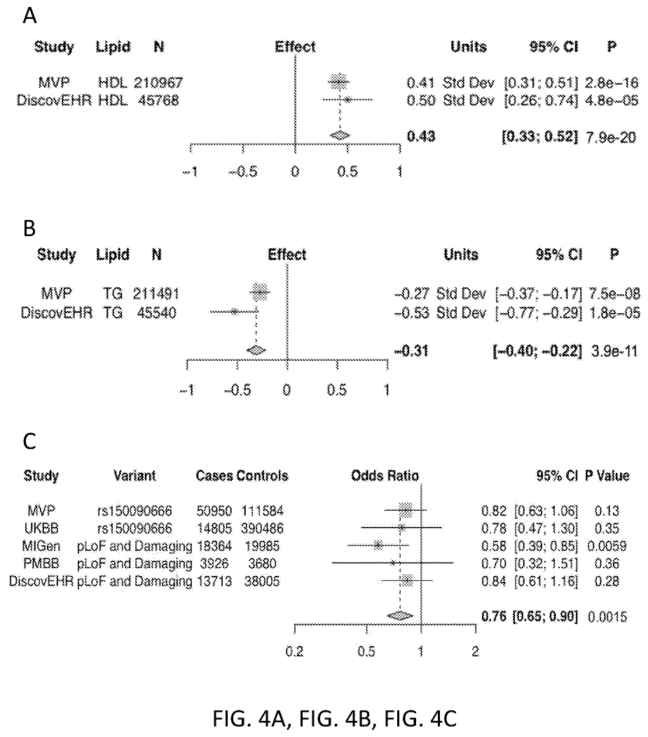

One novel pLoF association was identifed. Among white MVP participants, carriers of a rare stop-gain mutation in PDE3B (p.Arg783Ter; carrier frequency of 1 in 625), exhibited a 4.72 mg/dL (0.41 standard deviations) higher blood HDL cholesterol (P<2.8×10⁻¹⁶) and 43.3 mg/dL (−0.27 standard deviations) lower blood TG (P=7.5×10⁻⁸). This signal is independent of the previously reported PDE3B genome-wide significant lead variant, rs103737811 (p.Arg783Ter conditional analysis P=8.91×10⁻⁸ for TG and 6.3×10⁻¹⁶ for HDL cholesterol, respectively). One individual was also identified who was homozygous for p.Arg783Ter. This PDE3B “human knockout” was in his sixth decade of life HDL cholesterol and TG levels of 73 and 56 mg/dL, respectively. He was not on lipid-lowering medication and was free of coronary artery disease (CAD). The TG and HDL associations were replicated for this pLoF variant in an independent sample of ˜45,000 participants of the DiscovEHR study (FIG. 4a,b).

vi. Loss of PDE3B Function and Risk of Coronary Artery Disease

Mutations damaging or causing a loss of function in PDE3B can protect against the development of CAD based on their association with lifelong lower TG levels in blood. A case-control analysis of CAD status was performed involving 5 cohorts: MVP, UK Biobank, Myocardial Infarction Genetics Consortium (MIGen), Penn Medicine Biobank (PMBB), and DiscovEHR. In studies with exome sequencing available (MIGen, PMBB, DiscovEHR), pLoF variants were combined with missense variants predicted to be damaging or possibly damaging by each of 5 computer prediction algorithms (LRT score, MutationTaster, PolyPhen-2, HumDiv, PolyPhen-2 HumVar, and SIFT) as performed previously. Because any damaging mutations were individually rare, they were aggregated in subsequent association analysis with CAD (Table 4). Among 103,580 individuals with CAD and 566,813 controls available for meta-analysis in these 5 cohorts, carriers of damaging PDE3B mutations were found to have a 24% decreased risk of CAD (OR=0.76, 95% CI=0.65-0.90, P=0.0015, FIG. 4c).

TABLE 4
Exemplar list of 47 rare damaging mutations in PDE3B from Myocardial Infarction
Genetics Consortium exome sequencing data used for CAD analysis. DiscovEHR
analysis was performed with 44 damaging mutations in PDE3B. Penn Medicine
Biobank analysis was performed with 34 damaging mutations in PDE3B

| | | | |
|---|---|---|---|
| Variant | | | Protein Change |
| (Chr:Pos REF/ALT) | rsid | Consequence | or Splice Site |

Loss Of Function Variants (n = 9)

| | | | |
|---|---|---|---|
| 11:14666481_C/CTG | rs772636547 | Frameshift | p.Ile289Ter |
| 11:14666497_G/GAGGA | . | Frameshift | p.Arg294LysfsTer47 |
| 11:14810650_A/G | . | Splice Acceptor | c.1279-2A > G |
| 11:14839865_AT/A | rs775466201 | Frameshift | p.Ser554LeufsTer31 |
| 11:14840680_A/T | rs757322376 | Splice Acceptor | c.1734-2A > T |
| 11:14840732_CA/C | rs750097841 | Frameshift | p.Asp596IlefsTer46 |
| 11:14854367_C/T | rs775044623 | Stop Gained | p.Arg732Ter |
| 11:14865399_C/T | rs150090666 | Stop Gained | p.Arg783Ter |
| 11:14880600_C/A | . | Stop Gained | p.Tyr844Ter |

Predicted Damaging Missense Variants (n = 38)

| | | | |
|---|---|---|---|
| 11:14853280_T/A | rs768823210 | missense | p.Leu684His |
| 11:14853312_A/G | rs762702362 | missense | p.Ile695Val |
| 11:14854287_A/G | rs746323697 | missense | p.Gln705Arg |
| 11:14854344_T/G | rs551949989 | missense | p.Phe724Cys |
| 11:14854373_A/C | . | missense | p.Ile734Leu |
| 11:14854377_C/T | rs760056319 | missense | p.Pro735Leu |
| 11:14856536_C/T | rs771878367 | missense | p.Arg739Cys |
| 11:14856537_G/A | . | missense | p.Arg739His |
| 11:14856551_G/C | rs760668695 | missense | p.Asp744His |
| 11:14856584_C/T | . | missense | p.Arg755Trp |
| 11:14865418_C/T | . | missense | p.Ser789Leu |
| 11:14865460_C/T | rs746865798 | missense | p.Ser803Phe |
| 11:14865498_G/A | rs769373319 | missense | p.Val816Met |
| 11:14865514_A/G | rs374190636 | missense | p.His821Arg |
| 11:14865529_C/G | rs767804586 | missense | p.Pro826Arg |
| 11:14865543_G/A | . | missense | p.Ala831Thr |
| 11:14865553_T/C | rs750628998 | missense | p.Val834Ala |
| 11:14865556_C/G | . | missense | p.Ala835Gly |
| 11:14880599_A/G | . | missense | p.Tyr844Cys |
| 11:14880623_A/G | . | missense | p.Asn852Ser |
| 11:14880627_T/A | rs200861692 | missense | p.His853Gln |
| 11:14880640_G/C | rs781891436 | missense | p.Ala858Pro |
| 11:14880667_G/C | rs376052497 | missense | p.Glu867Gln |
| 11:14880692_A/G | . | missense | p.Asp875Gly |
| 11:14880729_T/G | rs111436102 | missense | p.Ile887Met |
| 11:14880746_C/T | rs201854538 | missense | p.Thr893Met |
| 11:14880775_G/A | rs199971236 | missense | p.Ala903Thr |
| 11:14880785_A/G | rs781883242 | missense | p.Asn906Ser |
| 11:14882808_G/A | rs548256441 | missense | p.Val928Ile |
| 11:14882823_A/T | rs376523505 | missense | p.Ile933Phe |
| 11:14882877_T/C | rs139772242 | missense | p.Trp951Arg |
| 11:14882905_A/G | rs781795919 | missense | p.Tyr960Cys |
| 11:14889100_C/G | . | missense | p.Arg979Gly |
| 11:14889101_G/A | rs782472054 | missense | p.Arg979His |
| 11:14889118_G/C | . | missense | p.Ala985Pro |
| 11:14889173_A/C | rs202088348 | missense | p.Tyr1003Ser |
| 11:14889173_A/G | rs202088348 | missense | p.Tyr1003Cys |
| 11:14891076_A/T | . | missense | p.Lys1070Met |

vii. Novel Lipid Loci and Association with Coronary Disease

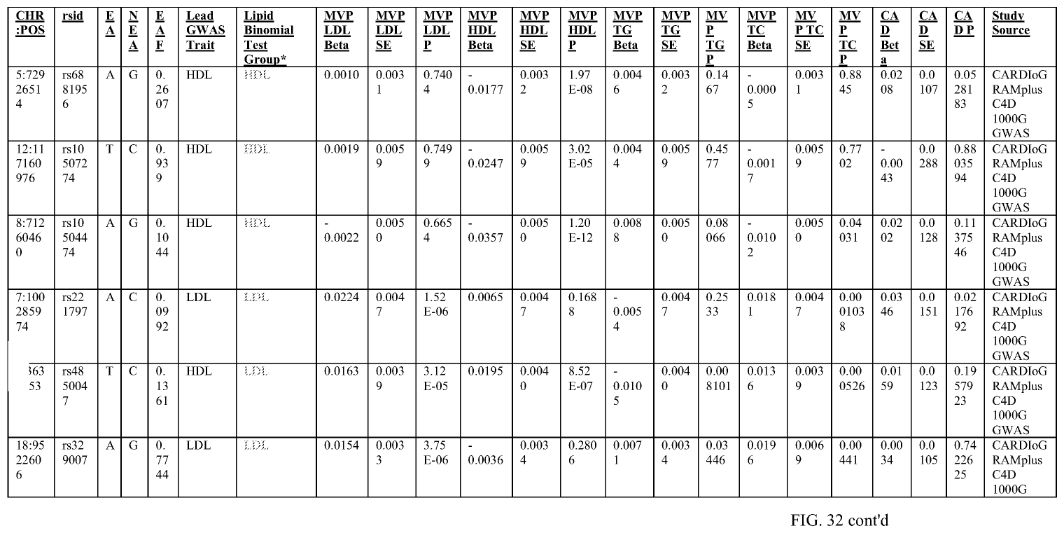

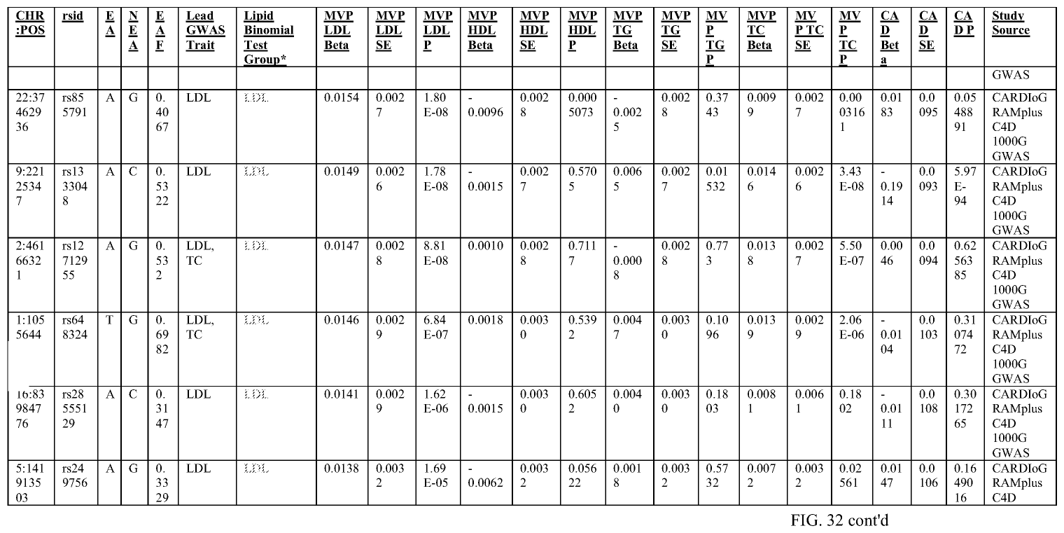

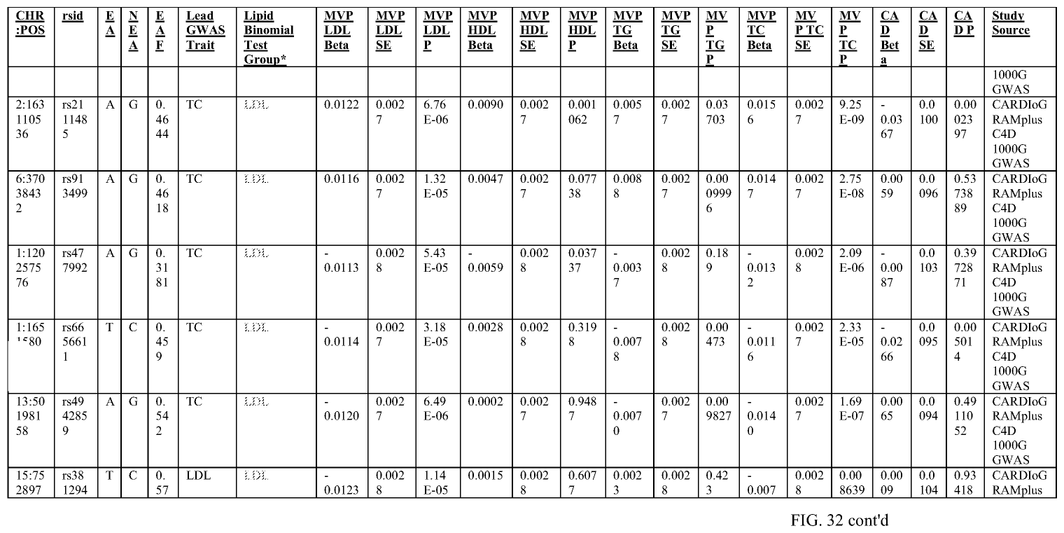

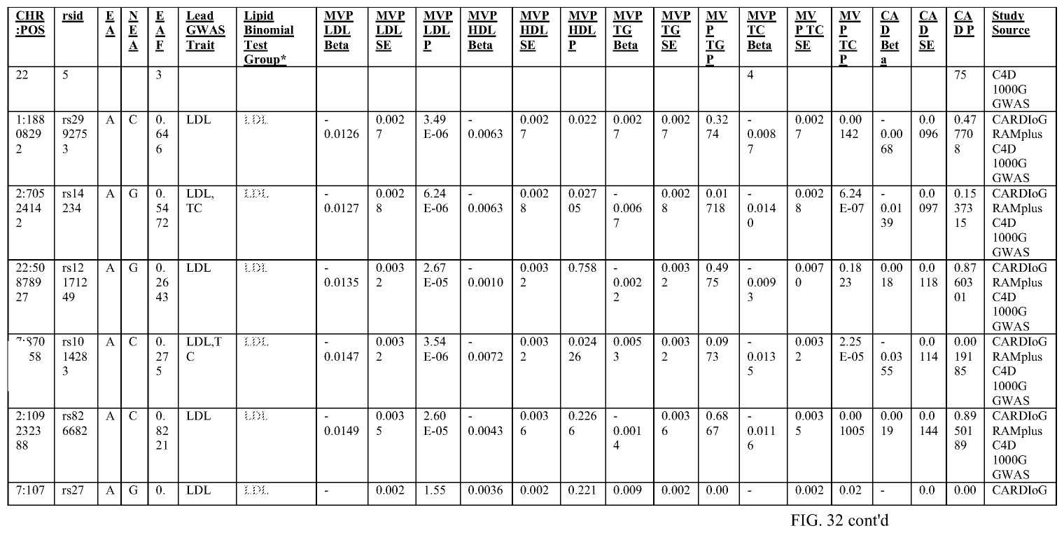

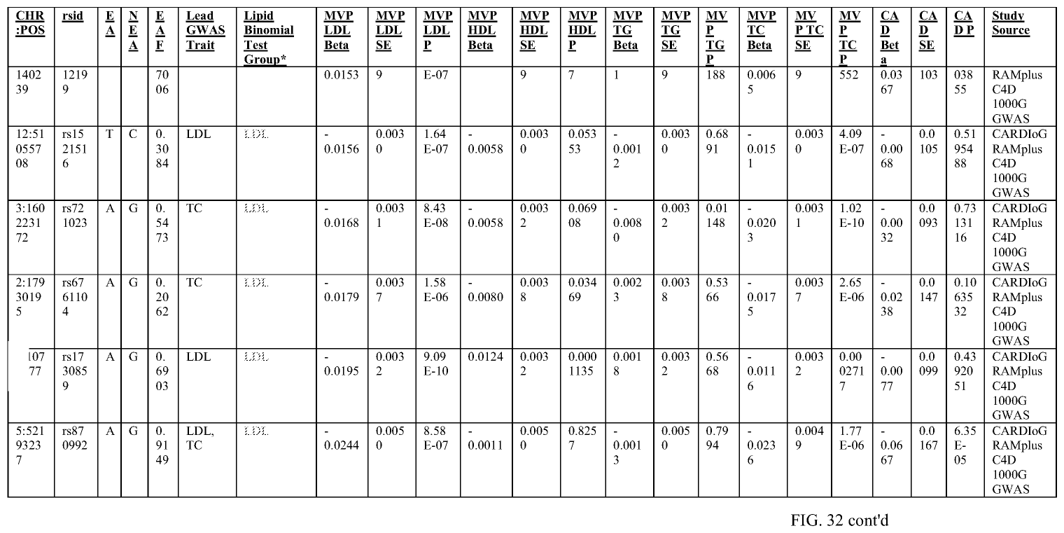

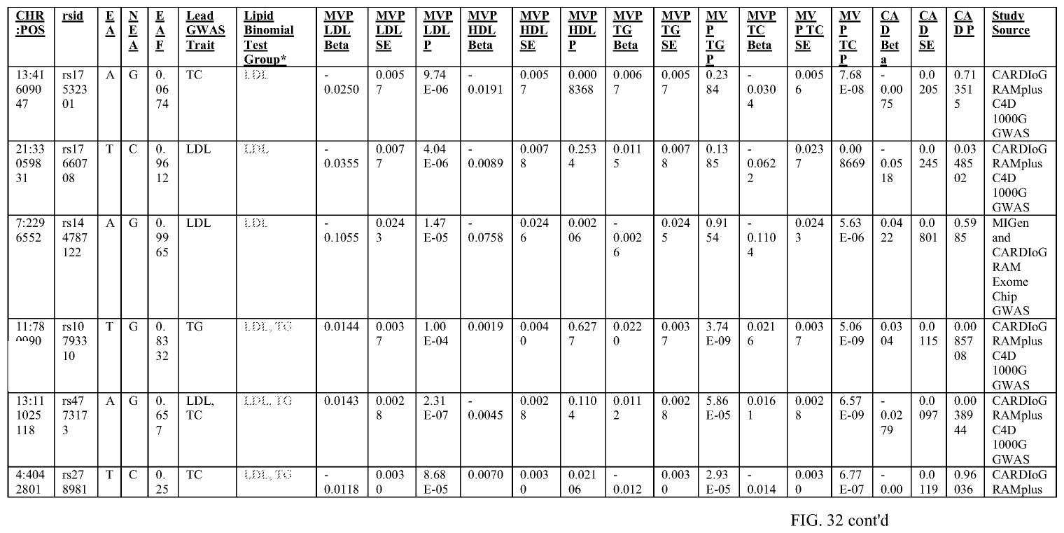

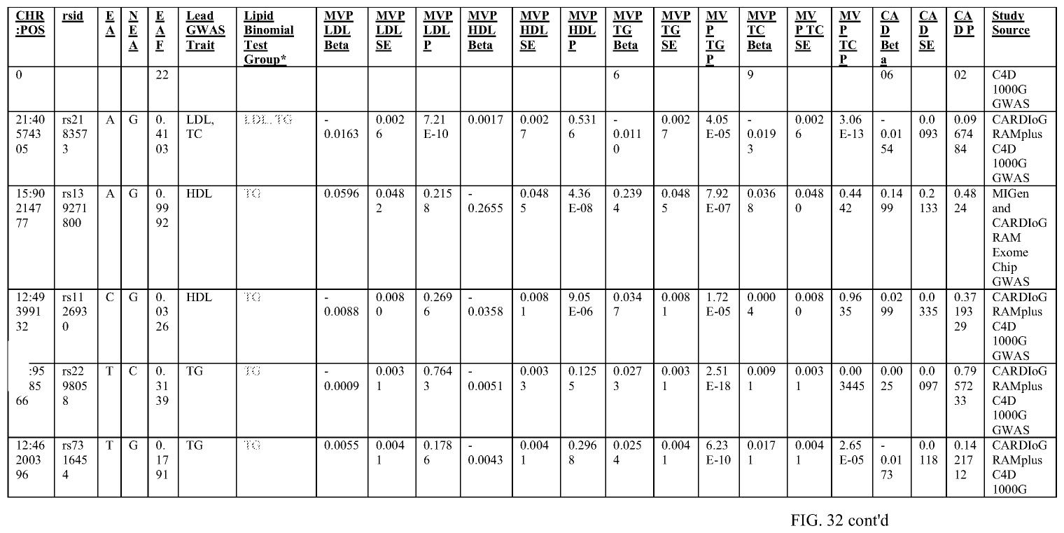

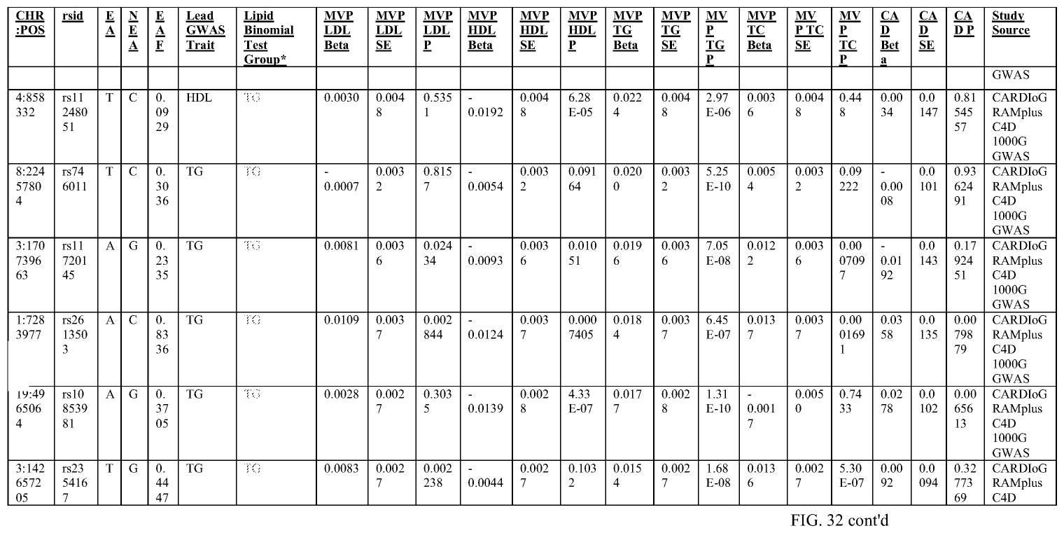

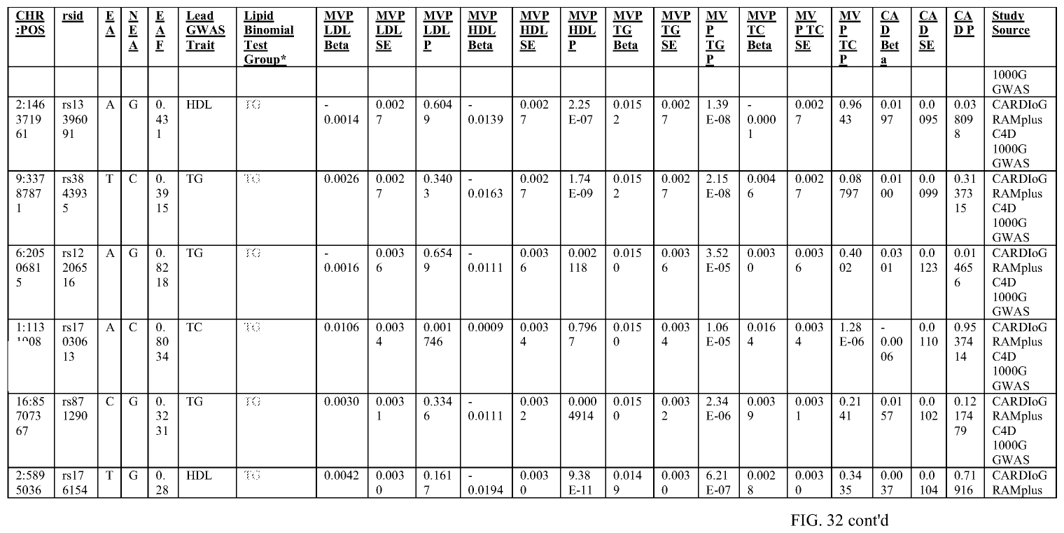

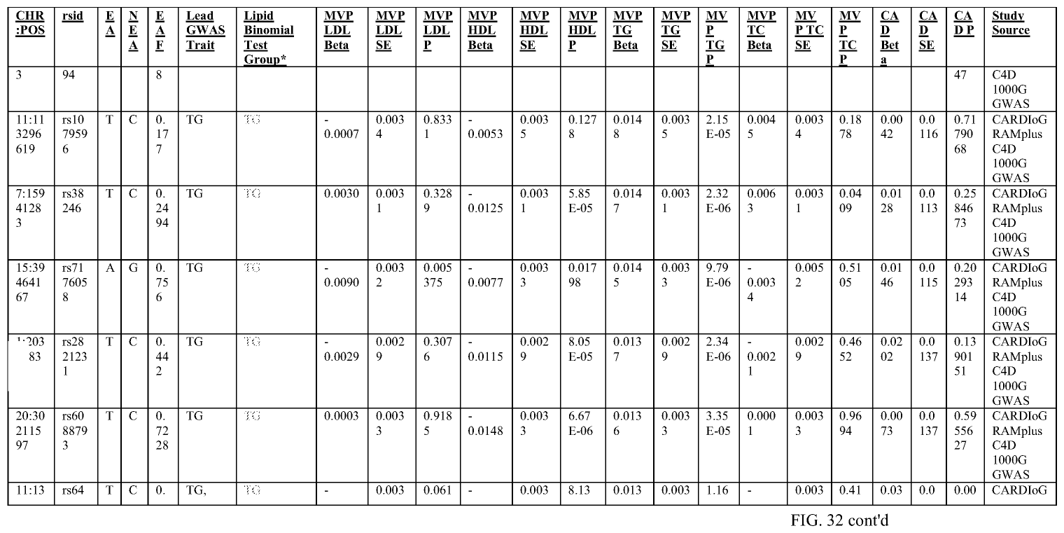

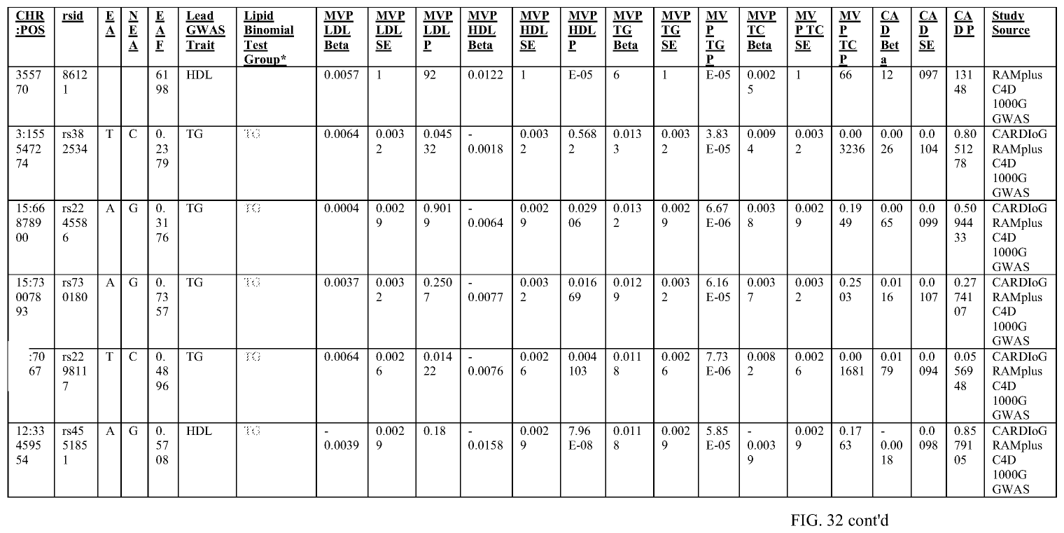

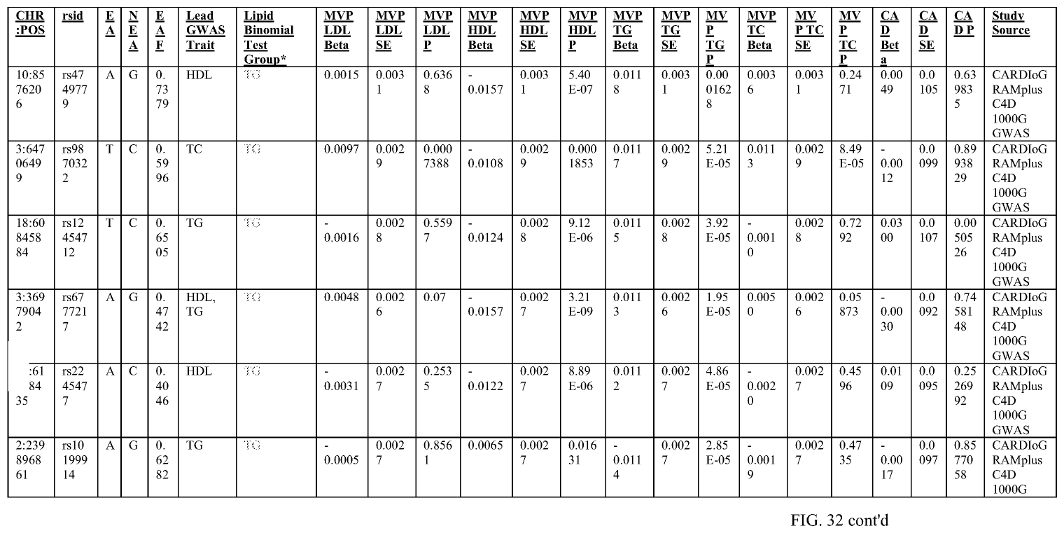

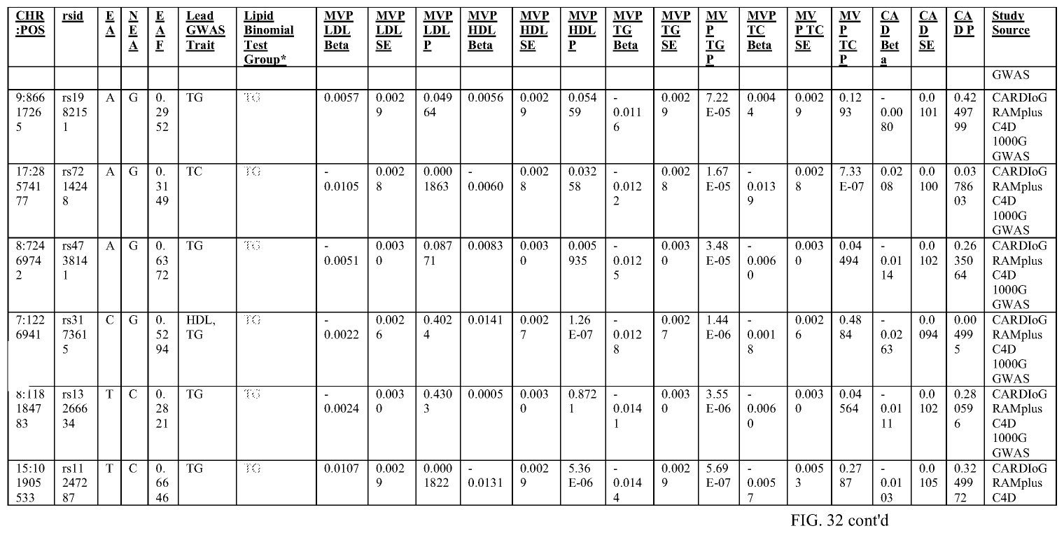

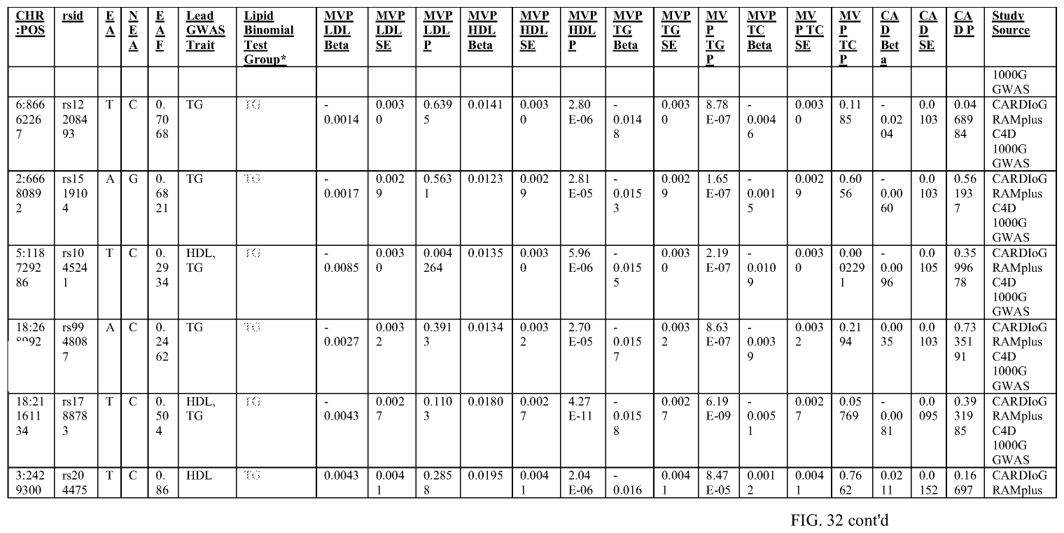

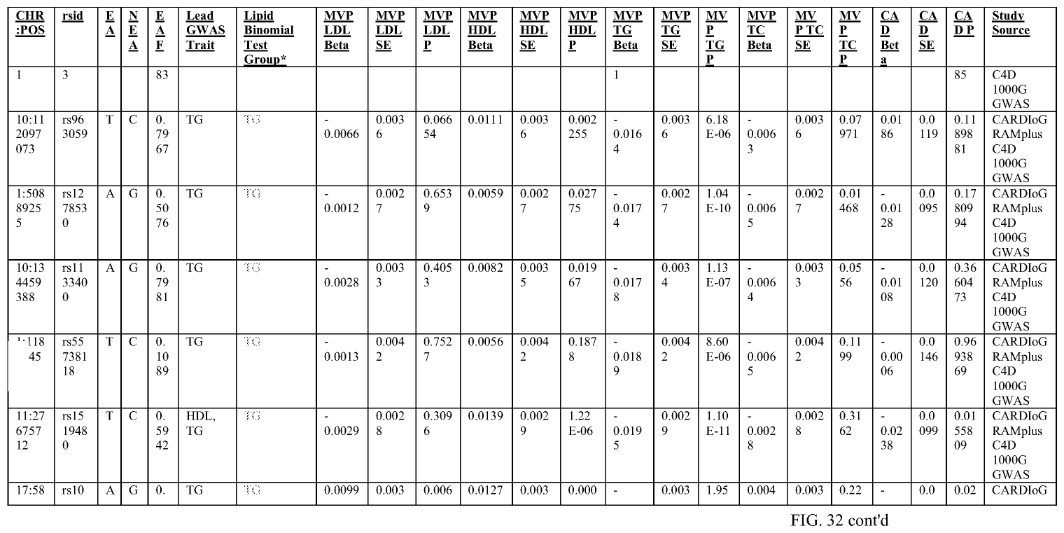

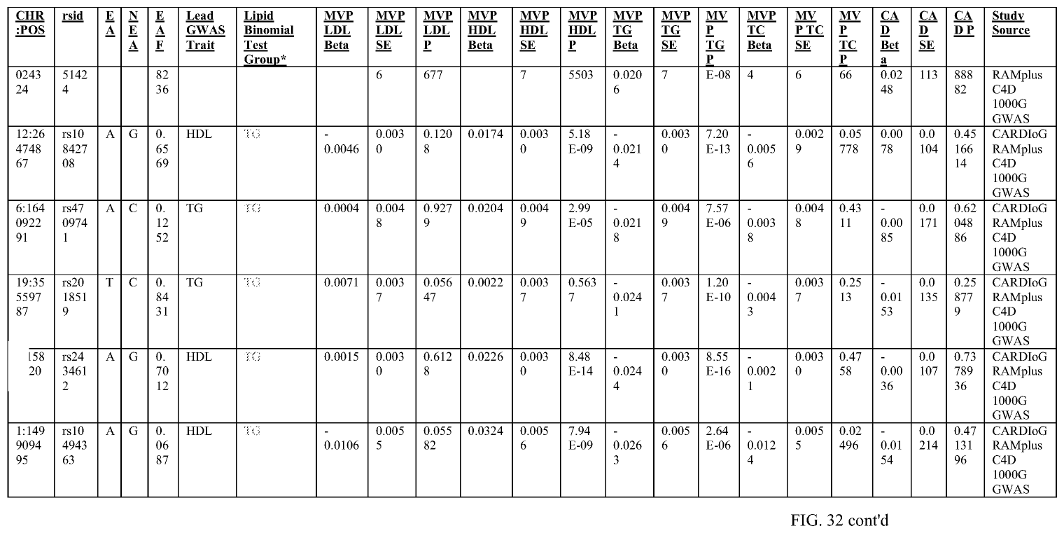

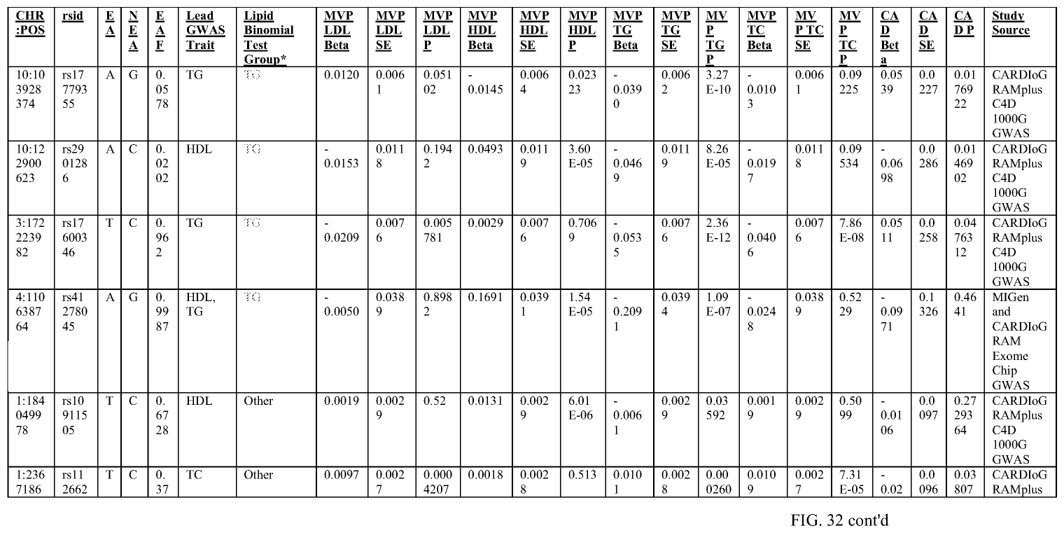

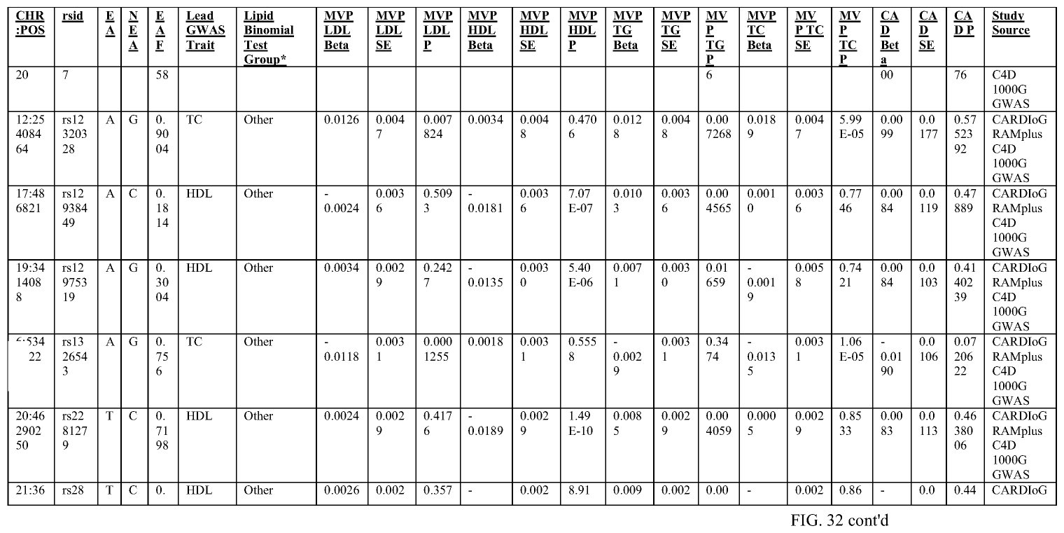

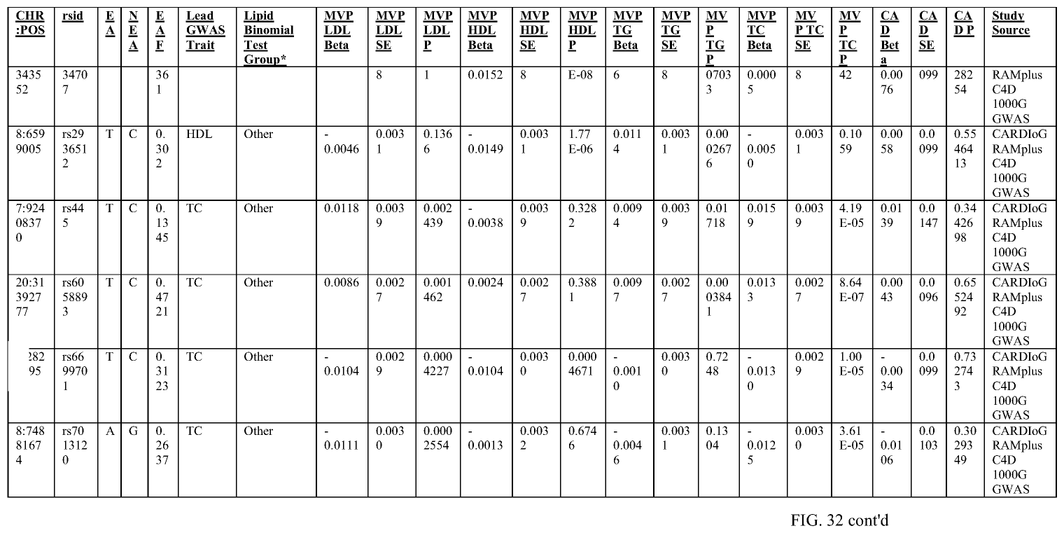

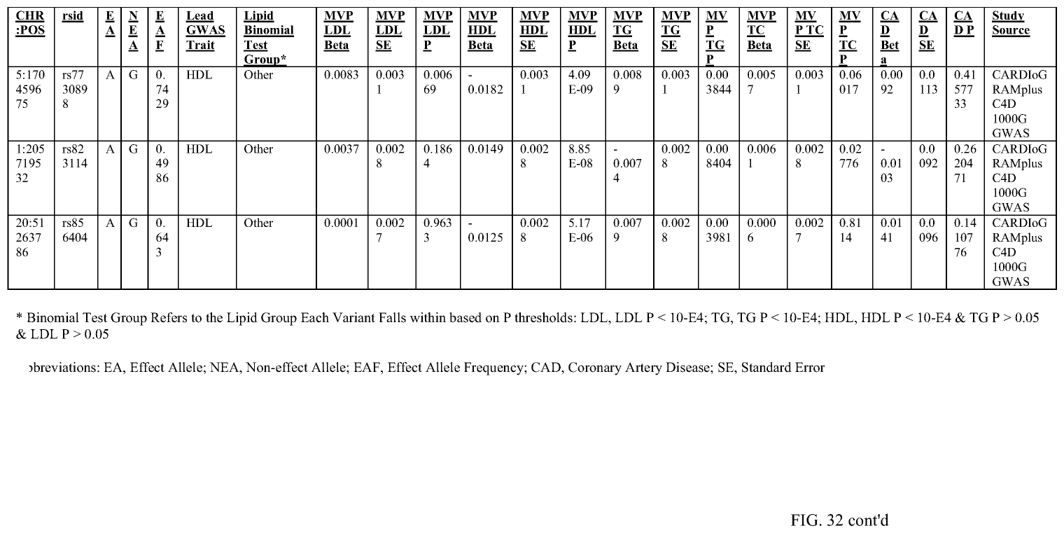

To further evaluate whether novel lipid variants identified in the analysis also influence the risk of CAD, the association of lead variants was examined within the 118 novel lipid loci identified in the study with CAD. 115/118 of the lead variants were present in the CARDIoGRAMplusC4D 1000 Genomes GWAS; the remaining 3 (MAF<0.0035 for each) were present the MIGen and CARDIoGRAM exome chip GWAS analysis. In total, 25 of the 118 loci showed at least nominal (P<0.05) association with CAD in the CARDIoGRAM studies (FIG. 32). Notably, the previously identified lead CAD 9p21 locus (rs1333048, CAD P=5.7×10⁻⁹⁴) is also associated with LDL-C and TC at genome-wide significance. However, the LDL-C raising allele is in the opposite direction of the CAD effect estimate, indicating that the causal variant(s) at 9p21 can confer CAD risk outside of a lipid pathway as implied by preliminary functional work at the locus. The direction of effect for LDL-C, TG, and HDL-C raising alleles on CAD for the 118 novel loci was then examined. Consistent with prior observations, the 32 LDL-C and 63 TG raising alleles (lipid P<10⁻⁴) were more likely to be associated with an increased risk of CAD (two-tailed binomial P=0.05 and 3.8×10⁻⁵ for LDL and TG, respectively). The same was not true for 9 alleles associated with a higher HDL-C(P<10⁻⁴) but not also associated with LDL-C or TG (two-tailed binomial P=0.25).

### 2. Discussion

Data was leveraged from the Million Veteran Program to investigate the inherited basis of blood lipids using EHR-based laboratory measures in nearly 300,000 U.S. veterans. First, 188 previously identified loci were confirmed; furthermore, an additional 118 novel genome-wide significant loci were uncovered. Next, a total of 826 independent lipid associated variants were identified increasing the phenotypic variance explained by nearly 2%. A TWAS was performed in four tissues identifying 5 additional novel lipid loci at a genome-wide level of significance, and a pathway analysis was performed highlighting lipid transport mechanisms in the GWAS results. Ancestry-specific effects of rare coding variation on lipids among white, black, and Hispanic participants were identified and 15 pLoF mutations associated with lipids at a genome-wide level of significance were identified, including a protein-truncating variant in PDE3B that lowers TG, raises HDL cholesterol, and protects against CAD. Finally, the full spectrum of phenotypic consequences for mutations in lipid genes emerging as therapeutic targets, identifying protective effects of pLoF mutations in PCSK9 for abdominal aortic aneurysm and in ANGPTL4 for type 2 diabetes were examined.

There is enormous potential of a large-scale multi-ethnic biobank built within an integrated health care system in the discovery of the genetic basis of a broad spectrum of human traits. Specifically, the VA's mature nationwide EHR was leveraged to efficiently extract existing repeated laboratory measures of lipids collected during the course of clinical care in nearly 300,000 veterans over a median of 10 years for GWAS analysis. Subsequent meta-analysis (combined N>600,000) with existing datasets increased the number of known independent genetic lipid associations to nearly 400. These results highlight an increase in variance explained with multiple lipid measurements, and multiple lipid pathways with links to human disease. For example, common variants near genes such as COL4A2 and ITGA1 identified for LDL cholesterol/TC indicate links to extracellular matrix and cell adhesion biology, two pathways recently implicated by GWAS of CAD. Carriers of a rare missense mutation in the gene encoding Perilipin-1 (PLIN1 p.Leu90Pro) possess a markedly higher plasma HDL cholesterol (0.243 standard deviations). In humans, Perilipin-1 is required for lipid droplet formation, triglyceride storage, as well as free fatty acid metabolism, and frameshift pLoF mutations Perilipin-1 have been reported to result in severe lipodystrophy. A variant downstream of BDNF (encoding Brain-Derived Neurotrophic Factor) was found to be associated with HDL cholesterol and TG levels, supporting recent evidence linking this gene with metabolic syndrome and diabetes. These findings not only improve the understanding of the genetic basis of dyslipidemia, but also provide insights into targets for the development of novel therapeutic agents.

There is a benefit of studying individuals with a diverse ethnic background. Such a design can provide valuable incremental information on the nature of previously identified human genetic associations. In MVP, nearly 60,000 black and 25,000 Hispanic veterans were examined for analysis, representing one of the largest—if not the largest—single-cohort GWAS to date for these ethnic groups for any trait. Among these individuals, we compared the effect estimates and allele frequencies of lipid-associated variants across ancestral group and identified 7 novel, low-frequency coding variants associated with lipids only in non-European populations. Conversely, a shared genetic architecture across all three racial groups for pLoF variation at the LPL and APOC3 loci was confirmed. Previous work identifying low-frequency missense and pLoF variation in lipid genes have led to the development of the next generation of pharmaceutical agents for cardiovascular disease. Expansion of these efforts to larger sample sizes and additional ancestries may help explain differences in blood lipid levels and risk of atherosclerosis among select populations.

These findings lend human genetic support to PDE3B inhibition as a therapeutic strategy for atherosclerosis. Cilostazol, an inhibitor of both the 3A and 3B isoforms of the phosphodiesterase enzyme, is known to have anti-platelet, vasodilatory, and inotropic effects via inhibition of PDE3A, and also has well documented substantial effects on TG and HDL cholesterol levels—likely through antagonism of PDE3B. A PDE3B pLoF variant recapitulates the known lipid effects of cilostazol and damaging PDE3B mutations are also associated with reduced risk of CAD. Randomized control trials to date have demonstrated cilostazol's efficacy in intermittent claudication and prevention of restenosis following percutaneous coronary intervention. The drug is also currently used off-label for the prevention of stroke recurrence through a presumed anti-platelet effect. Mice genetically deficient in Pde3b display reduced atherosclerosis as well as decreased infarct size and improved cardiac function following experimental coronary artery ligation.

In conclusion, >100 new genetic signals were identified for blood lipid levels utilizing a biobank that exploits existing EHRs of U.S. veterans.

3. Methods

The design of the Million Veteran Program (MVP) has been previously described. Briefly, individuals aged 19 to 104 years have been recruited from more than 50 VA Medical Centers nationwide since 2011. Each veteran's EHR data are being integrated into the MVP biorepository, including inpatient International Classification of Diseases (ICD-9) diagnosis codes, Current Procedural Terminology (CPT) procedure codes, clinical laboratory measurements, and reports of diagnostic imaging modalities. The MVP received ethical and study protocol approval from the VA Central Institutional Review Board (IRB) in accordance with the principles outlined in the Declaration of Helsinki.

i. Genetic Data

DNA extracted from whole blood was genotyped using a customized Affymetrix Axiom biobank array, the MVP 1.0 Genotyping Array. With 723,305 total DNA sequence variants, the array is enriched for both common and rare variants of clinical significance in different ethnic backgrounds. Veterans of three mutually exclusive ethnic groups were identified for analysis: 1) non-Hispanic whites, 2) non-Hispanic blacks, and 3) Hispanics. Quality-control procedures used to assign ancestry, remove low-quality samples and variants, and perform genotype imputation to the 1000 Genomes reference panel were performed.

ii. Variant Quality Control

Prior to imputation, variants that were poorly called (genotype missingness >5%) or that deviated from their expected allele frequency based on reference data from the 1000 Genomes Project were excluded. After pre-phasing using EAGLE v2, genotypes from the 1000 Genomes Project phase 3, version 5 reference panel were imputed into Million Veteran Program (MVP) participants via Minimac3 software. Ethnicity-specific principal component analysis was performed using the EIGENSOFT software.

Following imputation, variant level quality control was performed using the EasyQC R package (www.R-project.org), and exclusion metrics included: ancestry specific Hardy-Weinberg equilibrium P<1×10-20, posterior call probability <0.9, imputation quality/INFO <0.3, minor allele frequency (MAF)<0.0003, call rate <97.5% for common variants (MAF>1%), and call rate <99% for rare variants (MAF<1%). Variants were also excluded if they deviated >10% from their expected allele frequency based on reference data from the 1000 Genomes Project.

iii. EHR-Based Lipid Phenotypes

EHR clinical laboratory data were available for MVP participants from as early as 2003. The maximum LDL cholesterol/TG/TC, and minimum HDL cholesterol was extracted for each participant for analysis. These extreme values were selected to approximate plasma lipid concentrations in the absence of lipid lowering therapy. For each phenotype (LDL cholesterol, natural log transformed TG, HDL cholesterol, and TC), residuals were obtained after regressing on age, age2, sex, and 10 principal components of ancestry. Residuals were subsequently inverse normal transformed for association analysis. Statin therapy prescription at enrollment was defined as the presence of a statin prescription in the EHR within 90 days before or after enrollment in MVP. Statin therapy prescription at the maximum lipid measurement was defined as the presence of a statin prescription in the EHR within 90 days prior to the maximum lipid laboratory measurement used in the GWAS analysis.

iv. MVP Association Analysis

Genotyped and imputed DNA sequence variants with a MAF>0.0003 were tested for association with the inverse normal transformed residuals of lipid values through linear regression assuming an additive genetic model. In a discovery analysis, association testing was performed separately among individuals of each of three genetic ancestries (whites, blacks, and Hispanics) and then meta-analyzed results across ethnic groups using an inverse variance-weighted fixed effects method. For variants with suggestive associations (association P<10-4), replication was sought of the findings in one of two independent studies: the 2017 GLGC exome array meta-analysis or the 2013 GLGC “joint meta-analysis.” Replication was first performed using summary statistics from the 2017 GLGC exome array study. A total of 242,289 variants in up to 319,677 individuals were analyzed after quality control and were available for replication.

If a DNA sequence variant was not available for replication in the above exome array-focused study, replication was sought from publicly available summary statistics from the 2013 GLGC “joint meta-analysis.” An additional 2,044,165 variants in up to 188,587 individuals were available for replication in this study. In total, 2,286,454 DNA sequence variants in up to 319,677 individuals were available for independent replication. If a variant was available for replication in both studies, replication was prioritized using summary statistics from the 2017 GLGC exome array study given its larger sample size. Significant novel associations were defined as those that were at least nominally significant in replication (P<0.05) and had an overall P<5×10-8 (genome-wide significance) in the discovery and replication cohorts combined. Novel loci were defined as being greater than 1 mB away from a known lipid genome-wide associated lead variant. Additionally, linkage disequilibrium information from the 1000 Genomes Project was used to determine independent variants where a locus extended beyond 1 mB.

v. Conditional Analysis

Given that individual level data for the prior GLGC lipid analyses are not publicly available, we used the COJO-GCTA software to perform an approximate, stepwise conditional analysis to identify independent variants within lipid-associated loci. We used summary statistics after a meta-analysis of 1.9 million overlapping variants across the GLGC (predominantly European) and European MVP datasets (FIG. 11). An LD-matrix obtained from 10,000 unrelated European individuals randomly sampled from the UK Biobank interim release was used for this analysis.

vi. Variance Explained and Gain Using Multiple Lipid Measurements

The proportion of variance explained by the set of 444 previously mapped independent lipid variants, the 118 novel lipid loci identified in the study, and the 826 independent lipid variants identified from conditional analysis using ridge regression with the glmnet R package were estimated. The variance explained was determined after tuning the hyperparameter (lambda) to approximate an optimal value, and then calculating the model R2 after performing linear regression with the inverse normal transformed lipid outcome and each set (444, 118, 826) of independent genome-wide variants as predictors.

To assess the impact of multiple lipid measurements, the variance explained for a GRS of 223 previously described GWAS lipid variants weighted by their previously reported effect sizes as a function of the number of lipid measurements was estimated (FIG. 22). This analysis was performed using one, two, three, four, and five lipid measurements for each individual starting with their measurement closest to enrollment and moving backward in time. To account for the use of statin therapy, individuals with evidence of a statin prescription in their EHR at the time of enrollment had their LDL-C/TC values adjusted by dividing by 0.7/0.8, respectively as previously described. In addition, the variance explained by the maximal TG, LDL-C/TC, and minimal HDL-C from the EHR was calculated without adjustment for lipid lowering therapy. A set of 171,314 European MVP participants was focused on with >5 lipid measurements available for this analysis.

vii. Lipids Transcriptome-wide Association Study

A TWAS was performed using summary statistics after a meta-analysis of 1.9 million overlapping variants among GLGC (predominantly European) and European MVP datasets (FIG. 11) and four gene-expression reference panels (NTR whole blood, METSIM adipose tissue, and tibial artery and liver from GTEx) in independent samples as previously described. In brief, for a given gene, variant-expression weights in the 1-mB cis locus were first computed with the BSLMM, which models effects on expression as a mixture of normal distributions to account for the sparse expression architecture. Given weights w, lipid Z scores Z, and variant-correlation (LD) matrix D; the association between predicted expression and lipids (i.e., the TWAS statistic) was estimated as ZTWAS=w′Z/(w′Dw)½. TWAS statistics were computed by using either the variants genotyped in each expression reference panel or imputed HapMap3 [symbol] variants. To account for multiple hypotheses a genome-wide significant P value threshold (P<5×10-8) was applied, significantly more stringent than previously used Bonferroni corrections in prior TWAS26. Novel TWAS loci were defined as a TWAS gene falling outside of a previously identified lipid GWAS region (±1 mB around a mapped sentinel GWAS variant).

viii. Tissue Expression Analysis and Competitive Gene Set Pathway Analysis

MAGMA was used as implemented in the FUMA pipeline to perform a competitive gene set analysis for 10,655 gene sets (curated gene sets: 4,738, GO terms: 5,917) present in the Molecular Signature Database (MsigDB 6.1) and a gene-property analysis for gene expression in GTEx v7 with 53 tissue types. The input for these analyses was the 1000 Genomes imputed summary statistics from Stage 1 for LDL-C, TG, and HDL-C. The combined trans-ethnic summary statistics were run and then the summary statistics in the European subgroup of participants alone. For the gene-set analyses, a P adjusted for the number of total gene sets tested was calculated and output for gene-sets with P bon <0.05. MAGMA gene-set and gene-property analyses uses the full distribution of SNP p values and differs from pathway enrichment tests that only tests for enrichment of prioritized genes.

ix. Identification of Independent Low-Frequency Coding Variant Lipid Associations Specific to Blacks and Hispanics

The P value and linkage disequilibrium-driven clumping procedure in PLINK version 1.90b (-clump) was used to identify associations between low-frequency coding variants and lipids specific to blacks and Hispanics. Input included summary lipid association statistics from our MVP 1000 Genomes imputed genome-wide association study of black and Hispanic individuals, and reference linkage disequilibrium panels of 661 African (AFR) and 347 Ad Mixed American (AMR) samples from 1000 Genomes phase 3 whole genome sequencing data. Variants were clumped with stringent r2 (<0.01) and P (<5×10⁻⁸) thresholds in a 1 mega-base region surrounding the lead variant at each locus to reveal independent index variants at genome-wide significance. From this list of independent variants, we report novel protein-altering variants specific to blacks and Hispanics at a MAF<0.05.

x. Loss of Gene Function Analysis

The Variant Effect Predictor software was used to identify pLoF DNA sequence variants defined as: premature stop (nonsense), canonical splice-sites (splice-donor or splice-acceptor) or insertion/deletion variants that shifted frame (frameshift). These variants were then merged with data from the Exome Aggregation Consortium24 (Version 0.3.1), a publicly available catalogue of exome sequence data to confirm consistency in variant annotation. pLoF DNA sequence variants were required to be observed in at least 50 individuals, and set a statistical significance threshold of P<5×10⁻⁸ (genome-wide significance).

xi. Loss of PDE3B Gene Function and Coronary Artery Disease

A novel lipid association was identified for a pLoF mutation in the PDE3B gene (rs150090666, p.Arg783Ter). For carriers of damaging mutations in Phosphodiesterase 3B, the mutation's effects on risk for CAD were examined using logistic regression in five separate cohorts: MVP, UK Biobank, and 3 cohorts with exome sequencing: the Myocardial Infarction Genetics Consortium (MIGen), the Penn Medicine Biobank (PMBB), and DiscovEHR. In studies with exome sequencing, pLoF variants were combined with missense variants predicted to be damaging or possibly damaging by each of 5 computer prediction algorithms (LRT score, MutationTaster, PolyPhen-2, HumDiv, PolyPhen-2 HumVar, and SIFT) as performed previously. Because any individual damaging mutation was rare, variants were aggregated together for subsequent phenotypic analysis. Logistic regression on disease status was performed, adjusting for age, sex, and principal components of ancestry as appropriate. Effects of PDE3B damaging mutations were pooled across studies using an inverse-variance weighted fixed effects meta-analysis. A P<0.05 threshold for statistical significance was set.

xii. Novel Lipid Loci and Association with Coronary Disease

To assess whether novel lipid loci in our study modulate the risk of CAD, association results were extracted for the lead variant at each locus from either the CARDIoGRAMplusC4D 1000 Genomes imputed CAD GWAS37 (115/118 variants) or from the MIGen and CARDIoGRAM Exome Chip GWAS analysis for 3 variants not available in the former. A two-tailed exact binomial test for goodness of fit was performed examining the expected and observed distributions of 1) LDL-C and 2) TG raising alleles (P<10⁻⁴), and 3) HDL-C raising alleles (P<10⁻⁴) not also associated with LDL-C or TG (P>0.05) and their effect on CAD risk. The null hypothesis was tested that the lipid-associated variants were equally likely to increase or decrease CAD risk and set a P<0.05 threshold for statistical significance.

xiii. Lipids and Abdominal Aortic Aneurysm Mendelian Randomization Analysis

Summary-level data for 223 genome-wide lipids-associated variants were obtained from the publicly available data from the Global Lipids Genetics Consortium. Results were utilized from a GWAS of 5,002 AAA cases and 139,968 controls performed in white MVP participants using the definition proposed by Denny et al. The effect alleles were matched with all lipid and AAA summary data and 3 different Mendelian randomization analyses were performed: 1) inverse variance-weighted; 2) multivariable; 3) MR-Egger to account for pleiotropic bias. First, inverse-variance-weighted Mendelian randomization was performed using each set of variants for each lipid trait as instrumental variables. This method, however, does not account for possible pleiotropic bias. Therefore, inverse-variance-weighted multivariable Mendelian randomization was next performed. This method adjusts for possible pleiotropic effects across the included lipid traits in our analyses using effect estimates from the variant-AAA outcome and effect estimates from variant-LDL-C, variant-HDL-C, and variant-TG as predictors in 1 multivariable model. MR-Egger was additionally performed. This technique can be used to detect bias secondary to unbalanced pleiotropy in Mendelian randomization studies. In contrast to inverse variance-weighted analysis, the regression line is unconstrained, and the intercept represents the average pleiotropic effects across all variants. Bonferroni-corrected 2-sided P values (P=0.016; 0.05/3) for 3 tests were used to declare statistical significance.

Those skilled in the art will recognize, or be able to ascertain using no more than routine experimentation, many equivalents to the specific embodiments of the method and compositions described herein. Such equivalents are intended to be encompassed by the following claims.

TABLE 5
Association results of previously reported genome-wide significant loci for HDL cholesterol in the MVP lipids discovery (trans-ethnic) analysis

| | | | | Known | | | | | | |
|---|---|---|---|---|---|---|---|---|---|---|
| Chr:Pos | rsid | Annotation | Gene* | Locus** | EA | NEA | EAF | Beta | SE | P |
| 1:109818158 | rs3832016 | 3_prime_UTR_variant | CELSR2 | SORT1 | D | I | 0.2119 | 0.0421 | 0.0038 | 4.01E−29 |
| 1:150940625 | rs267738 | missense_variant | CERS2 | ANXA9 | T | G | 0.8083 | −0.0248 | 0.0036 | 6.22E−12 |
| 1:178533832 | rs4077194 | downstream_gene_variant | (RNA5SP69) | ANGPTL1 | T | G | 0.4801 | −0.0186 | 0.0027 | 8.85E−12 |
| 1:182154990 | rs61805076 | intron_variant | GS1-122H1.2 | ZNF648 | T | C | 0.6945 | 0.0251 | 0.003 | 1.22E−16 |
| 1:214992980 | rs4655268 | intergenic_variant | (CENPF) | PROX1 | C | G | 0.4562 | 0.0166 | 0.0028 | 1.95E−09 |
| 1:219664030 | rs2066152 | intergenic_variant | (ZC3H11B) | LYPLAL1 | A | G | 0.4025 | 0.0205 | 0.0028 | 9.04E−14 |
| 1:220970028 | rs2642438 | missense_variant | MARC1 | MOSC1 | A | G | 0.2715 | −0.0224 | 0.0031 | 5.94E−13 |
| 1:230295691 | rs4846914 | intron_variant | GALNT2 | GALNT2 | A | G | 0.5456 | 0.0409 | 0.0028 | 4.20E−48 |
| 1:27284913 | rs79598313 | intron_variant | C1orf172 | HDGF | T | C | 0.0216 | −0.0898 | 0.01 | 2.69E−19 |
| 1:40035928 | rs3768321 | intron_variant | PABPC4 | PABPC4 | T | G | 0.1737 | −0.0505 | 0.004 | 1.42E−36 |
| 1:63153199 | rs68148663 | intron_variant | DOCK7 | ANGPTL3 | D | I | 0.3422 | −0.0195 | 0.0029 | 8.77E−12 |
| 1:93862020 | rs10874777 | intergenic_variant | (FNBP1L) | EVI5 | T | C | 0.4369 | 0.0244 | 0.0028 | 7.03E−18 |
| 10:101912194 | rs1408579 | intron_variant | ERLIN1 | CHUK | T | C | 0.4351 | 0.0185 | 0.0028 | 6.89E−11 |
| 10:113921825 | rs2792735 | intron_variant | GPAM | GPAM | A | G | 0.7206 | −0.0306 | 0.0033 | 5.79E−21 |
| 10:115786233 | rs72823013 | regulatory_region_variant | (ADRB1) | ADRB1 | A | G | 0.1124 | 0.0266 | 0.0045 | 3.74E−09 |
| 10:46060433 | rs553682607 | intron_variant | MARCH8 | MARCH8 | D | I | 0.2484 | 0.027 | 0.0034 | 9.99E−16 |
| 11:110012143 | rs689183 | intron_variant | ZC3H12C | ZC3H12C | T | G | 0.7461 | −0.0182 | 0.0031 | 3.96E−09 |
| 11:116701354 | rs138326449 | splice_donor_variant | APOC3 | APOA1 | A | G | 0.0033 | 0.7285 | 0.0296 | 4.95E−134 |
| 11:122520291 | rs19453 91 | regulatory_region_variant | (UBASH3B) | UBASH3B | A | T | 0.5525 | −0.0228 | 0.0028 | 1.60E−16 |
| 11:14865399 | rs150090666 | stop_gained | PDE3B | PDE3B | T | C | 8.00E−04 | 0.3987 | 0.0487 | 2.51E−16 |
| 11:18067020 | rs11434755 | upstream_gene_variant | TPH1 | SPTY2D1 | D | I | 0.5737 | 0.0152 | 0.0027 | 1.63E−08 |
| 11:47266471 | rs75393320 | intron_variant | ACP2 | LRP4 | C | G | 0.1212 | 0.0647 | 0.0046 | 7.92E−45 |
| 11:61552680 | rs174537 | intron_variant | MYRF | FAS1-2-3 | T | G | 0.3123 | −0.0305 | 0.0031 | 1.94E−22 |
| 11:64018104 | rs71468663 | upstream_gene_variant | (PLCB3) | PLCB3 | D | I | 0.9555 | 0.0537 | 0.0067 | 1.73E−15 |
| 11:65405600 | rs2306363 | upstream_gene_variant | SIPA1 | KAT5 | T | G | 0.1834 | 0.0227 | 0.0036 | 4.56E−10 |
| 11:75552579 | rs34696509 | intron_variant | UVRAG | MOGAT-DGAT2 | D | I | 0.2128 | −0.0323 | 0.0044 | 2.21E−13 |
| 12:110015893 | rs7954144 | intron_variant | MVK | MVK | A | G | 0.4301 | −0.0273 | 0.0027 | 7.61E−24 |
| 12:111904371 | rs4766578 | intron_variant | ATXN2 | BRAP | A | T | 0.5425 | 0.0219 | 0.0028 | 1.14E−14 |
| 12:123796238 | rs4759375 | intron_variant | SBNO1 | SBNO1 | T | C | 0.0919 | 0.0487 | 0.0048 | 1.67E−24 |
| 12:125338529 | rs10773112 | intron_variant | SCARB1 | ZNF664 | T | C | 0.6429 | 0.039 | 0.0028 | 7.59E−43 |
| 12:20470199 | rs11045171 | intergenic_variant | (PDE3A) | PDE3A | A | G | 0.8048 | −0.0318 | 0.0036 | 2.27E−18 |
| 12:57848639 | rs3809114 | upstream_gene_variant | INHBE | LRP1 | A | G | 0.5061 | 0.0177 | 0.0027 | 6.94E−11 |
| 12:7725583 | ss1388044873 | intergenic_variant | NA | CD163 | A | G | 0.8269 | −0.026 | 0.0047 | 3.49E−08 |
| 14:105258892 | rs2494748 | intron_variant | AKT1 | ZBTB42 | T | C | 0.5435 | −0.0303 | 0.0029 | 3.31E−26 |
| 14:74250126 | rs13379043 | upstream_gene_variant | RP5-1021120.1 | C14orf43 | T | C | 0.6293 | −0.0168 | 0.0029 | 1.17E−08 |
| 15:43674430 | rs148149124 | intron_variant | TUBGCP4 | CAPN3 | D | I | 0.0281 | −0.1018 | 0.0097 | 5.59E−26 |
| 15:43933941 | rs13 9097404 | intron_variant | CATSPER2 | FRMD5 | T | C | 0.9746 | 0.0988 | 0.0094 | 9.05E−26 |
| 15:58673449 | rs77250403 | intron_variant | ALDH1A2 | LIPC | D | I | 0.3343 | 0.0913 | 0.0029 | 5.63E−216 |
| 15:63395428 | rs55703462 | intron_variant | RP11-69G7.1 | LACTB | A | G | 0.5027 | −0.0187 | 0.0031 | 3.10E−09 |
| 16:53818460 | rs3751812 | intron_variant | FTO | FTO | T | G | 0.3619 | −0.0253 | 0.0029 | 1.88E−18 |
| 16:56993886 | rs821840 | upstream_gene_variant | (CETP) | CETP | A | G | 0.6902 | −0.2211 | 0.0028 | <1E−300 |
| 16:67942320 | rs56070533 | intron_variant | PSKH1 | LCAT | A | G | 0.1512 | 0.0779 | 0.0038 | 8.82E−95 |
| 16:69357406 | rs16958751 | intron_variant | VPS4A | TMED6 | A | G | 0.0376 | 0.0508 | 0.0081 | 3.48E−10 |
| 16:72088461 | rs5471 | upstream_gene_variant | (HP) | HPR | A | C | 0.9039 | −0.0743 | 0.0111 | 2.17E−11 |
| 16:81534790 | rs2925979 | intron_variant | CMIP | CMIP | T | C | 0.2955 | −0.0309 | 0.0029 | 6.14E−27 |
| 17:26722039 | rs34879232 | 3_prime_UTR_variant | SLC46A1 | VTN | D | I | 0.4208 | 0.0155 | 0.0027 | 1.52E−08 |
| 17:37746359 | rs11078917 | intergenic_variant | (NEUROD2) | STARD3 | A | C | 0.3803 | −0.0336 | 0.0029 | 3.41E−30 |
| 17:41926126 | rs72836561 | missense_variant | CD300LG | CD300LG | T | C | 0.029 | −0.1987 | 0.0084 | 1.40E−123 |
| 17:45766771 | rs56325564 | downstream_gene_variant | KPNB1 | OSBPL7 | A | G | 0.4569 | 0.0148 | 0.0027 | 4.45E−08 |
| 17:65892507 | rs61676547 | intron_variant | BPTF | ABCA8 | C | G | 0.3144 | −0.0208 | 0.0031 | 3.01E−11 |
| 17:76400329 | rs12601079 | intron_variant | PGS1 | PGS1 | A | G | 0.6042 | 0.0334 | 0.0027 | 7.61E−35 |
| 18:47109955 | rs77960347 | missense_variant | LIPG | LIPG | A | G | 0.9878 | −0.2326 | 0.0128 | 2.90E−73 |
| 19:11350488 | rs2278426 | missense_variant | C19orf80 | ICAM1 | T | C | 0.1249 | −0.0756 | 0.0049 | 9.66E−53 |
| 19:11414706 | rs56121005 | intron_variant | TSPAN16 | LDLR | T | C | 0.0129 | −0.1052 | 0.0168 | 4.03E−10 |
| 19:33940662 | rs34940240 | intron_variant | PEPD | PEPD | D | I | 0.5407 | 0.0194 | 0.0027 | 4.32E−13 |
| 19:45411941 | rs429358 | missense_variant | APOE | APOE | T | C | 0.8416 | 0.093 | 0.0037 | 1.26E−142 |
| 19:50161091 | rs61743199 | missense_variant | SCAF1 | FLJ36070 | A | G | 0.933 | 0.0343 | 0.0057 | 1.96E−09 |
| 19:52304069 | rs74256604 | intron_variant | FPR3 | HAS1 | A | G | 0.1298 | −0.0284 | 0.0043 | 3.12E−11 |
| 19:54799083 | rs380267 | downstream_gene_variant | (LILRA3) | LILRA3 | A | G | 0.805 | −0.0622 | 0.0035 | 4.24E−71 |
| 19:7242261 | rs56149994 | intron_variant | INSR | INSR | T | C | 0.2862 | −0.0229 | 0.0031 | 1.18E−13 |
| 19:8429323 | rs116843064 | missense_variant | ANGPTL4 | ANGPTL4 | A | G | 0.0191 | 0.2576 | 0.0103 | 3.32E−137 |
| 2:165501927 | rs5835988 | intergenic_variant | (GRB14) | COBLL1 | D | I | 0.5473 | −0.03 | 0.0028 | 2.42E−26 |
| 2:203477868 | rs72926946 | upstream_gene_variant | (MTND4P30) | FAM117B | A | C | 0.2784 | −0.0228 | 0.003 | 6.10E−14 |
| 2:21231524 | rs676210 | missense_variant | APOB | APOB | A | G | 0.2052 | 0.0546 | 0.0033 | 4.81E−63 |
| 2:219720952 | rs200513066 | upstream_gene_variant | (WNT6) | PRKAG3 | D | I | 0.0509 | −0.0892 | 0.0095 | 5.08E−21 |
| 2:227094758 | rs2203452 | intergenic_variant | (IRS1) | IRS1 | A | G | 0.3482 | 0.0417 | 0.0028 | 8.82E−52 |
| 2:239597 | rs6710091 | intron_variant | SH3YL1 | ACP1 | C | G | 0.686 | −0.0165 | 0.003 | 4.63E−08 |
| 2:65282708 | rs6728523 | upstream_gene_variant | (CEP68) | CEP68 | C | G | 0.2803 | 0.0239 | 0.003 | 2.37E−15 |
| 20:33719183 | rs3746428 | intron_variant | EDEM2 | ERGIC3 | A | G | 0.1592 | −0.024 | 0.0038 | 2.84E−10 |
| 20:43042364 | rs1800961 | missense_variant | HNF4A | HNF4A | T | C | 0.0303 | −0.1408 | 0.0082 | 2.08E−65 |
| 20:44557215 | rs562306828 | intergenic_variant | (PLTP) | PLTP | D | I | 0.7873 | 0.0398 | 0.0033 | 1.02E−33 |
| 21:46271452 | rs235314 | 3_prime_UTR_variant | PTTG1IP | COL18A1 | T | C | 0.4952 | −0.0174 | 0.0028 | 3.59E−10 |
| 22:21976934 | rs7444 | downstream_gene_variant | (UBE2L3) | UBE2L3 | T | C | 0.7049 | 0.034 | 0.0031 | 1.01E−28 |
| 22:29400515 | rs8142788 | intron_variant | ZNRF3 | MTMR3 | A | G | 0.1559 | −0.023 | 0.004 | 1.16E−08 |
| 22:38594668 | rs2899297 | upstream_gene_variant | MAFF | PLA2G6 | A | G | 0.5479 | −0.0225 | 0.0027 | 9.89E−17 |
| 22:44340904 | rs2294915 | intron_variant | PNPLA3 | PNPLA3 | T | C | 0.246 | −0.0177 | 0.0032 | 2.81E−08 |
| 3:12379351 | rs35240997 | intron_variant | PPARG | PPARG | A | G | 0.7924 | −0.0256 | 0.0035 | 1.25E−13 |
| 3:136125678 | rs151105710 | intron_variant | STAG1 | MSL2L1 | D | I | 0.8089 | −0.0256 | 0.0035 | 1.62E−13 |
| 3:156795414 | rs9817452 | upstream_gene_variant | RP11-6F2.5 | LOC100498859 | T | G | 0.3746 | 0.0283 | 0.0029 | 6.78E−22 |
| 3:185931174 | rs2268840 | intron_variant | DGKG | ETV5 | T | C | 0.7876 | −0.0215 | 0.0034 | 1.66E−10 |
| 3:47097985 | rs62246406 | intron_variant | SETD2 | SETD2 | A | G | 0.1629 | −0.0237 | 0.0038 | 4.53E−10 |
| 3:48767877 | rs6808104 | intron_variant | IP6K2 | CDC25A | A | G | 0.534 | 0.0158 | 0.0027 | 7.30E−09 |
| 3:50024038 | rs111439884 | intron_variant | RBM6 | RBM5 | A | C | 0.5043 | 0.0221 | 0.0027 | 2.77E−16 |
| 3:52372366 | rs11706108 | intron_variant | DNAH1 | STAB1 | T | C | 0.7059 | −0.026 | 0.0034 | 2.11E−14 |
| 4:100517324 | rs12509976 | intron_variant | MTTP | ADH5 | T | C | 0.0995 | 0.0394 | 0.0055 | 8.49E−13 |
| 4:103188709 | rs13107325 | missense_variant | SLC39A8 | SLC39A8 | T | C | 0.0765 | −0.0798 | 0.0053 | 1.54E−50 |
| 4:157720124 | rs4691380 | intron_variant | PDGFC | PDGFC | T | C | 0.4071 | 0.0159 | 0.0028 | 1.15E−08 |
| 4:26050450 | rs10713774 | intergenic_variant | (SMIM20) | C4orf52 | D | I | 0.188 | −0.0223 | 0.0035 | 1.32E−10 |
| 4:69349018 | rs1117816 | intron_variant | TMPRSS11E | TMPRSS11E | A | C | 0.78 | −0.0212 | 0.0033 | 2.07E−10 |
| 4:89740128 | rs13133548 | intron_variant | FAM13A | FAM13A | A | G | 0.4745 | −0.0216 | 0.0026 | 1.60E−16 |
| 5:132467373 | rs10479024 | intergenic_variant | (RPL6P15) | SLC22A5 | A | C | 0.0726 | 0.0376 | 0.0058 | 1.09E−10 |
| 5:53274467 | rs28499105 | intron_variant | ARL15 | ARL15 | A | G | 0.708 | −0.0171 | 0.0029 | 4.53E−09 |
| 5:55806751 | rs459193 | downstream_gene_variant | AC022431.2 | MAP3K1 | A | G | 0.2967 | 0.029 | 0.0029 | 2.01E−23 |
| 5:75003678 | rs2307111 | missense_variant | POC5 | HMGCR | T | C | 0.5474 | −0.023 | 0.0028 | 3.00E−16 |
| 6:127476717 | rs2489629 | intron_variant | RSPO3 | RSPO3 | T | C | 0.5744 | −0.0175 | 0.0027 | 6.23E−11 |
| 6:139835418 | rs199607859 | intergenic_variant | (LOC645434) | CITED2 | T | G | 0.548 | 0.0203 | 0.0027 | 7.04E−14 |
| 6:161092438 | rs11751347 | intron_variant | RP1-81D8.4 | LPA | T | C | 0.0857 | −0.0636 | 0.0054 | 2.36E−32 |
| 6:34668635 | rs9368830 | upstream_gene_variant | (C6orf106) | C6orf106 | T | C | 0.574 | 0.0277 | 0.0029 | 4.07E−21 |
| 6:43757896 | rs998584 | downstream_gene_variant | (VEGFA) | VEGFA | A | C | 0.4455 | −0.0368 | 0.0028 | 5.16E−40 |
| 7:130432481 | rs6971365 | intergenic_variant | (KLF14) | KLF14 | T | C | 0.7147 | 0.0262 | 0.003 | 2.29E−18 |
| 7:150529449 | rs17173637 | intron_variant | AOC1 | TMEM176A | T | C | 0.9156 | 0.0294 | 0.0048 | 8.65E−10 |
| 7:17911752 | rs1917368 | intron_variant | SNX13 | SNX13 | T | G | 0.465 | −0.028 | 0.0027 | 1.72E−24 |
| 7:26370190 | rs4722593 | intron_variant | SNX10 | MIR148A | A | G | 0.3238 | 0.0209 | 0.0032 | 3.88E−11 |
| 7:6461310 | rs79949326 | intron_variant | DAGLB | DAGLB | T | C | 0.2327 | 0.024 | 0.0033 | 3.19E−13 |
| 7:73037366 | rs55747707 | intron_variant | MLXIPL | TYW1B | A | G | 0.1796 | 0.0314 | 0.0035 | 5.85E−19 |
| 7:80300449 | rs3211938 | stop_gained | CD36 | CD36 | T | G | 0.9109 | −0.122 | 0.0113 | 2.66E−27 |
| 8:116603103 | rs2721954 | intron_variant | TRPS1 | TRPS1 | T | C | 0.609 | 0.0305 | 0.0029 | 1.43E−26 |
| 8:126507389 | rs2954038 | intron_variant | RP11-136O12.2 | TRIB1 | A | C | 0.7182 | 0.041 | 0.003 | 1.14E−41 |
| 8:144297020 | rs78123380 | intron_variant | GPIHBP1 | PLEC1 | A | G | 0.1585 | 0.0473 | 0.008 | 2.67E−09 |
| 8:19850099 | rs79407615 | intergenic_variant | (LPL) | LPL | T | G | 0.9025 | −0.1799 | 0.0049 | 3.05E−293 |
| 8:9183596 | rs4841132 | non_coding_transcript_exon_variant | RP11-115J16.1 | PPP1R3B | A | G | 0.1054 | −0.0954 | 0.0044 | 1.20E−105 |
| 9:107589744 | rs4149307 | intron_variant | ABCA1 | ABCA1 | T | C | 0.3233 | 0.0685 | 0.0033 | 2.95E−93 |
| 9:15304782 | rs686030 | intron_variant | TTC39B | TTC39B | A | C | 0.8696 | 0.0431 | 0.004 | 1.34E−27 |
| 9:17295541 | rs10963012 | intron_variant | CNTLN | BNC2 | C | G | 0.6314 | −0.0362 | 0.0065 | 3.15E−08 |
| 9:5073770 | rs77375493 | missense_variant | JAK2 | JAK2 | T | G | 5.00E−04 | −0.5576 | 0.0614 | 1.15E−19 |

TABLE 6
Association results of previously reported genome-wide significant loci for TC in the MVP lipids discovery (trans-ethnic) analysis

| | | | | Known | | | | | | |
|---|---|---|---|---|---|---|---|---|---|---|
| Chr:Pos | rsid | Annotation | Gene* | Locus** | EA | NEA | EAF | Beta | SE | P |
| 1:109817192 | rs7528419 | 3_prime_UTR_variant | CELSR2 | SORT1 | A | G | 0.7715 | 0.1364 | 0.0031 | <1E−300 |
| 1:220972343 | rs867772 | intron_variant | MARC1 | MOSC1 | A | G | 0.2868 | −0.0204 | 0.003 | 2.00E−11 |
| 1:234853059 | rs556107 | intron_variant | RP4-781K5.7 | IRF2BP2 | T | C | 0.5912 | 0.0369 | 0.003 | 1.43E−35 |
| 1:23747996 | rs11340914 | intron_variant | TCEA3 | ASAP3 | D | I | 0.6579 | 0.0163 | 0.003 | 3.58E−08 |
| 1:25797832 | rs34293609 | intron_variant | TMEM57 | LDLRAP1 | D | I | 0.4797 | −0.0174 | 0.0027 | 5.76E−11 |
| 1:55505647 | rs11591147 | missense_variant | PCSK9 | PCSK9 | T | G | 0.015 | −0.325 | 0.0115 | 7.58E−175 |
| 1:63107526 | rs995000 | intron_variant | DOCK7 | ANGPTL3 | T | C | 0.3454 | −0.0618 | 0.0028 | 6.32E−112 |
| 1:93137529 | rs145955280 | intron_variant | EVI5 | EVI5 | D | I | 0.9397 | −0.084 | 0.012 | 2.48E−12 |
| 10:113933006 | rs77987196 | intron_variant | GPAM | GPAM | D | I | 0.7101 | −0.0207 | 0.0029 | 1.03E−12 |
| 10:17268839 | rs3758413 | upstream_gene_variant | (VIM) | VIM | T | C | 0.6103 | −0.0222 | 0.0027 | 2.26E−16 |
| 10:45979232 | rs145976573 | intron_variant | MARCH8 | MARCH8 | D | I | 0.7798 | −0.022 | 0.0034 | 6.60E−11 |
| 10:52573772 | rs41274050 | missense_variant | A1CF | A1CF | T | C | 0.0088 | 0.1069 | 0.0149 | 6.41E−13 |
| 10:94839642 | rs2068888 | downstream_gene_variant | (CYP26A1) | CYP26A1 | A | G | 0.4266 | −0.0222 | 0.0027 | 6.55E−17 |
| 11:116662407 | rs3135506 | missense_variant | APOA5 | APOA1 | C | G | 0.071 | 0.1053 | 0.0051 | 2.14E−93 |
| 11:118480285 | rs12225399 | intron_variant | PHLDB1 | PHLDB1 | C | G | 0.3783 | 0.0176 | 0.0027 | 1.41E−10 |
| 11:122506970 | rs7927208 | intergenic_variant | (UBASH3B) | UBASH3B | T | C | 0.6135 | −0.022 | 0.0028 | 3.32E−15 |
| 11:126239143 | rs68055275 | intron_variant | ST3GAL4 | ST3GAL4 | D | I | 0.8648 | −0.0349 | 0.004 | 3.20E−18 |
| 11:47488114 | rs555328608 | downstream_gene_variant | CELF1 | LRP4 | D | I | 0.6733 | 0.0183 | 0.003 | 1.23E−09 |
| 11:75474195 | rs72997616 | intron_variant | CTD-2530H12.1 | MOGAT2-DGAT2 | A | C | 0.1232 | −0.0243 | 0.0044 | 4.11E−08 |
| 12:109939641 | rs2241212 | intron_variant | UBE3B | MVK | A | T | 0.5581 | 0.0152 | 0.0027 | 1.52E−08 |
| 12:112007756 | rs653178 | intron_variant | ATXN2 | BRAP | T | C | 0.5505 | 0.0273 | 0.0028 | 2.08E−22 |
| 12:121416650 | rs1169288 | missense_variant | HNF1A | HNF1A | A | C | 0.6937 | −0.0344 | 0.003 | 1.56E−30 |
| 12:123867994 | rs28516750 | upstream_gene_variant | (SETD8) | SBNO1 | A | G | 0.8985 | −0.0303 | 0.0043 | 2.37E−12 |
| 12:125261593 | rs838880 | downstream_gene_variant | (SCARB1) | ZNF664 | T | C | 0.6042 | −0.0174 | 0.0028 | 3.16E−10 |
| 12:9098995 | 12:9098995 | inframe_insertion | M6PR | PHC1 | D | I | 0.9042 | 0.0314 | 0.0048 | 4.34E−11 |
| 13:32988865 | rs151330264 | intron_variant | N4BP2L1 | BRCA2 | A | T | 0.9705 | −0.1111 | 0.0182 | 1.09E−09 |
| 14:64235556 | rs7157785 | regulatory_region_variant | (SYNE2) | SGPP1 | T | G | 0.2401 | 0.0206 | 0.0032 | 1.66E−10 |
| 14:94847262 | rs17580 | missense_variant | SERPINA1 | SERPINA1 | A | T | 0.0412 | 0.0571 | 0.007 | 2.50E−16 |
| 15:58679668 | rs7350789 | intron_variant | ALDH1A2 | LIPC | A | G | 0.3451 | 0.0522 | 0.0028 | 3.56E−78 |
| 15:63793873 | rs11636917 | upstream_gene_variant | (USP3) | LACTB | T | C | 0.6695 | −0.0206 | 0.003 | 3.31E−12 |
| 16:56991363 | rs183130 | upstream_gene_variant | (CETP) | CEIP | T | C | 0.3073 | 0.0549 | 0.0028 | 1.01E−84 |
| 16:67976851 | rs35673026 | missense_variant | LCAT | LCAT | T | C | 0.0042 | 0.3485 | 0.0451 | 1.14E−14 |
| 16:72088461 | rs5471 | upstream_gene_variant | (HP) | HPR | A | C | 0.9039 | −0.1961 | 0.011 | 1.00E−70 |
| 17:18125877 | rs62072497 | upstream_gene_variant | (LLGL1) | PEMT | A | G | 0.1882 | 0.0208 | 0.0034 | 1.17E−09 |
| 17:27047243 | rs529619980 | intron_variant | RPL23A | VTN | D | I | 0.8917 | 0.0271 | 0.0045 | 1.33E−09 |
| 17:28632119 | rs11291804 | intergenic_variant | (BLMH) | NF1 | D | I | 0.4053 | −0.0199 | 0.0034 | 4.86E−09 |
| 17:45734210 | 17:45734210 | NA | NA | OSBPL7 | D | I | 0.5016 | −0.0166 | 0.0026 | 2.09E−10 |
| 17:4693902 | rs73339979 | upstream_gene_variant | (VMO1) | TM4SF5 | C | G | 0.1787 | −0.0588 | 0.0076 | 7.82E−15 |
| 17:67081278 | rs77542162 | missense_variant | ABCA6 | ABCA8 | A | G | 0.9817 | −0.1283 | 0.0107 | 2.17E−33 |
| 17:7012254 | rs146261845 | intron_variant | ASGR2 | DLG4 | T | C | 0.0037 | −0.3173 | 0.033 | 6.41E−22 |
| 17:76392144 | rs17561950 | intron_variant | PGS1 | PGS1 | A | G | 0.4593 | 0.0196 | 0.0027 | 4.58E−13 |
| 17:8219478 | rs2270445 | intron_variant | ARHGEF15 | ARHGEF15 | A | G | 0.4596 | −0.0153 | 0.0027 | 2.47E−08 |
| 18:47109955 | rs77960347 | missense_variant | LIPG | LIPG | A | G | 0.9878 | −0.1674 | 0.0127 | 1.48E−39 |
| 19:11196886 | rs8106503 | upstream_gene_variant | (LDLR) | LDLR | T | C | 0.8354 | 0.1334 | 0.0037 | 1.64E−281 |
| 19:11388713 | rs140402167 | intergenic_variant | (DOCK6) | LDLR | T | G | 0.0308 | −0.181 | 0.0162 | 5.52E−29 |
| 19:19686071 | rs199768142 | intron_variant | PBX4 | CILP2 | D | I | 0.0407 | −0.1458 | 0.0212 | 6.10E−12 |
| 19:45422160 | rs12721051 | intron_variant | APOC1 | APOE | C | G | 0.8427 | −0.1061 | 0.0037 | 1.12E−179 |
| 2:118859159 | rs149369311 | intron_variant | INSIG2 | INSIG2 | D | I | 0.0914 | −0.0312 | 0.005 | 3.87E−10 |
| 2:121306440 | rs17050272 | upstream_gene_variant | (AC073257.2) | LOC84931 | A | G | 0.389 | −0.0181 | 0.0028 | 1.26E−10 |
| 2:158437683 | rs4377290 | intron_variant | ACVR1C | ACVR1C | T | C | 0.5033 | 0.0174 | 0.0026 | 2.30E−11 |
| 2:169830798 | rs10177080 | intron_variant | ABCB11 | ABCB11 | A | G | 0.5435 | −0.0189 | 0.0027 | 2.59E−12 |
| 2:203519264 | rs72926986 | intron_variant | FAM117B | FAM117B | T | G | 0.278 | −0.0326 | 0.003 | 2.35E−27 |
| 2:21293335 | rs10692845 | regulatory_region_variant | (LOC100287183) | APOB | D | I | 0.3038 | −0.0972 | 0.0032 | 5.62E−209 |
| 2:234664586 | rs35754645 | intron_variant | UGT1A6 | UGT1A1 | D | I | 0.3516 | −0.0199 | 0.0027 | 3.16E−13 |
| 2:27730940 | rs1260326 | missense_variant | GCKR | GCKR | T | C | 0.3798 | 0.0748 | 0.0028 | 2.92E−160 |
| 2:44074431 | rs4245791 | intron_variant | ABCG8 | ABCG5 | T | C | 0.7073 | −0.053 | 0.0029 | 1.12E−72 |
| 2:65652156 | rs7572922 | intron_variant | SPRED2 | CEP68 | T | C | 0.3733 | −0.0171 | 0.0028 | 6.18E−10 |
| 20:17843968 | rs2618568 | intergenic_variant | (SNX5) | SNX5 | A | C | 0.6004 | −0.0186 | 0.0031 | 1.47E−09 |
| 20:34131396 | rs12481365 | intron_variant | ERGIC3 | ERGIC3 | T | C | 0.1592 | −0.032 | 0.0036 | 2.17E−19 |
| 20:40078085 | rs4142393 | intron_variant | CHD6 | MAFB | T | C | 0.543 | 0.0172 | 0.003 | 8.98E−09 |
| 20:40101541 | rs1010305 | intron_variant | CHD6 | TOP1 | A | G | 0.4561 | −0.0168 | 0.003 | 1.42E−08 |
| 21:46916204 | rs77974343 | intron_variant | COL18A1 | COL18A1 | T | C | 0.0197 | −0.1304 | 0.0222 | 4.20E−09 |
| 22:21916272 | rs5754102 | intron_variant | UBE2L3 | UBE2L3 | A | C | 0.1892 | −0.027 | 0.0038 | 1.24E−12 |
| 22:44324727 | rs738409 | missense_variant | PNPLA3 | PNPLA3 | C | G | 0.7355 | 0.0341 | 0.0062 | 3.60E−08 |
| 3:119529113 | rs3732356 | intron_variant | NR1I2 | GSK3B | T | G | 0.8483 | −0.0291 | 0.0046 | 2.31E−10 |
| 3:122258056 | rs9825383 | intron_variant | PARP9 | ADCY5 | A | G | 0.6077 | 0.0152 | 0.0028 | 4.16E−08 |
| 3:12268604 | rs7641325 | intergenic_variant | (SYN2) | PPARG | A | G | 0.4591 | −0.0225 | 0.0026 | 1.12E−17 |
| 3:132188163 | rs78946096 | intron_variant | DNAJC13 | ACAD11 | A | G | 0.9501 | 0.0414 | 0.0065 | 1.76E−10 |
| 3:32533010 | rs7640978 | intron_variant | CMTM6 | CMTM6 | T | C | 0.1604 | −0.033 | 0.004 | 2.43E−16 |
| 3:58301460 | rs9985315 | intron_variant | RPP14 | PXK | A | G | 0.9179 | 0.0272 | 0.0047 | 9.17E−09 |
| 4:100260545 | rs62307295 | intron_variant | ADH1C | ADH5 | A | C | 0.0958 | 0.0277 | 0.0046 | 2.47E−09 |
| 4:103198082 | rs13135092 | intron_variant | SLC39A8 | SLC39A8 | A | G | 0.9185 | 0.0308 | 0.0052 | 3.53E−09 |
| 4:3452345 | rs59950280 | downstream_gene_variant | (HGFAC) | LRPAP1 | A | G | 0.3883 | 0.0198 | 0.0029 | 4.10E−12 |
| 4:69338311 | rs969114 | intron_variant | TMPRSS11E | TMPRSS11E | A | G | 0.5722 | 0.029 | 0.0027 | 1.34E−26 |
| 4:88160140 | rs10029254 | intron_variant | KLHL8 | KLHL8 | T | C | 0.2088 | 0.0264 | 0.0035 | 2.06E−14 |
| 5:131408842 | rs1469149 | upstream_gene_variant | (CSF2) | SLC22A5 | A | C | 0.586 | 0.0148 | 0.0027 | 2.39E−08 |
| 5:156392248 | rs12517431 | upstream_gene_variant | (TIMD4) | TIMD4 | T | C | 0.6045 | 0.0365 | 0.0027 | 7.63E−42 |
| 5:74656539 | rs12916 | 3_prime_UTR_variant | HMGCR | HMGCR | T | C | 0.6225 | −0.0556 | 0.0027 | 6.54E−94 |
| 6:116393727 | rs72951954 | regulatory_region_variant | (FRK) | FRK | A | C | 0.3924 | −0.0196 | 0.0027 | 3.51E−13 |
| 6:135421067 | rs34208856 | intron_variant | HBS1L | HBS1L | D | I | 0.2988 | −0.0322 | 0.0034 | 6.61E−21 |
| 6:139299618 | 6:139299618 | NA | (REPS1) | CITED2 | D | I | 0.6143 | −0.0197 | 0.0029 | 9.70E−12 |
| 6:161010118 | rs10455872 | intron_variant | LPA | LPA | A | G | 0.9351 | −0.0762 | 0.0057 | 4.08E−41 |
| 6:16126934 | rs7746081 | upstream_gene_variant | (MYLIP) | MYLIP | A | G | 0.4055 | −0.0163 | 0.0028 | 5.62E−09 |
| 6:25715657 | rs116009877 | intergenic_variant | (SCGN) | HFE | A | G | 0.0533 | −0.0533 | 0.0067 | 1.68E−15 |
| 6:27122444 | rs71559014 | intergenic_variant | (TRNAH3) | HIST1H1B | A | G | 0.9285 | 0.0331 | 0.0056 | 3.64E−09 |
| 6:32590735 | rs35062987 | regulatory_region_variant | (HLA) | HLA | T | C | 0.2102 | 0.0418 | 0.0032 | 1.91E−39 |
| 6:35133074 | rs3800406 | regulatory_region_variant | (SCUBE3) | C6orf106 | A | G | 0.8941 | 0.0342 | 0.0048 | 7.01E−13 |
| 7:1067906 | rs2362529 | intron_variant | C7orf50 | GPR146 | T | C | 0.7754 | 0.0284 | 0.0032 | 8.09E−19 |
| 7:21611399 | rs66476925 | intron_variant | DNAH11 | DNAH11 | C | G | 0.1868 | 0.0316 | 0.0036 | 8.70E−19 |
| 7:25991826 | rs4722551 | upstream_gene_variant | (CTD-2227E11.1) | MIR148A | T | C | 0.8459 | −0.0218 | 0.0038 | 7.03E−09 |
| 7:44581986 | rs17725246 | upstream_gene_variant | (NPC1L1) | NPC1L1 | T | C | 0.7925 | −0.03 | 0.0033 | 2.96E−19 |
| 7:73026151 | rs13234378 | intron_variant | MLXIPL | TYW1B | A | T | 0.8858 | 0.0235 | 0.0043 | 4.49E−08 |
| 8:116667634 | rs2737265 | intron_variant | TRPS1 | TRPS1 | A | G | 0.7419 | 0.0183 | 0.0031 | 2.83E−09 |
| 8:126507389 | rs2954038 | intron_variant | RP11-136O12.2 | TRIB1 | A | C | 0.7181 | −0.0776 | 0.003 | 6.50E−148 |
| 8:145031968 | rs55831924 | intron_variant | PLEC | PLEC1 | T | C | 0.3341 | 0.0166 | 0.0029 | 1.59E−08 |
| 8:18274443 | rs34987019 | intergenic_variant | (NAT2) | NAT2 | T | C | 0.754 | −0.033 | 0.0059 | 2.30E−08 |
| 8:59392324 | rs9297994 | intergenic_variant | (CYP7A1) | CYP7A1 | A | G | 0.6903 | −0.0363 | 0.003 | 1.29E−33 |
| 8:9181395 | rs2169387 | upstream_gene_variant | (RP11-115J16.1) | PPP1R3B | A | G | 0.1311 | −0.053 | 0.0041 | 7.20E−39 |
| 9:107665739 | rs2575876 | intron_variant | ABCA1 | ABCA1 | A | G | 0.25 | −0.0371 | 0.0031 | 1.20E−32 |
| 9:117144795 | rs2763193 | intron_variant | AKNA | DFNB31 | T | C | 0.5751 | 0.0156 | 0.0028 | 3.49E−08 |
| 9:136149830 | rs532436 | intron_variant | ABO | ABO | A | G | 0.181 | 0.068 | 0.0034 | 1.50E−87 |
| 9:19212560 | rs13300056 | intergenic_variant | (DENND4C) | RPS6 | T | C | 0.0825 | 0.0392 | 0.0064 | 1.00E−09 |
| 9:2640759 | rs3780181 | intron_variant | VLDLR | VLDLR | A | G | 0.8776 | 0.0358 | 0.0046 | 1.18E−14 |
| 9:5073770 | rs77375493 | missense_variant | JAK2 | JAK2 | T | G | 5.00E−04 | −0.5328 | 0.0603 | 1.02E−18 |

### REFERENCES

1. Collins, R. What makes UK Biobank special? The Lancet 379, 1173-1174 (2012).
2. Gaziano, J. M. et al. Million Veteran Program: A mega-biobank to study genetic influences on health and disease. J Clin Epidemiol 70, 214-23 (2016).
3. Di Angelantonio, E. et al. Major lipids, apolipoproteins, and risk of vascular disease. Jama 302, 1993-2000 (2009).
4. Teslovich, T. M. et al. Biological, clinical and population relevance of 95 loci for blood lipids. Nature 466, 707-13 (2010).
5. Global Lipids Genetics Consortium et al. Discovery and refinement of loci associated with lipid levels. Nat Genet 45, 1274-83 (2013).
6. Chasman, D. I. et al. Forty-three loci associated with plasma lipoprotein size, concentration, and cholesterol content in genome-wide analysis. PLoS Genet 5, e1000730 (2009).
7. Albrechtsen, A. et al. Exome sequencing-driven discovery of coding polymorphisms associated with common metabolic phenotypes. Diabetologia 56, 298-310 (2013).
8. Peloso, G. M. et al. Association of low-frequency and rare coding-sequence variants with blood lipids and coronary heart disease in 56,000 whites and blacks. Am J Hum Genet 94, 223-32 (2014).
9. Asselbergs, F. W. et al. Large-scale gene-centric meta-analysis across 32 studies identifies multiple lipid loci. Am J Hum Genet 91, 823-38 (2012).
10. Below, J. E. et al. Meta-analysis of lipid-traits in Hispanics identifies novel loci, population-specific effects, and tissue-specific enrichment of eQTLs. Sci Rep 6, 19429 (2016).
11. Liu, D. J. et al. Exome-wide association study of plasma lipids in >300,000 individuals. Nat Genet (2017).
12. Lu, X. et al. Exome chip meta-analysis identifies novel loci and East Asian-specific coding variants that contribute to lipid levels and coronary artery disease. Nat Genet (2017).
13. Sabatine, M. S. et al. Evolocumab and Clinical Outcomes in Patients with Cardiovascular Disease. N Engl J Med (2017).
14. Myocardial Infarction Genetics and CARDIoGRAM Exome Consortia Investigators. Coding Variation in ANGPTL4, LPL, and SVEP1 and the Risk of Coronary Disease. N Engl J Med 374, 1134-44 (2016).
15. Dewey, F. E. et al. Inactivating Variants in ANGPTL4 and Risk of Coronary Artery Disease. N Engl J Med 374, 1123-33 (2016).
16. Barter, P. J. et al. Effects of torcetrapib in patients at high risk for coronary events. N Engl J Med 357, 2109-22 (2007).
17. Denny, J. C. et al. Systematic comparison of phenome-wide association study of electronic medical record data and genome-wide association study data. Nat Biotechnol 31, 1102-10 (2013).
18. Crosby, J. et al. Loss-of-function mutations in APOC3, triglycerides, and coronary disease. N Engl J Med 371, 22-31 (2014).
19. Cohen, J. C., Boerwinkle, E., Mosley, T. H., Jr. & Hobbs, H. H. Sequence variations in PCSK9, low LDL, and protection against coronary heart disease. N Engl J Med 354, 1264-72 (2006).
20. Abul-Husn, N. S. et al. Genetic identification of familial hypercholesterolemia within a single U.S. health care system. Science 354(2016).
21. The 1000 Genomes Project Consortium. A global reference for human genetic variation. Nature 526, 68-74 (2015).
22. Tishkoff, S. A. et al. The genetic structure and history of Africans and African Americans. Science 324, 1035-44 (2009).
23. Frazer, K. A. et al. A second generation human haplotype map of over 3.1 million SNPs. Nature 449, 851-61 (2007).
24. Lek, M. et al. Analysis of protein-coding genetic variation in 60,706 humans. Nature 536, 285-91 (2016).
25. Marouli, E. et al. Rare and low-frequency coding variants alter human adult height. Nature (2017).
26. Khera, A. V. et al. Association of Rare and Common Variation in the Lipoprotein Lipase Gene With Coronary Artery Disease. JAMA 317, 937-946 (2017).
27. Dewey, F. E. et al. Distribution and clinical impact of functional variants in 50,726 whole-exome sequences from the DiscovEHR study. Science 354(2016).
28. Sidore, C. et al. Genome sequencing elucidates Sardinian genetic architecture and augments association analyses for lipid and blood inflammatory markers. Nat Genet 47, 1272-81 (2015).
29. Purcell, S. M. et al. A polygenic burden of rare disruptive mutations in schizophrenia. Nature 506, 185-90 (2014).
30. Diogo, D. et al. Phenome-wide association studies (PheWAS) across large “real-world data” population cohorts support drug target validation. bioRxiv (2017).
31. Klarin, D. et al. Genetic analysis in UK Biobank links insulin resistance and transendothelial migration pathways to coronary artery disease. Nat Genet (2017).
32. Nelson, C. P. et al. Association analyses based on false discovery rate implicate new loci for coronary artery disease. Nat Genet 49, 1385-1391 (2017).
33. Gandotra, S. et al. Perilipin deficiency and autosomal dominant partial lipodystrophy. N Engl J Med 364, 740-8 (2011).
34. Rani, J. et al. T2DiACoD: A Gene Atlas of Type 2 Diabetes Mellitus Associated Complex Disorders. Sci Rep 7, 6892 (2017).
35. Musunuru, K. et al. Exome Sequencing, ANGPTL3 Mutations, and Familial Combined Hypolipidemia New England Journal of Medicine (2010).
36. Graham, M. J. et al. Cardiovascular and Metabolic Effects of ANGPTL3 Antisense Oligonucleotides. N Engl J Med (2017).
37. Zhang, W. & Colman, R. W. Thrombin regulates intracellular cyclic AMP concentration in human platelets through phosphorylation/activation of phosphodiesterase 3A. Blood 110, 1475-82 (2007).
38. Maass, P. G. et al. PDE3A mutations cause autosomal dominant hypertension with brachydactyly. Nat Genet 47, 647-53 (2015).
39. Vandeput, F. et al. Selective regulation of cyclic nucleotide phosphodiesterase PDE3A isoforms. Proc Natl Acad Sci USA 110, 19778-83 (2013).
40. Bedenis, R. et al. Cilostazol for intermittent claudication. Cochrane Database Syst Rev, Cd003748 (2014).
41. Tsuchikane, E. et al. Impact of cilostazol on restenosis after percutaneous coronary balloon angioplasty. Circulation 100, 21-6 (1999).
42. Shinohara, Y. et al. Cilostazol for prevention of secondary stroke (CSPS 2): an aspirin-controlled, double-blind, randomised non-inferiority trial. Lancet Neurol 9, 959-68 (2010).
43. Ahmad, F. et al. Phosphodiesterase 3B (PDE3B) regulates NLRP3 inflammasome in adipose tissue. Sci Rep 6, 28056 (2016).
44. Chung, Y. W. et al. Targeted disruption of PDE3B, but not PDE3A, protects murine heart from ischemia/reperfusion injury. Proc Natl Acad Sci USA 112, E2253-62 (2015).
45. McLaren, W. et al. Deriving the consequences of genomic variants with the Ensembl API and SNP Effect Predictor. Bioinformatics 26, 2069-70 (2010).

We claim:

1. A method for determining a subject's susceptibility to having or developing coronary artery disease comprising determining in the subject the presence of one or more PDE3B loss of function or damaging variants, and wherein the presence of the variant indicates the subject's decreased susceptibility for having or developing coronary artery disease.

2. The method of claim 1, wherein the PDE3B loss of function or damaging variant is Arg783Ter or rs150090666.

3. The method of claim 1, wherein the PDE3B loss of function or damaging variant results in a truncated PDE3B protein.

4. The method of claim 3, wherein the truncated PDE3B protein has the mutation Arg783Ter.

5. The method of claims 1-4, wherein the PDE3B loss of function or damaging variant is determined from a sample obtained from the subject.

6. The method of claims 1-5, wherein the PDE3B loss of function or damaging variant is determined by amplifying or sequencing a nucleic acid sample obtained from the subject.

7. The method of claim 6, wherein the amplifying is performed using polymerase chain reaction (PCR).

8. The method of claims 6-7, wherein the amplifying or sequencing comprises using primers having a sequences complementary to a portion of the PDE3B nucleic acid sequence found in accession number NM_000922.3.

9. A method of detecting one or more PDE3B loss of function or damaging variants in a subject, said method comprising:
a. obtaining a biological sample from a subject;
b. detecting whether a PDE3B loss of function variant is present in the biological sample by performing whole genome or whole exome sequencing.

10. A method comprising:
a. obtaining a biological sample from a subject;
b. detecting whether one or more PDE3B loss of function variants are present in the sample;
c. diagnosing the subject as having a greater likelihood of responding to PDE3B inhibitors when there is an absence of the one or more PDE3B loss of function variants; and
administering an effective amount of a PDE3B inhibitor/antagonist to the subject.

11. The method of claim 10, wherein the PDE3B loss of function variant results in a truncated PDE3B protein.

12. The method of claim 11, wherein the truncated PDE3B protein has the mutation Arg783Ter.

13. The method of claims 9-12, wherein the sample is DNA or protein.

14. The method of claims 9-13, wherein the PDE3B inhibitor is a compound, protein, DNA, RNAi, CRISPR, or siRNA.

15. The method of claim 14, wherein the compound is cilostazol.

16. A method of treating a patient with coronary artery disease comprising administering an effective amount of a PDE3B inhibitor/antagonist.

17. The method of claim 16, further comprising determining whether the subject lacks a PDE3B loss of function variant (Arg783Ter) prior to administering an effective amount of a PDE3B inhibitor/antagonist.

18. The method of claims 16-17, wherein the PDE3B inhibitor/antagonist is a compound, protein, DNA, RNAi, CRISPR, or siRNA.

19. The method of claim 16, wherein the compound is cilostazol.

20. A method of treating/preventing coronary artery disease in a subject comprising administering a composition that antagonizes/inhibits PDE3B to the subject, wherein the subject has been determined to lack one or more loss of function mutations in PDE3B.

21. The method of claim 20, wherein the composition is a compound, protein, DNA, RNAi, CRISPR, or siRNA.

22. The method of claims 20-21, wherein the compound is cilostazol.

23. The method of claims 20-22, wherein the loss of function mutation in PDE3B results in a truncated PDE3B protein.

24. The method of claim 23, wherein the truncated PDE3B protein has the mutation Arg783Ter.

25. A method of screening for test compositions that cause a loss of function mutation in PDE3B comprising:
a. contacting a PDE3B gene with a test composition;
b. detecting the presence of one or more mutations in the PDE3B gene; and
c. determining if the one or more mutations are loss of function mutations, wherein the presence of one or more loss of function mutations in PDE3B indicates a test composition that causes a loss of function in PDE3B.

26. The method of claim 25, wherein the loss of function or damaging mutation in PCSK9 results in a a truncated PDE3B protein.

27. The method of claim 26, wherein the truncated PDE3B protein has the mutation Arg783Ter.

28. A method of screening for therapeutic candidates for treating coronary artery disease compositions comprising:
a. contacting a cell lacking one or more loss of function or damaging mutations in PDE3B with a test composition; and
b. determining if the test composition inhibits PDE3B in the cell,
wherein if the test composition inhibits PDE3B then it is a therapeutic candidate for treating coronary artery disease.

29. A method of inducing a loss of function or damaging mutation in PDE3B comprising administering a test composition determined from the method of claims 25-28.

30. A vector comprising a loss of function or damaging PDE3B variant, wherein the PDE3B variant comprises a mutation that results in a truncated PDE3B protein.

31. The vector of claim 30, wherein the truncated PDE3B protein has the mutation Arg783Ter.

32. A cell comprising the vector of claim 31.

33. A method for identifying a subject in need of treatment for coronary artery disease comprising determining in the subject the presence of a PDE3B loss of function or damaging variant, wherein the presence of a PDE3B loss of function or damaging variant indicates that the subject is not in need of treatment for a coronary artery disease.

34. A method of identifying a subject in need of screening for the development of a coronary artery disease comprising determining in the subject the absence of a PDE3B loss of function or damaging variant, wherein the absence of a a PDE3B loss of function or damaging variant indicates a subject in need of screening for the development of coronary artery disease.

35. An engineered, non-naturally occurring CRISPR-CAS system comprising:
a) a guide RNA that hybridizes with a target sequence, wherein the target sequence comprises a PDE3B loss of function variant, and
b) a Cas protein or gene encoding a Cas protein.

36. The engineered, non-naturally occurring CRISPR-CAS system of claim 35, wherein the Cas protein is a Type-II Cas9 protein or a gene encoding a Type-II Cas9 protein.

37. The engineered, non-naturally occurring CRISPR-CAS system of claim 36, wherein the Cas9 protein and the guide RNA do not naturally occur together.

38. The engineered, non-naturally occurring CRISPR-CAS system of claims 35-37, wherein the PDE3B loss of function variant comprises the mutation Arg783Ter in the PDE3B protein.

39. A method of altering expression of at least one gene product, wherein the at least one gene product is a gene product from a PDE3B loss of function variant, wherein the method comprises administering a) a guide RNA that hybridizes with a target sequence, wherein the target sequence comprises the PDE3B loss of function variant, and b) a Cas protein or gene encoding a Cas protein, whereby the guide RNA targets the target sequence and the Cas9 protein cleaves the nucleic acid molecule which comprises the PDE3B loss of function variant, whereby expression of the at least one gene product is altered.

40. The of altering expression of at least one gene product of claim 39, wherein the PDE3B loss of function variant comprises the mutation Arg783Ter in the PDE3B protein.

41. A method of altering expression of at least one gene product, wherein the at least one gene product is a gene product from a PDE3B loss of function variant, wherein the method comprises administering a vector that comprises a) a first regulatory element operable in a eukaryotic cell operably linked to at least one nucleotide sequence encoding a CRISPR-Cas system guide RNA that hybridizes with a target sequence, wherein the target sequence comprises the PDE3B loss of function variant, and b) a second regulatory element operable in a eukaryotic cell operably linked to a nucleotide sequence encoding a Cas9 protein, whereby the guide RNA targets the target sequence and the Cas9 protein cleaves the target sequence, whereby expression of the at least one gene product is altered.

42. The method of altering expression of at least one gene product of claim 41, wherein the PDE3B loss of function variant comprises the mutation Arg783Ter in the PDE3B protein.

43. A method of silencing or inhibiting expression of wild type PDE3B in a cell comprising providing at least one silencing agent to the cell, wherein said silencing agent silences or inhibits expression of the wild type PDE3B in the cell.

44. The method of silencing or inhibiting expression of wild type PDE3B in a cell of claim 43, wherein the cell is inside a subject and thus the method occurs in vivo.

45. The method of silencing or inhibiting expression of wild type PDE3B in a cell of claim 43, wherein the silencing or inhibiting expression of PDE3B in a cell occurs in vitro.

46. The method of silencing or inhibiting expression of wild type PDE3B in a cell of claims 43-45, wherein the silencing agent is RNAi, CRISPR, or siRNA.

47. A method of silencing or inhibiting expression of wild type PDE3B in a cell comprising providing at least one RNA to the cell in an amount sufficient to inhibit the expression of PDE3B, wherein the RNA comprises or forms a double-stranded structure containing a first strand comprising a ribonucleotide sequence which corresponds to a nucleotide sequence of PDE3B and a second strand comprising a ribonucleotide sequence which is complementary to the nucleotide sequence of PDE3B, wherein the first and the second ribonucleotide sequences are separate complementary sequences that hybridize to each other to form said double-stranded structure, and the RNA comprising the double-stranded structure inhibits expression of PDE3B.

48. The method of silencing or inhibiting expression of wild type PDE3B in a cell of claim 47, wherein the first strand comprises a sequence which corresponds to a portion of the PDE3B nucleic acid sequence found in accession number NM_000922.3.

49. The method of silencing or inhibiting expression of wild type PDE3B in a cell of claims 47-48, wherein the second strand comprises a sequence that can bind to, or is complementary to, a portion of the PDE3B nucleic acid sequence found in accession number NM_000922.3.

50. A RNA comprising a double-stranded structure containing a first strand comprising a ribonucleotide sequence which corresponds to a nucleotide sequence of PDE3B and a second strand comprising a ribonucleotide sequence which is complementary to the nucleotide sequence of PDE3B, wherein the first and the second ribonucleotide sequences are separate complementary sequences that hybridize to each other to form said double-stranded structure.

51. The RNA of claim 50, wherein the first strand comprises a sequence which corresponds to a portion of the PDE3B nucleic acid sequence found in accession number NM_000922.3.

52. The RNA of claims 50-51, wherein the second strand comprises a sequence that can bind to, or is complementary to, a portion of the PDE3B nucleic acid sequence found in accession number NM_000922.3.

53. A method of inhibiting expression of PDE3B in a cell comprising: (a) isolating the cell; (b) contacting the cell with a RNA comprising a double-stranded structure comprising a first strand comprising a ribonucleotide sequence which corresponds to a nucleotide sequence of PDE3B and a second strand comprising a ribonucleotide sequence which is complementary to the nucleotide sequence of PDE3B, wherein the first and the second ribonucleotide sequences are separate sequences that hybridize to each other to form said double-stranded structure, and (c) subsequently introducing the cell into a host, wherein said RNA comprising the double-stranded structure inhibits expression of the target gene in the cell in the host.
